(19) United States
(12) Patent Application Publication
(10) Pub. No.: US 2025/0081967 A1
(43) Pub. Date: Mar. 13, 2025

(54) PESTICIDALLY ACTIVE HETEROCYCLIC DERIVATIVES WITH SULFOXIMINE CONTAINING SUBSTITUENTS

(71) Applicant: SYNGENTA CROP PROTECTION AG, Basel (CH)

(72) Inventors: Michel MUEHLEBACH, Stein (CH); Sebastian RENDLER, Basel (CH); Andrew EDMUNDS, Stein (CH); Anke BUCHHOLZ, Stein (CH); Daniel EMERY, Stein (CH); Indira SEN, Corlim, Ilhas (IN); Girish RAWAL, Corlim, Ilhas (IN); Vikas SIKERVAR, Bracknell, Berkshire (GB); Andre STOLLER, Stein (CH); Simon WILLIAMS, Stein (CH); Helmars SMITS, Stein (CH); Julia COMAS-BARCELO, Bracknell, Berkshire (GB)

(73) Assignee: SYNGENTA CROP PROTECTION AG, Basel (CH)

(21) Appl. No.: 18/566,449

(22) PCT Filed: May 31, 2022
(86) PCT No.: PCT/EP2022/064778

§ 371 Date: Dec. 1, 2023

(30) Foreign Application Priority Data
Jun. 2, 2021 (IN) 202111024601

(51) Int. Cl. A01N 43/90; A01N 47/02; A01P 7/02; A01P 7/04; C07D 471/04; C07D 487/04

(52) U.S. Cl. CPC A01N 43/90; A01N 47/02; A01P 7/02; A01P 7/04; C07D 471/04; C07D 487/04

(57) ABSTRACT

Compounds of the formula (I), are disclosed wherein the substituents are as defined in claim 1. Furthermore, the present invention relates to agrochemical compositions which comprise compounds of formula (I), to preparation of these compositions, and to the use of the compounds or compositions in agriculture or horticulture for combating, preventing or controlling animal pests, including arthropods and in particular insects or representatives of the order Acarina.

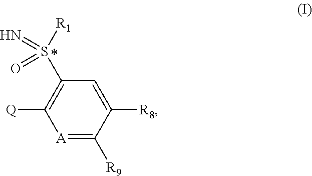

The present invention relates to pesticidally active, in particular insecticidally active heterocyclic derivatives containing sulfoximine substituents, to processes for their preparation, to compositions comprising those compounds, and to their use for controlling animal pests, including arthropods and in particular insects or representatives of the order Acarina.

Pesticidally active heterocyclic sulfoximine derivatives have previously been described in the literature, for example, in WO 2015/071180, WO 2016/039441, WO 2018/206348, WO 2019/219689, WO 2019/229089, WO 2019/234158, WO 2020/084075 and WO2020/141136.

It has now surprisingly been found that certain novel sulfoximine-containing phenyl and pyridyl derivatives with a stereogenic sulfur and a cyanoisopropoxy, cyanoisopropyl or cyanocyclopropyl group have favorable properties as pesticides and are obtainable in a stereospecific manner by means of a stereoselective oxidation, followed by a stereospecific imination reaction.

The present invention therefore provides compounds of formula I,

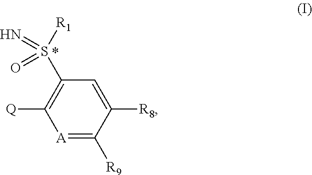

wherein
A is CH or N;
R₁ is C₁-C₄alkyl;
S* is a stereogenic sulfur atom which is in R- or S-configuration;
R₈ is cyanoisopropoxy, cyanoisopropyl or cyanocyclopropyl;
R₉ is hydrogen or C₁-C₄alkyl;
Q is a radical selected from the group consisting of formula Q₁ to Q₅

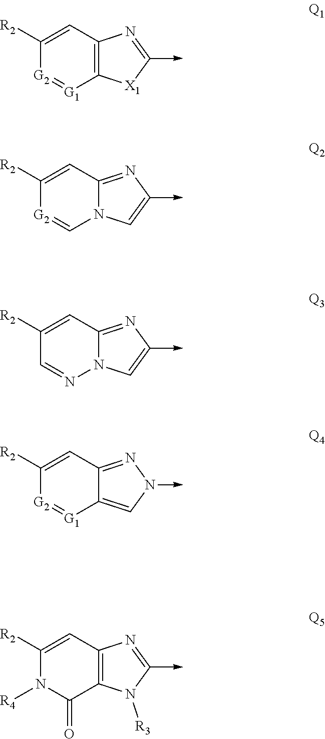

wherein the arrow denotes the point of attachment to the ring incorporating the radical A;
and wherein
X₁ is O, S or NR₃;
R₃ is C₁-C₄alkyl;
R₂ is halogen, C₁-C₆haloalkyl, C₁-C₄haloalkylsulfanyl, C₁-C₄haloalkylsulfinyl, C₁-C₄haloalkylsulfonyl or C₁-C₆haloalkoxy;
G₁ and G₂ are, independently from each other, N or CH;
R₄ is C₁-C₄alkyl, C₁-C₄haloalkyl, C₃-C₆cycloalkyl or C₁-C₄alkoxy; or
an agrochemically acceptable salt, stereoisomer, enantiomer, tautomer or N-oxide of a compound of formula I.

Compounds of formula I which have at least one basic centre can form, for example, acid addition salts, for example with strong inorganic acids such as mineral acids, for example perchloric acid, sulfuric acid, nitric acid, nitrose acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl and their branched isomers. Haloalkylsulfanyl, haloalkylsulfinyl, haloalkylsulfonyl and alkoxy radicals are derived from the alkyl radicals mentioned.

The term “cyanoisopropyl” as used herein refers to an isopropyl group (as mentioned above), where one of the hydrogen atoms in this radical may be replaced by a cyano group. Cyanoisopropyl is, for example, 1-cyano-1-methylethyl or 2-cyano-1-methylethyl.

Halogen is generally fluorine, chlorine, bromine or iodine. This also applies, correspondingly, to halogen in combination with other meanings, such as haloalkyl.

Haloalkyl groups refer to a straight-chain or branched saturated C₁-Cₙalkyl radical having 1 to n carbon atoms, preferably have a chain length of from 1 to 6 carbon atoms (as mentioned above), where some or all of the hydrogen atoms in these radicals may be replaced by fluorine, chlorine, bromine and/or iodine. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloromethyl, difluorochloromethyl, difluoromethyl, trifluoromethyl and dichlorofluoromethyl.

Alkoxy groups preferably have a preferred chain length of from 1 to 6 carbon atoms. Alkoxy is, for example, methoxy, ethoxy, propoxy, i-propoxy, n-butoxy, isobutoxy, sec-butoxy and tert-butoxy and also the isomeric pentyloxy and hexyloxy radicals; preferably methoxy and ethoxy.

The term “cyanoisopropoxy” as used herein refers to an i-propoxy group (as mentioned above), where one of the hydrogen atoms in this radical may be replaced by a cyano group. Cyanoisopropoxy is, for example, 1-cyano-1-methylethoxy or 2-cyano-1-methylethoxy.

The term “C₁-Cₙhaloalkoxy” as used herein refers to a straight-chain or branched saturated C₁-Cₙhaloalkyl radical having 1 to n carbon atoms (as mentioned above) which is attached via an oxygen atom similar to C₁-Cₙalkoxy.

Alkylsulfanyl is for example methylsulfanyl, ethylsulfanyl, propylsulfanyl, isopropylsulfanyl, butylsulfanyl, pentylsulfanyl, and hexylsulfanyl.

Alkylsulfinyl is for example methylsulfinyl, ethylsulfinyl, propylsulfinyl, isopropylsulfinyl, a butylsulfinyl, pentylsulfinyl, and hexylsulfinyl.

Alkylsulfonyl is for example methylsulfonyl, ethylsulfonyl, propylsulfonyl, isopropylsulfonyl, butylsulfonyl, pentylsulfonyl, and hexylsulfonyl.

The term “C₁-Cₙhaloalkylsulfanyl” as used herein refers to an alkylsulfanyl radical as mentioned above which is partially or fully substituted by fluorine, chlorine, bromine and/or iodine. Haloalkylsulfanyl groups preferably have a chain length of from 1 to 4 carbon atoms, for example, any one of fluoromethylthio, difluoromethylthio, trifluoromethylthio, chlorodifluoromethylthio, bromodifluoromethylthio, 2-fluoroethylthio, 2-chloroethylthio, 2-bromoethylthio, 2-iodoethylthio, 2,2-difluoroethylthio, 2,2,2-trifluoroethylthio, 2,2,2-trichloroethylthio, 2-chloro-2-fluoroethylthio, 2-chloro-2,2-difluoroethylthio, 2,2-dichloro-2-fluoroethylthio, pentafluoroethylthio, 2-fluoropropylthio, 3-fluoropropylthio, 2-chloropropylthio, 3-chloropropylthio, 2-bromopropylthio, 3-bromopropylthio, 2,2-difluoropropylthio, 2,3-difluoropropylthio, 2,3-dichloropropylthio, 3,3,3-trifluoropropylthio, 3,3,3-trichloropropylthio, 2,2,3,3,3-pentafluoropropylthio, heptafluoropropylthio, 1-(fluoromethyl)-2-fluoroethylthio, 1-(chloromethyl)-2-chloroethylthio, 1-(bromomethyl)-2-bromoethylthio, 4-fluorobutylthio, 4-chlorobutylthio, or 4-bromobutylthio.

Similar considerations apply to the terms “C₁-Cₙhaloalkylsulfinyl” and “C₁-Cₙhaloalkylsulfonyl” which refer to the C₁-Cₙhaloalkylsulfanyl (as mentioned above), but with the sulfur in a different oxidation state, for example, sulfoxide —S(O)C₁-Cₙhaloalkyl or sulfone —S(O)₂C₁-Cₙhaloalkyl, respectively. Accordingly, for example, groups such as trifluoromethylsulfinyl, trifluoromethylsulfonyl or 2,2,2-trifluoroethylsulfonyl.

The cycloalkyl groups preferably have from 3 to 6 ring carbon atoms, for example cyclopropyl, cyclobutyl, cyclopentyl and cyclohexyl.

The term “cyanocyclopropyl” as used herein refers to a cyclopropyl (as mentioned above), where one of the hydrogen atoms in this radical may be replaced by a cyano group. Cyanocyclopropyl is, for example, 1-cyanocyclopropyl or 2-cyanocyclopropyl.

The compounds of formula I according to the invention also include hydrates which may be formed during the salt formation.

The presence of a sulfur stereogenic center (S*) in compounds of formula (I) means that the compounds may occur in optically isomeric forms, i.e. enantiomeric or diastereomeric forms. Preferably, and in absence of an additional asymmetric carbon or sulfur atom, the present invention therefore refers to both enantiomers that result from the presence of the chiral sulfur atom S*, i.e. the present invention covers compounds of formula (I) with either (R) or (S) configuration at said stereogenic sulfur atom, and mixtures thereof (such as pure enantiomers or mixtures of enantiomers, i.e., single enantiomers having an enantiomeric excess). The present invention also refers to individual enantiomers obtained either after separation of a racemic mixture using known resolution methods or obtained by means of a stereoselective synthesis. For example, first and second eluting enantiomers obtained by chromatographic separation using a chiral stationary phase (such as amylose- or cellulose-based CHIRALPAK® columns); or enantiomers that are obtainable in a stereospecific manner by imination of stereogenic sulfinyl derivatives that are produced by steeoselective oxidation of the corresponding sulfanyl compounds are also subject matter of the present invention.

Certain embodiments according to the invention are provided as set out below.

Embodiment 1 provides compounds of formula I, or an agrochemically acceptable salt, stereoisomer, enantiomer, tautomer or N-oxide thereof, as defined above.

Embodiment 2 provides compounds, or an agrochemically acceptable salt, stereoisomer, enantiomer, tautomer or N-oxide thereof, according to embodiment 1 wherein:
A is CH or N;
R₁ is ethyl, propyl or isopropyl;
R₈ is cyanoisopropoxy, cyanoisopropyl or cyanocyclopropyl; and
R₉ is hydrogen, methyl or ethyl.

Embodiment 3a provides compounds, or an agrochemically acceptable salt, stereoisomer, enantiomer, tautomer or N-oxide thereof, according to embodiment 1 wherein:
A is CH or N;
R₁ is ethyl;
R₈ is cyanoisopropoxy, cyanoisopropyl or cyanocyclopropyl; and
R₉ is hydrogen or methyl.

Embodiment 3b provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl; and
R₉ is hydrogen or methyl.

Embodiment 3c provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl; and
R₉ is hydrogen or methyl.

Embodiment 4a provides compounds, or an agrochemically acceptable salt, stereoisomer, enantiomer, tautomer or N-oxide thereof, according to embodiment 1 wherein:
A is CH or N;
R₁ is ethyl;
R₈ is 1-cyano-1-methyl-ethoxy, 1-cyano-1-methyl-ethyl or 1-cyanocyclopropyl; and
R₉ is hydrogen.

Embodiment 4b provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl; and
R₉ is hydrogen.

Embodiment 4c provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl; and
R₉ is hydrogen.

Embodiment 5a provides compounds, or an agrochemically acceptable salt, stereoisomer, enantiomer, tautomer or N-oxide thereof, according to embodiment 1 wherein:
A is CH or N;
R₁ is ethyl;
R₈ is 1-cyano-1-methyl-ethoxy, 1-cyano-1-methyl-ethyl or 1-cyanocyclopropyl; and
R₉ is methyl.

Embodiment 5b provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl; and
R₉ is methyl.

Embodiment 5c provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl; and
R₉ is methyl.

Embodiment 6a provides compounds, or an agrochemically acceptable salt, stereoisomer, enantiomer, tautomer or N-oxide thereof, according to embodiment 1 wherein:
Q is a radical selected from the group consisting of formula Q₁ to Q₅
wherein the arrow denotes the point of attachment to the ring incorporating the radical A;
and wherein
R₂ is C₁-C₂haloalkyl, C₁-C₂haloalkylsulfanyl, C₁-C₂haloalkylsulfinyl or C₁-C₂haloalkylsulfonyl;
X₁ is oxygen or NCH₃;
R₃ is C₁-C₂alkyl;
R₄ is C₁-C₂alkyl, C₁-C₂haloalkyl, C₁-C₂alkoxy or cyclopropyl; and
G₁ and G₂ are, independently from each other, N or CH.

Embodiment 6b provides compounds, or an agrochemically acceptable salt, stereoisomer, enantiomer, tautomer or N-oxide thereof, according to embodiment 1 wherein:
Q is a radical selected from the group consisting of formula Q₁ to Q₅
wherein the arrow denotes the point of attachment to the ring incorporating the radical A;
and wherein
R₂ is C₁-C₂haloalkyl or C₁-C₂haloalkylsulfanyl;
X₁ is NCH₃;
R₃ is C₁-C₂alkyl;
R₄ is C₁-C₂alkyl, C₁-C₂alkoxy or cyclopropyl; and
G₁ and G₂ are, independently from each other, N or CH.

Embodiment 7 provides compounds, or an agrochemically acceptable salt, stereoisomer, enantiomer, tautomer or N-oxide thereof, according to embodiment 1 wherein:
Q is a radical selected from Q₁, Q₂ and Q₅

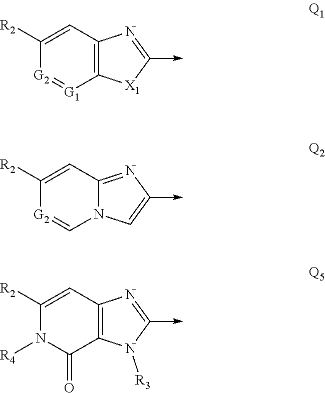

wherein the arrow denotes the point of attachment to the ring incorporating the radical A;
and wherein
R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl;
X₁ is NCH₃;
R₃ is methyl;
R₄ is methyl, ethyl, methoxy or cyclopropyl; and
G₁ and G₂ are, independently from each other, N or CH.

Embodiment 8a provides compounds, or an agrochemically acceptable salt, stereoisomer, enantiomer, tautomer or N-oxide thereof, according to embodiment 1 wherein:
Q is a radical selected from Q₁, Q₂ and Q₅

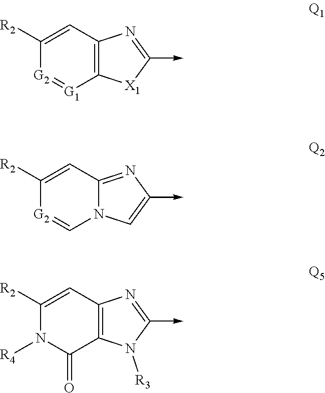

wherein the arrow denotes the point of attachment to the ring incorporating the radical A;
and wherein
R₂ is trifluoromethyl, pentafluoroethyl, trifluoromethylsulfanyl, trifluoromethylsulfinyl or trifluoromethylsulfonyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and when Q is Q₂, G₂ is CH or N.

Embodiment 8b provides compounds, or an agrochemically acceptable salt, stereoisomer, enantiomer, tautomer or N-oxide thereof, according to embodiment 1 wherein:
Q is a radical selected from Q₁, Q₂ and Q₅

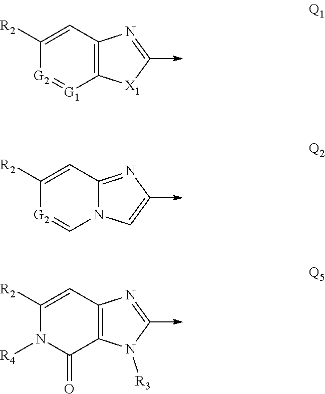

wherein the arrow denotes the point of attachment to the ring incorporating the radical A;
and wherein
R₂ is trifluoromethyl, pentafluoroethyl or trifluoromethylsulfanyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂, G₂ is CH or N.

Embodiment 8c provides compounds, or an agrochemically acceptable salt, stereoisomer, enantiomer, tautomer or N-oxide thereof, according to embodiment 1 wherein:
Q is a radical selected from Q₁, Q₂ and Q₅

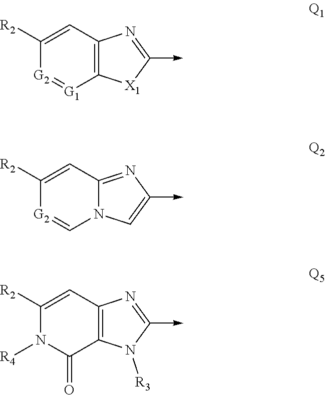

wherein the arrow denotes the point of attachment to the ring incorporating the radical A;
and wherein
R₂ is trifluoromethyl or trifluoromethylsulfanyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂, G₂ is CH or N.

Embodiment 9a provides compounds, or an agrochemically acceptable salt, stereoisomer, enantiomer, tautomer or N-oxide thereof, according to embodiment 1 wherein:
Q is radical Q₁

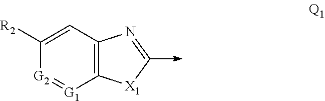

wherein the arrow denotes the point of attachment to the ring incorporating the radical A;
and wherein
R₂ is trifluoromethyl, pentafluoroethyl, trifluoromethylsulfanyl, trifluoromethylsulfinyl or trifluoromethylsulfonyl;
X₁ is NCH₃; and
G₁ is N and G₂ is CH or G₁ is CH and G₂ is N or both G₁ and G₂ are N.

Embodiment 9b provides compounds, or an agrochemically acceptable salt, stereoisomer, enantiomer, tautomer or N-oxide thereof, according to embodiment 1 wherein:
Q is radical Q₁

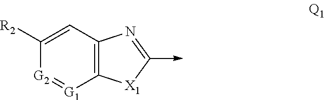

wherein the arrow denotes the point of attachment to the ring incorporating the radical A;
and wherein
R₂ is trifluoromethyl, pentafluoroethyl or trifluoromethylsulfanyl;
X₁ is NCH₃; and
G₁ is N and G₂ is CH or G₁ is CH and G₂ is N or both G₁ and G₂ are N.

Embodiment 9c provides compounds, or an agrochemically acceptable salt, stereoisomer, enantiomer, tautomer or N-oxide thereof, according to embodiment 1 wherein:
Q is radical Q₁

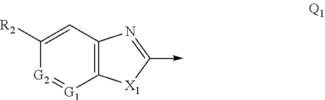

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N or both G₁ and G₂ are N.

Embodiment 10a provides compounds, or an agrochemically acceptable salt, stereoisomer, enantiomer, tautomer or N-oxide thereof, according to embodiment 1 wherein:
Q is radical Q₂

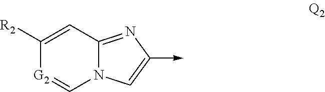

wherein the arrow denotes the point of attachment to the ring incorporating the radical A;
and wherein
R₂ is trifluoromethyl, pentafluoroethyl, trifluoromethylsulfanyl, trifluoromethylsulfinyl or trifluoromethylsulfonyl; and
G₂ is CH or N.

Embodiment 10b provides compounds, or an agrochemically acceptable salt, stereoisomer, enantiomer, tautomer or N-oxide thereof, according to embodiment 1 wherein:
Q is radical Q₂

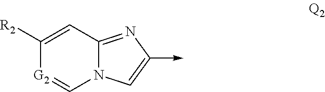

wherein the arrow denotes the point of attachment to the ring incorporating the radical A;
and wherein
R₂ is trifluoromethyl, pentafluoroethyl or trifluoromethylsulfanyl; and
G₂ is CH or N.

Embodiment 10c provides compounds, or an agrochemically acceptable salt, stereoisomer, enantiomer, tautomer or N-oxide thereof, according to embodiment 1 wherein:
Q is radical Q₂

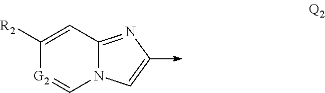

wherein the arrow denotes the point of attachment to the ring incorporating the radical A;
and wherein
R₂ is trifluoromethyl or trifluoromethylsulfanyl; and
G₂ is CH or N.

Embodiment 10d provides compounds, or an agrochemically acceptable salt, stereoisomer, enantiomer, tautomer or N-oxide thereof, according to embodiment 1 wherein:
Q is radical Q₂

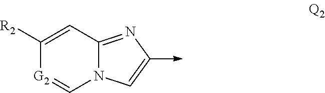

wherein the arrow denotes the point of attachment to the ring incorporating the radical A;
and wherein
R₂ is trifluoromethyl; and
G₂ is CH or N.

Embodiment 11a provides compounds, or an agrochemically acceptable salt, stereoisomer, enantiomer, tautomer or N-oxide thereof, according to embodiment 1 wherein:
Q is radical Q₅

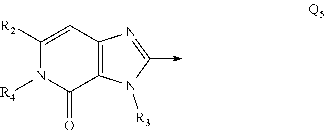

wherein the arrow denotes the point of attachment to the ring incorporating the radical A;
and wherein
R₂ is trifluoromethyl, pentafluoroethyl, trifluoromethylsulfanyl, trifluoromethylsulfinyl or trifluoromethylsulfonyl;
R₃ is methyl; and
R₄ is ethyl, methoxy or cyclopropyl.

Embodiment 11 b provides compounds, or an agrochemically acceptable salt, stereoisomer, enantiomer, tautomer or N-oxide thereof, according to embodiment 1 wherein:
Q is radical Q₅

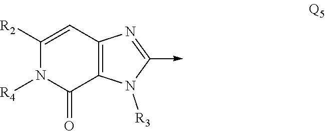

wherein the arrow denotes the point of attachment to the ring incorporating the radical A;
and wherein
R₂ is trifluoromethyl, pentafluoroethyl or trifluoromethylsulfanyl;
R₃ is methyl; and
R₄ is ethyl, methoxy or cyclopropyl.

Embodiment 11c provides compounds, or an agrochemically acceptable salt, stereoisomer, enantiomer, tautomer or N-oxide thereof, according to embodiment 1 wherein:
Q is radical Q₅

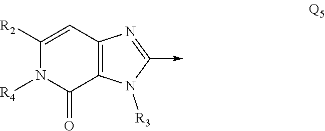

wherein the arrow denotes the point of attachment to the ring incorporating the radical A;
and wherein
R₂ is trifluoromethyl;
R₃ is methyl; and
R₄ is ethyl, methoxy or cyclopropyl.

Embodiment 12 provides compounds, or an agrochemically acceptable salt, stereoisomer, enantiomer, tautomer or N-oxide thereof, according to embodiment 1 wherein:
A is CH or N;
R₁ is ethyl, propyl or isopropyl;
R₈ is cyanoisopropoxy, cyanoisopropyl or cyanocyclopropyl;
R₉ is hydrogen, methyl or ethyl;
Q is a radical selected from the group consisting of formula Q₁ to Q₅
wherein the arrow denotes the point of attachment to the ring incorporating the radical A;
and wherein
R₂ is C₁-C₂haloalkyl, C₁-C₂haloalkylsulfanyl, C₁-C₂haloalkylsulfinyl or C₁-C₂haloalkylsulfonyl;
X₁ is oxygen or NCH₃;
R₃ is C₁-C₂alkyl;
R₄ is C₁-C₂alkyl, C₁-C₂haloalkyl, C₁-C₂alkoxy or cyclopropyl; and
G₁ and G₂ are, independently from each other, N or CH.

Embodiment 13 provides compounds, or an agrochemically acceptable salt, stereoisomer, enantiomer, tautomer or N-oxide thereof, according to embodiment 1 wherein:
A is CH or N;
R₁ is ethyl;
R₈ is cyanoisopropoxy, cyanoisopropyl or cyanocyclopropyl;
R₉ is hydrogen or methyl;
Q is a radical selected from Q₁, Q₂ and Q₅

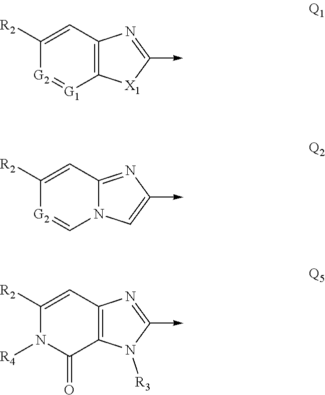

wherein the arrow denotes the point of attachment to the ring incorporating the radical A;
and wherein
R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl;
X₁ is NCH₃;
R₃ is methyl;
R₄ is methyl, ethyl, methoxy or cyclopropyl; and
G₁ and G₂ are, independently from each other, N or CH.

Embodiment 14 provides compounds, or an agrochemically acceptable salt, stereoisomer, enantiomer, tautomer or N-oxide thereof, according to embodiment 1 wherein:
A is CH or N;
R₁ is ethyl;
R₈ is 1-cyano-1-methyl-ethoxy, 1-cyano-1-methyl-ethyl or 1-cyanocyclopropyl;
R₉ is hydrogen or methyl;
Q is a radical selected from Q₁, Q₂ and Q₅

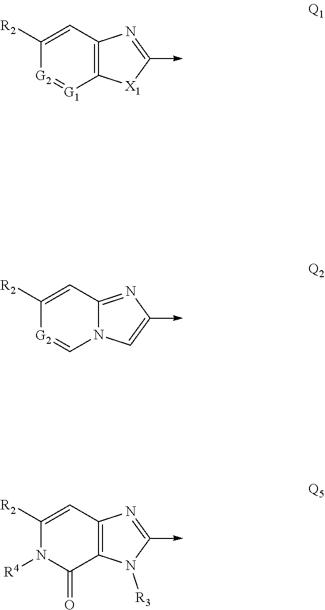

wherein the arrow denotes the point of attachment to the ring incorporating the radical A;
and wherein
R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl;
X₁ is NCH₃;
R₃ is methyl;
R₄ is methyl, ethyl, methoxy or cyclopropyl; and
G₁ and G₂ are, independently from each other, N or CH.

Embodiment 15a provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl; R₉ is hydrogen or methyl;
Q is a radical selected from Q₁, Q₂ and Q₅

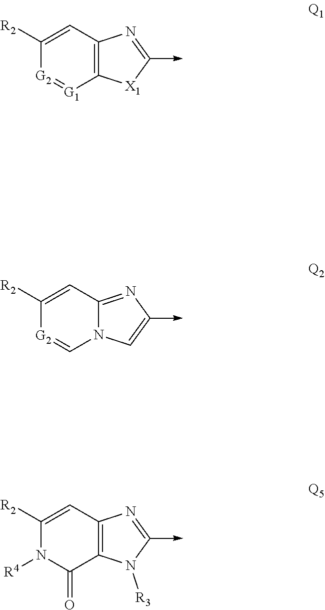

wherein the arrow denotes the point of attachment to the ring incorporating the radical A;
and wherein
R₂ is trifluoromethyl, pentafluoroethyl, trifluoromethylsulfanyl, trifluoromethylsulfinyl or trifluoromethylsulfonyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂. G₂ is CH or N.

Embodiment 15b provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is hydrogen or methyl;
Q is a radical selected from Q₁, Q₂ and Q₅

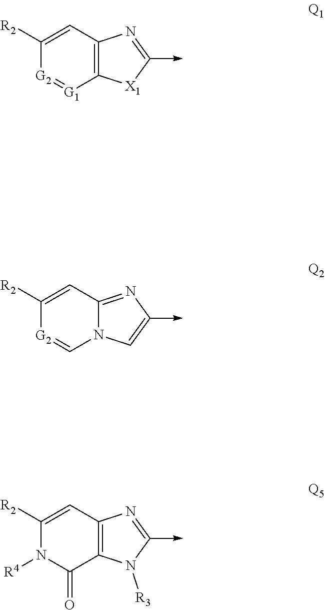

wherein the arrow denotes the point of attachment to the ring incorporating the radical A;
and wherein
R₂ is trifluoromethyl, pentafluoroethyl, trifluoromethylsulfanyl, trifluoromethylsulfinyl or trifluoromethylsulfonyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂ G₂ is CH or N.

Embodiment 16a provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen or methyl;
Q is a radical selected from Q₁, Q₂ and Q₅

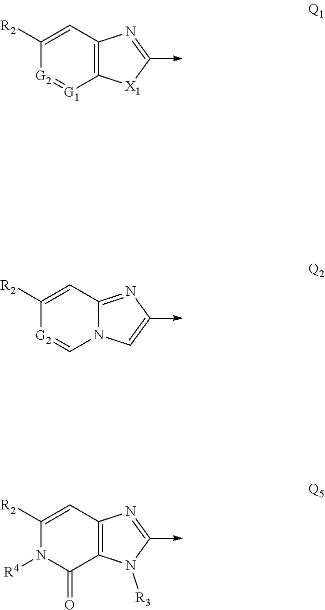

wherein the arrow denotes the point of attachment to the ring incorporating the radical A;
and wherein
R₂ is trifluoromethyl, pentafluoroethyl or trifluoromethylsulfanyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂, G₂ is CH or N.

Embodiment 16b provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is hydrogen or methyl;
Q is a radical selected from Q₁, Q₂ and Q₅

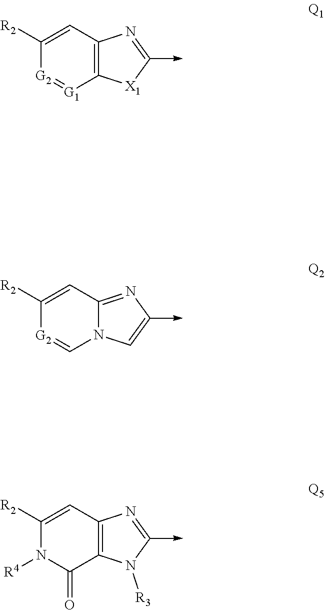

wherein the arrow denotes the point of attachment to the ring incorporating the radical A;
and wherein
R₂ is trifluoromethyl, pentafluoroethyl or trifluoromethylsulfanyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂, G₂ is CH or N.

Embodiment 17a provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen;
Q is a radical selected from Q₁, Q₂ and Q₅

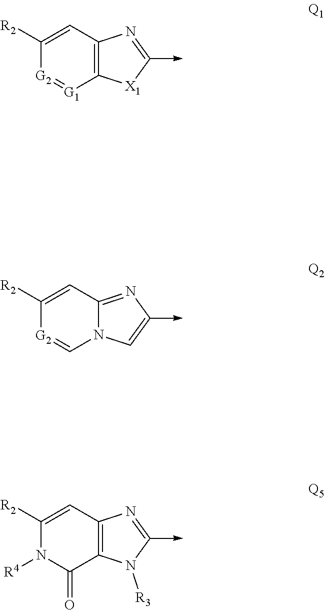

wherein the arrow denotes the point of attachment to the ring incorporating the radical A;
and wherein
R₂ is trifluoromethyl or trifluoromethylsulfanyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂, G₂ is CH or N.

Embodiment 17b provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is hydrogen;
Q is a radical selected from Q₁, Q₂ and Q₅

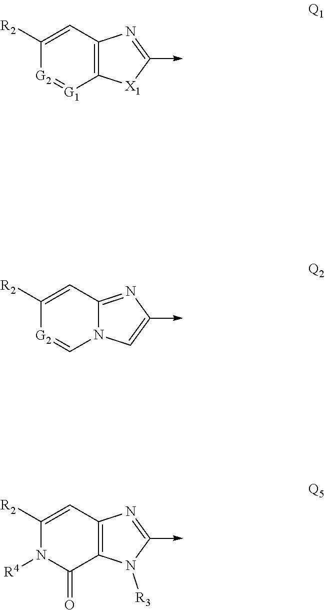

wherein the arrow denotes the point of attachment to the ring incorporating the radical A;
and wherein
R₂ is trifluoromethyl or trifluoromethylsulfanyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂, G₂ is CH or N.

Embodiment 18a provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is methyl;
Q is a radical selected from Q₁, Q₂ and Q₅

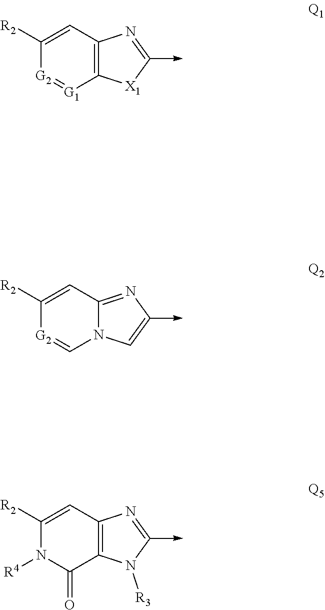

wherein the arrow denotes the point of attachment to the ring incorporating the radical A;
and wherein
R₂ is trifluoromethyl or trifluoromethylsulfanyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂, G₂ is CH or N.

Embodiment 18b provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is methyl;
Q is a radical selected from Q₁, Q₂ and Q₅

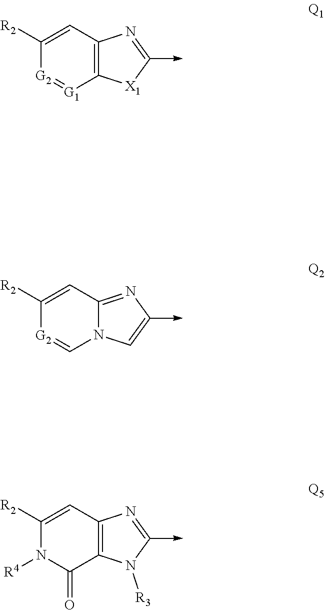

wherein the arrow denotes the point of attachment to the ring incorporating the radical A;
and wherein
R₂ is trifluoromethyl or trifluoromethylsulfanyl;
X₁ is NCH₃;
R₃ is methyl;
R₄ is ethyl, methoxy or cyclopropyl;
when Q is Q₁, G₁ is N and G₂ is CH or G₁ is CH and G₂ is N or both G₁ and G₂ are N; and
when Q is Q₂, G₂ is CH or N.

Embodiment 19a provides compounds, or an agrochemically acceptable salt, stereoisomer, enantiomer, tautomer or N-oxide thereof, according to embodiment 1 wherein:
A is N or CH;
R₁ is ethyl;
R₈ is 1-cyano-1-methyl-ethoxy, 1-cyano-1-methyl-ethyl or 1-cyanocyclopropyl;
R₉ is hydrogen or methyl;
Q is radical Q₁

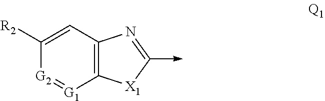

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N or both G₁ and G₂ are N.

Embodiment 19b provides compounds, or an agrochemically acceptable salt, stereoisomer, enantiomer, tautomer or N-oxide thereof, according to embodiment 1 wherein:
A is N or CH;
R₁ is ethyl;
R₈ is 1-cyano-1-methyl-ethoxy, 1-cyano-1-methyl-ethyl or 1-cyanocyclopropyl;
R₉ is hydrogen or methyl;
Q is radical Q₁

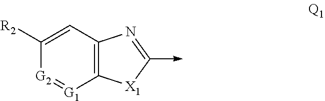

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N.

Embodiment 19c provides compounds, or an agrochemically acceptable salt, stereoisomer, enantiomer, tautomer or N-oxide thereof, according to embodiment 1 wherein:
A is N or CH;
R₁ is ethyl;
R₈ is 1-cyano-1-methyl-ethoxy or 1-cyanocyclopropyl;
R₉ is hydrogen or methyl;
Q is radical Q₁

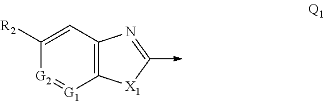

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N or both G₁ and G₂ are N.

Embodiment 20a provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₁

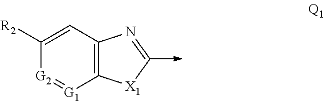

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N or both G₁ and G₂ are N.

Embodiment 20b provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₁

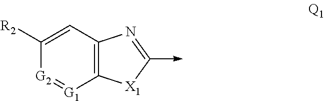

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N.

Embodiment 20c provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy or 1-cyanocyclopropyl;
R₉ is hydrogen;
Q is radical Q₁

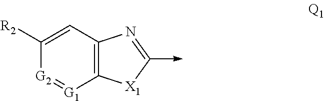

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N or both G₁ and G₂ are N.

Embodiment 21a provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is methyl;
Q is radical Q₁

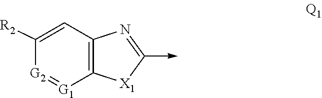

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N or both G₁ and G₂ are N.

Embodiment 21 b provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is methyl;
Q is radical Q₁

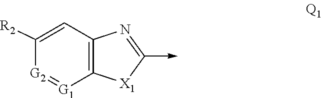

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N.

Embodiment 21c provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy;
R₉ is methyl;
Q is radical Q₁

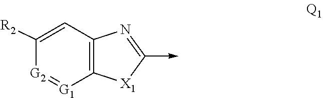

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N.

Embodiment 22a provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₁

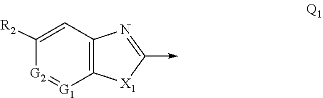

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N or both G₁ and G₂ are N.

Embodiment 22b provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₁

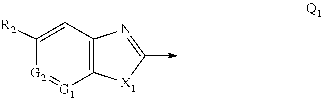

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N.

Embodiment 22c provides compounds, or an agrochemically acceptable salt, stereoisomer, enantiomer, tautomer or N-oxide thereof, according to embodiment 1 wherein:
A is CH;
R₁ is ethyl;
R₈ is 1-cyanocyclopropyl;
R₉ is hydrogen;
Q is radical Q₁

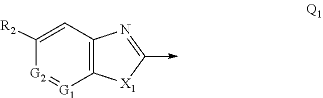

wherein the arrow denotes the point of attachment to the ring incorporating the radical A;
and wherein
R₂ is trifluoromethyl;
X₁ is NCH₃; and
G₁ is N and G₂ is CH or G₁ is CH and G₂ is N.

Embodiment 23a provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₂

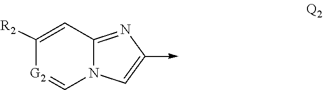

wherein the arrow denotes the point of attachment to the ring incorporating the radical A;
and wherein
R₂ is trifluoromethyl, pentafluoroethyl, trifluoromethylsulfanyl, trifluoromethylsulfinyl or
trifluoromethylsulfonyl; and
G₂ is CH or N.

Embodiment 23b provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₂

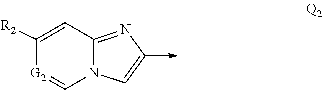

wherein the arrow denotes the point of attachment to the ring incorporating the radical A;
and wherein
R₂ is trifluoromethyl, pentafluoroethyl or trifluoromethylsulfanyl; and
G₂ is CH or N.

Embodiment 23c provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₂

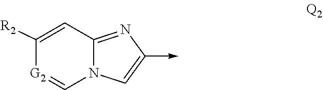

wherein the arrow denotes the point of attachment to the ring incorporating the radical A;
and wherein
R₂ is trifluoromethyl or trifluoromethylsulfanyl; and
G₂ is CH or N.

Embodiment 23d provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₂

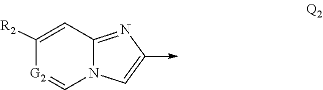

wherein the arrow denotes the point of attachment to the ring incorporating the radical A;
and wherein
R₂ is trifluoromethyl; and
G₂ is CH or N.

Embodiment 23e provides compounds, or an agrochemically acceptable salt, stereoisomer, enantiomer, tautomer or N-oxide thereof, according to embodiment 1 wherein:
A is N
R₁ is ethyl;
R₈ is 1-cyano-1-methyl-ethoxy;
R₉ is hydrogen;
Q is radical Q₂

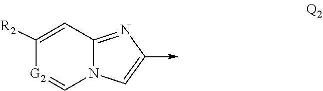

wherein the arrow denotes the point of attachment to the ring incorporating the radical A;
and wherein
R₂ is trifluoromethyl; and
G₂ is CH or N.

Embodiment 24a provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₂

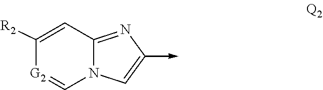

wherein the arrow denotes the point of attachment to the ring incorporating the radical A;
and wherein
R₂ is trifluoromethyl or trifluoromethylsulfanyl; and
G₂ is CH or N.

Embodiment 24b provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₂

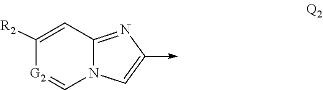

wherein the arrow denotes the point of attachment to the ring incorporating the radical A;
and wherein
R₂ is trifluoromethyl; and
G₂ is CH or N.

Embodiment 24c provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy;
R₉ is hydrogen;
Q is radical Q₂

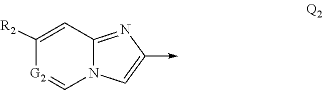

wherein the arrow denotes the point of attachment to the ring incorporating the radical A;
and wherein
R₂ is trifluoromethyl; and
G₂ is CH or N.

Embodiment 25a provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₅

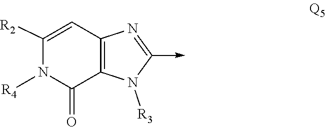

wherein the arrow denotes the point of attachment to the ring incorporating the radical A;
and wherein
R₂ is trifluoromethyl;
R₃ is methyl; and
R₄ is ethyl, methoxy or cyclopropyl.

Embodiment 25b provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy or 1-cyanocyclopropyl;
R₉ is hydrogen;
Q is radical Q₅

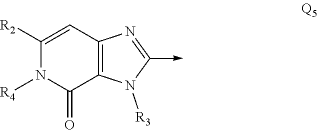

wherein the arrow denotes the point of attachment to the ring incorporating the radical A;
and wherein
R₂ is trifluoromethyl;
R₃ is methyl; and
R₄ is ethyl, methoxy or cyclopropyl.

Embodiment 25c provides compounds, or an agrochemically acceptable salt, stereoisomer, enantiomer, tautomer or N-oxide thereof, according to embodiment 1 wherein:
A is N;
R₁ is ethyl;
R₈ is 1-cyano-1-methyl-ethoxy;
R₉ is hydrogen;
Q is radical Q₅

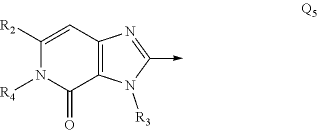

wherein the arrow denotes the point of attachment to the ring incorporating the radical A;
and wherein
R₂ is trifluoromethyl;
R₃ is methyl; and
R₄ is ethyl or cyclopropyl.

Embodiment 25d provides compounds, or an agrochemically acceptable salt, stereoisomer, enantiomer, tautomer or N-oxide thereof, according to embodiment 1 wherein:
A is N;
R₁ is ethyl;
R₈ is 1-cyanocyclopropyl;
R₉ is hydrogen;
Q is radical Q₅

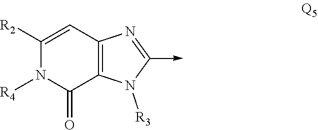

wherein the arrow denotes the point of attachment to the ring incorporating the radical A;
and wherein
R₂ is trifluoromethyl;
R₃ is methyl; and
R₄ is ethyl or methoxy.

Embodiment 26a provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy, 1-cyano-1-methyl-ethyl or 1-cyanocyclopropyl;
R₉ is hydrogen;
Q is radical Q₅

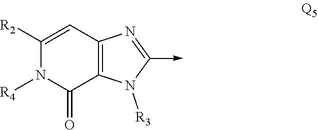

wherein the arrow denotes the point of attachment to the ring incorporating the radical A;
and wherein
R₂ is trifluoromethyl;
R₃ is methyl; and
R₄ is ethyl, methoxy or cyclopropyl.

Embodiment 26b provides compounds, or an agrochemically acceptable salt, stereoisomer, enantiomer, tautomer or N-oxide thereof, according to embodiment 1 wherein:
A is CH;
R₁ is ethyl;
R₈ is 1-cyano-1-methyl-ethoxy or 1-cyanocyclopropyl;
R₉ is hydrogen;
Q is radical Q₅

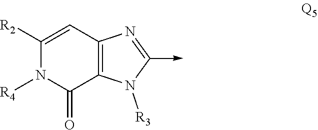

wherein the arrow denotes the point of attachment to the ring incorporating the radical A;
and wherein
R₂ is trifluoromethyl;
R₃ is methyl; and
R₄ is ethyl, methoxy or cyclopropyl.

Embodiment 27 provides compounds, or an agrochemically acceptable salt, stereoisomer, enantiomer, tautomer or N-oxide thereof, according to any one of the previous embodiments 1-26b wherein S* is in the R-configuration.

Embodiment 28 provides compounds, or an agrochemically acceptable salt, stereoisomer, enantiomer, tautomer or N-oxide thereof, according to embodiment 27 in which said S* center is in either enantiomerically pure or in an enantiomerically enriched form that is enantiomerically enriched with an (S*) R-enantiomeric excess (e.e.) of at least 40%, for example, at least 50%, 60%, 70% or 80%, preferably at least 90%, more preferably at least 95%, yet more preferably at least 98% and most preferably at least 99%.

Embodiment 29 provides compounds, or an agrochemically acceptable salt, stereoisomer, enantiomer, tautomer or N-oxide thereof, according to any one of the previous embodiments 1-26b wherein S* is in the S-configuration.

Embodiment 30 provides compounds, or an agrochemically acceptable salt, stereoisomer, enantiomer, tautomer or N-oxide thereof, according to embodiment 29 in which said S* center is in either enantiomerically pure or in an enantiomerically enriched form that is enantiomerically enriched with (S*) S-enantiomeric excess (e.e.) of at least 40%, for example, at least 50%, 60%, 70% or 80%, preferably at least 90%, more preferably at least 95%, yet more preferably at least 98% and most preferably at least 99%.

Embodiment 31 provides compounds, or an agrochemically acceptable salt, stereoisomer, enantiomer, tautomer or N-oxide thereof, according to any one of the previous embodiments 1-30 whenever prepared or that are obtainable by a process comprising (A) stereoselectively oxidizing a sulfanyl compound of formula (II)

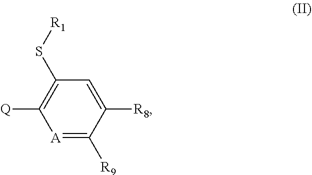

wherein Q, R₁, R₂, G₁, G₂, X₁, R₃, R₄, R₈, R₉ and A are as defined under formula (I),
in the presence of an oxidant, in the presence of a metal catalyst, in the presence of a chiral ligand, optionally in the presence of a suitable additive, in an appropriate solvent (or diluent);
to produce a sulfinyl compound of formula (III)

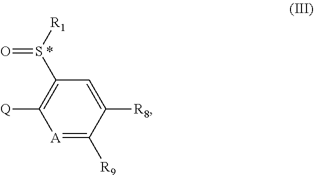

wherein Q, R₁, R₂, G₁, G₂, X₁, R₃, R₄, R₈, R₉ and A are as defined under formula (I), and
wherein S* is a stereogenic sulfur atom in R- or S-configuration, in which said S* center is in either enantiomerically pure or in enantiomerically enriched form; and
(B) reacting a sulfinyl compound of formula (III)

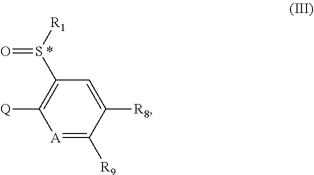

wherein Q, R₁, R₂, G₁, G₂, X₁, R₃, R₄, R₈, R₉ and A are as defined under formula (I), and
wherein S* is a stereogenic sulfur atom in R- or S-configuration, in which said S* center is in either enantiomerically pure or in enantiomerically enriched form;
with an imination reagent, in the presence of a catalyst, optionally in the presence of a suitable additive, in an appropriate solvent (or diluent);
to produce the sulfoximine compound of formula (I) in a stereospecific manner. The preferences and preferred embodiments related to the process for the preparation of compounds of formula (I) involving steps (A) and (B), the reaction conditions, and the compounds of formula (II) and (III) as further described below are also valid for this embodiment 31.

Embodiment 31a provides compounds, or an agrochemically acceptable salt, stereoisomer, enantiomer, tautomer or N-oxide thereof, according to any one of embodiments 1-26b comprising compounds of formula I that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

Embodiment 31 b provides compounds, or an agrochemically acceptable salt, stereoisomer, enantiomer, tautomer or N-oxide thereof, according to any one of embodiments 1-26b comprising compounds of formula I that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

Embodiment 31c provides compounds, or an agrochemically acceptable salt, stereoisomer, enantiomer, tautomer or N-oxide thereof, according to any one of embodiments 1-26b comprising compounds of formula I that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Embodiment 32 provides compounds, or an agrochemically acceptable salt, stereoisomer, enantiomer, tautomer or N-oxide thereof, according to any one of embodiments 31a-31c either in enantiomerically pure form or having an enantiomeric excess (e.e.) of the first eluting enantiomer of at least 40%, for example, at least 50%, 60%, 70% or 80%, preferably at least 90%, more preferably at least 95%, yet more preferably at least 98% and most preferably at least 99%.

Embodiment 33a provides compounds, or an agrochemically acceptable salt, stereoisomer, enantiomer, tautomer or N-oxide thereof, according to any one of embodiments 1-26b comprising compounds of formula I that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

Embodiment 33b provides compounds, or an agrochemically acceptable salt, stereoisomer, enantiomer, tautomer or N-oxide thereof, according to any one of embodiments 1-26b comprising compounds of formula I that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

Embodiment 33c provides compounds, or an agrochemically acceptable salt, stereoisomer, enantiomer, tautomer or N-oxide thereof, according to any one of embodiments 1-26b comprising compounds of formula I that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Embodiment 34 provides compounds, or an agrochemically acceptable salt, stereoisomer, enantiomer, tautomer or N-oxide thereof, according to any one of embodiments 33a-33c either in enantiomerically pure form or having an enantiomeric excess (e.e.) of the second eluting enantiomer of at least 40%, for example, at least 50%, 60%, 70% or 80%, preferably at least 90%, more preferably at least 95%, yet more preferably at least 98% and most preferably at least 99%.

A preferred group of compounds of formula I is represented by the compounds of formula I-1

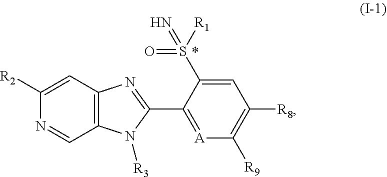

wherein R₁, R₂, R₃, R₈, R₉, S* and A are as defined under formula I above.

In one preferred group of compounds of formula I-1, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₃ is C₁-C₂alkyl; R₉ is hydrogen, methyl or ethyl; R₈ is cyanoisopropoxy, cyanoisopropyl or cyanocyclopropyl.

In another preferred group of compounds of formula I-1, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₃ is methyl; R₉ is hydrogen or methyl, preferably R₉ is hydrogen; R₈ is cyanoisopropoxy, cyanoisopropyl or cyanocyclopropyl.

In compounds of formula I-1 and all of the preferred embodiments of compounds of formula I-1 mentioned above, unless otherwise specified, R₁, R₂, R₃, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₃ is methyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-1a) which are compounds of formula (I-1), or any of the preferred embodiments of compounds of formula (I-1), wherein S* is in the R-configuration.

One group of compounds according to this embodiment are compounds of formula (I-1b) which are compounds of formula (I-1), or any of the preferred embodiments of compounds of formula (I-1), wherein S* is in the S-configuration.

One group of compounds according to this embodiment are compounds of formula (I-1c) which are compounds of formula (I-1), or any of the preferred embodiments of compounds of formula (I-1), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-1d) which are compounds of formula (I-1), or any of the preferred embodiments of compounds of formula (I-1), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-1e) which are compounds of formula (I-1), or any of the preferred embodiments of compounds of formula (I-1), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-1f) which are compounds of formula (I-1), or any of the preferred embodiments of compounds of formula (I-1), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-1g) which are compounds of formula (I-1), or any of the preferred embodiments of compounds of formula (I-1), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-1h) which are compounds of formula (I-1), or any of the preferred embodiments of compounds of formula (I-1), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-1i) which are compounds of formula (I-1), or any of the preferred embodiments of compounds of formula (I-1), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-2

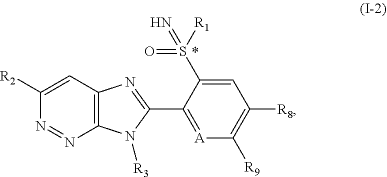

wherein R₁, R₂, R₃, R₈, R₉, S* and A are as defined under formula I above.

In one preferred group of compounds of formula I-2, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₃ is C₁-C₂alkyl; R₉ is hydrogen, methyl or ethyl; R₈ is cyanoisopropoxy, cyanoisopropyl or cyanocyclopropyl.

In another preferred group of compounds of formula I-2, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₃ is methyl; R₉ is hydrogen or methyl, preferably R₉ is hydrogen; R₈ is cyanoisopropoxy, cyanoisopropyl or cyanocyclopropyl.

In compounds of formula I-2 and all of the preferred embodiments of compounds of formula I-2 mentioned above, unless otherwise specified, R₁, R₂, R₃, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₃ is methyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-2a) which are compounds of formula (I-2), or any of the preferred embodiments of compounds of formula (I-2), wherein S* is in the R-configuration.

One group of compounds according to this embodiment are compounds of formula (I-2b) which are compounds of formula (I-2), or any of the preferred embodiments of compounds of formula (I-2), wherein S* is in the S-configuration.

One group of compounds according to this embodiment are compounds of formula (I-2c) which are compounds of formula (I-2), or any of the preferred embodiments of compounds of formula (I-2), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-2d) which are compounds of formula (I-2), or any of the preferred embodiments of compounds of formula (I-2), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-2e) which are compounds of formula (I-2), or any of the preferred embodiments of compounds of formula (I-2), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-20 which are compounds of formula (I-2), or any of the preferred embodiments of compounds of formula (I-2), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-2g) which are compounds of formula (I-2), or any of the preferred embodiments of compounds of formula (I-2), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-2h) which are compounds of formula (I-2), or any of the preferred embodiments of compounds of formula (I-2), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-2i) which are compounds of formula (I-2), or any of the preferred embodiments of compounds of formula (I-2), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-3

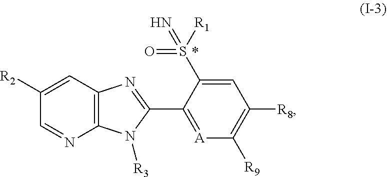

wherein R₁, R₂, R₃, R₈, R₉, S* and A are as defined under formula I above.

In one preferred group of compounds of formula I-3, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₃ is C₁-C₂alkyl; R₉ is hydrogen, methyl or ethyl; R₈ is cyanoisopropoxy, cyanoisopropyl or cyanocyclopropyl.

In another preferred group of compounds of formula I-3, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₃ is methyl; R₉ is hydrogen or methyl, preferably R₉ is hydrogen; R₈ is cyanoisopropoxy, cyanoisopropyl or cyanocyclopropyl.

In compounds of formula I-3 and all of the preferred embodiments of compounds of formula I-3 mentioned above, unless otherwise specified, R₁, R₂, R₃, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₃ is methyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-3a) which are compounds of formula (I-3), or any of the preferred embodiments of compounds of formula (I-3), wherein S* is in the R-configuration.

One group of compounds according to this embodiment are compounds of formula (I-3b) which are compounds of formula (I-3), or any of the preferred embodiments of compounds of formula (I-3), wherein S* is in the S-configuration.

One group of compounds according to this embodiment are compounds of formula (I-3c) which are compounds of formula (I-3), or any of the preferred embodiments of compounds of formula (I-3), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-3d) which are compounds of formula (I-3), or any of the preferred embodiments of compounds of formula (I-3), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-3e) which are compounds of formula (I-3), or any of the preferred embodiments of compounds of formula (I-3), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-3f which are compounds of formula (I-3), or any of the preferred embodiments of compounds of formula (I-3), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-3g) which are compounds of formula (I-3), or any of the preferred embodiments of compounds of formula (I-3), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-3h) which are compounds of formula (I-3), or any of the preferred embodiments of compounds of formula (I-3), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-3i) which are compounds of formula (I-3), or any of the preferred embodiments of compounds of formula (I-3), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-4

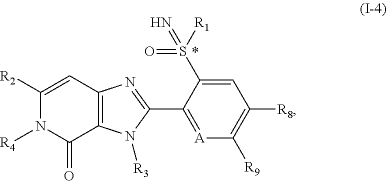

wherein R₁, R₂, R₃, R₄, R₈, R₉, S* and A are as defined under formula I above.

In one preferred group of compounds of formula I-4, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₃ is C₁-C₂alkyl; R₄ is C₁-C₂alkyl, C₁-C₂haloalkyl, C₁-C₂alkoxy or cyclopropyl; R₉ is hydrogen, methyl or ethyl; R₈ is cyanoisopropoxy, cyanoisopropyl or cyanocyclopropyl.

In another preferred group of compounds of formula I-4, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₃ is methyl; R₄ is methyl, ethyl, methoxy or cyclopropyl; R₉ is hydrogen or methyl, preferably R₉ is hydrogen; R₈ is cyanoisopropoxy, cyanoisopropyl or cyanocyclopropyl.

In another preferred group of of compounds of formula I-4, R₄ is ethyl, methoxy or cyclopropyl.

In compounds of formula I-4 and all of the preferred embodiments of compounds of formula I-4 mentioned above, unless otherwise specified, R₁, R₂, R₃, R₄, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₃ is methyl; R₄ is ethyl, methoxy or cyclopropyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-4a) which are compounds of formula (I-4), or any of the preferred embodiments of compounds of formula (I-4), wherein S* is in the R-configuration.

One group of compounds according to this embodiment are compounds of formula (I-4b) which are compounds of formula (I-4), or any of the preferred embodiments of compounds of formula (I-4), wherein S* is in the S-configuration.

One group of compounds according to this embodiment are compounds of formula (I-4c) which are compounds of formula (I-4), or any of the preferred embodiments of compounds of formula (I-4), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-4d) which are compounds of formula (I-4), or any of the preferred embodiments of compounds of formula (I-4), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-4e) which are compounds of formula (I-4), or any of the preferred embodiments of compounds of formula (I-4), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-40 which are compounds of formula (I-4), or any of the preferred embodiments of compounds of formula (I-4), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-4g) which are compounds of formula (I-4), or any of the preferred embodiments of compounds of formula (I-4), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-4h) which are compounds of formula (I-4), or any of the preferred embodiments of compounds of formula (I-4), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-4i) which are compounds of formula (I-4), or any of the preferred embodiments of compounds of formula (I-4), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-5

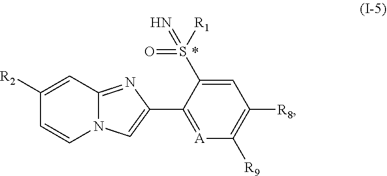

wherein R₁, R₂, R₈, R₉, S* and A are as defined under formula I above.

In one preferred group of compounds of formula I-5, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl; R₈ is cyanoisopropoxy, cyanoisopropyl or cyanocyclopropyl.

In another preferred group of compounds of formula I-5, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen; R₈ is cyanoisopropoxy, cyanoisopropyl or cyanocyclopropyl.

In compounds of formula I-5 and all of the preferred embodiments of compounds of formula I-5 mentioned above, unless otherwise specified, R₁, R₂, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-5a) which are compounds of formula (I-5), or any of the preferred embodiments of compounds of formula (I-5), wherein S* is in the R-configuration.

One group of compounds according to this embodiment are compounds of formula (I-5b) which are compounds of formula (I-5), or any of the preferred embodiments of compounds of formula (I-5), wherein S* is in the S-configuration.

One group of compounds according to this embodiment are compounds of formula (I-5c) which are compounds of formula (I-5), or any of the preferred embodiments of compounds of formula (I-5), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-5d) which are compounds of formula (I-5), or any of the preferred embodiments of compounds of formula (I-5), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-5e) which are compounds of formula (I-5), or any of the preferred embodiments of compounds of formula (I-5), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-5f which are compounds of formula (I-5), or any of the preferred embodiments of compounds of formula (I-5), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-5g) which are compounds of formula (I-5), or any of the preferred embodiments of compounds of formula (I-5), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-5h) which are compounds of formula (I-5), or any of the preferred embodiments of compounds of formula (I-5), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-5i) which are compounds of formula (I-5), or any of the preferred embodiments of compounds of formula (I-5), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-6

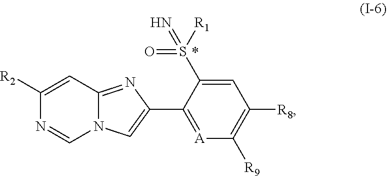

wherein R₁, R₂, R₈, R₉, S* and A are as defined under formula I above.

In one preferred group of compounds of formula I-6, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl; R₈ is cyanoisopropoxy, cyanoisopropyl or cyanocyclopropyl.

In another preferred group of compounds of formula I-6, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen; R₈ is cyanoisopropoxy, cyanoisopropyl or cyanocyclopropyl.

In compounds of formula I-6 and all of the preferred embodiments of compounds of formula I-6 mentioned above, unless otherwise specified, R₁, R₂, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-6a) which are compounds of formula (I-6), or any of the preferred embodiments of compounds of formula (I-6), wherein S* is in the R-configuration.

One group of compounds according to this embodiment are compounds of formula (I-6b) which are compounds of formula (I-6), or any of the preferred embodiments of compounds of formula (I-6), wherein S* is in the S-configuration.

One group of compounds according to this embodiment are compounds of formula (I-6c) which are compounds of formula (I-6), or any of the preferred embodiments of compounds of formula (I-6), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-6d) which are compounds of formula (I-6), or any of the preferred embodiments of compounds of formula (I-6), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-6e) which are compounds of formula (I-6), or any of the preferred embodiments of compounds of formula (I-6), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-60 which are compounds of formula (I-6), or any of the preferred embodiments of compounds of formula (I-6), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-6g) which are compounds of formula (I-6), or any of the preferred embodiments of compounds of formula (I-6), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-6h) which are compounds of formula (I-6), or any of the preferred embodiments of compounds of formula (I-6), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-6i) which are compounds of formula (I-6), or any of the preferred embodiments of compounds of formula (I-6), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-7

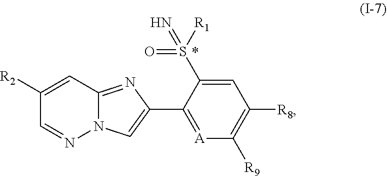

wherein R₁, R₂, R₈, R₉, S* and A are as defined under formula I above.

In one preferred group of compounds of formula I-7, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl; R₈ is cyanoisopropoxy, cyanoisopropyl or cyanocyclopropyl.

In another preferred group of compounds of formula I-7, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen; R₈ is cyanoisopropoxy, cyanoisopropyl or cyanocyclopropyl.

In compounds of formula I-7 and all of the preferred embodiments of compounds of formula I-7 mentioned above, unless otherwise specified, R₁, R₂, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-7a) which are compounds of formula (I-7), or any of the preferred embodiments of compounds of formula (I-7), wherein S* is in the R-configuration.

One group of compounds according to this embodiment are compounds of formula (I-7b) which are compounds of formula (I-7), or any of the preferred embodiments of compounds of formula (I-7), wherein S* is in the S-configuration.

One group of compounds according to this embodiment are compounds of formula (I-7c) which are compounds of formula (I-7), or any of the preferred embodiments of compounds of formula (I-7), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-7d) which are compounds of formula (I-7), or any of the preferred embodiments of compounds of formula (I-7), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-7e) which are compounds of formula (I-7), or any of the preferred embodiments of compounds of formula (I-7), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-70 which are compounds of formula (I-7), or any of the preferred embodiments of compounds of formula (I-7), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-7g) which are compounds of formula (I-7), or any of the preferred embodiments of compounds of formula (I-7), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-7h) which are compounds of formula (I-7), or any of the preferred embodiments of compounds of formula (I-7), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-6i) which are compounds of formula (I-6), or any of the preferred embodiments of compounds of formula (I-6), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-8

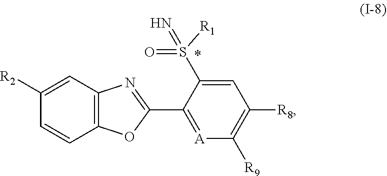

wherein R₁, R₂, R₈, R₉, S* and A are as defined under formula I above.

In one preferred group of compounds of formula I-8, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl; R₈ is cyanoisopropoxy, cyanoisopropyl or cyanocyclopropyl.

In another preferred group of compounds of formula I-8, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen; R₈ is cyanoisopropoxy, cyanoisopropyl or cyanocyclopropyl.

In compounds of formula I-8 and all of the preferred embodiments of compounds of formula I-8 mentioned above, unless otherwise specified, R₁, R₂, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-8a) which are compounds of formula (I-8), or any of the preferred embodiments of compounds of formula (I-8), wherein S* is in the R-configuration.

One group of compounds according to this embodiment are compounds of formula (I-8b) which are compounds of formula (I-8), or any of the preferred embodiments of compounds of formula (I-8), wherein S* is in the S-configuration.

One group of compounds according to this embodiment are compounds of formula (I-8c) which are compounds of formula (I-8), or any of the preferred embodiments of compounds of formula (I-8), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-8d) which are compounds of formula (I-8), or any of the preferred embodiments of compounds of formula (I-8), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-8e) which are compounds of formula (I-8), or any of the preferred embodiments of compounds of formula (I-8), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-80 which are compounds of formula (I-8), or any of the preferred embodiments of compounds of formula (I-8), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-8g) which are compounds of formula (I-8), or any of the preferred embodiments of compounds of formula (I-8), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-8h) which are compounds of formula (I-8), or any of the preferred embodiments of compounds of formula (I-8), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-8i) which are compounds of formula (I-8), or any of the preferred embodiments of compounds of formula (I-8), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-9

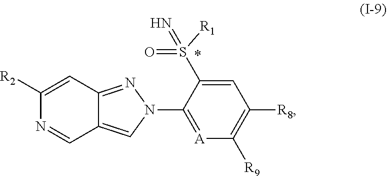

wherein R₁, R₂, R₈, R₉, S* and A are as defined under formula I above.

In one preferred group of compounds of formula I-9, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl; R₈ is cyanoisopropoxy, cyanoisopropyl or cyanocyclopropyl.

In another preferred group of compounds of formula I-9, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen; R₈ is cyanoisopropoxy, cyanoisopropyl or cyanocyclopropyl.

In compounds of formula I-9 and all of the preferred embodiments of compounds of formula I-9 mentioned above, unless otherwise specified, R₁, R₂, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-9a) which are compounds of formula (I-9), or any of the preferred embodiments of compounds of formula (I-9), wherein S* is in the R-configuration.

One group of compounds according to this embodiment are compounds of formula (I-9b) which are compounds of formula (I-9), or any of the preferred embodiments of compounds of formula (I-9), wherein S* is in the S-configuration.

One group of compounds according to this embodiment are compounds of formula (I-9c) which are compounds of formula (I-9), or any of the preferred embodiments of compounds of formula (I-9), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-9d) which are compounds of formula (I-9), or any of the preferred embodiments of compounds of formula (I-9), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-9e) which are compounds of formula (I-9), or any of the preferred embodiments of compounds of formula (I-9), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-90 which are compounds of formula (I-9), or any of the preferred embodiments of compounds of formula (I-9), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-9g) which are compounds of formula (I-9), or any of the preferred embodiments of compounds of formula (I-9), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-9h) which are compounds of formula (I-9), or any of the preferred embodiments of compounds of formula (I-9), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-9i) which are compounds of formula (I-9), or any of the preferred embodiments of compounds of formula (I-9), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I is represented by the compounds of formula I-10

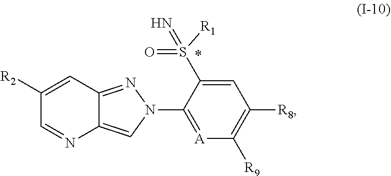

wherein R₁, R₂, R₈, R₉, S* and A are as defined under formula I above.

In one preferred group of compounds of formula I-10, A is CH or N; R₁ is ethyl, propyl or isopropyl; R₂ is C₁-C₂haloalkyl, C₁-C₂haloalkylsulfanyl, C₁-C₂haloalkylsulfinyl or C₁-C₂haloalkylsulfonyl; R₉ is hydrogen, methyl or ethyl; R₈ is cyanoisopropoxy, cyanoisopropyl or cyanocyclopropyl.

In another preferred group of compounds of formula I-10, A is CH or N; R₁ is ethyl; R₂ is C₁-C₂fluoroalkyl, trifluoromethylsulfanyl, trifluoromethylsulfinyl, trifluoromethylsulfonyl, difluoromethylsulfanyl, difluoromethylsulfinyl, or difluoromethylsulfonyl; R₉ is hydrogen or methyl, preferably R₉ is hydrogen; R₈ is cyanoisopropoxy, cyanoisopropyl or cyanocyclopropyl.

In compounds of formula I-10 and all of the preferred embodiments of compounds of formula I-10 mentioned above, unless otherwise specified, R₁, R₂, R₈, R₉, S* and A are as defined under formula I above; preferably A is CH or N, more preferably A is N; R₁ is ethyl; R₂ is trifluoromethyl, pentafluoroethyl or trifluoromethylsulfanyl; preferably R₂ is trifluoromethyl; R₉ is hydrogen; R₈ is 1-cyano-1-methyl-ethoxy, 1-cyano-1-methyl-ethyl or 1-cyanocyclopropyl.

One group of compounds according to this embodiment are compounds of formula (I-10a) which are compounds of formula (I-10), or any of the preferred embodiments of compounds of formula (I-10), wherein S* is in the R-configuration.

One group of compounds according to this embodiment are compounds of formula (I-10b) which are compounds of formula (I-10), or any of the preferred embodiments of compounds of formula (I-10), wherein S* is in the S-configuration.

One group of compounds according to this embodiment are compounds of formula (I-10c) which are compounds of formula (I-10), or any of the preferred embodiments of compounds of formula (I-10), that are the first eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-10d) which are compounds of formula (I-10), or any of the preferred embodiments of compounds of formula (I-10), that are the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-10e) which are compounds of formula (I-10), or any of the preferred embodiments of compounds of formula (I-10), that are the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

One group of compounds according to this embodiment are compounds of formula (I-100 which are compounds of formula (I-10), or any of the preferred embodiments of compounds of formula (I-10), that are the second eluting enantiomers upon chiral resolution of the racemate by preparative chromatography using immobilized amylose-based (CHIRALPAK® IA, CHIRALPAK® IG) or cellulose-based (CHIRALPAK® IC) chiral phases.

One group of compounds according to this embodiment are compounds of formula (I-10g) which are compounds of formula (I-10), or any of the preferred embodiments of compounds of formula (I-10), that are the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

One group of compounds according to this embodiment are compounds of formula (I-10h) which are compounds of formula (I-10), or any of the preferred embodiments of compounds of formula (I-10), that are the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another group of compounds according to this embodiment are compounds of formula (I-10i) which are compounds of formula (I-10), or any of the preferred embodiments of compounds of formula (I-10), whenever prepared or that are obtainable in a stereospecific manner by imination of stereogenic sulfinyl derivatives that are produced by stereoselective oxidation of the corresponding sulfanyl compounds as further set forth and described in embodiment 31.

Another preferred group of compounds of formula I are those wherein Q, R₃, R₄ and X₁ are as defined under formula I (above);
A is CH or N, preferably A is N;
S* is a stereogenic sulfur atom which is in R- or S-configuration;
R₁ is ethyl, propyl or isopropyl; preferably R₁ is ethyl;
R₂ is trifluoromethyl, pentafluoroethyl or trifluoromethylsulfanyl; preferably R₂ is trifluoromethyl;
R₈ is 1-cyano-1-methyl-ethoxy, 1-cyano-1-methyl-ethyl or 1-cyanocyclopropyl;
R₉ is hydrogen or methyl; preferably R₉ is hydrogen; and
in the case of compounds wherein Q is Q₁ or Q₄, G₁ is N and G₂ is CH or G₁ is CH and G₂ is N or both G₁ and G₂ are N; and
in the case of the compounds wherein Q is Q₂, G₂ is N or CH; or
an agrochemically acceptable salt, stereoisomer, enantiomer, tautomer or N-oxide thereof.

Another especially preferred group of compounds of formula I are those represented by the compounds of formula I-1, I-2, I-3, I-4, I-5, I-6, I-7, I-8, I-9, or I-10 wherein
A is CH or N, preferably A is N;
S* is a stereogenic sulfur atom which is in R- or S-configuration;
R₁ is ethyl, propyl or isopropyl; preferably R₁ is ethyl;
R₂ is trifluoromethyl, pentafluoroethyl or trifluoromethylsulfanyl; preferably R₂ is trifluoromethyl;
R₈ is 1-cyano-1-methyl-ethoxy, 1-cyano-1-methyl-ethyl or 1-cyanocyclopropyl;
R₉ is hydrogen or methyl; preferably R₉ is hydrogen; and
in the case of the compounds of formula I-1, 1-2, 1-3, and 1-4 R₃ is methyl; and in the case of the compounds of formula I-4 R₄ is ethyl, methoxy or cyclopropyl.

A preferred group of compounds of formula I are those represented by the (S) absolute configuration at the stereogenic sulfur center (S*).

Another preferred group of compounds of formula I are those either in (S) enantiomerically pure form or with an S-enantiomeric excess (e.e.) of at least 40%, for example, at least 50%, 60%, 70% or 80%, preferably at least 90%, more preferably at least 95%, yet more preferably at least 98% and most preferably at least 99%.

Another preferred group of compounds of formula I are those represented by the (R) absolute configuration at the stereogenic sulfur center (S*).

Another preferred group of compounds of formula I are those either in (R) enantiomerically pure form or with an R-enantiomeric excess (e.e.) of at least 40%, for example, at least 50%, 60%, 70% or 80%, preferably at least 90%, more preferably at least 95%, yet more preferably at least 98% and most preferably at least 99%.

An outstanding preferred group of compounds of formula I are those represented by the (S)- or (R)-enantiomer compounds P1 to P19 as defined in the table Y below:

TABLE Y

| | | | Configuration |
|---|---|---|---|
| No. | IUPAC name | Structures | at sulfur |
| (S)-P1 | (S)-2-[6-[5-cyclopropyl-3-methyl-4- oxo-6-(trifluoromethyl)imidazo[4,5- c]pyridin-2-yl]-5-(ethylsulfonimidoyl)- 3-pyridyl]oxy]-2-methyl-propanenitrile | 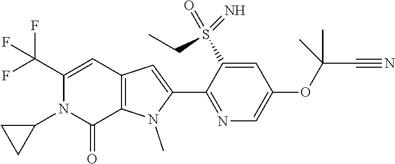 [SMILES: CC[S@](=N)(=O)c1cc(OC(C)(C)C#N)cnc1-c1cc2cc(C(F)(F)F)n(C3CC3)c(=O)c2n1C] | S |
| (R)-P1 | (R)-2-[[6-[5-cyclopropyl-3-methyl-4- oxo-6-(trifluoromethyl)imidazo[4,5- c]pyridin-2-yl]-5-(ethylsulfonimidoyl)- 3-pyridyl]oxy]-2-methyl-propanenitrile | 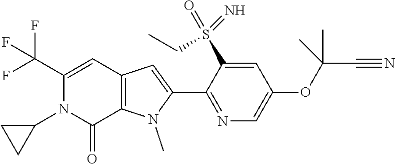 [SMILES: CC[S@@](=N)(=O)c1cc(OC(C)(C)C#N)cnc1-c1cc2cc(C(F)(F)F)n(C3CC3)c(=O)c2n1C] | R |
| (S)-P2 | (S)-1-[5-(ethylsulfonimidoyl)-6-[7- methyl-3-(trifluoromethyl)imidazo[4,5- c]pyridazin-6-yl]-3- pyridyl]cyclopropanecarbonitrile | 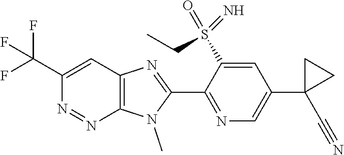 [SMILES: CC[S@](=N)(=O)c1cc(C2(C#N)CC2)cnc1-c1nc2cc(C(F)(F)F)nnc2n1C] | S |
| (R)-P2 | (R)-1-[5-(ethylsulfonimidoyl)-6-[7- methyl-3-(trifluoromethyl)imidazo[4,5- c]pyridazin-6-yl]-3- pyridyl]cyclopropanecarbonitrile | 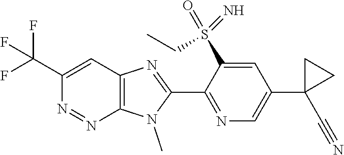 [SMILES: CC[S@@](=N)(=O)c1cc(C2(C#N)CC2)cnc1-c1nc2cc(C(F)(F)F)nnc2n1C] | R |
| (S)-P3 | (S)-2-[5-(ethylsulfonimidoyl)-6-[7- (trifluoromethyl)imidazo[1,2-a]pyridin- 2-yl]-3-pyridyl]oxy]-2-methyl- propanenitrile | 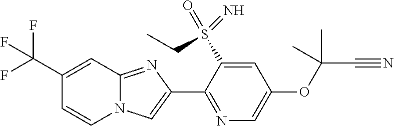 [SMILES: CC[S@](=N)(=O)c1cc(OC(C)(C)C#N)cnc1-c1cn2ccc(C(F)(F)F)cc2n1] | S |
| (R)-P3 | (R)-2-[5-(ethylsulfonimidoyl)-6-[7- (trifluoromethyl)imidazo[1,2-a]pyridin- 2-yl]-3-pyridyl]oxy]-2-methyl- propanenitrile | 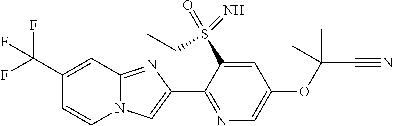 [SMILES: CC[S@@](=N)(=O)c1cc(OC(C)(C)C#N)cnc1-c1cn2ccc(C(F)(F)F)cc2n1] | R |
| (S)-P4 | (S)-2-[5-(ethylsulfonimidoyl)-6-[3- methyl-6-(trifluoromethyl)imidazo[4,5- c]pyridin-2-yl]-3-pyridyl]-2-methyl- propanenitrile | 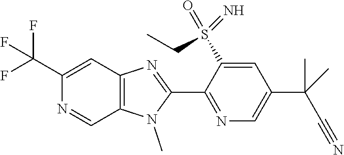 [SMILES: CC[S@](=N)(=O)c1cc(C(C)(C)C#N)cnc1-c1nc2cc(C(F)(F)F)ncc2n1C] | S |
| (R)-P4 | (R)-2-[5-(ethylsulfonimidoyl)-6-[3- methyl-6-(trifluoromethyl)imidazo[4,5- c]pyridin-2-yl]-3-pyridyl]-2-methyl- propanenitrile | 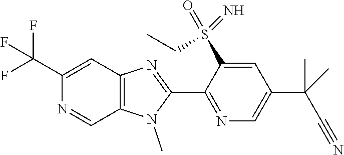 [SMILES: CC[S@@](=N)(=O)c1cc(C(C)(C)C#N)cnc1-c1nc2cc(C(F)(F)F)ncc2n1C] | R |
| (S)-P5 | (S)-2-[5-(ethylsulfonimidoyl)-2- methyl-6-[3-methyl-6- (trifluoromethyl)imidazo[4,5-b]pyridin- 2-yl]-3-pyridyl]oxy]-2-methyl- propanenitrile | 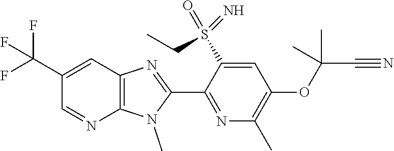 [SMILES: CC[S@](=N)(=O)c1cc(OC(C)(C)C#N)c(C)nc1-c1nc2cc(C(F)(F)F)cnc2n1C] | S |
| (R)-P5 | (R)-2-[5-(ethylsulfonimidoyl)-2- methyl-6-[3-methyl-6- (trifluoromethyl)imidazo[4,5-b]pyridin- 2-yl]-3-pyridyl]oxy]-2-methyl- propanenitrile | 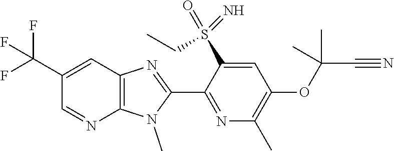 [SMILES: CC[S@@](=N)(=O)c1cc(OC(C)(C)C#N)c(C)nc1-c1nc2cc(C(F)(F)F)cnc2n1C] | R |
| (S)-P6 | (S)-1-[3-(ethylsulfonimidoyl)-4-[3- methyl-6-(trifluoromethyl)imidazo[4,5- b]pyridin-2- yl]phenyl]cyclopropanecarbonitrile | 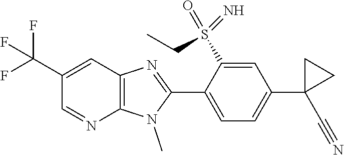 [SMILES: CC[S@](=N)(=O)c1cc(C2(C#N)CC2)ccc1-c1nc2cc(C(F)(F)F)cnc2n1C] | S |
| (R)-P6 | (R)-1-[3-(ethylsulfonimidoyl)-4-[3- methyl-6-(trifluoromethyl)imidazo[4,5- b]pyridin-2- yl]phenyl]cyclopropanecarbonitrile | 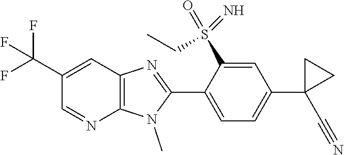 [SMILES: CC[S@@](=N)(=O)c1cc(C2(C#N)CC2)ccc1-c1nc2cc(C(F)(F)F)cnc2n1C] | R |
| (S)-P7 | (S)-2-[[5-(ethylsulfonimidoyl)-6-[7- (trifluoromethylsulfanyl)imidazo[1,2- c]pyrimidin-2-yl]-3-pyridyl]oxy]-2- methyl-propanenitrile | 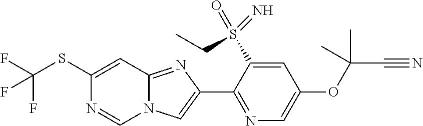 [SMILES: CC[S@](=N)(=O)c1cc(OC(C)(C)C#N)cnc1-c1cn2cnc(SC(F)(F)F)cc2n1] | S |
| (R)-P7 | (R)-2-[[5-(ethylsulfonimidoyl)-6-[7- (trifluoromethylsulfanyl)imidazo[1,2- c]pyrimidin-2-yl]-3-pyridyl]oxy]-2- methyl-propanenitrile | 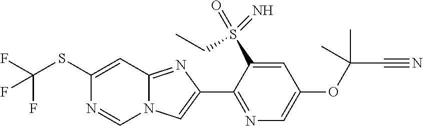 [SMILES: CC[S@@](=N)(=O)c1cc(OC(C)(C)C#N)cnc1-c1cn2cnc(SC(F)(F)F)cc2n1] | R |
| (S)-P8 | (S)-1-[5-(ethylsulfonimidoyl)-6-[5- methoxy-3-methyl-4-oxo-6- (trifluoromethyl)imidazo[4,5-c]pyridin- 2-yl]-3- pyridyl]cyclopropanecarbonitrile | 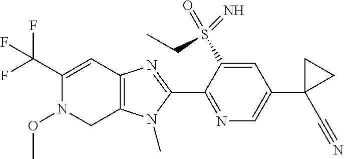 [SMILES: CC[S@](=N)(=O)c1cc(C2(C#N)CC2)cnc1-c1nc2c(n1C)CN(OC)C(C(F)(F)F)=C2] | S |
| (R)-P8 | (R)-1-[5-(ethylsulfonimidoyl)-6-[5- methoxy-3-methyl-4-oxo-6- (trifluoromethyl)imidazo [4,5-c]pyridin- 2-yl]-3- pyridyl]cyclopropanecarbonitrile | 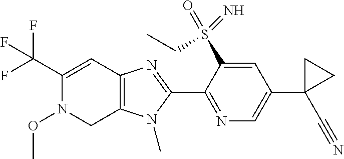 [SMILES: CC[S@@](=N)(=O)c1cc(C2(C#N)CC2)cnc1-c1nc2c(n1C)CN(OC)C(C(F)(F)F)=C2] | R |
| (S)-P9 | (S)-2-[5-(ethylsulfonimidoyl)-6-[3- methyl-6-(trifluoromethyl)imidazo[4,5- b]pyridin-2-yl]-3-pyridyl]oxy]-2- methyl-propanenitrile | 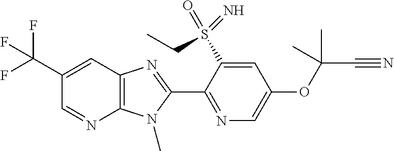 [SMILES: CC[S@](=N)(=O)c1cc(OC(C)(C)C#N)cnc1-c1nc2cc(C(F)(F)F)cnc2n1C] | S |
| (R)-P9 | (R)-2-[5-(ethylsulfonimidoyl)-6-[3- methyl-6-(trifluoromethyl)imidazo[4,5- b]pyridin-2-yl]-3-pyridyl]oxy]-2- methyl-propanenitrile | 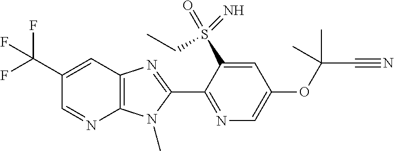 [SMILES: CC[S@@](=N)(=O)c1cc(OC(C)(C)C#N)cnc1-c1nc2cc(C(F)(F)F)cnc2n1C] | R |
| (S)-P10 | (S)-1-[5-(ethylsulfonimidoyl)-6-[3- methyl-6-(trifluoromethyl)imidazo[4,5- c]pyridin-2-yl]-3- pyridyl]cyclopropanecarbonitrile | 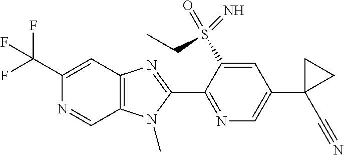 [SMILES: CC[S@](=N)(=O)c1cc(C2(C#N)CC2)cnc1-c1nc2cc(C(F)(F)F)ncc2n1C] | S |
| (R)-P10 | (R)-1-[5-(ethylsulfonimidoyl)-6-[3- methyl-6-(trifluoromethyl)imidazo[4,5- c]pyridin-2-yl]-3- pyridyl]cyclopropanecarbonitrile | 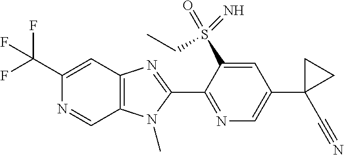 [SMILES: CC[S@@](=N)(=O)c1cc(C2(C#N)CC2)cnc1-c1nc2cc(C(F)(F)F)ncc2n1C] | R |
| (S)-P11 | (S)-2-[5-(ethylsulfonimidoyl)-6-[7- (trifluoromethyl)imidazo[1,2- c]pyrimidin-2-yl]-3-pyridyl]oxy]-2- methyl-propanenitrile | 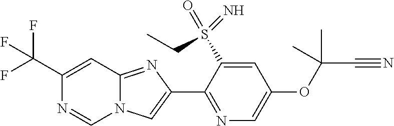 [SMILES: CC[S@](=N)(=O)c1cc(OC(C)(C)C#N)cnc1-c1cn2cnc(C(F)(F)F)cc2n1] | S |
| (R)-P11 | (R)-2-[[5-(ethylsulfonimidoyl)-6-[7- (trifluoromethyl)imidazo[1,2- c]pyrimidin-2-yl]-3-pyridyl]oxy]-2- methyl-propanenitrile | 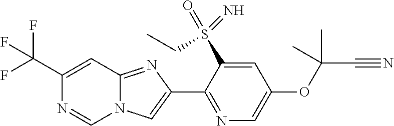 [SMILES: CC[S@@](=N)(=O)c1cc(OC(C)(C)C#N)cnc1-c1cn2cnc(C(F)(F)F)cc2n1] | R |
| (S)-P12 | (S)-2-[5-(ethylsulfonimidoyl)-6-[3- b]pyridin-2-yl]-3-pyridyl]-2-methyl- methyl-6-(trifluoromethyl)imidazo[4,5- propanenitrile | 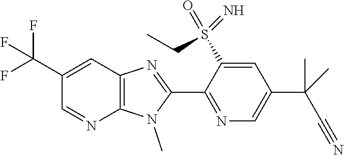 [SMILES: CC[S@](=N)(=O)c1cc(C(C)(C)C#N)cnc1-c1nc2cc(C(F)(F)F)cnc2n1C] | S |
| (R)-P12 | (R)-2-[5-(ethylsulfonimidoyl)-6-[3- b]pyridin-2-yl]-3-pyridyl]-2-methyl- methyl-6-(trifluoromethyl)imidazo[4,5- propanenitrile | 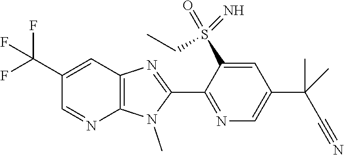 [SMILES: CC[S@@](=N)(=O)c1cc(C(C)(C)C#N)cnc1-c1nc2cc(C(F)(F)F)cnc2n1C] | R |
| (S)-P13 | (S)-2-[5-(ethylsulfonimidoyl)-6-[3- methyl-6-(trifluoromethyl)imidazo[4,5- c]pyridin-2-yl]-3-pyridyl]oxy]-2- methyl-propanenitrile | 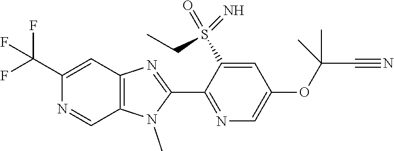 [SMILES: CC[S@](=N)(=O)c1cc(OC(C)(C)C#N)cnc1-c1nc2cc(C(F)(F)F)ncc2n1C] | S |
| (R)-P13 | (R)-2-[[5-(ethylsulfonimidoyl)-6-[3- methyl-6-(trifluoromethyl)imidazo[4,5- [c]pyridin-2-yl]-3-pyridyl]oxy]-2- methyl-propanenitrile | 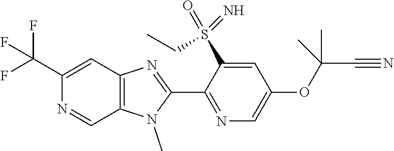 [SMILES: CC[S@@](=N)(=O)c1cc(OC(C)(C)C#N)cnc1-c1nc2cc(C(F)(F)F)ncc2n1C] | R |
| (S)-P14 | (S)-1-[6-[5-ethyl-3-methyl-4-oxo-6- (trifluoromethyl)imidazo[4,5-c]pyridin- 2-yl]-5-(ethylsulfonimidoyl)-3- pyridyl]cyclopropanecarbonitrile | 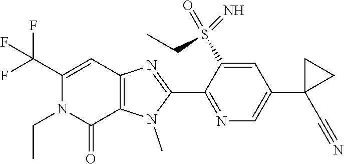 [SMILES: CCn1c(C(F)(F)F)cc2nc(-c3ncc(C4(C#N)CC4)cc3[S@@](=N)(=O)CC)n(C)c2c1=O] | S |
| (R)-P14 | (R)-1-[6-[5-ethyl-3-methyl-4-oxo-6- (trifluoromethyl)imidazo[4,5-c]pyridin- 2-yl]-5-(ethylsulfonimidoyl)-3- pyridyl]cyclopropanecarbonitrile | 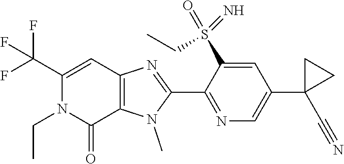 [SMILES: CCn1c(C(F)(F)F)cc2nc(-c3ncc(C4(C#N)CC4)cc3[S@](=N)(=O)CC)n(C)c2c1=O] | R |
| (S)-P15 | (S)-2-[5-(ethylsulfonimidoyl)-6-[7- methyl-3-(trifluoromethyl)imidazo[4,5- c]pyridazin-6-yl]-3-pyridyl]oxy]-2- methyl-propanenitrile | 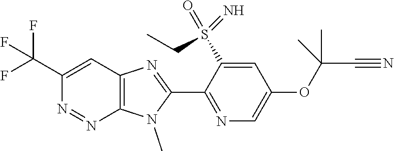 [SMILES: CC[S@](=N)(=O)c1cc(OC(C)(C)C#N)cnc1-c1nc2cc(C(F)(F)F)nnc2n1C] | S |
| (R)-P15 | (R)-2-[[5-(ethylsulfonimidoyl)-6-[7- methyl-3-(trifluoromethyl)imidazo[4,5- c]pyridazin-6-yl]-3-pyridyl]oxy]-2- methyl-propanenitrile | 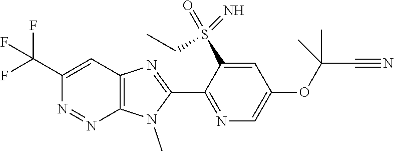 [SMILES: CC[S@@](=N)(=O)c1cc(OC(C)(C)C#N)cnc1-c1nc2cc(C(F)(F)F)nnc2n1C] | R |
| (S)-P16 | (S)-1-[5-(ethylsulfonimidoyl)-6-[3- methyl-6-(trifluoromethyl)imidazo[4,5- [b]pyridin-2-yl]-3- pyridyl]cyclopropanecarbonitrile | 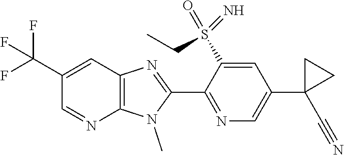 [SMILES: CC[S@](=N)(=O)c1cc(C2(C#N)CC2)cnc1-c1nc2cc(C(F)(F)F)cnc2n1C] | S |
| (R)-P16 | (R)-1-[5-(ethylsulfonimidoyl)-6-[3- methyl-6-(trifluoromethyl)imidazo[4,5- b]pyridin-2-yl]-3- pyridyl]cyclopropanecarbonitrile | 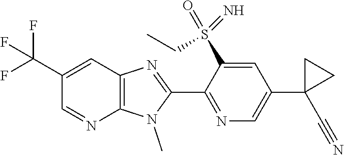 [SMILES: CC[S@@](=N)(=O)c1cc(C2(C#N)CC2)cnc1-c1nc2cc(C(F)(F)F)cnc2n1C] | R |
| (S)-P17 | (S)-2-[5-(ethylsulfonimidoyl)-2- methyl-6-[3-methyl-6- (trifluoromethyl)imidazo[4,5-c]pyridin- 2-yl]-3-pyridyl]oxy]-2-methyl- propanenitrile | 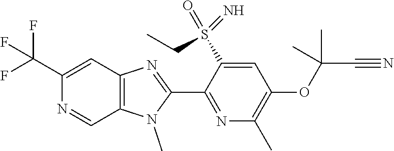 [SMILES: CC[S@](=N)(=O)c1cc(OC(C)(C)C#N)c(C)nc1-c1nc2cc(C(F)(F)F)ncc2n1C] | S |
| (R)-P17 | (R)-2-[[5-(ethylsulfonimidoyl)-2- methyl-6-[3-methyl-6- (trifluoromethyl)imidazo[4,5-c]pyridin- 2-yl]-3-pyridyl]oxy]-2-methyl- propanenitrile | 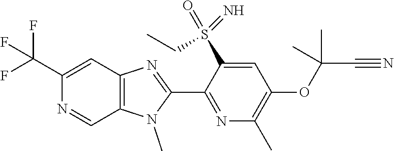 [SMILES: CC[S@@](=N)(=O)c1cc(OC(C)(C)C#N)c(C)nc1-c1nc2cc(C(F)(F)F)ncc2n1C] | R |
| (S)-P18 | (S)-1-[3-(ethylsulfonimidoyl)-4-[3- methyl-6-(trifluoromethyl)imidazo[4,5- c]pyridin-2- yl]phenyl]cyclopropanecarbonitrile | 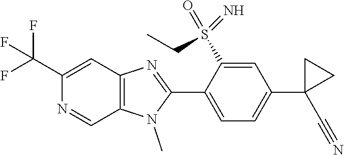 [SMILES: CC[S@](=N)(=O)c1cc(C2(C#N)CC2)ccc1-c1nc2cc(C(F)(F)F)ncc2n1C] | S |
| (R)-P18 | (R)-1-[3-(ethylsulfonimidoyl)-4-[3- methyl-6-(trifluoromethyl)imidazo[4,5- c]pyridin-2- yl]phenyl]cyclopropanecarbonitrile | 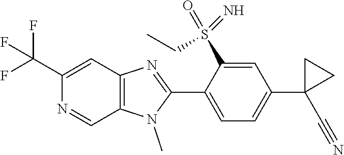 [SMILES: CC[S@@](=N)(=O)c1cc(C2(C#N)CC2)ccc1-c1nc2cc(C(F)(F)F)ncc2n1C] | R |
| (S)-P19 | (S)-2-[6-[5-ethyl-3-methyl-4-oxo-6- (trifluoromethyl)imidazo[4,5-c]pyridin- 2-yl]-5-(ethylsulfonimidoyl)-3- pyridyl]oxy]-2-methyl-propanenitrile | 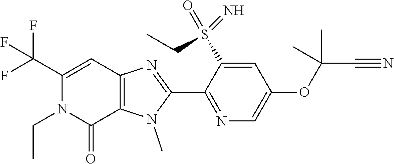 [SMILES: CCn1c(C(F)(F)F)cc2nc(-c3ncc(OC(C)(C)C#N)cc3[S@@](=N)(=O)CC)n(C)c2c1=O] | S |
| (R)-P19 | (R)-2-[[6-[5-ethyl-3-methyl-4-oxo-6- (trifluoromethyl)imidazo[4,5-c]pyridin- 2-yl]-5-(ethylsulfonimidoyl)-3- pyridyl]oxy]-2-methyl-propanenitrile | 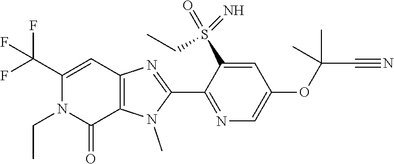 [SMILES: CCn1c(C(F)(F)F)cc2nc(-c3ncc(OC(C)(C)C#N)cc3[S@](=N)(=O)CC)n(C)c2c1=O] | R |

Another preferred group of compounds of formula I are those comprising the first eluting enantiomers upon chiral resolution by preparative chromatography using immobilized amylose-based (CHIRALPAK® IA, CHIRALPAK® IG) or cellulose-based (CHIRALPAK® IC) chiral phases.

Another preferred group of compounds of formula I are those comprising the first eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

Another preferred group of compounds of formula I are those comprising the first eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another preferred group of compounds of formula I are those having an enantiomeric excess (e.e.) of the first eluting enantiomer of at least 40%, for example, at least 50%, 60%, 70% or 80%, preferably at least 90%, more preferably at least 95%, yet more preferably at least 98% and most preferably at least 99%.

Another preferred group of compounds of formula I are those comprising the second eluting enantiomers upon chiral resolution by preparative chromatography using immobilized amylose-based (CHIRALPAK® IA, CHIRALPAK® IG) or cellulose-based (CHIRALPAK® IC) chiral phases.

Another preferred group of compounds of formula I are those comprising the second eluting enantiomers upon chiral resolution of the racemate by preparative SFC (supercritical fluid chromatography) using immobilized amylose-based (CHIRALPAK® IA, CHIRALPAK® IG) or cellulose-based (CHIRALPAK® IC) chiral phases.

Another preferred group of compounds of formula I are those comprising the second eluting enantiomers upon chiral resolution of the racemate by preparative SFC using immobilized amylose-based (CHIRALPAK® IA, CHIRALPAK® IG) or cellulose-based (CHIRALPAK® IC) chiral phases and using supercritical CO₂ and an alcohol cosolvent, such as preferably methanol, ethanol or isopropyl alcohol, as the mobile phase.

Another preferred group of compounds of formula I are those having an enantiomeric excess (e.e.) of the second eluting enantiomer of at least 40%, for example, at least 50%, 60%, 70% or 80%, preferably at least 90%, more preferably at least 95%, yet more preferably at least 98% and most preferably at least 99%.

An outstanding preferred group of compounds of formula I are those represented by the first eluting (P1-A through P19-A) or the second eluting (P1-B through P19-B) enantiomer compounds P1 to P19 as defined in the table Z below:

TABLE Z

| | | | First eluting | Second eluting |
|---|---|---|---|---|
| No. | IUPAC name | Structures | enantiomer | enantiomer |
| P1 | 2-[6-[5-cyclopropyl-3- methyl-4-oxo-6- (trifluoromethyl)imidazo[4, 5-c]pyridin-2-yl]-5- (ethylsulfonimidoyl)-3- pyridyl]oxy]-2-methyl- propanenitrile | 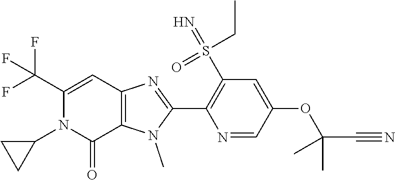 [SMILES: CCS(=N)(=O)c1cc(OC(C)(C)C#N)cnc1-c1nc2cc(C(F)(F)F)n(C3CC3)c(=O)c2n1C] | P1-A | P1-B |
| P2 | 1-[5-(ethylsulfonimidoyl)-6- [7-methyl-3- (trifluoromethyl)imidazo[4, 5-c]pyridazin-6-yl]-3- pyridyl] cyclopropanecarbonitrile | 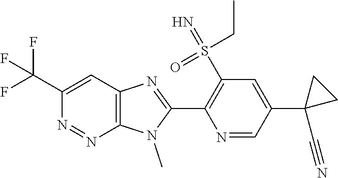 [SMILES: CCS(=N)(=O)c1cc(C2(C#N)CC2)cnc1-c1nc2cc(C(F)(F)F)nnc2n1C] | P2-A | P2-B |
| P3 | 2-[5-(ethylsulfonimidoyl)- 6-[7- (trifluoromethyl)imidazo[1, 2-a]pyridin-2-yl]-3- pyridyl]oxy]-2-methyl- propanenitrile | 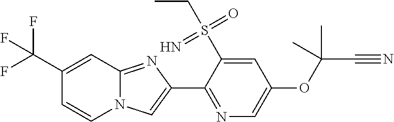 [SMILES: CCS(=N)(=O)c1cc(OC(C)(C)C#N)cnc1-c1cn2ccc(C(F)(F)F)cc2n1] | P3-A | P3-B |
| P4 | 2-[5-(ethylsulfonimidoyl)-6- [3-methyl-6- (trifluoromethyl)imidazo[4, 5-c]pyridin-2-yl]-3-pyridyl]- 2-methyl-propanenitrile | 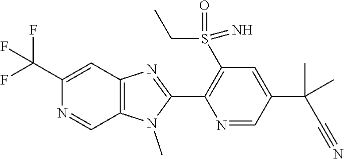 [SMILES: CCS(=N)(=O)c1cc(C(C)(C)C#N)cnc1-c1nc2cc(C(F)(F)F)ncc2n1C] | P4-A | P4-B |
| P5 | 2-[5-(ethylsulfonimidoyl)- 2-methyl-6-[3-methyl-6- (trifluoromethyl)imidazo[4, 5-b]pyridin-2-yl]-3- pyridyl]oxy]-2-methyl- propanenitrile | 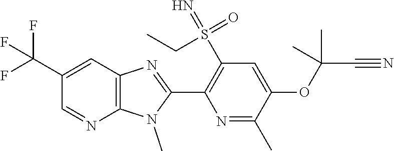 [SMILES: CCS(=N)(=O)c1cc(OC(C)(C)C#N)c(C)nc1-c1nc2cc(C(F)(F)F)cnc2n1C] | P5-A | P5-B |
| P6 | 1-[3-(ethylsulfonimidoyl)-4- [3-methyl-6- (trifluoromethyl)imidazo[4, 5-b]pyridin-2- yl]phenyl] cyclopropanecarbonitrile | 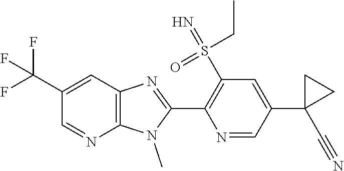 [SMILES: CCS(=N)(=O)c1cc(C2(C#N)CC2)cnc1-c1nc2cc(C(F)(F)F)cnc2n1C] | P6-A | P6-B |
| P7 | 2-[5-(ethylsulfonimidoyl)- 6-[7- (trifluoromethylsulfanyl) imidazo[1,2-c]pyrimidin-2-yl]- 3-pyridyl]oxy]-2-methyl- propanenitrile | 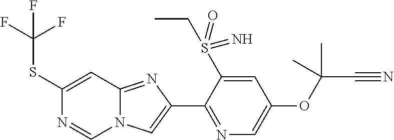 [SMILES: CCS(=N)(=O)c1cc(OC(C)(C)C#N)cnc1-c1cn2cnc(SC(F)(F)F)cc2n1] | P7-A | P7-B |
| P8 | 1-[5-(ethylsulfonimidoyl)-6- [5-methoxy-3-methyl-4- oxo-6- (trifluoromethyl)imidazo[4, 5-c]pyridin-2-yl]-3- pyridyl] cyclopropanecarbonitrile | 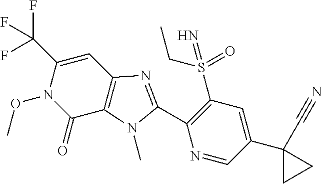 [SMILES: CCS(=N)(=O)c1cc(C2(C#N)CC2)cnc1-c1nc2cc(C(F)(F)F)n(OC)c(=O)c2n1C] | P8-A | P8-B |
| P9 | 2-[5-(ethylsulfonimidoyl)- 6-[3-methyl-6- (trifluoromethyl)imidazo[4, 5-b]pyridin-2-yl]-3- pyridyl]oxy]-2-methyl- propanenitrile | 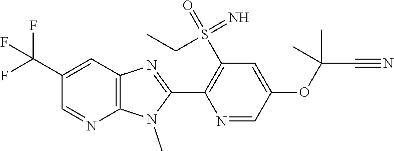 [SMILES: CCS(=N)(=O)c1cc(OC(C)(C)C#N)cnc1-c1nc2cc(C(F)(F)F)cnc2n1C] | P9-A | P9-B |
| P10 | 1-[5-(ethylsulfonimidoyl)-6- [3-methyl-6- (trifluoromethyl)imidazo[4, 5-c]pyridin-2-yl]-3- pyridyl] cyclopropanecarbonitrile | 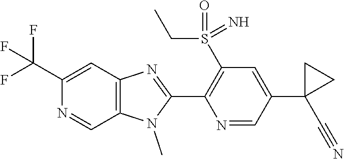 [SMILES: CCS(=N)(=O)c1cc(C2(C#N)CC2)cnc1-c1nc2cc(C(F)(F)F)ncc2n1C] | P10-A | P10-B |
| P11 | 2-[5-(ethylsulfonimidoyl)- 6-[7- (trifluoromethyl)imidazo[1, 2-c]pyrimidin-2-yl]-3- pyridyl]oxy]-2-methyl- propanenitrile | 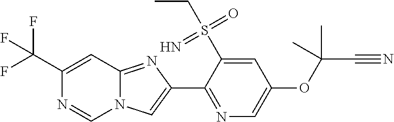 [SMILES: CCS(=N)(=O)c1cc(OC(C)(C)C#N)cnc1-c1cn2cnc(C(F)(F)F)cc2n1] | P11-A | P11-B |
| P12 | 2-[5-(ethylsulfonimidoyl)-6- [3-methyl-6- (trifluoromethyl)imidazo[4, 5-b]pyridin-2-yl]-3-pyridyl]- 2-methyl-propanenitrile | 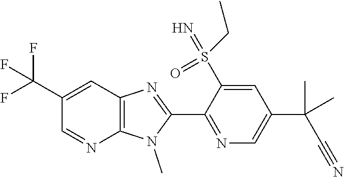 [SMILES: CCS(=N)(=O)c1cc(C(C)(C)C#N)cnc1-c1nc2cc(C(F)(F)F)cnc2n1C] | P12-A | P12-B |
| P13 | 2-[5-(ethylsulfonimidoyl)- 6-[3-methyl-6- (trifluoromethyl)imidazo[4, 5-c]pyridin-2-yl]-3- pyridyl]oxy]-2-methyl- propanenitrile | 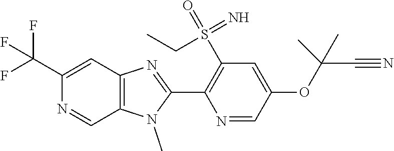 [SMILES: CCS(=N)(=O)c1cc(OC(C)(C)C#N)cnc1-c1nc2cc(C(F)(F)F)ncc2n1C] | P13-A | P13-B |
| P14 | 1-[6-[5-ethyl-3-methyl-4- oxo-6- (trifluoromethyl)imidazo[4, 5-c]pyridin-2-yl]-5- (ethylsulfonimidoyl)-3- pyridyl] cyclopropanecarbonitrile | 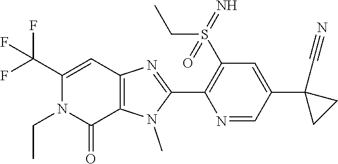 [SMILES: CCn1c(C(F)(F)F)cc2nc(-c3ncc(C4(C#N)CC4)cc3S(=N)(=O)CC)n(C)c2c1=O] | P14-A | P14-B |
| P15 | 2-[5-(ethylsulfonimidoyl)- 6-[7-methyl-3- (trifluoromethyl)imidazo[4, 5-c]pyridazin-6-yl]-3- pyridyl]oxy]-2-methyl- propanenitrile | 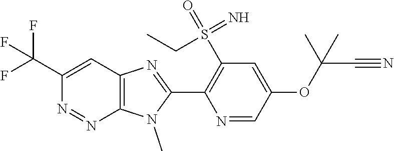 [SMILES: CCS(=N)(=O)c1cc(OC(C)(C)C#N)cnc1-c1nc2cc(C(F)(F)F)nnc2n1C] | P15-A | P15-B |
| P16 | 1-[5-(ethylsulfonimidoyl)-6- [3-methyl-6- (trifluoromethyl)imidazo[4, 5-b]pyridin-2-yl]-3- pyridyl] cyclopropanecarbonitrile | 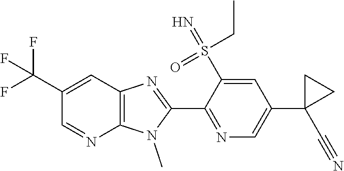 [SMILES: CCS(=N)(=O)c1cc(C2(C#N)CC2)cnc1-c1nc2cc(C(F)(F)F)cnc2n1C] | P16-A | P16-B |
| P17 | 2-[5-(ethylsulfonimidoyl)- 2-methyl-6-[3-methyl-6- (trifluoromethyl)imidazo[4, 5-c]pyridin-2-yl]-3- pyridyl]oxy]-2-methyl- propanenitrile | 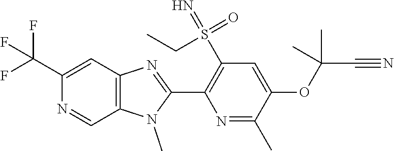 [SMILES: CCS(=N)(=O)c1cc(OC(C)(C)C#N)c(C)nc1-c1nc2cc(C(F)(F)F)ncc2n1C] | P17-A | P17-B |
| P18 | 1-[3-(ethylsulfonimidoyl)-4- [3-methyl-6- (trifluoromethyl)imidazo[4, 5-c]pyridin-2- yl]phenyl] cyclopropanecarbonitrile | 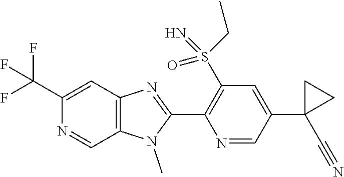 [SMILES: CCS(=N)(=O)c1cc(C2(C#N)CC2)cnc1-c1nc2cc(C(F)(F)F)ncc2n1C] | P18-A | P18-B |
| P19 | 2-[6-[5-ethyl-3-methyl-4- oxo-6- (trifluoromethyl)imidazo[4, 5-c]pyridin-2-yl]-5- (ethylsulfonimidoyl)-3- pyridyl]oxy]-2-methyl- propanenitrile | 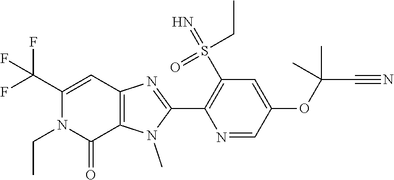 [SMILES: CCn1c(C(F)(F)F)cc2nc(-c3ncc(OC(C)(C)C#N)cc3S(=N)(=O)CC)n(C)c2c1=O] | P19-A | P19-B |

Compounds according to the invention may possess any number of benefits including, inter alia, advantageous levels of biological activity for protecting plants against insects or superior properties for use as agrochemical active ingredients (for example, greater biological activity, differential biological activity of enantiomer or enantiomerically enriched composition and racemate, differential biological activity of (R) enantiomer or (R) enantiomerically enriched composition and (S) enantiomer or (S) enantiomerically enriched composition, an advantageous spectrum of activity, an increased safety profile, improved physico-chemical properties, or increased biodegradability or environmental profile). In particular, it has been surprisingly found that certain compounds of formula (I) may show an advantageous safety profile with respect to non-target arthropods, in particular pollinators such as honey bees, solitary bees, and bumble bees. Most particularly, Apis mellifera.

In another aspect the present invention provides a composition comprising an insecticidally, acaricidally, nematicidally or molluscicidally effective amount of a compound of formula (I), or an agrochemically acceptable salt, stereoisomer, enantiomer, tautomer or N-oxide thereof, as defined in any of embodiments 1-34 (above) or any of the embodiments under compounds of formulae I-1, I-2, I-3, I-4, I-5, I-6, I-7, I-8, I-9, or I-10 and, optionally, an auxiliary or diluent.

In a further aspect the present invention provides a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in any of embodiments 1-34 (above) or any of the embodiments under compounds of formula I-1, I-2, I-3, I-4, I-5, I-6, I-7, I-8, I-9, or I-10 (above) or a composition as defined above.

In a yet further aspect, the present invention provides a method for the protection of plant propagation material from the attack by insects, acarines, nematodes or molluscs, which comprises treating the propagation material or the site, where the propagation material is planted, with a composition as defined above.

The process according to the invention for preparing compounds of formula I is carried out by methods known to those skilled in the art. Individual enantiomers can be prepared, for example, by either i) enantioselective transformations, ii) resolution of a racemic or partially enriched mixture by fractional crystallization with an enantiomerically enriched reagent, iii) chromatographic separation of the enantiomers using an enantiomerically enriched stationary phase.

Individual enantiomers can be obtained by chromatographic separation of a racemic mixture on a chiral stationary phase using preparative high-performance liquid chromatography (HPLC, in normal or reversed phase mode), or using preparative supercritical fluid chromatography (SFC).

Compounds of the formula I in form of a first or second eluting enantiomer, wherein Q, R₁, R₂, G₁, G₂, X₁, R₃, R₄, R₈, R₉, S* and A are as defined in formula I above,

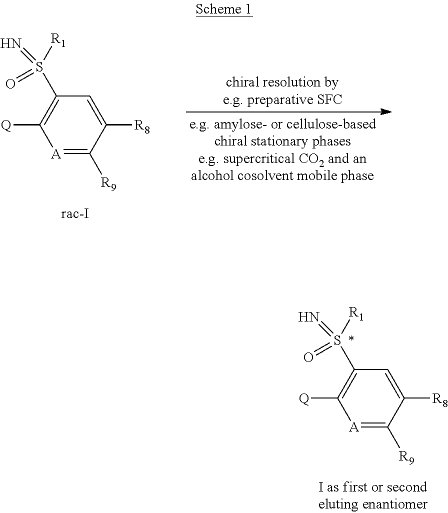

can be obtained (scheme 1) upon chiral resolution of the racemic mixture of compounds of the formula I (rac-1), wherein Q, R₁, R₂, G₁, G₂, X₁, R₃, R₄, R₈, R₉ and A are as defined in formula I above, for example by preparative SFC using immobilized amylose-based (such as CHIRALPAK® IA, CHIRALPAK® IG) or cellulose-based (such as CHIRALPAK® IC) chiral phases and using supercritical CO₂ and an alcohol cosolvent, such as preferably methanol, ethanol or isopropyl alcohol, as the mobile phase.

Racemic mixtures of compounds of the formula I (rac-1), wherein Q, R₁, R₂, G₁, G₂, X₁, R₃, R₄, R₈, R₉ and A are as defined in formula I above,

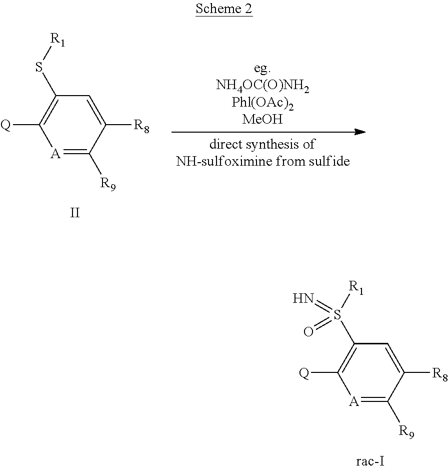

can be prepared (scheme 2) by reacting sulfide compounds of formula II, wherein Q, R₁, R₂, G₁, G₂, X₁, R₃, R₄, R₈, R₉ and A are as defined in formula I, with a suitable nitrogen source such as, for example, ammonia, ammonium carbamate or ammonium acetate (preferably ammonium carbamate), in the presence of hypervalent iodine reagents, such as diacetoxyiodobenzene, in solvents such as toluene, acetonitrile or methanol, at temperatures between 0 and 100° C., preferably around room temperature, in analogy to descriptions found, for example, in Chem. Commun. 53, 348-351; 2017 (and references cited therein).

Alternatively, racemic mixtures of compounds of the formula I (rac-1), wherein Q, R₁, R₂, G₁, G₂, X₁, R₃, R₄, R₈, R₉ and A are as defined in formula I above,

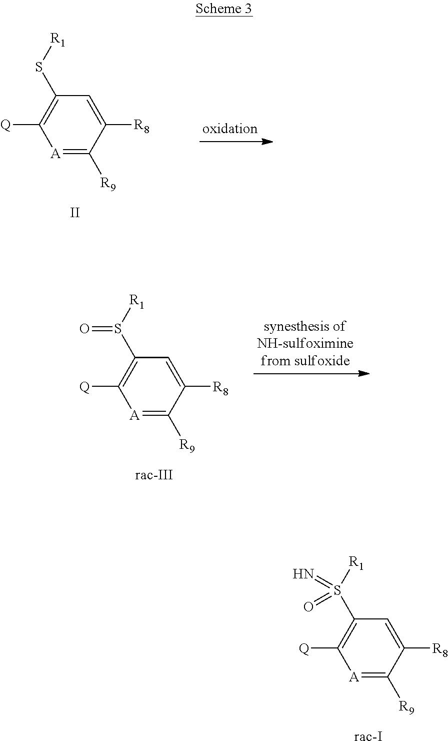

may be prepared (scheme 3) by reacting racemic sulfoxide compounds of formula rac-III, wherein Q, R₁, R₂, G₁, G₂, X₁, R₃, R₄, R₈, R₉ and A are as defined in formula I above, with a suitable nitrogen source, optionally in the presence of an oxidant, optionally in the presence of a metal catalyst and optionally in a solvent such as acetonitrile, dichloromethane or methanol. Examples of typical imination conditions include, O-dinitrophenylhydroxylamine/Rh₂(esp)₄, NH₂COONH₄/Phl(OAc)₂, NaN₃/H₂SO₄ or O-mesitylenesulfonyl-hydroxylamine (MSH). Examples of such transformations are described in Chemistry—A European Journal 2021, 27, 17293-17321 (and references therein), Chemical Communications 2014, 50, 9687-9689 and Angewandte Chemie, International Edition 2016, 55, 7203-7207 (and references cited therein).

Of particular interest are methods using a hydroxylamine derivative such as O-(4-nitrobenzoyl)-hydroxylamine triflic acid (also known as O-(4-nitrobenzoyl)-hydroxylammonium triflate or O-(4-nitrobenzoyl)-hydroxylammonium trifluoromethanesulfonate) and an iron catalyst, such as iron(II) sulfate (FeSO₄) or iron(II)phthalocyanine (Fe(II)phthalocyanine, FePc), in a solvent such as acetonitrile or dichloromethane, as described in Angewandte Chemie International Edition 2018, 57 324-327.

Compounds of formula rac-III, wherein Q, R₁, R₂, G₁, G₂, X₁, R₃, R₄, R₈, R₉ and A are as defined in formula I above, may be obtained by means of an oxidation reaction of the corresponding sulfide compounds of formula II, wherein Q, R₁, R₂, G₁, G₂, X₁, R₃, R₄, R₈, R₉ and A are as defined in formula I, involving reagents such as, for example, m-chloroperoxybenzoic acid (mCPBA), hydrogen peroxide, oxone, sodium periodate, sodium hypochlorite or tert-butyl hypochlorite amongst other oxidants. The oxidation reaction is generally conducted in the presence of a solvent. Examples of the solvent to be used in the reaction include aliphatic halogenated hydrocarbons such as dichloromethane and chloroform; alcohols such as methanol and ethanol; acetic acid; water; and mixtures thereof. The amount of the oxidant to be used in the reaction is preferably 1 to 1.2 moles, relative to 1 mole of the sulfide compounds II to produce the sulfoxide compounds rac-III.

Compounds of the formula rac-Ia may also serve for the preparation of compounds of formula rac-I as illustrated in scheme 4. Such compounds of the formula rac-Ia, wherein Q, R₁, R₂, G₁, G₂, X₁, R₃, R₄, R₈, R₉ and A are as defined in formula I above, and wherein R₁₀ is cyano or —C(O)R₂₅, in which R₂₅ is hydrogen, C₁-C₆alkyl, C₁-C₆haloalkyl, C₁-C₆alkoxy or C₁-C₆haloalkoxy,

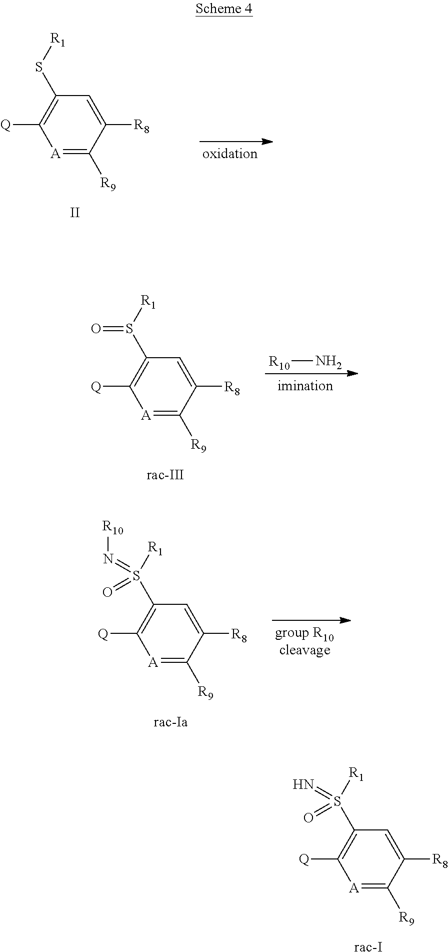

may be prepared (scheme 4) by submitting compounds of formula rac-III, wherein Q, R₁, R₂, G₁, G₂, X₁, R₃, R₄, R₈, R₉ and A are as defined in formula I above, to imination reaction conditions using a reagent R₁₀—NH₂ (R₁₀ as defined above), as described for example in H. Okamura, C. Bolm, Org. Lett. 2004, 6, 1305-1307; H. Okamura, C. Bolm, Chem. Lett. 2004, 33, 482-487; D. Leca, K. Song, M. Amatore, L. Fensterbank, E. Lacôte, M. Malacria, Chem. Eur. J. 2004, 10, 906-916; or M. Reggelin, C. Zur, Synthesis, 2000, 1-64. Typical imination reagents/conditions may include metal-catalyzed methods [see O. G. Mancheno, C. Bolm, Chem. Eur. J. 2007, 13, 6674-6681] such as R₁₀—N₃/FeCl₂, R₁₀—NH₂/Fe(acac)₃/Phl=0, Phl=N—R₁₀/Fe(OTf)₂, Phl=N—R₁₀/CuOTf, Phl=N—R₁₀/Cu(OTf)₂, Phl=N—R₁₀/CuPF₆, Phl(OAc)₂/R₁₀—NH₂/MgO/Rh₂(OAc)₄, R₀₁NHOMs/FeCl₂ or oxaziridines (e.g. 3-(4-cyano-phenyl)-oxaziridine-2-carboxylic acid tert-butyl ester).

Of particular interest are metal-free imination methods involving R₁₀—NH₂ and an oxidant, for example, Phl(OAc)₂/R₁₀—NH₂ as described in G. Y. Cho, C. Bolm, Tetrahedron Lett. 2005, 46, 8007-8008; or N-bromosuccinimide (NBS)/R₁₀—NH₂ and a base such as sodium or potassium ter-butoxide as described in C. Bolm et al., Synthesis 2010, No 17, 2922-2925. Oxidants such as N-iodosuccinimide (NIS) or iodine may be also used alternatively as described, for example, in O. G. Mancheno, C. Bolm, Org. Lett. 2007, 9, 3809-3811. An example of hypochlorite salts being used as oxidant, such as sodium hypochlorite NaOCl or calcium hypochlorite Ca(OCl)₂, was described in WO2008/1060.

A compound of the formula rac-Ia, wherein Q, R₁, R₂, G₁, G₂, X₁, R₃, R₄, R₈, R₉ and A are as defined in formula I above, and wherein R₁₀ is CN, may be transformed into a compound of the formula rac-la wherein R₁₀ is C(O)CF₃, by treatment with trifluoroacetic anhydride in a solvent such as dichloromethane as described, for example, in O. G. Mancheno, C. Bolm, Org. Lett. 2007, 9, 3809-3811.

A compound of the formula rac-Ia, wherein Q, R₁, R₂, G₁, G₂, X₁, R₃, R₄, R₈, R₉ and A are as defined in formula I above, and wherein R₁₀ is C(O)CF₃, may be transformed into a compound of the formula rac-Ia (group R₁₀ cleavage) by treatment with a base such as sodium or potassium carbonate in a polar protic solvent such as methanol or ethanol as described, for example, in H. Okamura, C. Bolm, Org. Lett. 2004, 6, 1305-1307.

Conversely, the order of the two oxidation/imination steps disclosed in scheme 4 to prepare compounds of the formula rac-Ia may be inverted as shown in scheme 5.

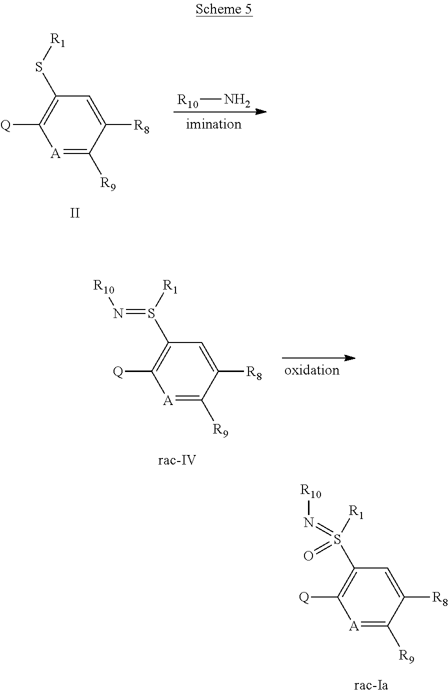

Oxidation of compounds of formula rac-IV, wherein Q, R₁, R₂, G₁, G₂, X₁, R₃, R₄, R₈, R₉ and A are as defined in formula I above, and in which R₁₀ is as defined in scheme 4, to provide the compounds of formula rac-Ia (substituents as defined in scheme 4), may be achieved under conditions already described above or may alternatively involve, for example, KMnO₄, NaMnO₄, mCPBA, NalO₄/RuO₂, NalO₄/RuCl₃, H₂O₂, or oxone. In particular, the use of ruthenium salts in combination with alkali metal periodates and alternatively the use of alkali metal permanganates was described in WO2008/097235 and WO2008/106006.

Compounds of formula rac-IV, wherein Q, R₁, R₂, G₁, G₂, X₁, R₃, R₄, R₈, R₉ and A are as defined in formula I above, and in which R₁₀ is cyano or —C(O)R₂₅, in which R₂₅ is hydrogen, C₁-C₆alkyl, C₁-C₆haloalkyl, C₁-C₆alkoxy or C₁-C₆haloalkoxy, may be prepared by submitting compounds of formula II, wherein Q, R₁, R₂, G₁, G₂, X₁, R₃, R₄, R₈, R₉ and A are as defined in formula I, to imination reaction conditions, as described above in scheme 4.

The subgroup of compounds of formula II, wherein R₈ is cyanoisopropoxy, more particularly 1-cyano-1-methyl-ethoxy, and wherein Q, R₁, R₂, G₁, G₂, X₁, R₃, R₄, R₉ and A are as defined in formula I above, may be defined as compounds of formula II-a (scheme 6). Such compounds II-a are either known or may be prepared by methods, or in analogy to methods, described for example in WO2020/084075, JP2019/081800, WO2018/206348 and WO2018/197315.

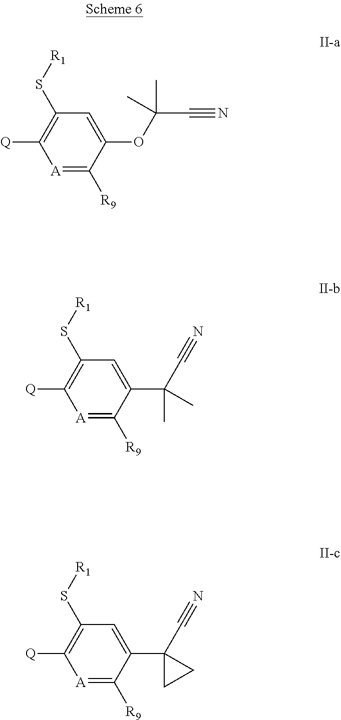

The subgroup of compounds of formula II, wherein R₈ is cyanoisopropyl, more particularly 1-cyano-1-methyl-ethyl, and wherein Q, R₁, R₂, G₁, G₂, X₁, R₃, R₄, R₉ and A are as defined in formula I above, may be defined as compounds of formula II-b (scheme 6). Such compounds II-b are either known or may be prepared by methods, or in analogy to methods, described for example in WO2019/053182, WO2018/153778 and WO2018/077565.

The subgroup of compounds of formula II, wherein R₈ is cyanocyclopropyl, more particularly 1-cyanocyclopropyl, and wherein Q, R₁, R₂, G₁, G₂, X₁, R₃, R₄, R₉ and A are as defined in formula I above, may be defined as compounds of formula II-c (scheme 6). Such compounds II-c are either known or may be prepared by methods, or in analogy to methods, described for example in WO2019/234158, WO2019/059244, WO2018/108726, WO2018/077565, WO2017/089190, WO2016/121997 and WO2016/071214.

Alternatively, individual enantiomers may be obtained by means of a stereoselective synthesis. Compounds of the formula I in form of an individual enantiomer, wherein Q, R₁, R₂, G₁, G₂, X₁, R₃, R₄, R₈, R₉ and A are as defined in formula I above, and wherein S* is a stereogenic sulfur atom in R- or S-configuration, in which said S* center is in either enantiomerically pure or in enantiomerically enriched form,

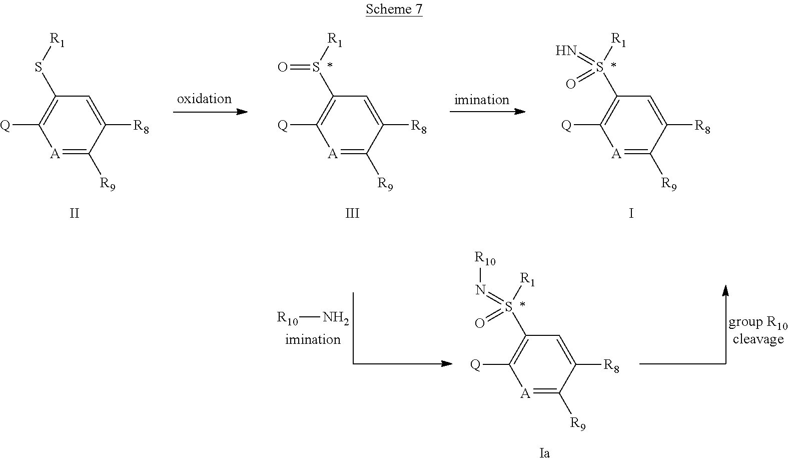

may be prepared (scheme 7) by adapting conditions already described in schemes 3 and 4. Compounds of formula III in form of an individual enantiomer, wherein Q, R₁, R₂, G₁, G₂, X₁, R₃, R₄, R₈, R₉ and A are as defined in formula I above, and wherein S* is a stereogenic sulfur atom in R- or S-configuration, in which said S* center is in either enantiomerically pure or in enantiomerically enriched form, may be obtained from compounds of the formula II (substituents as defined in schemes 3 and 4) by methods of stereoselective synthesis of chiral sulfinyl compounds, preferably in form of a catalytic enantioselective sulfoxide synthesis, by treatment with an oxidizing agent, for example H₂O₂ or tBuOOH, in the presence of a metal salt and a chiral ligand. Examples of appropriate metal salt and ligand combinations include Fe(acac)₃, V(O)(acac)₂ or Cu(acac)₂ with a Schiff base formed from salicaldehyde derivatives and chiral amino-alcohols, or salen complexes or Ti(OiPr)₄ in combination with a tartrate ester such as diisopropyl or diethyl tartrate. The reaction can be carried out in a solvent or mixture of solvents such as dichloromethane, toluene, chlorobenzene or methanol and optionally in the presence additives such as 4-methyoxybenzoic acid, benzoic acid, triethylamine, diisopropylethylamine or water. Examples of such reactions are described in Chemical Reviews 2020, 120, 4578-4611, Chemistry—A European Journal 2005, 11, 1086-1092, Angewandte Chemie (International Edition in English) 1996, 34, 2640-2642, Journal of Organic Chemistry 2012, 3288-3296 and Synlett 1996, 404-406. Alternatively, a chiral acid such as a binol derived chiral phosphoric acid can be uses as a catalyst in place of a metal complex and ligand as described in Journal of the American Chemical Society 2012, 134, 10765-10768.

Compounds of formula III in form of an individual enantiomer,

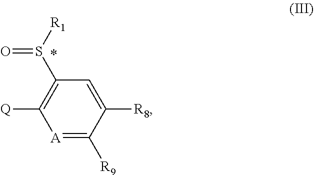

wherein
Q, R₁, R₂, G₁, G₂, X₁, R₃, R₄, R₈, R₉ and A are as defined in formula I; and
S* is a stereogenic sulfur atom in R- or S-configuration, in which said S* center is in either enantiomerically pure or in enantiomerically enriched form,
are novel, especially developed for the preparation of the compounds of formula I according to the invention and therefore represent a further object of the invention. The preferences and preferred embodiments of the substituents of the compounds of formula I are also valid for the compounds of formula III. Particularly preferred are those sulfinyl enantiomer compounds of formula III listed in Table P(SO).

Compounds of the formula I in form of an individual enantiomer, wherein Q, R₁, R₂, G₁, G₂, X₁, R₃, R₄, R₈, R₉ and A are as defined in formula I above, and wherein S* is a stereogenic sulfur atom in R- or S-configuration, in which said S* center is in either enantiomerically pure or in enantiomerically enriched form, may be obtained from compounds of the formula III in form of an individual enantiomer, wherein Q, R₁, R₂, G₁, G₂, X₁, R₃, R₄, R₈, R₉ and A are as defined in formula I above, and wherein S* is a stereogenic sulfur atom in R- or S-configuration, in which said S* center is in either enantiomerically pure or in enantiomerically enriched form, by means of an imination step via stereospecific nitrogen transfer, either in the direct mode (conditions analogous as in scheme 3), or by involving a reagent of the formula R₁₀—NH₂ (R₁₀ as defined above) and through the intermediacy of compounds of formula Ia, wherein Q, R₁, R₂, G₁, G₂, X₁, R₃, R₄, R₈, R₉ and A are as defined in formula I above, and wherein S* is a stereogenic sulfur atom in R- or S-configuration, in which said S* center is in either enantiomerically pure or in enantiomerically enriched form (conditions analogous as in scheme 4).

In another aspect, the present invention provides a process for the preparation of compound of formula (I)

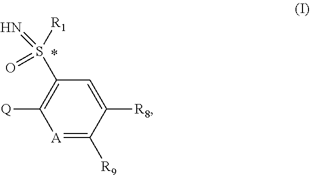

wherein Q, R₁, R₂, G₁, G₂, X₁, R₃, R₄, R₈, R₉ and A are as defined under formula (I), and
wherein S* is a stereogenic sulfur atom in R- or S-configuration, in which said S* center is in either enantiomerically pure or in enantiomerically enriched form;
which process comprises:
(A) stereoselectively oxidizing a sulfanyl compound of formula (II)

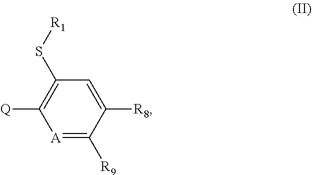

wherein Q, R₁, R₂, G₁, G₂, X₁, R₃, R₄, R₈, R₉ and A are as defined under formula (I),
in the presence of an oxidant, in the presence of a metal catalyst, in the presence of a chiral ligand, optionally in the presence of a suitable additive, in an appropriate solvent (or diluent);
to produce a sulfinyl compound of formula (III)

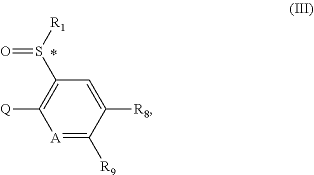

wherein Q, R₁, R₂, G₁, G₂, X₁, R₃, R₄, R₈, R₉ and A are as defined under formula (I), and
wherein S* is a stereogenic sulfur atom in R- or S-configuration, in which said S* center is in either enantiomerically pure or in enantiomerically enriched form; and
(B) reacting a sulfinyl compound of formula (III)

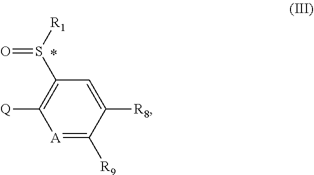

wherein Q, R₁, R₂, G₁, G₂, X₁, R₃, R₄, R₈, R₉ and A are as defined under formula (I), and
wherein S* is a stereogenic sulfur atom in R- or S-configuration, in which said S* center is in either enantiomerically pure or in enantiomerically enriched form;
with an imination reagent, in the presence of a catalyst, optionally in the presence of a suitable additive, in an appropriate solvent (or diluent);
to produce the sulfoximine compound of formula (I) in a stereospecific manner.

Related to a process for the preparation of compounds of formula (I) involving steps (A) and (B) above, the preferences and preferred embodiments of the substituents of the compounds of formula (I) described above are also valid for the compounds of formula (II) and (III).

In one particularly preferred embodiment, step (A) comprises oxidation of sulfanyl compounds of formula (II) listed in each step 1 of the Preparatory Examples P1 to P19.

In another particularly preferred embodiment, step (B) comprises reacting sulfinyl enantiomer compounds of formula III listed in Table P(SO) with an imination reagent.

Related to a process for the preparation of compounds of formula (III), step (A) above, examples of suitable and preferred oxidants, suitable and preferred metal catalysts, suitable and preferred chiral ligands, suitable and preferred additives, as well as examples of suitable and preferred reaction conditions (such as solvent (or diluent) and temperature), are given below.

In one embodiment, step (A) comprises
(A-1) oxidizing a sulfanyl compound of formula (II), in the presence of an oxidant, in the presence of a metal catalyst, in the presence of a chiral ligand, in an appropriate solvent (or diluent).

In another embodiment, step (A) comprises
(A-2) oxidizing a sulfanyl compound of formula (II), in the presence of an oxidant, in the presence of a metal catalyst, in the presence of a chiral ligand, in the presence of a suitable additive, in an appropriate solvent (or diluent).

Example of suitable and preferred oxidants for steps (A-1) and (A-2) are inorganic peroxides, such as hydrogen peroxide or organic peroxides, such as tert-butyl hydroperoxide. Preferably the oxidant is hydrogen peroxide or tert-butyl hydroperoxide, even more preferably hydrogen peroxide. The ratio of the oxidant used, compared to the sulfanyl compound of formula (II), is in the range from 8:1 to 0.8:1, preferably between 5:1 and 1:1, more preferably between 3:1 and 1:1.

Example of suitable and preferred metal catalysts for steps (A-1) and (A-2) are iron(III) acetylacetonate (Fe(acac)₃) or vanadylacetylacetonat (vanadium(IV)-oxyacetylacetonat, VO(acac)₂). Preferably the metal catalyst is iron(III) acetylacetonate. The amount of the metal salt used, compared to the sulfanyl compound of formula (II), is in the range from 0.01 to 10 mol %, preferably from 0.1 to 8 mol %, most preferably from 1 to 6 mol %.

Example of suitable and preferred chiral ligands for steps (A-1) and (A-2) are derived from N,N′-bis(salicylidene)ethylenediamine (salen ligand) or chosen from Schiff bases formed from salicaldehyde derivatives and chiral amino-alcohols. Preferably the chiral ligand is a Schiff base formed from salicaldehyde derivatives and chiral amino-alcohols represented by a compound of formula (R)-Xa or (S)-Xa,

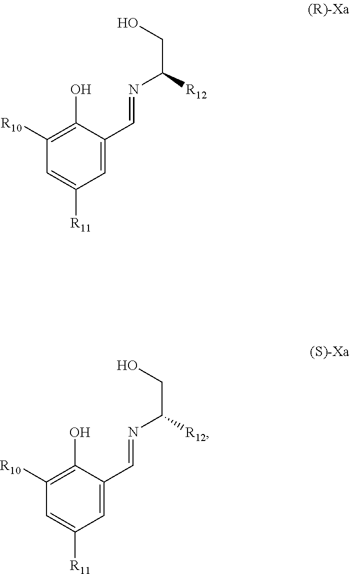

wherein R₁₀ and R₁₁ are, independently of each other, chosen from C₁-C₄alkyl and halogen, and R₁₂ is tert-butyl, isopropyl, optionally substituted phenyl or optionally substituted benzyl. More preferably the chiral ligand is a compound of formula (R)-Xb or (S)-Xb,

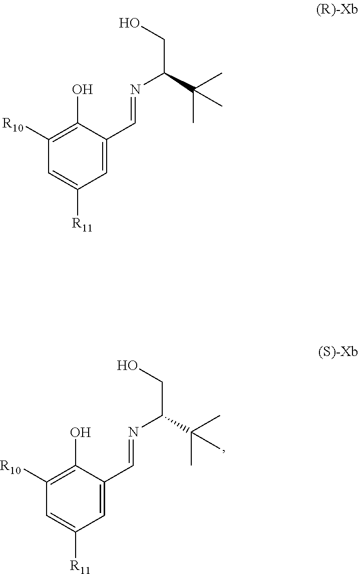

wherein R₁₀ and R₁₁ are, independently of each other, chosen from t-butyl, chloro, bromo and iodo; even more preferably chosen from chloro, bromo and iodo. Particularly preferred ia a chiral ligand compound of formula (R)-Xb or (S)-Xb, wherein R₁₀ equals R₁₁ and is chosen from chloro, bromo and iodo. The ratio of the chiral ligand (preferably a compound of formula (R)-Xb or (S)-Xb) used, compared to the metal catalyst (preferably iron(III) acetylacetonate), is in the range from 10:1 to 0.5:1, preferably 3:1 to 1:1, more preferably around 2:1.

Example of suitable and preferred additives for step (A-2) are carbocyclic acids. Preferably the additive is a benzoic acid, optionally mono-, di- or tri-substituted by methyl, ethyl, isopropyl, methoxy or dimethylamino, optionally in form of a lithium, sodium or potassium salt. More preferably the additive is a methoxybenzoic acid (optionally in form of a lithium, sodium or potassium salt), even more preferably 4-methoxybenzoic acid. The amount of the additive used, compared to the sulfanyl compound of formula (II), is in the range from 0.01 to 10 mol %, preferably from 0.1 to 8 mol %, most preferably from 1 to 5 mol %.

In one embodiment related to the process according to the invention of making compounds of formula (III), step (A), examples of appropriate solvents (or diluents) are aliphatic halogenated hydrocarbons such as dichloromethane, 1,2-dichloroethane or chloroform, or aromatic hydrocarbons, halohydrocarbons or alkoxyhydrocarbons such as toluene, xylene, chlorobenzene, methoxybenzene or benzotrifluoride, or mixtures thereof. Preferably the solvent (or diluent) is toluene or chlorobenzene, even more preferably toluene.

In one embodiment related to the process according to the invention of making compounds of formula (III), step (A), the reaction is advantageously carried out in a temperature range from approximately −20° C. to approximately 50° C., preferably from approximately −5° C. to approximately 30° C. In a preferred embodiment, the reaction is carried out in the range between 0° C. and 25° C.

Related to a process for the preparation of compounds of formula (I), step (B) above, examples of suitable and preferred imination reagents, examples of suitable and preferred catalysts, suitable and preferred additives, as well as examples of suitable and preferred reaction conditions (such as solvent (or diluent) and temperature), are given below.

In one embodiment, step (B) comprises
(B-1) reacting a sulfinyl compound of formula (III) with an imination reagent, in the presence of a catalyst, in an appropriate solvent (or diluent).

Example of suitable and preferred imination reagents for step (B-1) are O-mesitylenesulfonyl-hydroxylamine (MSH) or hydroxylamine derivatives. Preferably the imination reagent is a hydroxylamine derivative, more preferably an O-acylated hydroxylamine salt represented by a compound of formula (XX),

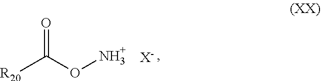

wherein R₂O is tert-butyl, or phenyl mono- or di-substituted by nitro, and X⁻ is a sulfonate or hydrogenosulfate group. More preferably the imination reagent is a compound of formula (XX), wherein R₂O is 4-nitrophenyl or 2,4-dinitrophenyl, and X—is a sulfonate group. Even more preferably the imination reagent compound of formula (XX) is chosen from O-(4-nitrobenzoyl)-hydroxylammonium trifluoromethanesulfonate and O-(4-nitrobenzoyl)-hydroxylammonium methanesulfonate. Particularly preferred as imination reagent compound of formula (XX) is O-(4-nitrobenzoyl)-hydroxylammonium trifluoromethanesulfonate. The ratio of the imination reagent used, compared to the sulfinyl compound of formula (III), is in the range from 8:1 to 0.8:1, preferably between 5:1 and 1:1, more preferably between 3:1 and 1:1.

Example of suitable and preferred catalysts for steps (B-1) are iron(II) sulfate (FeSO4), iron(II) acetate (Fe(OAc)₂) or iron(II) acetylacetonate (Fe(acac)₂) each in combination with either 2,2′-bipyridine or 1,10-phenanthroline, or iron(II)phthalocyanine (Fe(II)phthalocyanine, FePc). Preferably the metal catalyst is iron(II)phthalocyanine. The amount of the catalyst used, compared to the sulfinyl compound of formula (III), is in the range from 0.01 to 10 mol %, preferably from 0.1 to 8 mol %, most preferably from 1 to 5 mol %.

In one embodiment related to the process according to the invention of making compounds of formula (I), step (B), examples of appropriate solvents (or diluents) are acetonitrile, methanol, ethanol, isopropanol, 2,2,2-trifluoroethanol (TFE), hexafluoroisopropanol (HFIP), dichloromethane (DCM), toluene, ethyl acetate, acetic acid, water, or mixtures thereof. Preferably the solvent (or diluent) is acetonitrile, acetic acid or dichloromethane, even more preferably dichloromethane.

In one embodiment related to the process according to the invention of making compounds of formula (I), step (B), the reaction is advantageously carried out in a temperature range from approximately −20° C. to approximately 50° C., preferably from approximately −5° C. to approximately 30° C. In a preferred embodiment, the reaction is carried out in the range between 10° C. and 25° C.

Products obtained by the process according to the invention wherein S* is a stereogenic sulfur atom in R- or S-configuration, in which said S* center is in enantiomerically enriched form have an enantiomer ratio (R:S or S:R, as the case may be) of 50.5:49.5 to 99.5:0.5, preferably of 75:25 to 99:1, more preferably of 85:15 to 98:2.

In one embodiment, the sulfinyl compounds of formula (III) obtained by step (A) of the process according to the invention wherein S* is a stereogenic sulfur atom in R- or S-configuration, in which said S* center is in enantiomerically enriched form have an enantiomer ratio (R:S or S:R, as the case may be) of 50.5:49.5 to 99.5:0.5, preferably of 75:25 to 99:1, more preferably of 85:15 to 98:2.

In another embodiment, the sulfoximine compounds of formula (I) obtained by step (B) of the process according to the invention wherein S* is a stereogenic sulfur atom in R- or S-configuration, in which said S* center is in enantiomerically enriched form have an enantiomer ratio (R:S or S:R, as the case may be) of 50.5:49.5 to 99.5:0.5, preferably of 75:25 to 99:1, more preferably of 85:15 to 98:2.

In a further embodiment, the enantiomer ratio (R:S or S:R, as the case may be) of such sulfinyl compounds of formula (III) obtained by step (A) and the enantiomer ratio (R:S or S:R, as the case may be) of such sulfoximine compounds of formula (I) obtained by step (B) are substantially the same. In one embodiment, the enantiomer ratio of the sulfinyl compounds of formula (III) obtained by step (A) and the enantiomer ratio of sulfoximine compounds of formula (I) obtained by step (B) are within (±) plus or minus one percent of each other; preferably are within (±) plus or minus 0.5 percent of each other; more preferably are within (±) plus or minus 0.1 percent of each other.

Optionally, the enantiomeric purity of such products, the sulfinyl compounds of formula (III) and/or the sulfoximine compounds of formula (I) can be increased by a crystallization process known to those skilled in the art, preferably via crystallization from an organic solvent or a mixture of an organic solvent and water.

Methods for determining the enantiomeric excess are known to those skilled in the art and include for example the use of HPLC on chiral stationary phases and NMR with chiral shift reagents.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an)other substituent(s) according to the invention, and by post modification of compounds of with reactions such as oxidation, alkylation, reduction, acylation and other methods known by those skilled in the art.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl celulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

Several ways of absolute configuration determination of chiral compounds are known and include many spectroscopic and diffraction methods. Amongst them for example, derivatization with a chiral reagent and analysis by chromatographic techniques, NMR with chiral shift reagents, optical rotatory dispersion, circular dichroism, chemical correlation, and X-ray crystallography, in particular single-crystal X-ray diffraction (XRD).

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 00/15615.

Compounds wherein R₂ is C₁-C₄haloalkylsulfinyl or C₁-C₄haloalkylsulfonyl may be prepared from the corresponding compounds wherein R₂ is C₁-C₄haloalkylsulfanyl with suitable oxidation methods described, for example, in WO 19/008115.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds of formula I according to the following Tables X, A-1 to A-20 and B-1 to B-20 below can be prepared according to the methods described above. The examples which follow are intended to illustrate the invention and show preferred compounds of formula I.

The Tables A-1 to A-20 below illustrate specific compounds of the invention wherein the stereogenic sulfur atom is in the R-configuration.

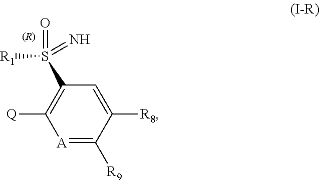

Table A-1 provides 12 compounds A-1.001 to A-1.012 of formula (I-R) wherein R₁ is ethyl, and A, R₈ and R₉ are as defined in Table X, and Q is taken from the group of formula Q₁ as

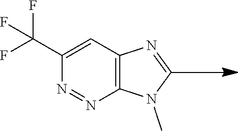

TABLE X
Substituent definitions of A, R₈ and R₉

| Index | A | R₉ | R₈ |
|---|---|---|---|
| 1 | N | H | —OC(CH₃)₂CN |
| 2 | N | CH₃ | |
| 3 | CH | H | |
| 4 | CH | CH₃ | |
| 5 6 7 8 | N N CH CH | H CH₃ H CH₃ | 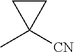 |
| 9 | N | H | —C(CH₃)₂CN |
| 10 | N | CH₃ | |
| 11 | CH | H | |
| 12 | CH | CH₃ | |

For example, compound A-1.004 has the following structure:

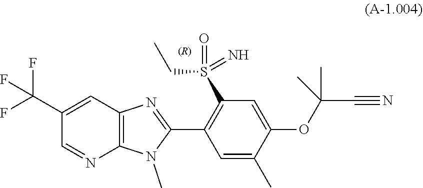

wherein (R) denotes the R-configuration at the stereogenic sulfur center.

Table A-2 provides 12 compounds A-2.001 to A-2.012 of formula (I-R) wherein R₁ is ethyl, and A, R₈ and R₉ are as defined in Table X, and Q is taken from the group of formula Q₁ as

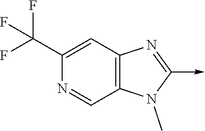

Table A-3 provides 12 compounds A-3.001 to A-3.012 of formula (I-R) wherein R₁ is ethyl, and A, R₈ and R₉ are as defined in Table X, and Q is taken from the group of formula Q₁ as

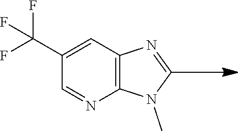

Table A-4 provides 12 compounds A-4.001 to A-4.012 of formula (I-R) wherein R₁ is ethyl, and A, R₈ and R₉ are as defined in Table X, and Q is taken from the group of formula Q₂ as

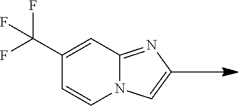 [SMILES: FC(F)(F)c1ccn2ccnc2c1]

Table A-5 provides 12 compounds A-5.001 to A-5.012 of formula (I-R) wherein R₁ is ethyl, and A, R₈ and R₉ are as defined in Table X, and Q is taken from the group of formula Q₂ as

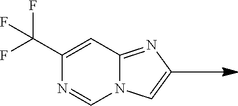 [SMILES: FC(F)(F)c1cc2nccn2cn1]

Table A-6 provides 12 compounds A-6.001 to A-6.012 of formula (I-R) wherein R₁ is ethyl, and A, R₈ and R₉ are as defined in Table X, and Q is taken from the group of formula Q₃ as

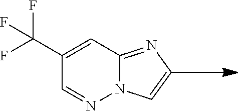 [SMILES: FC(F)(F)c1cnn2ccnc2c1]

Table A-7 provides 12 compounds A-7.001 to A-7.012 of formula (I-R) wherein R₁ is ethyl, and A, R₈ and R₉ are as defined in Table X, and Q is taken from the group of formula Q₁ as

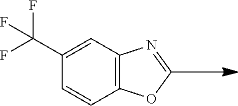 [SMILES: FC(F)(F)c1ccc2ocnc2c1]

Table A-8 provides 12 compounds A-8.001 to A-8.012 of formula (I-R) wherein R₁ is ethyl, and A, R₈ and R₉ are as defined in Table X, and Q is taken from the group of formula Q₁ as

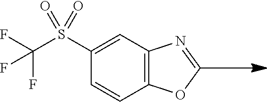 [SMILES: O=S(=O)(c1ccc2ocnc2c1)C(F)(F)F]

Table A-9 provides 12 compounds A-9.001 to A-9.012 of formula (I-R) wherein R₁ is ethyl, and A, R₈ and R₉ are as defined in Table X, and Q is taken from the group of formula Q₄ as

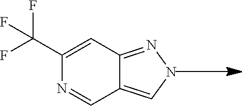 [SMILES: FC(F)(F)c1cc2n[nH]cc2cn1]

Table A-10 provides 12 compounds A-10.001 to A-10.012 of formula (I-R) wherein R₁ is ethyl, and A, R₈ and R₉ are as defined in Table X, and Q is taken from the group of formula Q₄ as

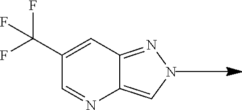 [SMILES: FC(F)(F)c1cnc2c[nH]nc2c1]

Table A-11 provides 12 compounds A-11.001 to A-11.012 of formula (I-R) wherein R₁ is ethyl, and A, R₈ and R₉ are as defined in Table X, and Q is taken from the group of formula Q₅ as

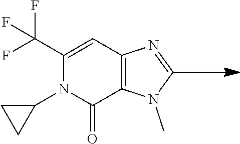 [SMILES: Cn1cnc2cc(C(F)(F)F)n(C3CC3)c(=O)c21]

Table A-12 provides 12 compounds A-12.001 to A-12.012 of formula (I-R) wherein R₁ is ethyl, and A, R₈ and R₉ are as defined in Table X, and Q is taken from the group of formula Q₅ as

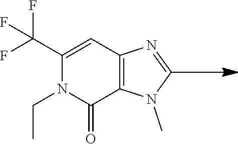 [SMILES: CCn1c(C(F)(F)F)cc2ncn(C)c2c1=O]

Table A-13 provides 12 compounds A-13.001 to A-13.012 of formula (I-R) wherein R₁ is ethyl, and A, R₈ and R₉ are as defined in Table X, and Q is taken from the group of formula Q₅ as

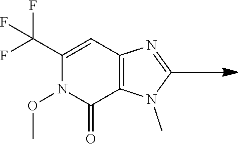 [SMILES: COn1c(C(F)(F)F)cc2ncn(C)c2c1=O]

Table A-14 provides 12 compounds A-14.001 to A-14.012 of formula (I-R) wherein R₁ is ethyl, and A, R₈ and R₉ are as defined in Table X, and Q is taken from the group of formula Q₂ as

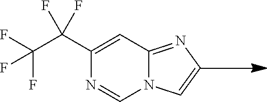 [SMILES: FC(F)(F)C(F)(F)c1cc2nccn2cn1]

Table A-15 provides 12 compounds A-15.001 to A-15.012 of formula (I-R) wherein R₁ is ethyl, and A, R₈ and R₉ are as defined in Table X, and Q is taken from the group of formula Q₂ as

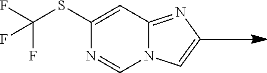 [SMILES: FC(F)(F)Sc1cc2nccn2cn1]

Table A-16 provides 12 compounds A-16.001 to A-16.012 of formula (I-R) wherein R₁ is ethyl, and A, R₈ and R₉ are as defined in Table X, and Q is taken from the group of formula Q₂ as

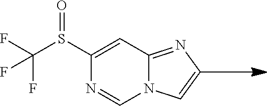 [SMILES: O=S(c1cc2nccn2cn1)C(F)(F)F]

Table A-17 provides 12 compounds A-17.001 to A-17.012 of formula (I-R) wherein R₁ is ethyl, and A, R₈ and R₉ are as defined in Table X, and Q is taken from the group of formula Q₂ as

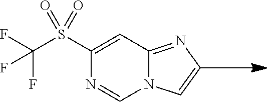 [SMILES: O=S(=O)(c1cc2nccn2cn1)C(F)(F)F]

Table A-18 provides 12 compounds A-18.001 to A-18.012 of formula (I-R) wherein R₁ is ethyl, and A, R₈ and R₉ are as defined in Table X, and Q is taken from the group of formula Q₁ as

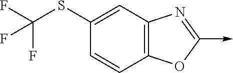

Table A-19 provides 12 compounds A-19.001 to A-19.012 of formula (I-R) wherein R₁ is ethyl, and A, R₈ and R₉ are as defined in Table X, and Q is taken from the group of formula Q₁ as

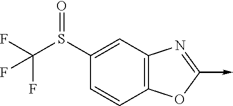

Table A-20 provides 12 compounds A-20.001 to A-20.012 of formula (I-R) wherein R₁ is ethyl, and A, R₈ and R₉ are as defined in Table X, and Q is taken from the group of formula Q₅ as

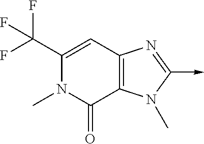

The Tables B-1 to B-20 below further illustrate specific compounds of the invention wherein the stereogenic sulfur atom is in the S-configuration.

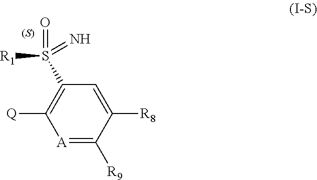

Table B-1 provides 12 compounds B-1.001 to B-1.012 of formula (I-S) wherein R₁ is ethyl, and A, R₈ and R₉ are as defined in Table X, and Q is taken from the group of formula Q₁ as

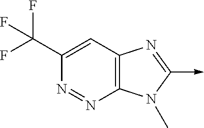

For example, compound B-17.005 has the following structure:

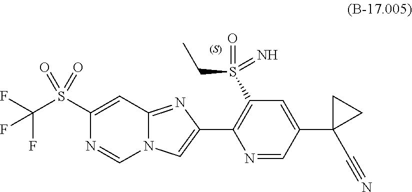

wherein (S) denotes the S-configuration at the stereogenic sulfur center.

Table B-2 provides 12 compounds B-2.001 to B-2.012 of formula (I-S) wherein R₁ is ethyl, and A, R₈ and R₉ are as defined in Table X, and Q is taken from the group of formula Q₁ as

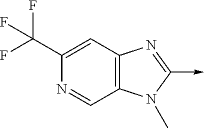

Table B-3 provides 12 compounds B-3.001 to B-3.012 of formula (I-S) wherein R₁ is ethyl, and A, R₈ and R₉ are as defined in Table X, and Q is taken from the group of formula Q₁ as

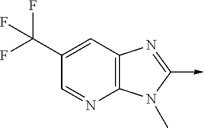

Table B-4 provides 12 compounds B-4.001 to B-4.012 of formula (I-S) wherein R₁ is ethyl, and A, R₈ and R₉ are as defined in Table X, and Q is taken from the group of formula Q₂ as

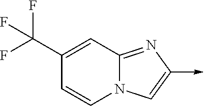

Table B-5 provides 12 compounds B-5.001 to B-5.012 of formula (I-S) wherein R₁ is ethyl, and A, R₈ and R₉ are as defined in Table X, and Q is taken from the group of formula Q₂ as

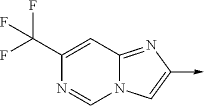

Table B-6 provides 12 compounds B-6.001 to B-6.012 of formula (I-S) wherein R₁ is ethyl, and A, R₈ and R₉ are as defined in Table X, and Q is taken from the group of formula Q₃ as

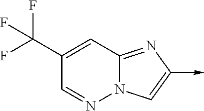

Table B-7 provides 12 compounds B-7.001 to B-7.012 of formula (I-S) wherein R₁ is ethyl, and A, R₈ and R₉ are as defined in Table X, and Q is taken from the group of formula Q₁ as

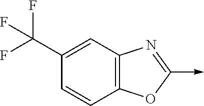

Table B-8 provides 12 compounds B-8.001 to B-8.012 of formula (I-S) wherein R₁ is ethyl, and A, R₈ and R₉ are as defined in Table X, and Q is taken from the group of formula Q₁ as

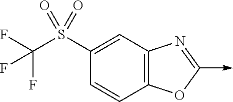

Table B-9 provides 12 compounds B-9.001 to B-9.012 of formula (I-S) wherein R₁ is ethyl, and A, R₈ and R₉ are as defined in Table X, and Q is taken from the group of formula Q₄ as

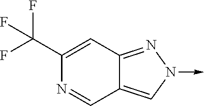

Table B-10 provides 12 compounds B-10.001 to B-10.012 of formula (I-S) wherein R₁ is ethyl, and A, R₈ and R₉ are as defined in Table X, and Q is taken from the group of formula Q₄ as

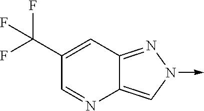

Table B-11 provides 12 compounds B-11.001 to B-11.012 of formula (I-S) wherein R₁ is ethyl, and A, R₈ and R₉ are as defined in Table X, and Q is taken from the group of formula Q₅ as

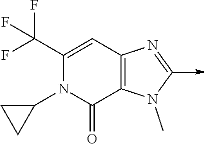

Table B-12 provides 12 compounds B-12.001 to B-12.012 of formula (I-S) wherein R₁ is ethyl, and A, R₈ and R₉ are as defined in Table X, and Q is taken from the group of formula Q₅ as

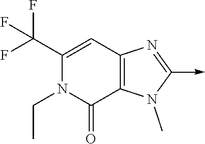

Table B-13 provides 12 compounds B-13.001 to B-13.012 of formula (I-S) wherein R₁ is ethyl, and A, R₈ and R₉ are as defined in Table X, and Q is taken from the group of formula Q₅ as

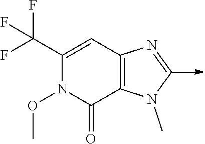

Table B-14 provides 12 compounds B-14.001 to B-14.012 of formula (I-S) wherein R₁ is ethyl, and A, R₈ and R₉ are as defined in Table X, and Q is taken from the group of formula Q₂ as

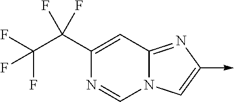

Table B-15 provides 12 compounds B-15.001 to B-15.012 of formula (I-S) wherein R₁ is ethyl, and A, R₈ and R₉ are as defined in Table X, and Q is taken from the group of formula Q₂ as

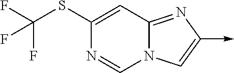

Table B-16 provides 12 compounds B-16.001 to B-16.012 of formula (I-S) wherein R₁ is ethyl, and A, R₈ and R₉ are as defined in Table X, and Q is taken from the group of formula Q₂ as

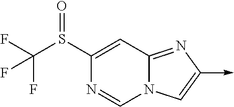

Table B-17 provides 12 compounds B-17.001 to B-17.012 of formula (I-S) wherein R₁ is ethyl, and A, R₈ and R₉ are as defined in Table X, and Q is taken from the group of formula Q₂ as

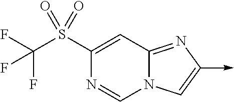

Table B-18 provides 12 compounds B-18.001 to B-18.012 of formula (I-S) wherein R₁ is ethyl, and A, R₈ and R₉ are as defined in Table X, and Q is taken from the group of formula Q₁ as

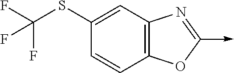

Table B-19 provides 12 compounds B-19.001 to B-19.012 of formula (I-S) wherein R₁ is ethyl, and A, R₈ and R₉ are as defined in Table X, and Q is taken from the group of formula Q₁ as

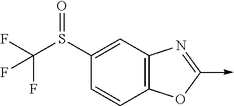

Table B-20 provides 12 compounds B-20.001 to B-20.012 of formula (I-S) wherein R₁ is ethyl, and A, R₈ and R₉ are as defined in Table X, and Q is taken from the group of formula Q₅ as

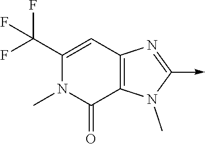

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina, nematodes or molluscs. The insecticidal, nematicidal, molluscicidal or acaricidal activity of the active ingredients according to the invention can manifest itself directly, i.e. in mortality or destruction of the pests, which takes place either immediately or only after some time has elapsed, for example during ecdysis, or indirectly, for example in a reduced oviposition and/or hatching rate, anti-feedant effect, and/or growth inhibition.

Compounds of formula (I) according to the invention may possess any number of benefits including, inter alia, advantageous levels of biological activity for protecting plants against insects or superior properties for use as agrochemical active ingredients (for example, greater biological activity, differential biological activity of enantiomer or enantiomerically enriched composition and racemate, differential biological activity of (R) enantiomer or (R) enantiomerically enriched composition and (S) enantiomer or (S) enantiomerically enriched composition, an advantageous spectrum of activity, an increased safety profile, improved physico-chemical properties, or increased biodegradability or environmental profile). In particular, it has been surprisingly found that certain compounds of formula (I) show an advantageous safety profile with respect to non-target organisms, for example, non-target arthropods, in particular pollinators such as honey bees, solitary bees, and bumble bees. Most particularly, Apis mellifera.

In this regard, certain compounds of formula (I) of the invention can be distinguished from known compounds by virtue of greater efficacy at low application rates, which can be verified by the person skilled in the art using experimental procedures similar to or adapted from those outlined in the biological examples, using lower application rates if necessary, for example 50 ppm, 12.5 ppm, 6 ppm, 3 ppm, 1.5 ppm, 0.8 ppm or 0.2 ppm.

Further it has surprisingly found that that compounds of formula (I) of the invention show advantageous physico-chemical properties for application in crop protection, in particular reduced melting point, reduced lipophilicity and increased water solubility. Such properties have been found to be advantageous for plant uptake and systemic distribution, see for example A. Buchholz, S. Trapp, Pest Manag Sci 2016; 72: 929-939) in order to control certain pest species named below.

Putative metabolites of the compounds of the formula I which may be formed in the practice of the invention in conjunction with one or more of the methods, pests, crops and/or targets described below include the amide compounds of formula I-M1, I-M2, I-M3 and the acid compounds of formula I-M4, I-M5, I-M6, each corresponding to a parent nitrile compound of formula I:

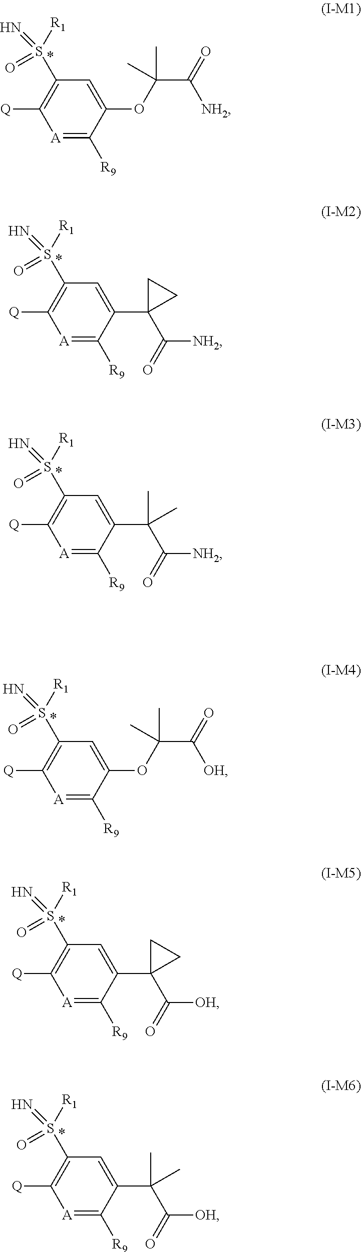

wherein Q, R₁, R₂, R₃, R₄, R₉, X₁, G₁, G₂, S* and A are as defined under formula I above, or an agrochemically acceptable salt, stereoisomer, enantiomer, tautomer or N-oxide thereof. Among the specific putative metabolites there may be mentioned: (1) an amide compound of formula I-M1, I-M2, or I-M3 that corresponds to a parent nitrile selected from the group consisting of the compounds described in Tables A-1 through A-20, Tables B-1 through B-20, Table Y, Table Z and Table P(E); and (2) an acid compound of formula I-M4, I-M5, or I-M6 that corresponds to a parent nitrile selected from the group consisting of the compounds described in Tables A-1 through A-20, Tables B-1 through B-20, Table Y, Table Z and Table P(E).

Examples of the abovementioned animal pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., sChrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euschistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

The active ingredients according to the invention can be used for controlling, i.e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp orjute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1 Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF—YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirexjuvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether | 4% |
| (35 mol of ethylene oxide) | |
| Cyclohexanone | 30% |
| xylene mixture | 50% |

Emulsions of any require dilution, which can be use in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol | 6% |
| ether (15 mol of ethylene oxide) | |
| Sodium lignosulfonate | 10% |
| carboxymethylcellulose | 1% |
| silicone oil (in the form of a | 1% |
| 75% emulsion in water) | |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form | 0.5% |
| of a 20% solution in water) | |
| monoazo-pigment calcium salt | 5% |
| Silicone oil (in the form of a 75% | 0.2% |
| emulsion in water) | |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### Preparatory Examples

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻. Specific rotation [α]: samples were measured on an Autopol IV polarimeter from Rudolph Research Analytical.

### LCMS Methods:

### Method 1:

Spectra were recorded on a Mass Spectrometer from Waters (ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: 0 min 0% B, 100% A; 1.2-1.5 min 100% B; Flow (ml/min) 0.85.

### Method 2:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85.

### Method 3:

Spectra were recorded on a Mass Spectrometer from Agilent Technologies (6410 Triple Quadruple Mass Spectrometer) equipped with an electrospray source (Polarity: Positive and Negative Polarity Switch, Capillary: 4.00 kV, Fragmentor: 100.00 V, Gas Temperature: 350° C., Gas Flow: 11 L/min, Nebulizer Gas: 45 psi, Mass range: 110-1000 Da, DAD Wavelength range: 210-400 nm). Column: KINETEX EVO C18, length 50 mm, diameter 4.6 mm, particle size 2.6 μm. Column oven temperature 40° C. Solvent gradient: A=Water with 0.1% formic acid: Acetonitrile (95:5 v/v). B=Acetonitrile with 0.1% formic acid. Gradient=0 min 90% A, 10% B; 0.9-1.8 min 0% A, 100% B, 2.2-2.5 min 90% A, 10% B. Flow rate 1.8 mL/min.

### Method 4:

Spectra were recorded on a Mass Spectrometer from Waters (Acquity SDS Mass Spectrometer) equipped with an electrospray source (Polarity: Positive and Negative Polarity Switch, Capillary: 3.00 kV, Cone Voltage: 41.00 V, Source temperature: 150° C., Desolvation Gas Flow: 1000 L/Hr, Desolvation temperature: 500° C., Gas Flow® Cone: 50 L/hr, Mass range: 110-800 Da, PDA wavelength range: 210-400 nm. Column: Acquity UPLC HSS T3 C18, length 30 mm, diameter 2.1 mm, particle size 1.8 μm. Column oven temperature 40° C. Solvent gradient: A=Water with 0.1% formic acid: Acetonitrile (95:5 v/v). B=Acetonitrile with 0.05% formic acid. Gradient=0 min 90% A, 10% B; 0.2 min 50% A, 50% B; 0.7-1.3 min 0% A, 100% B; 1.4-1.6 min 90% A, 10% B. Flow rate 0.8 mL/min.

### Method 5:

Spectra were recorded on a Mass Spectrometer from Waters (Acquity SDS Mass Spectrometer) equipped with an electrospray source (Polarity: Positive and Negative Polarity Switch, Capillary: 3.00 kV, Cone Voltage: 41.00 V, Source temperature: 150° C., Desolvation Gas Flow: 1000 L/Hr., Desolvation temperature: 500° C., Gas Flow @Cone: 50 L/hr., Mass range: 110-800 Da, PDA wavelength range: 210-400 nm. Column: Acquity UPLC HSS T3 C18, length 30 mm, diameter 2.1 mm, particle size 1.8 μm. Column oven temperature 40° C. Solvent gradient: A=Water with 0.1% formic acid: Acetonitrile (95:5 v/v). B=Acetonitrile with 0.05% formic acid. Gradient=0 min 90% A, 10% B; 0.2 min 50% A, 50% B; 0.7-1.3 min 0% A, 100% B; 1.4-1.6 min 90% A, 10% B. Flow rate 0.6 mL/min.

### Method 6:

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 2.7 min; Flow (ml/min) 0.85.

### PREPARATION OF EXAMPLES OF COMPOUNDS OF FORMULA (I)

### Example P1: Preparation of racemic 2-[[6-[5-cyclopropyl-3-methyl-4-oxo-6-(trifluoromethyl) imidazo[4,5-c]pyridin-2-yl]-5-(ethylsulfonimidoyl)-3-pyridyl]oxy]-2-methyl-propanenitrile (Compound P1) and its Individual Enantiomers (Compounds P1-A and P1-B)

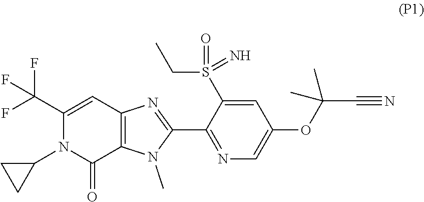 [SMILES: CCS(=N)(=O)c1cc(OC(C)(C)C#N)cnc1-c1nc2cc(C(F)(F)F)n(C3CC3)c(=O)c2n1C]

### Step 1: Preparation of 2-[[6-[5-cyclopropyl-3-methyl-4-oxo-6-(trifluoromethyl)imidazo[4,5-c]pyridin-2-yl]-5-ethylsulfanyl-3-pyridyl]oxy]-2-methyl-propanenitrile

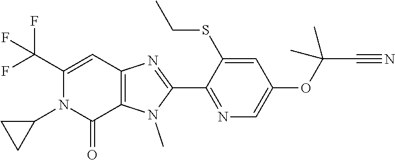 [SMILES: CCSc1cc(OC(C)(C)C#N)cnc1-c1nc2cc(C(F)(F)F)n(C3CC3)c(=O)c2n1C]

This compound was prepared in analogy to methods described in WO2020/084075.

LCMS (method 3): m/z 478 [M+H]⁺; retention time: 1.54 min.

### Step 2: Preparation of racemic 2-[[6-[5-cyclopropyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile (compound P1)

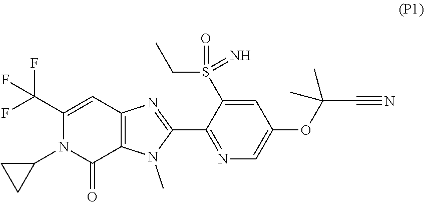 [SMILES: CCS(=N)(=O)c1cc(OC(C)(C)C#N)cnc1-c1nc2cc(C(F)(F)F)n(C3CC3)c(=O)c2n1C]

2-[[6-[5-Cyclopropyl-3-methyl-4-oxo-6-(trifluoromethyl) imidazo[4,5-c]pyridin-2-yl]-5-ethylsulfanyl-3-pyridyl]oxy]-2-methyl-propanenitrile (prepared as described above) was treated under analogous conditions as described in step 2 of Example P12 and in analogy to methods described in WO2020/084075 to afford the desired compound P1. LCMS (method 4): m/z 509 [M+H]⁺; retention time: 0.92 min.

### Step 3: Preparation of the Individual Enantiomer Compounds P1-A and P1-B

The racemic 2-[[6-[5-cyclopropyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile (compound P1) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IA, 3 m, 0.3 cm×10 cm, 40° C.
Mobile phase: A: CO₂ B: iPrOH isocratic: 20% B in 4.8 min
ABPR: 1800 psi
Flow rate: 2.0 ml/min
Detection: 220 nm
Sample concentration: 1 mg/mL
Injection: 1 μL

### Preparative SFC Method:

Sepiatec Prep SFC 100
Column: Daicel CHIRALPAK® IA, 5 m, 2.0 cm×25 cm
Mobile phase: A: CO₂ B: iPrOH isocratic: 20% B in 14 min
Backpressure: 150 bar
Flow rate: 60 ml/min
GLS pump: 2 ml MeOH
Detection: UV 220 nm
Sample: in MeOH/DCM

### Results:

| First eluting enantiomer P1-A | Second eluting enantiomer P1-B |
|---|---|
| Retention time (min) ~1.81 | Retention time (min) ~3.75 |
| Chemical purity (area % at | Chemical purity (area % |
| 220 nm) 99 | at 220 nm) 99 |
| Enantiomeric excess (%) >99 | Enantiomeric excess (%) >99 |

### Example P2: Preparation of racemic 1-[5-(ethylsulfonimidoyl)-6-[7-methyl-3-(trifluoromethyl)imidazo[4,5-c]pyridazin-6-yl]-3-pyridyl]cyclopropanecarbonitrile (Compound P2) and its Individual Enantiomers (Compounds P2-A and P2-B)

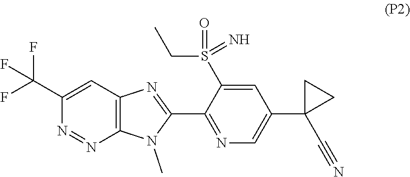 [SMILES: CCS(=N)(=O)c1cc(C2(C#N)CC2)cnc1-c1nc2cc(C(F)(F)F)nnc2n1C]

### Step 1: Preparation of 1-[5-ethylsulfanyl-6-[7-methyl-3-(trifluoromethyl)imidazo[4,5-c]pyridazin-6-yl]-3-pyridyl]cyclopropanecarbonitrile

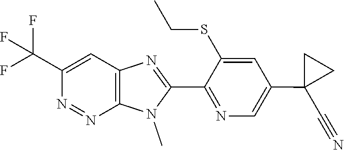 [SMILES: CCSc1cc(C2(C#N)CC2)cnc1-c1nc2cc(C(F)(F)F)nnc2n1C]

This compound was prepared in analogy to methods described in WO2019/234158.

LCMS (method 6): m/z 405 [M+H]⁺; retention time: 1.05 min.

### Step 2: Preparation of racemic 1-[5-(ethylsulfonimidoyl)-6-[7-methyl-3-(trifluoromethyl)imidazo[4,5-c]pyridazin-6-yl]-3-pyridyl]cyclopropanecarbonitrile (Compound P2)

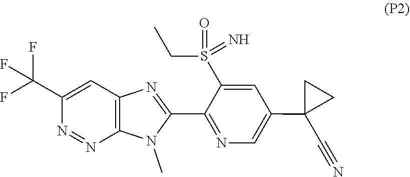 [SMILES: CCS(=N)(=O)c1cc(C2(C#N)CC2)cnc1-c1nc2cc(C(F)(F)F)nnc2n1C]

1-[5-Ethylsulfanyl-6-[7-methyl-3-(trifluoromethyl)imidazo[4,5-c]pyridazin-6-yl]-3-pyridyl]cyclopropanecarbonitrile (prepared as described above) was treated under analogous conditions as described in step 2 of Example P12 and in analogy to methods described in WO2019/234158 to afford the desired compound P2. LCMS (method 5): m/z 436 [M+H]⁺; retention time: 0.82 min.

### Step 3: Preparation of the Individual Enantiomer Compounds P2-A and P2-B

The racemic 1-[5-(ethylsulfonimidoyl)-6-[7-methyl-3-(trifluoromethyl)imidazo[4,5-c]pyridazin-6-yl]-3-pyridyl]cyclopropanecarbonitrile (compound P2) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IC, 3 m, 0.46 cm×10 cm, 40° C.
Mobile phase: A: CO₂ B: EtOH isocratic: 20% B in 4.8 min
ABPR: 1800 psi
Flow rate: 2.0 ml/min
Detection: 265 nm
Sample concentration: 1 mg/mL
Injection: 1 mL

### Preparative SFC Method:

Sepiatec Prep SFC 100
Column: Daicel CHIRALPAK® IC, 5 m, 2.0 cm×25 cm
Mobile phase: A: CO₂ B: EtOH isocratic: 25% B
Backpressure: 150 bar
Flow rate: 75 ml/min
GLS pump: -
Detection: UV 265 nm
Sample: in DCM/ACN

### Results:

| First eluting enantiomer P2-A | Second eluting enantiomer P2-B |
|---|---|
| Retention time (min) ~1.36 | Retention time (min) ~3.54 |
| Chemical purity (area % at | Chemical purity (area % at |
| 265 nm) >99 | 265 nm) >99 |
| Enantiomeric excess (%) >99 | Enantiomeric excess (%) >99 |

### Example P3: Preparation of racemic 2-[[5-(ethylsulfonimidoyl)-6-[7-(trifluoromethyl)imidazo[1,2-a]pyridin-2-yl]-3-pyridyl]oxy]-2-methyl-propanenitrile (Compound P3) and its Individual Enantiomers (Compounds P3-A and P3-B)

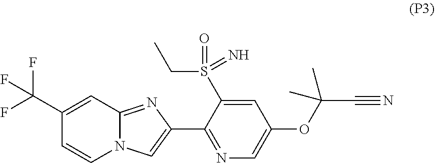 [SMILES: CCS(=N)(=O)c1cc(OC(C)(C)C#N)cnc1-c1cn2ccc(C(F)(F)F)cc2n1]

### Step 1: Preparation of 2-[[5-ethylsulfanyl-6-[7-(trifluoromethyl)imidazo[1,2-a]pyridin-2-yl]-3-pyridyl]oxy]-2-methyl-propanenitrile

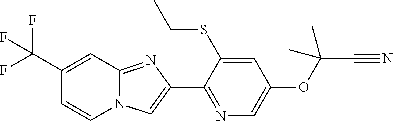 [SMILES: CCSc1cc(OC(C)(C)C#N)cnc1-c1cn2ccc(C(F)(F)F)cc2n1]

This compound was prepared in analogy to methods described in WO2020/084075.

¹H NMR (400 MHz, chloroform-d) δ ppm 1.44 (t, J=7.34 Hz, 3H) 1.81 (s, 6H) 3.04 (q, J=7.34 Hz, 2H) 7.02 (dd, J1=7.34; J2=1.65 Hz, 1H) 7.65 (d, J=2.57 Hz, 1H) 8.06 (s, 1H) 8.29 (d, J=7.34 Hz, 1H) 8.32 (d, J=2.57 Hz, 1H) 8.37 (d, J=1.65 Hz, 1H).

### Step 2: Preparation of racemic 2-[[5-(ethylsulfonimidoyl)-6-[7-(trifluoromethyl)imidazo[1,2-a]pyridin-2-yl]-3-pyridyl]oxy]-2-methyl-propanenitrile (Compound P3)

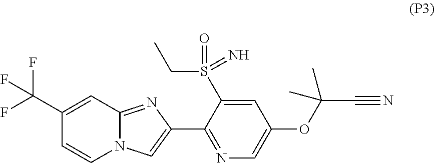 [SMILES: CCS(=N)(=O)c1cc(OC(C)(C)C#N)cnc1-c1cn2ccc(C(F)(F)F)cc2n1]

2-[[5-Ethylsulfanyl-6-[7-(trifluoromethyl)imidazo[1,2-a]pyridin-2-yl]-3-pyridyl]oxy]-2-methyl-propanenitrile (prepared as described above) was treated under analogous conditions as described in step 2 of Example P12 and in analogy to methods described in WO2020/084075 to afford the desired compound P3. LCMS (method 1): m/z 438 [M+H]⁺; retention time: 0.88 min.

### Step 3: Preparation of the Individual Enantiomer Compounds P3-A and P3-B

The racemic 2-[[5-(ethylsulfonimidoyl)-6-[7-(trifluoromethyl)imidazo[1,2-a]pyridin-2-yl]-3-pyridyl]oxy]-2-methyl-propanenitrile (compound P3) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IC, 3 m, 0.3 cm×10 cm, 40° C.
Mobile phase: A: CO₂ B: MeOH isocratic: 20% B in 4.8 min
ABPR: 1800 psi
Flow rate: 2.0 ml/min
Detection: 250 nm
Sample concentration: 1 mg/mL
Injection: 1 μL

### Preparative SFC Method:

Sepiatec Prep SFC 100
Column: Daicel CHIRALPAK® IC, 5 m, 2.0 cm×25 cm
Mobile phase: A: CO₂ B: MeOH isocratic: 20% B in 14 min
Backpressure: 150 bar
Flow rate: 60 ml/min
GLS pump: 2 ml MeOH
Detection: UV 250 nm
Sample: in MeOH/DCM

### Results:

| First eluting enantiomer P3-A | Second eluting enantiomer P3-B |
|---|---|
| Retention time (min) ~1.97 | Retention time (min) ~3.19 |
| Chemical purity (area % at | Chemical purity (area % at |
| 240 nm) 99 | 240 nm) 99 |
| Enantiomeric excess (%) >99 | Enantiomeric excess (%) >99 |

### Example P4: Preparation of racemic 2-[5-(ethylsulfonimidoyl)-6-[3-methyl-6-(trifluoromethyl)imidazo[4,5-c]pyridin-2-yl]-3-pyridyl]-2-methyl-propanenitrile (Compound P4) and its Individual Enantiomers (Compounds P4-A and P4-B)

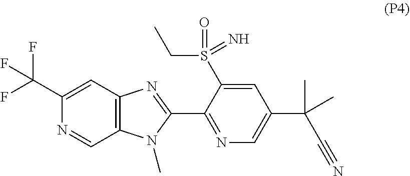 [SMILES: CCS(=N)(=O)c1cc(C(C)(C)C#N)cnc1-c1nc2cc(C(F)(F)F)ncc2n1C]

### Step 1: Preparation of 2-[5-ethylsulfanyl-6-[3-methyl-6-(trifluoromethyl)imidazo[4,5-c]pyridin-2-yl]-3-pyridyl]-2-methyl-propanenitrile

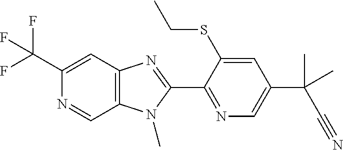 [SMILES: CCSc1cc(C(C)(C)C#N)cnc1-c1nc2cc(C(F)(F)F)ncc2n1C]

This compound was prepared in analogy to methods described in WO2018/153778.

LCMS (method 2): m/z 406 [M+H]⁺; retention time: 1.02 min.

### Step 2: Preparation of racemic 2-[5-(ethylsulfonimidoyl)-6-[3-methyl-6-(trifluoromethyl)imidazo[4,5-c]pyridin-2-yl]-3-pyridyl]-2-methyl-propanenitrile (compound P4)

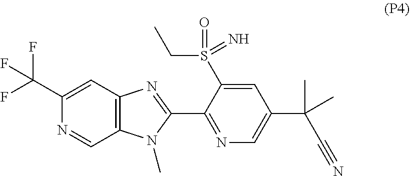 [SMILES: CCS(=N)(=O)c1cc(C(C)(C)C#N)cnc1-c1nc2cc(C(F)(F)F)ncc2n1C]

2-[5-Ethylsulfanyl-6-[3-methyl-6-(trifluoromethyl)imidazo[4,5-c]pyridin-2-yl]-3-pyridyl]-2-methyl-propanenitrile (prepared as described above) was treated under analogous conditions as described in step 2 of Example P12 and in analogy to methods described in WO2019/234158 to afford the desired compound P4. LCMS (method 2): m/z 437 [M+H]⁺; retention time: 0.83 min.

### Step 3: Preparation of the Individual Enantiomer Compounds P4-A and P4-B

The racemic 2-[5-(ethylsulfonimidoyl)-6-[3-methyl-6-(trifluoromethyl)imidazo[4,5-c]pyridin-2-yl]-3-pyridyl]-2-methyl-propanenitrile (compound P4) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.46 cm×10 cm, 40° C.
Mobile phase: A: CO₂ B: MeOH isocratic: 30% B in 4.8 min
ABPR: 1800 psi
Flow rate: 2.0 ml/min
Detection: 265 nm
Sample concentration: 1 mg/mL
Injection: 1 μL

### Preparative SFC Method:

Sepiatec Prep SFC 100
Column: Daicel CHIRALPAK® IG, 5 m, 2.0 cm×25 cm
Mobile phase: A: CO₂ B: MeOH isocratic: 30% B
Backpressure: 150 bar
Flow rate: 60 ml/min
GLS pump: -
Detection: UV 265 nm
Sample: in DCM/MeOH

### Results:

| First eluting enantiomer P4-A | Second eluting enantiomer P4-B |
|---|---|
| Retention time (min) ~1.76 | Retention time (min) ~2.82 |
| Chemical purity (area % at | Chemical purity (area % at |
| 265 nm) >99 | 265 nm) >99 |
| Enantiomeric excess (%) >99 | Enantiomeric excess (%) >98.6 |

### Example P5: Preparation of racemic 2-[[5-(ethylsulfonimidoyl)-2-methyl-6-[3-methyl-6-(trifluoromethyl)imidazo[4,5-b]pyridin-2-yl]-3-pyridyl]oxy]-2-methyl-propanenitrile (Compound P5) and its Individual Enantiomers (Compounds P5-A and P5-B)

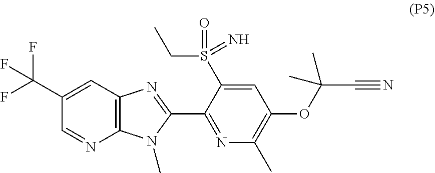 [SMILES: CCS(=N)(=O)c1cc(OC(C)(C)C#N)c(C)nc1-c1nc2cc(C(F)(F)F)cnc2n1C]

### Step 1: Preparation of 2-[[5-ethylsulfanyl-2-methyl-6-[3-methyl-6-(trifluoromethyl)imidazo[4,5-b]pyridin-2-yl]-3-pyridyl]oxy]-2-methyl-propanenitrile

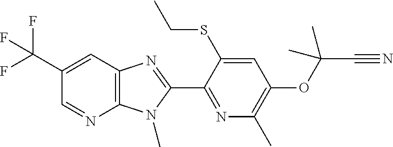 [SMILES: CCSc1cc(OC(C)(C)C#N)c(C)nc1-c1nc2cc(C(F)(F)F)cnc2n1C]

This compound was prepared in analogy to methods described in WO2020/084075.

LCMS (method 1): m/z 436 [M+H]⁺; retention time: 1.16 min.

### Step 2: Preparation of racemic 2-[[5-(ethylsulfonimidoyl)-2-methyl-6-[3-methyl-6-(trifluoromethyl)imidazo[4,5-b]pyridin-2-yl]-3-pyridyl]oxy]-2-methyl-propanenitrile (Compound P5)

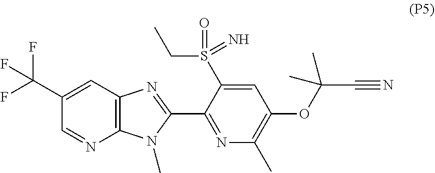 [SMILES: CCS(=N)(=O)c1cc(OC(C)(C)C#N)c(C)nc1-c1nc2cc(C(F)(F)F)cnc2n1C]

2 [[5-Ethylsulfanyl-2-methyl-6-[3-methyl-6-(trifluoromethyl)imidazo[4,5-b]pyridin-2-yl]-3-pyridyl]oxy]-2-methyl-propanenitrile (prepared as described above) was treated under analogous conditions as described in step 2 of Example P12 and in analogy to methods described in WO2020/084075 to afford the desired compound P5. LCMS (method 1): m/z 467 [M+H]⁺; retention time: 0.97 min.

### Step 3: Preparation of the Individual Enantiomer Compounds P5-A and P5-B

The racemic 2-[[5-(ethylsulfonimidoyl)-2-methyl-6-[3-methyl-6-(trifluoromethyl)imidazo[4,5-b]pyridin-2-yl]-3-pyridyl]oxy]-2-methyl-propanenitrile (compound P5) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.3 cm×10 cm, 40° C.
Mobile phase: A: CO₂ B: MeOH isocratic: 15% B in 4.8 min
ABPR: 1800 psi
Flow rate: 2.0 ml/min
Detection: 280 nm
Sample concentration: 1 mg/mL
Injection: 1 μL

### Preparative SFC Method:

Sepiatec Prep SFC 100
Column: Daicel CHIRALPAK® IG, 5 m, 2.0 cm×25 cm
Mobile phase: A: CO₂ B: MeOH isocratic: 15% B in 14 min
Backpressure: 150 bar
Flow rate: 60 ml/min
GLS pump: 5 ml
Detection: UV 280 nm
Sample: in MeOH/DCM

### Results:

| First eluting enantiomer P5-A | Second eluting enantiomer P5-B |
|---|---|
| Retention time (min) ~1.92 | Retention time (min) ~3.12 |
| Chemical purity (area % at | Chemical purity (area % |
| 280 nm) 99 | at 280 nm) 99 |
| Enantiomeric excess (%) >99 | Enantiomeric excess (%) >99 |

### Example P6: Preparation of racemic 1-[3-(ethylsulfonimidoyl)-4-[3-methyl-6-(trifluoromethyl)imidazo[4,5-b]pyridin-2-yl]phenyl]cyclopropanecarbonitrile (Compound P6) and its Individual Enantiomers (Compounds P6-A and P6-B)

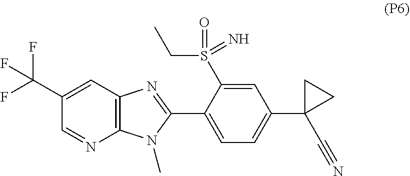 [SMILES: CCS(=N)(=O)c1cc(C2(C#N)CC2)ccc1-c1nc2cc(C(F)(F)F)cnc2n1C]

### Step 1: Preparation of 1-[3-ethylsulfanyl-4-[3-methyl-6-(trifluoromethyl)imidazo[4,5-b]pyridin-2-yl]phenyl]cyclooropanecarbonitrile

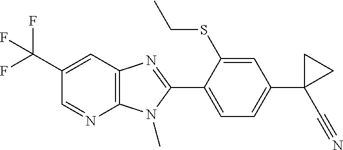 [SMILES: CCSc1cc(C2(C#N)CC2)ccc1-c1nc2cc(C(F)(F)F)cnc2n1C]

This compound was prepared in analogy to methods described in WO2019/234158.

LCMS (method 6): m/z 403 [M+H]⁺; retention time: 1.18 min.

### Step 2: Preparation of racemic 1-[3-(ethylsulfonimidoyl)-4-[3-methyl-6-(trifluoromethyl)imidazo[4,5-b]pyridin-2-yl]phenyl]cyclopropanecarbonitrile (Compound P6)

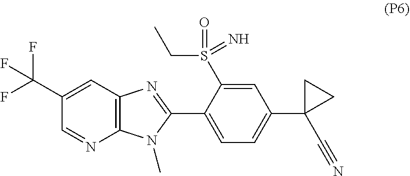 [SMILES: CCS(=N)(=O)c1cc(C2(C#N)CC2)ccc1-c1nc2cc(C(F)(F)F)cnc2n1C]

1-[3-Ethylsulfanyl-4-[3-methyl-6-(trifluoromethyl)imidazo[4,5-b]pyridin-2-yl]phenyl]cyclopropanecarbonitrile (prepared as described above) was treated under analogous conditions as described in step 2 of Example P12 and in analogy to methods described in WO2019/234158 to afford the desired compound P6. LCMS (method 2): m/z 434 [M+H]⁺; retention time: 0.88 min.

### Step 3: Preparation of the Individual Enantiomer Compounds P6-A and P6-B

The racemic 1-[3-(ethylsulfonimidoyl)-4-[3-methyl-6-(trifluoromethyl)imidazo[4,5-b]pyridin-2-yl]phenyl]cyclopropanecarbonitrile (compound P6) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.46 cm×10 cm, 40° C.
Mobile phase: A: CO₂ B: iPrOH isocratic: 35% B in 4.8 min
ABPR: 1800 psi
Flow rate: 2.0 ml/min
Detection: 290 nm
Sample concentration: 1 mg/mL
Injection: 1 mL

### Preparative SFC Method:

Sepiatec Prep SFC 100
Column: Daicel CHIRALPAK® IG, 5 m, 2.0 cm×25 cm
Mobile phase: A: CO₂ B: iPrOH isocratic: 35% B
Backpressure: 150 bar
Flow rate: 60 ml/min
GLS pump: -
Detection: UV 290 nm
Sample: in DCM/ACN

### Results:

| First eluting enantiomer P6-A | Second eluting enantiomer P6-B |
|---|---|
| Retention time (min) ~2.34 | Retention time (min) ~3.99 |
| Chemical purity (area % at | Chemical purity (area % at |
| 290 nm) >99 | 290 nm) >99 |
| Enantiomeric excess (%) >99 | Enantiomeric excess (%) >99 |

### Example P7: Preparation of racemic 2-[[5-(ethylsulfonimidoyl)-6-[7-(trifluoromethylsulfanyl)imidazo[1,2-c]pyrimidin-2-yl]-3-pyridyl]oxy]l-2-methyl-propanenitrile (Compound P7) and its Individual Enantiomers (Compounds P7-A and P7-B)

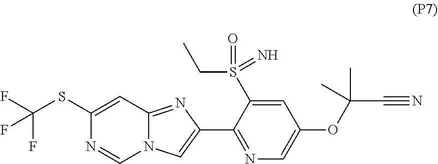 [SMILES: CCS(=N)(=O)c1cc(OC(C)(C)C#N)cnc1-c1cn2cnc(SC(F)(F)F)cc2n1]

### Step 1: Preparation of 2-[[5-ethylsulfanyl-6-[7-(trifluoromethylsulfanyl)imidazo[1,2-c]pyrimidin-2-yl]-3-pyridyl]oxy]-2-methyl-propanenitrile

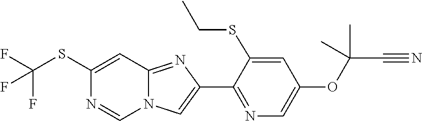 [SMILES: CCSc1cc(OC(C)(C)C#N)cnc1-c1cn2cnc(SC(F)(F)F)cc2n1]

This compound was prepared in analogy to methods described in WO2020/084075.

¹H NMR (400 MHz, chloroform-d) δ ppm 1.44 (m, 3H) 1.82 (s, 6H) 3.05 (m, 2H) 7.66 (d, J=2.20 Hz, 1H) 8.02 (s, 1H) 8.34 (d, J=2.20 Hz, 1H) 8.39 (s, 1H) 9.10 (s, 1H).

### Step 2: Preparation of racemic 2-[[5-(ethylsulfonimidoyl)-6-[7-(trifluoromethylsulfanyl)imidazo[1,2-c]pyrimidin-2-yl]-3-pyridyl]oxy]-2-methyl-propanenitrile (Compound P7)

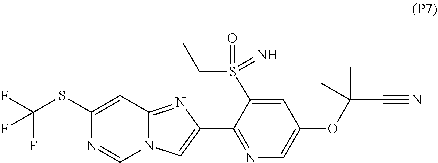 [SMILES: CCS(=N)(=O)c1cc(OC(C)(C)C#N)cnc1-c1cn2cnc(SC(F)(F)F)cc2n1]

2-[[5-Ethylsulfanyl-6-[7-(trifluoromethylsulfanyl)imidazo[1,2-c]pyrimidin-2-yl]-3-pyridyl]oxy]-2-methyl-propanenitrile (prepared as described above) was treated under analogous conditions as described in step 2 of Example P12 and in analogy to methods described in WO2020/084075 to afford the desired compound P7. LCMS (method 1): m/z 471 [M+H]⁺; retention time: 0.89 min.

### Step 3: Preparation of the Individual Enantiomer Compounds P7-A and P7-B

The racemic 2-[[5-(ethylsulfonimidoyl)-6-[7-(trifluoromethylsulfanyl)imidazo[1,2-c]pyrimidin-2-yl]-3-pyridyl]oxy]-2-methyl-propanenitrile (compound P7) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IC, 3 m, 0.3 cm×10 cm, 40° C.
Mobile phase: A: CO₂ B: MeOH isocratic: 30% B in 4.8 min
ABPR: 1800 psi
Flow rate: 2.0 ml/min
Detection: 250 nm
Sample concentration: 1 mg/mL
Injection: 1 μL

### Preparative SFC Method:

Sepiatec Prep SFC 100
Column: Daicel CHIRALPAK® IC, 5 m, 2.0 cm×25 cm
Mobile phase: A: CO₂ B: MeOH isocratic: 30% B in 14 min
Backpressure: 150 bar
Flow rate: 60 ml/min
GLS pump: 2 ml MeOH
Detection: UV 250 nm
Sample: in MeOH/DCM

### Results:

| First eluting enantiomer P7-A | Second eluting enantiomer P7-B |
|---|---|
| Retention time (min) ~1.28 | Retention time (min) ~3.63 |
| Chemical purity (area % at | Chemical purity (area % at |
| 250 nm) 99 | 250 nm) 99 |
| Enantiomeric excess (%) >99 | Enantiomeric excess (%) >99 |

### Example P8: Preparation of racemic 1-[5-(ethylsulfonimidoyl)-6-[5-methoxy-3-methyl-4-oxo-6-(trifluoromethyl)imidazo[4,5-c]pyridin-2-yl]-3-pyridyl]cyclopropanecarbonitrile (Compound P8) and its Individual Enantiomers (Compounds P8-A and P8-B)

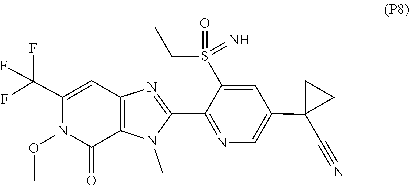 [SMILES: CCS(=N)(=O)c1cc(C2(C#N)CC2)cnc1-c1nc2cc(C(F)(F)F)n(OC)c(=O)c2n1C]

### Step 1: Preparation of 1-[5-ethylsulfanyl-6-[5-methoxy-3-methyl-4-oxo-6-(trifluoromethyl)imidazo[4,5-c]pyridin-2-yl]-3-pyridyl]cyclopropanecarbonitrile

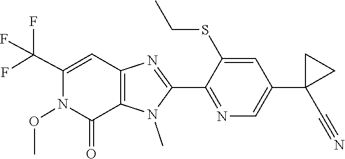 [SMILES: CCSc1cc(C2(C#N)CC2)cnc1-c1nc2cc(C(F)(F)F)n(OC)c(=O)c2n1C]

This compound was prepared in analogy to methods described in WO2019/234158.

LCMS (method 5): m/z 450 [M+H]⁺; retention time: 1.03 min.

### Step 2: Preparation of racemic 1-[5-(ethylsulfonimidoyl)-6-[5-methoxy-3-methyl-4-oxo-6-(trifluoromethyl)imidazo[4,5-c]pyridin-2-yl]-3-pyridyl]cyclopropanecarbonitrile (Compound P8)

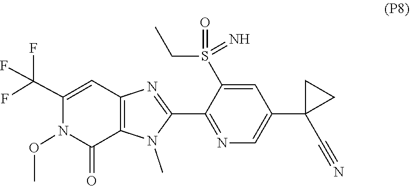 [SMILES: CCS(=N)(=O)c1cc(C2(C#N)CC2)cnc1-c1nc2cc(C(F)(F)F)n(OC)c(=O)c2n1C]

1-[5-Ethylsulfanyl-6-[5-methoxy-3-methyl-4-oxo-6-(trifluoromethyl)imidazo[4,5-c]pyridin-2-yl]-3-pyridyl]cyclopropanecarbonitrile (prepared as described above) was treated under analogous conditions as described in step 2 of Example P12 and in analogy to methods described in WO2019/234158 to afford the desired compound P8. LCMS (method 5): m/z 481 [M+H]⁺; retention time: 0.85 min.

### Step 3: Preparation of the Individual Enantiomer Compounds P8-A and P8-B

The racemic 1-[5-(ethylsulfonimidoyl)-6-[5-methoxy-3-methyl-4-oxo-6-(trifluoromethyl)imidazo[4,5-c]pyridin-2-yl]-3-pyridyl]cyclopropanecarbonitrile (compound P8) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IA, 3 m, 0.46 cm×10 cm, 40° C.
Mobile phase: A: CO₂ B: iPrOH isocratic: 25% B in 4.8 min
ABPR: 1800 psi
Flow rate: 2.0 ml/min
Detection: 220 nm
Sample concentration: 1 mg/mL
Injection: 1 mL

### Preparative SFC Method:

Sepiatec Prep SFC 100
Column: Daicel CHIRALPAK® IA, 5 m, 2.0 cm×25 cm
Mobile phase: A: CO₂ B: iPrOH isocratic: 25% B
Backpressure: 150 bar
Flow rate: 60 ml/min
GLS pump: -
Detection: UV 220 nm
Sample: in DCM/ACN

### Results:

| First eluting enantiomer P8-A | Second eluting enantiomer P8-B |
|---|---|
| Retention time (min) ~1.73 | Retention time (min) ~3.15 |
| Chemical purity (area % at | Chemical purity (area % at |
| 220 nm) 98.8 | 220 nm) >99 |
| Enantiomeric excess (%) >99 | Enantiomeric excess (%) >99 |

### Example P9: Preparation of racemic 2-[[5-(ethylsulfonimidoyl)-6-[3-methyl-6-(trifluoromethyl)imidazo[4,5-b]pyridin-2-yl]-3-pyridyl]oxy]-2-methyl-propanenitrile (Compound P9) and its Individual Enantiomers (Compounds P9-A and P9-B)

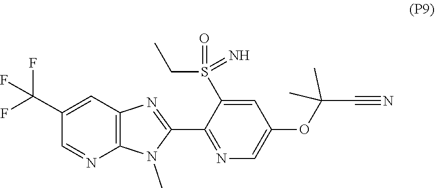 [SMILES: CCS(=N)(=O)c1cc(OC(C)(C)C#N)cnc1-c1nc2cc(C(F)(F)F)cnc2n1C]

### Step 1: Preparation of 2-[[5-ethylsulfanyl-6-[3-methyl-6-(trifluoromethyl)imidazo[4,5-b]pyridin-2-yl]-3-pyridyl]oxy]2-methyl-propanenitrile

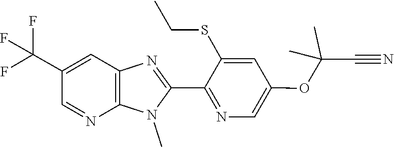 [SMILES: CCSc1cc(OC(C)(C)C#N)cnc1-c1nc2cc(C(F)(F)F)cnc2n1C]

This compound was prepared in analogy to methods described in WO2020/084075.

LCMS (method 1): m/z 422 [M+H]⁺; retention time: 1.11 min.

### Step 2: Preparation of racemic 2-[[5-(ethylsulfonimidoyl)-6-[3-methyl-6-(trifluoromethyl)imidazo[4,5-b]pyridin-2-yl]-3-pyridyl]oxy]-2-methyl-propanenitrile (Compound P9)

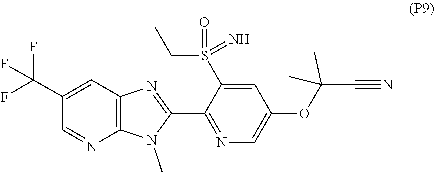 [SMILES: CCS(=N)(=O)c1cc(OC(C)(C)C#N)cnc1-c1nc2cc(C(F)(F)F)cnc2n1C]

2-[[5-Ethylsulfanyl-6-[3-methyl-6-(trifluoromethyl)imidazo[4,5-b]pyridin-2-yl]-3-pyridyl]oxy]-2-methyl-propanenitrile (prepared as described above) was treated under analogous conditions as described in step 2 of Example P12 and in analogy to methods described in WO2020/084075 to afford the desired compound P9. LCMS (method 1): m/z 453 [M+H]⁺; retention time: 0.93 min.

### Step 3: Preparation of the Individual Enantiomer Compounds P9-A and P9-B

The racemic 2-[[5-(ethylsulfonimidoyl)-6-[3-methyl-6-(trifluoromethyl)imidazo[4,5-b]pyridin-2-yl]-3-pyridyl]oxy]-2-methyl-propanenitrile (compound P9) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IA, 3 m, 0.3 cm×10 cm, 40° C.
Mobile phase: A: CO₂ B: EtOH isocratic: 10% B in 4.8 min
ABPR: 1800 psi
Flow rate: 2.0 ml/min
Detection: 290 nm
Sample concentration: 1 mg/mL
Injection: 1 μL

### Preparative SFC Method:

Sepiatec Prep SFC 100
Column: Daicel CHIRALPAK® IG, 5 m, 2.0 cm×25 cm
Mobile phase: A: CO₂ B: EtOH isocratic: 25% B in 14 min
Backpressure: 150 bar
Flow rate: 60 ml/min
GLS pump: 3 ml
Detection: UV 290 nm
Sample: in MeOH/ACN

### Results:

| First eluting enantiomer P9-A | Second eluting enantiomer P9-B |
|---|---|
| Retention time (min) ~1.49 | Retention time (min) ~2.01 |
| Chemical purity (area % at | Chemical purity (area % at |
| 290 nm) 99 | 290 nm) 99 |
| Enantiomeric excess (%) >99 | Enantiomeric excess (%) >99 |

A sample of the second eluting enantiomer compound P9-B (crystals from ethanol obtained according to Example P9 step 3, with chemical purity of 99% (%=290 nm) and enantiomeric excess of >99%) was subjected to analysis by single crystal X-ray diffraction. Single crystal intensity data was collected on an Rigaku Oxford Diffraction Supernova X-ray Generator using Cu-Kα radiation at a wavelength of 1.54184 Å, collected at 100K to a resolution of 0.81 Å.

The dataset was refined and reduced using the data collection and processing software CrysAlisPro, the structure solution was completed using SIR92 (Altomare A, Cascarano G, Giacovazzo C, Guagliardi A, Burla M C, Polidori G and Camalli M, J. Appl. Cryst. 27: 435 (1994)) and the structure refinement was competed using the CRYSTALS software package (Betteridge P W, Carruthers J R, Cooper R I, Prout K and Watkin D J, J. Appl. Cryst. 36:1487 (2003)).

Individual enantioner compound P9-B crystallized in the monoclinic space group C2. Unit cell parameters of the single crystal analysis are shown in Table 1.

TABLE 1
Crystal Structure Parameters^{#}

| Chemical formula | C₁₉H₁₉F₃N₆O₂S |
|---|---|
| Crystal system | monoclinic |
| Space group | C2 |
| Cell lengths (Å) | a = 27.2791, b = 11.79482, c = 12.4992 |
| Cell angles (°) | α = 90, β = 93.0334, γ = 90 |
| Unit cell volume (Å3) | 4016.00 |
| Z | 8 |

^{#}In Table 1: a, b, c = Length of the edges of the unit cell; α, β, γ = Angles of the unit cell; and Z = molecules per cell.

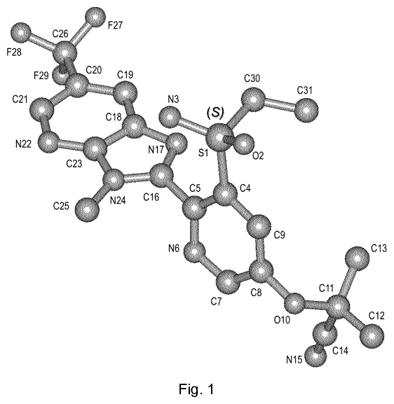

The X-ray crystal structure of compound P9-B is depicted in FIG. 1. The stereochemistry was unambiguously determined, the stereogenic sulfur atom (labeled S1 in FIG. 1) is in the S-configuration (indicated by the annotation (S) in FIG. 1). For technical reasons, the numbering scheme used in the structure of FIG. 1 does not correspond to systematic nomenclature.

Hence, the second eluting enantiomer P9-B was proved by x-ray crystallography to be (S)-2-[[5-(ethylsulfonimidoyl)-6-[3-methyl-6-(trifluoromethyl)imidazo[4,5-b]pyridin-2-yl]-3-pyridyl]oxy]-2-methyl-propanenitrile, corresponding to compound (S)—P9 (Table Y) obtained via enantioselective synthesis (Example E2 below).

[a]_{D}²⁰=+13.7° (MeOH, C=0.88).

Similarly, the first eluting enantiomer P9-A was proved by x-ray crystallography to be (R)-2-[[5-(ethylsulfonimidoyl)-6-[3-methyl-6-(trifluoromethyl)imidazo[4,5-b]pyridin-2-yl]-3-pyridyl]oxy]-2-methyl-propanenitrile, corresponding to compound (R)-P9 (Table Y) obtained via enantioselective synthesis (Example E1 below).

[a]_{D}²⁰=−13.2° (MeOH, C=0.87).

### Example P10: Preparation of racemic 1-[5-(ethylsulfonimidoyl)-6-[3-methyl-6-(trifluoromethyl)imidazo[4,5-c]pyridin-2-yl]-3-pyridyl]cyclopropanecarbonitrile (compound P10) and its Individual Enantiomers (Compounds P10-A and P10-B)

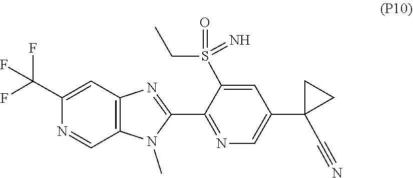 [SMILES: CCS(=N)(=O)c1cc(C2(C#N)CC2)cnc1-c1nc2cc(C(F)(F)F)ncc2n1C]

### Step 1: Preparation of 1-[5-ethylsulfanyl-6-[3-methyl-6-(trifluoromethyl)imidazo[4,5-c]pyridin-2-yl]-3-pyridyl]cyclopropanecarbonitrile

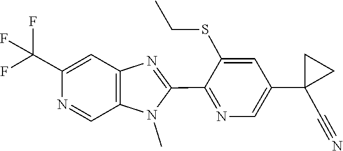 [SMILES: CCSc1cc(C2(C#N)CC2)cnc1-c1nc2cc(C(F)(F)F)ncc2n1C]

This compound was prepared in analogy to methods described in WO2019/234158.

LCMS (method 1): m/z 404 [M+H]⁺; retention time: 0.98 min.

### Step 2: Preparation of racemic 1-[5-(ethylsulfonimidoyl)-6-[3-methyl-6-(trifluoromethyl)imidazo[4,5-c]pyridin-2-yl]-3-pyridyl]cyclopropanecarbonitrile (Compound P10)

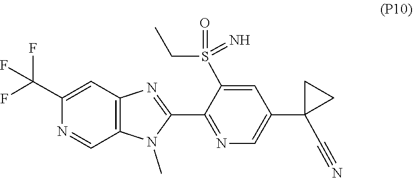 [SMILES: CCS(=N)(=O)c1cc(C2(C#N)CC2)cnc1-c1nc2cc(C(F)(F)F)ncc2n1C]

1-[5-Ethylsulfanyl-6-[3-methyl-6-(trifluoromethyl)imidazo[4,5-c]pyridin-2-yl]-3-pyridyl]cyclopropanecarbonitrile (prepared as described above) was treated under analogous conditions as described in step 2 of Example P12 and in analogy to methods described in WO2019/234158 to afford the desired compound P10. LCMS (method 1): m/z 435 [M+H]⁺; retention time: 0.82 min.

### Step 3: Preparation of the Individual Enantiomer Compounds P10-A and P10-B

The racemic 1-[5-(ethylsulfonimidoyl)-6-[3-methyl-6-(trifluoromethyl)imidazo[4,5-c]pyridin-2-yl]-3-pyridyl]cyclopropanecarbonitrile (compound P10) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.46 cm×10 cm, 40° C.
Mobile phase: A: CO₂ B: MeOH isocratic: 40% B in 4.8 min
ABPR: 1800 psi
Flow rate: 2.0 ml/min
Detection: 250 nm
Sample concentration: 1 mg/mL
Injection: 1 μL

### Preparative SFC Method:

Sepiatec Prep SFC 100
Column: Daicel CHIRALPAK® IG, 5 m, 2.0 cm×25 cm
Mobile phase: A: CO₂ B: MeOH isocratic: 40% B
Backpressure: 150 bar
Flow rate: 60 ml/min
GLS pump: -
Detection: UV 270 nm
Sample: in MeOH/DCM/ACN

### Results:

| First eluting enantiomer P10-A | Second eluting enantiomer P10-B |
|---|---|
| Retention time (min) ~1.68 | Retention time (min) ~2.99 |
| Chemical purity | Chemical purity |
| (area % at 270 nm) >99 | (area % at 270 nm) >99 |
| Enantiomeric excess (%) >99 | Enantiomeric excess (%) >99 |

### Example P11: Preparation of racemic 2-[[5-(ethylsulfonimidoyl)-6-[7-(trifluoromethyl)imidazo[1,2-c]pyrimidin-2-yl]-3-pyridyl]oxy]-2-methyl-propanenitrile (Compound P11) and its Individual Enantiomers (Compounds P11-A and P11-B)

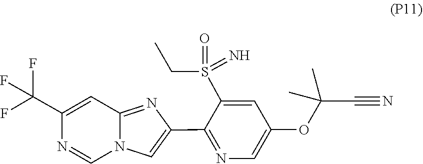 [SMILES: CCS(=N)(=O)c1cc(OC(C)(C)C#N)cnc1-c1cn2cnc(C(F)(F)F)cc2n1]

### Step 1: Preparation of 2-[[5-ethylsulfanyl-6-[7-(trifluoromethyl)imidazo[1,2-c]pyrimidin-2-yl]-3-pyridyl]oxy]-2-methyl-propanenitrile

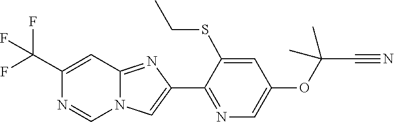 [SMILES: CCSc1cc(OC(C)(C)C#N)cnc1-c1cn2cnc(C(F)(F)F)cc2n1]

This compound was prepared in analogy to methods described in WO2020/084075.

¹H NMR (400 MHz, chloroform-d) δ ppm 1.44 (t, J=7.34 Hz, 3H) 1.81 (s, 6H) 3.04 (q, J=7.34 Hz, 2H) 7.67 (s, 1H) 8.05 (s, 1H) 8.34 (s, 1H) 8.45 (s, 1H) 9.18 (s, 1H).

### Step 2: Preparation of racemic 2-[[5-(ethylsulfonimidoyl)-6-[7-(trifluoromethyl)imidazo[1,2-c]pyrimidin-2-yl]-3-pyridyl]oxy]-2-methyl-propanenitrile (Compound P11)

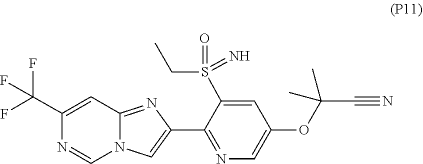 [SMILES: CCS(=N)(=O)c1cc(OC(C)(C)C#N)cnc1-c1cn2cnc(C(F)(F)F)cc2n1]

2-[[5-Ethylsulfanyl-6-[7-(trifluoromethyl)imidazo[1,2-c]pyrimidin-2-yl]-3-pyridyl]oxy]-2-methyl-propanenitrile (prepared as described above) was treated under analogous conditions as described in step 2 of Example P12 and in analogy to methods described in WO2020/084075 to afford the desired compound P11. LCMS (method 1): m/z 439 [M+H]⁺; retention time: 0.84 min.

### Step 3: Preparation of the Individual Enantiomer Compounds P11-A and P11-B

The racemic 2-[[5-(ethylsulfonimidoyl)-6-[7-(trifluoromethyl)imidazo[1,2-c]pyrimidin-2-yl]-3-pyridyl]oxy]-2-methyl-propanenitrile (compound P11) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IC, 3 m, 0.3 cm×10 cm, 40° C.
Mobile phase: A: CO₂ B: MeOH isocratic: 30% B in 4.8 min
ABPR: 1800 psi
Flow rate: 2.0 ml/min
Detection: 240 nm
Sample concentration: 1 mg/mL
Injection: 1 μL

### Preparative SFC Method:

Sepiatec Prep SFC 100
Column: Daicel CHIRALPAK® IC, 5 m, 2.0 cm×25 cm
Mobile phase: A: CO₂ B: MeOH isocratic: 30% B in 14 min
Backpressure: 150 bar
Flow rate: 60 ml/min
GLS pump: 2 ml MeOH
Detection: UV 250 nm
Sample: in MeOH/DCM

### Results:

| First eluting enantiomer P11-A | Second eluting enantiomer P11-B |
|---|---|
| Retention time (min) ~1.08 | Retention time (min) ~2.78 |
| Chemical purity | Chemical purity |
| (area % at 240 nm) 99 | (area % at 240 nm) 99 |
| Enantiomeric excess (%) >99 | Enantiomeric excess (%) >99 |

### Example P12: Preparation of racemic 2-[5-(ethylsulfonimidoyl)-6-[3-methyl-6-(trifluoromethyl)imidazo[4,5-b]pyridin-2-yl]-3-pyridyl]-2-methyl-propanenitrile (Compound P12) and its Individual Enantiomers (Compounds P12-A and P12-B)

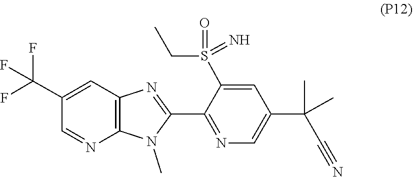 [SMILES: CCS(=N)(=O)c1cc(C(C)(C)C#N)cnc1-c1nc2cc(C(F)(F)F)cnc2n1C]

### Step 1: Preparation of 2-[5-ethylsulfanyl-6-[3-methyl-6-(trifluoromethyl)imidazo[4,5-b]pyridin-2-yl]-3-pyridyl]-2-methyl-propanenitrile

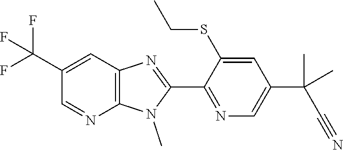 [SMILES: CCSc1cc(C(C)(C)C#N)cnc1-c1nc2cc(C(F)(F)F)cnc2n1C]

This compound was prepared in analogy to methods described in WO2018/153778.

LCMS (method 2): m/z 406 [M+H]⁺; retention time: 1.09 min.

### Step 2: Preparation of racemic 2-[5-(ethylsulfonimidoyl)-6-[3-methyl-6-(trifluoromethyl)imidazo[4,5-b]pyridin-2-yl]-3-pyridyl]-2-methyl-propanenitrile (Compound P12)

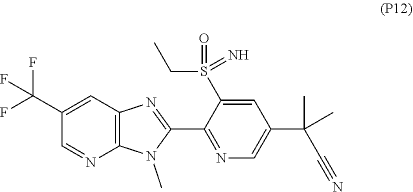 [SMILES: CCS(=N)(=O)c1cc(C(C)(C)C#N)cnc1-c1nc2cc(C(F)(F)F)cnc2n1C]

A solution of 2-[5-ethylsulfanyl-6-[3-methyl-6-(trifluoromethyl)imidazo[4,5-b]pyridin-2-yl]-3-pyridyl]-2-methyl-propanenitrile (prepared as described above) (300 mg, 0.74 mmol) in methanol (5 mL) was added at room temperature to a solution of diacetoxy iodobenzene (596 mg, 1.85 mmol) and ammonium carbamate (116 mg, 1.48 mmol) in methanol (4 mL). After stirring for 90 minutes at room temperature, the reaction mixture was evaporated and the residue diluted with dichloromethane. The organic phase was washed twice with water, dried over anhydrous sodium sulfate, filtered and concentrated under vacuum. The crude product was purified by flash chromatography over silica gel (ethyl acetate in cyclohexane) to afford the desired compound P12.

LCMS (method 2): m/z 437 [M+H]⁺; retention time: 0.91 min.

### Step 3: Preparation of the Individual Enantiomer Compounds P12-A and P12-B

The racemic 2-[5-(ethylsulfonimidoyl)-6-[3-methyl-6-(trifluoromethyl)imidazo[4,5-b]pyridin-2-yl]-3-pyridyl]-2-methyl-propanenitrile_(compound P12) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.46 cm×10 cm, 40° C.
Mobile phase: A: CO₂ B: EtOH isocratic: 20% B in 4.8 min
ABPR: 1800 psi
Flow rate: 2.0 ml/min
Detection: 220 nm
Sample concentration: 1 mg/mL
Injection: 1 mL

### Preparative SFC Method:

Sepiatec Prep SFC 100
Column: Daicel CHIRALPAK® IG, 5 m, 2.0 cm×25 cm
Mobile phase: A: CO₂ B: EtOH isocratic: 20% B
Backpressure: 150 bar
Flow rate: 60 ml/min
GLS pump: -
Detection: UV 220 nm
Sample: in DCM/ACN

### Results:

| First eluting enantiomer P12-A | Second eluting enantiomer P12-B |
|---|---|
| Retention time (min) ~2.42 | Retention time (min) ~3.17 |
| Chemical purity | Chemical purity |
| (area % at 220 nm) >99 | (area % at 220 nm) >99 |
| Enantiomeric purity (%) >99 | Enantiomeric purity (%) >99 |

### Example P13: Preparation of Racemic 2-[[5-(ethylsulfonimidoyl)-6-[3-methyl-6-(trifluoromethyl)imidazo[4,5-c]pyridin-2-yl]-3-pyridyl]oxy]-2-methyl-propanenitrile (Compound P13) and its Individual Enantiomers (Compounds P13-A and P13-B)

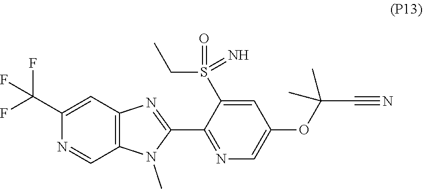 [SMILES: CCS(=N)(=O)c1cc(OC(C)(C)C#N)cnc1-c1nc2cc(C(F)(F)F)ncc2n1C]

### Step 1: Preparation of 2-[[5-ethylsulfanyl-6-[3-methyl-6-(trifluoromethyl)imidazo[4,5-c]pyridin-2-yl]-3-pyridyl]oxy]-2-methyl-propanenitrile

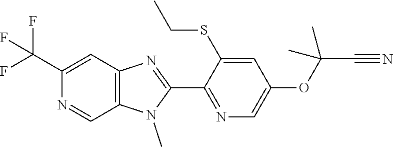 [SMILES: CCSc1cc(OC(C)(C)C#N)cnc1-c1nc2cc(C(F)(F)F)ncc2n1C]

This compound was prepared in analogy to methods described in WO2020/084075.

LCMS (method 1): m/z 422 [M+H]⁺; retention time: 1.02 min.

### Step 2: Preparation of racemic 2-[[5-(ethylsulfonimidoyl)-6-[3-methyl-6-(trifluoromethyl)imidazo[4,5-c]pyridin-2-yl]-3-pyridyl]oxy]-2-methyl-propanenitrile (Compound P13)

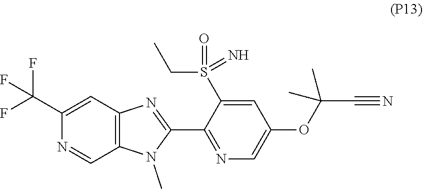 [SMILES: CCS(=N)(=O)c1cc(OC(C)(C)C#N)cnc1-c1nc2cc(C(F)(F)F)ncc2n1C]

2-[[5-Ethylsulfanyl-6-[3-methyl-6-(trifluoromethyl)imidazo[4,5-c]pyridin-2-yl]-3-pyridyl]oxy]-2-methyl-propanenitrile (prepared as described above) was treated under analogous conditions as described in step 2 of Example P12 and in analogy to methods described in WO2020/084075 to afford the desired compound P13. LCMS (method 1): m/z 453 [M+H]⁺; retention time: 0.86 min.

### Step 3: Preparation of the Individual Enantiomer Compounds P13-A and P13-B

The racemic 2-[[5-(ethylsulfonimidoyl)-6-[3-methyl-6-(trifluoromethyl)imidazo[4,5-c]pyridin-2-yl]-3-pyridyl]oxy]-2-methyl-propanenitrile (compound P13) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.3 cm×10 cm, 40° C.
Mobile phase: A: CO₂ B: MeOH isocratic: 30% B in 4.8 min
ABPR: 1800 psi
Flow rate: 2.0 ml/min
Detection: 270 nm
Sample concentration: 1 mg/mL
Injection: 1 μL

### Preparative SFC Method:

Sepiatec Prep SFC 100
Column: Daicel CHIRALPAK® IG, 5 m, 2.0 cm×25 cm
Mobile phase: A: CO₂ B: MeOH isocratic: 30% B in 14 min
Backpressure: 150 bar
Flow rate: 60 ml/min
GLS pump: 3 ml
Detection: UV 270 nm
Sample: in EtOH

### Results:

| First eluting enantiomer P13-A | Second eluting enantiomer P13-B |
|---|---|
| Retention time (min) ~1.70 | Retention time (min) ~3.89 |
| Chemical purity | Chemical purity |
| (area % at 270 nm) 99 | (area % at 270 nm) 99 |
| Enantiomeric excess (%) >99 | Enantiomeric excess (%) >99 |

### Example P14: Preparation of racemic 1-[6-[5-ethyl-3-methyl-4-oxo-6-(trifluoromethyl)imidazo[4,5-c]pyridin-2-yl]-5-(ethylsulfonimidoyl)-3-pyridyl]cyclopropanecarbonitrile (Compound P14) and its Individual Enantiomers (Compounds P14-A and P14-B)

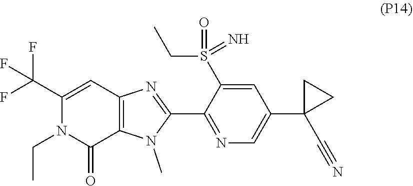 [SMILES: CCn1c(C(F)(F)F)cc2nc(-c3ncc(C4(C#N)CC4)cc3S(=N)(=O)CC)n(C)c2c1=O]

### Step 1: Preparation of 1-[6-[5-ethyl-3-methyl-4-oxo-6-(trifluoromethyl)imidazo[4,5-c]pyridin-2-yl]-5-ethylsulfanyl-3-pyridyl]cyclopropanecarbonitrile

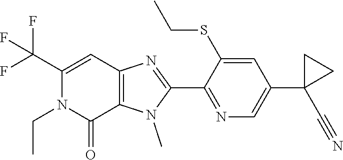 [SMILES: CCSc1cc(C2(C#N)CC2)cnc1-c1nc2cc(C(F)(F)F)n(CC)c(=O)c2n1C]

This compound was prepared in analogy to methods described in WO2019/234158.

LCMS (method 5): m/z 448 [M+H]⁺; retention time: 1.13 min.

### Step 2: Preparation of racemic 1-[6-[5-ethyl-3-methyl-4-oxo-6-(trifluoromethyl)imidazo[4,5-c]pyridin-2-yl]-5-(ethylsulfonimidoyl)-3-pyridyl]cyclopropanecarbonitrile (Compound P14)

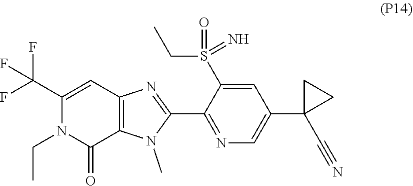 [SMILES: CCn1c(C(F)(F)F)cc2nc(-c3ncc(C4(C#N)CC4)cc3S(=N)(=O)CC)n(C)c2c1=O]

1-[6-[5-Ethyl-3-methyl-4-oxo-6-(trifluoromethyl)imidazo[4,5-c]pyridin-2-yl]-5-ethylsulfanyl-3-pyridyl]cyclopropanecarbonitrile (prepared as described above) was treated under analogous conditions as described in step 2 of Example P12 and in analogy to methods described in WO2019/234158 to afford the desired compound P14. LCMS (method 5): m/z 479 [M+H]⁺; retention time: 0.92 min.

### Step 3: Preparation of the Individual Enantiomer Compounds P14-A and P14-B

The racemic 1-[6-[5-ethyl-3-methyl-4-oxo-6-(trifluoromethyl)imidazo[4,5-c]pyridin-2-yl]-5-(ethylsulfonimidoyl)-3-pyridyl]cyclopropanecarbonitrile (compound P14) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.46 cm×10 cm, 40° C.
Mobile phase: A: CO₂ B: iPrOH isocratic: 35% B in 4.8 min
ABPR: 1800 psi
Flow rate: 2.0 ml/min
Detection: 220 nm
Sample concentration: 1 mg/mL
Injection: 1 mL

### Preparative SFC Method:

Sepiatec Prep SFC 100
Column: Daicel CHIRALPAK® IG, 5 m, 2.0 cm×25 cm
Mobile phase: A: CO₂ B: iPrOH isocratic: 35% B
Backpressure: 150 bar
Flow rate: 60 ml/min
GLS pump: -
Detection: UV 220 nm
Sample: in DCM/ACN

### Results:

| First eluting enantiomer P14-A | Second eluting enantiomer P14-B |
|---|---|
| Retention time (min) ~2.92 | Retention time (min) ~4.06 |
| Chemical purity | Chemical purity |
| (area % at 220 nm) >99 | (area % at 220 nm) >99 |
| Enantiomeric excess (%) >99 | Enantiomeric excess (%) >99 |

### Example P15: Preparation of racemic 2-[[5-(ethylsulfonimidoyl)-6-[7-methyl-3-(trifluoromethyl)imidazo[4,5-c]pyridazin-6-yl]-3-pyridyl]oxy]-2-methyl-propanenitrile (Compound P15) and its Individual Enantiomers (Compounds P15-A and P15-B)

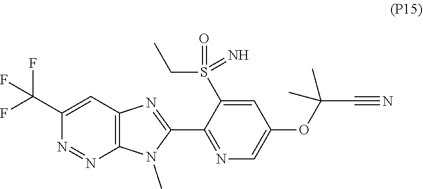 [SMILES: CCS(=N)(=O)c1cc(OC(C)(C)C#N)cnc1-c1nc2cc(C(F)(F)F)nnc2n1C]

### Step 1: Preparation of 2-[[5-ethylsulfanyl-6-[7-methyl-3-(trifluoromethyl)imidazo[4,5-c]pyridazin-6-yl]-3-pyridyl]oxy]-2-methyl-propanenitrile

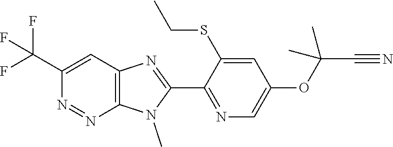 [SMILES: CCSc1cc(OC(C)(C)C#N)cnc1-c1nc2cc(C(F)(F)F)nnc2n1C]

This compound was prepared in analogy to methods described in WO2020/084075.

¹H NMR (400 MHz, chloroform-d) δ ppm 1.42 (t, J=7.34 Hz, 3H) 1.88 (s, 6H) 3.03 (q, J=7.34 Hz, 2H) 4.31 (s, 3H) 7.72 (d, J=2.57 Hz, 1H) 8.26 (s, 1H) 8.39 (d, J=2.57 Hz, 1H).

### Step 2: Preparation of racemic 2-[[5-(ethylsulfonimidoyl)-6-[7-methyl-3-(trifluoromethyl)imidazo[4,5-c]pyridazin-6-yl]-3-pyridyl]oxy]-2-methyl-propanenitrile (Compound P15)

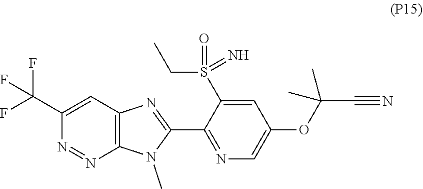 [SMILES: CCS(=N)(=O)c1cc(OC(C)(C)C#N)cnc1-c1nc2cc(C(F)(F)F)nnc2n1C]

2-[[5-Ethylsulfanyl-6-[7-methyl-3-(trifluoromethyl)imidazo[4,5-c]pyridazin-6-yl]-3-pyridyl]oxy]-2-methyl-propanenitrile (prepared as described above) was treated under analogous conditions as described in step 2 of Example P12 and in analogy to methods described in WO2020/084075 to afford the desired compound P15. LCMS (method 1): m/z 454 [M+H]⁺; retention time: 0.89 min.

### Step 3: Preparation of the Individual Enantiomer Compounds P15-A and P15-B

The racemic 2-[[5-(ethylsulfonimidoyl)-6-[7-methyl-3-(trifluoromethyl)imidazo[4,5-c]pyridazin-6-yl]-3-pyridyl]oxy]-2-methyl-propanenitrile (compound P15) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IC, 3 m, 0.3 cm×10 cm, 40° C.
Mobile phase: A: CO₂ B: EtOH isocratic: 15% B in 4.8 min
ABPR: 1800 psi
Flow rate: 2.0 ml/min
Detection: 270 nm
Sample concentration: 1 mg/mL
Injection: 1 μL

### Preparative SFC Method:

Sepiatec Prep SFC 100
Column: Daicel CHIRALPAK® IC, 5 m, 2.0 cm×25 cm
Mobile phase: A: CO₂ B: EtOH isocratic: 15% B in 14 min
Backpressure: 150 bar
Flow rate: 60 ml/min
GLS pump: 3 ml MeOH
Detection: UV 250 nm
Sample: in MeOH/DCM

### Results:

| First eluting enantiomer P15-A | Second eluting enantiomer P15-B |
|---|---|
| Retention time (min) ~1.32 | Retention time (min) ~3.86 |
| Chemical purity | Chemical purity |
| (area % at 267 nm) 99 | (area % at 267 nm) 99 |
| Enantiomeric excess (%) >99 | Enantiomeric excess (%) >99 |

### Example P16: Preparation of racemic 1-[5-(ethylsulfonimidoyl)-6-[3-methyl-6-(trifluoromethyl)imidazo[4,5-b]pyridin-2-yl]-3-pyridyl]cyclopropanecarbonitrile (Compound P16) and its Individual Enantiomers (Compounds P16-A and P16-B)

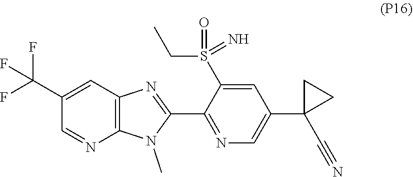 [SMILES: CCS(=N)(=O)c1cc(C2(C#N)CC2)cnc1-c1nc2cc(C(F)(F)F)cnc2n1C]

### Step 1: Preparation of 1-[5-ethylsulfanyl-6-[3-methyl-6-(trifluoromethyl)imidazo[4,5-b]pyridin-2-yl]-3-pyridyl]cyclopropanecarbonitrile

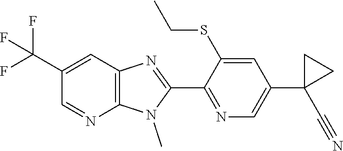 [SMILES: CCSc1cc(C2(C#N)CC2)cnc1-c1nc2cc(C(F)(F)F)cnc2n1C]

This compound was prepared in analogy to methods described in WO2019/234158.

LCMS (method 2): m/z 404 [M+H]⁺; retention time: 1.07 min.

### Step 2: Preparation of racemic 1-[5-(ethylsulfonimidoyl)-6-[3-methyl-6-(trifluoromethyl)imidazo[4,5-b]pyridin-2-yl]-3-pyridyl]cyclopropanecarbonitrile (Compound P16)

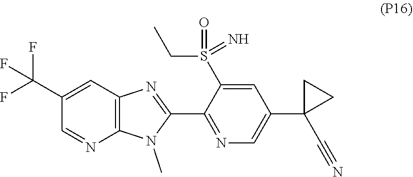 [SMILES: CCS(=N)(=O)c1cc(C2(C#N)CC2)cnc1-c1nc2cc(C(F)(F)F)cnc2n1C]

1-[5-Ethylsulfanyl-6-[3-methyl-6-(trifluoromethyl)imidazo[4,5-b]pyridin-2-yl]-3-pyridyl]cyclopropanecarbonitrile (prepared as described above) was treated under analogous conditions as described in step 2 of Example P12 and in analogy to methods described in WO2019/234158 to afford the desired compound P16. LCMS (method 2): m/z 435 [M+H]⁺; retention time: 0.87 min.

### Step 3: Preparation of the Individual Enantiomer Compounds P16-A and P16-B

The racemic 1-[5-(ethylsulfonimidoyl)-6-[3-methyl-6-(trifluoromethyl)imidazo[4,5-b]pyridin-2-yl]-3-pyridyl]cyclopropanecarbonitrile (compound P16) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.46 cm×10 cm, 40° C.
Mobile phase: A: CO₂ B: MeOH isocratic: 25% B in 4.8 min
ABPR: 1800 psi
Flow rate: 2.0 ml/min
Detection: 290 nm
Sample concentration: 1 mg/mL
Injection: 1 μL

### Preparative SFC Method:

Sepiatec Prep SFC 100
Column: Daicel CHIRALPAK® IG, 5 m, 2.0 cm×25 cm
Mobile phase: A: CO₂ B: MeOH isocratic: 17% B
Backpressure: 150 bar
Flow rate: 60 ml/min
GLS pump: -
Detection: UV 290 nm
Sample: in MeOH/DCM

### Results:

| First eluting enantiomer P16-A | Second eluting enantiomer P16-B |
|---|---|
| Retention time (min) ~2.35 | Retention time (min) ~3.37 |
| Chemical purity | Chemical purity |
| (area % at 290 nm) >99 | (area % at 290 nm) >99 |
| Enantiomeric excess (%) >99 | Enantiomeric excess (%) 99 |

### Example P17: Preparation of racemic 2-[[5-(ethylsulfonimidoyl)-2-methyl-6-[3-methyl-6-(trifluoromethyl)imidazo[4,5-c]pyridin-2-yl]-3-pyridyl]oxy]-2-methyl-propanenitrile (Compound P17) and its Individual Enantiomers (Compounds P17-A and P17-B)

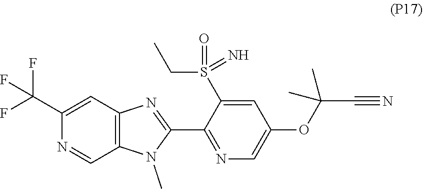 [SMILES: CCS(=N)(=O)c1cc(OC(C)(C)C#N)cnc1-c1nc2cc(C(F)(F)F)ncc2n1C]

### Step 1: Preparation of 2-[[5-ethylsulfanyl-2-methyl-6-[3-methyl-6-(trifluoromethyl)imidazo[4,5-c]pyridin-2-yl]-3-pyridyl]oxy]-2-methyl-propanenitrile

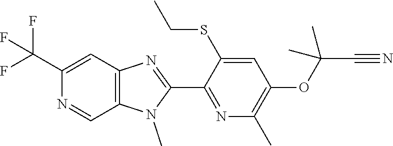 [SMILES: CCSc1cc(OC(C)(C)C#N)c(C)nc1-c1nc2cc(C(F)(F)F)ncc2n1C]

This compound was prepared in analogy to methods described in WO2020/084075.

LCMS (method 1): m/z 436 [M+H]⁺; retention time: 1.06 min.

### Step 2: Preparation of racemic 2-[[5-(ethylsulfonimidoyl)-2-methyl-6-[3-methyl-6-(trifluoromethyl)imidazo[4,5-c]pyridin-2-yl]-3-pyridyl]oxy]-2-methyl-propanenitrile (Compound P17)

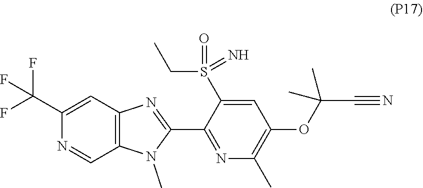 [SMILES: CCS(=N)(=O)c1cc(OC(C)(C)C#N)c(C)nc1-c1nc2cc(C(F)(F)F)ncc2n1C]

2-[[5-Ethylsulfanyl-2-methyl-6-[3-methyl-6-(trifluoromethyl)imidazo[4,5-c]pyridin-2-yl]-3-pyridyl]oxy]-2-methyl-propanenitrile (prepared as described above) was treated under analogous conditions as described in step 2 of Example P12 and in analogy to methods described in WO2020/084075 to afford the desired compound P17. LCMS (method 1): m/z 467 [M+H]⁺; retention time: 0.91 min.

### Step 3: Preparation of the Individual Enantiomer Compounds P17-A and P17-B

The racemic 2-[[5-(ethylsulfonimidoyl)-2-methyl-6-[3-methyl-6-(trifluoromethyl)imidazo[4,5-c]pyridin-2-yl]-3-pyridyl]oxy]-2-methyl-propanenitrile (compound P17) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IG, 3 m, 0.3 cm×10 cm, 40° C.
Mobile phase: A: CO₂ B: MeOH isocratic: 30% B in 4.8 min
ABPR: 1800 psi
Flow rate: 2.0 ml/min
Detection: 270 nm
Sample concentration: 1 mg/mL
Injection: 1 μL

### Preparative SFC Method:

Sepiatec Prep SFC 100
Column: Daicel CHIRALPAK® IG, 5 m, 2.0 cm×25 cm
Mobile phase: A: CO₂ B: MeOH isocratic: 30% B in 14 min
Backpressure: 150 bar
Flow rate: 60 ml/min
GLS pump: 3 ml
Detection: UV 270 nm
Sample: in MeOH/DCM

### Results:

| First eluting enantiomer P17-A | Second eluting enantiomer P17-B |
|---|---|
| Retention time (min) ~1.23 | Retention time (min) ~2.36 |
| Chemical purity | Chemical purity |
| (area % at 270 nm) 99 | (area % at 270 nm) 99 |
| Enantiomeric excess (%) >99 | Enantiomeric excess (%) >99 |

### Example P18: Preparation of racemic 1-[3-(ethylsulfonimidoyl)-4-[3-methyl-6-(trifluoromethyl)imidazo[4,5-c]pyridin-2-yl]phenyl]cyclopropanecarbonitrile (Compound P18) and its Individual Enantiomers (Compounds P18-A and P18-B)

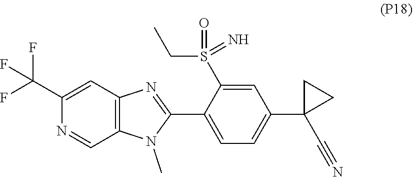 [SMILES: CCS(=N)(=O)c1cc(C2(C#N)CC2)ccc1-c1nc2cc(C(F)(F)F)ncc2n1C]

### Step 1: Preparation of 1-[3-ethylsulfanyl-4-[3-methyl-6-(trifluoromethyl)imidazo[4,5-c]pyridin-2-yl]phenyl]cyclopropanecarbonitrile

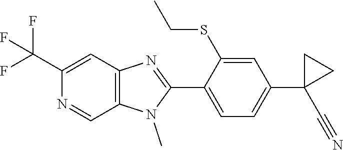 [SMILES: CCSc1cc(C2(C#N)CC2)ccc1-c1nc2cc(C(F)(F)F)ncc2n1C]

This compound was prepared in analogy to methods described in WO2019/234158.

LCMS (method 2): m/z 403 [M+H]1; retention time: 1.00 min.

### Step 2: Preparation of racemic 1-[3-(ethylsulfonimidoyl)-4-[3-methyl-6-(trifluoromethyl)imidazo[4,5-c]pyridin-2-yl]phenyl]cyclopropanecarbonitrile (Compound P18)

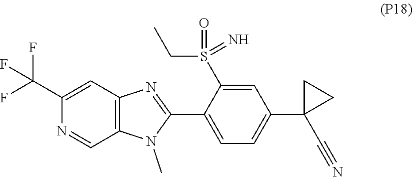 [SMILES: CCS(=N)(=O)c1cc(C2(C#N)CC2)ccc1-c1nc2cc(C(F)(F)F)ncc2n1C]

1-[3-Ethylsulfanyl-4-[3-methyl-6-(trifluoromethyl)imidazo[4,5-c]pyridin-2-yl]phenyl]cyclopropanecarbonitrile (prepared as described above) was treated under analogous conditions as described in step 2 of Example P12 and in analogy to methods described in WO2019/234158 to afford the desired compound P18. LCMS (method 2): m/z 434 [M+H]⁺; retention time: 0.83 min.

### Step 3: Preparation of the Individual Enantiomer Compounds P18-A and P18-B

The racemic 1-[3-(ethylsulfonimidoyl)-4-[3-methyl-6-(trifluoromethyl)imidazo[4,5-c]pyridin-2-yl]phenyl]cyclopropanecarbonitrile (compound P18) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IC, 3 m, 0.3 cm×10 cm, 40° C.
Mobile phase: A: CO₂ B: EtOH isocratic: 15% B in 4.8 min
ABPR: 1800 psi
Flow rate: 2.0 ml/min
Detection: 230 nm
Sample concentration: 1 mg/mL
Injection: 1 μL

### Preparative SFC Method:

Sepiatec Prep SFC 100
Column: Daicel CHIRALPAK® IC, 5 m, 2.0 cm×25 cm
Mobile phase: A: CO₂ B: EtOH isocratic: 15% B in 15 min
Backpressure: 150 bar
Flow rate: 60 ml/min
GLS pump: 5 ml EtOH
Detection: UV 230 nm
Sample: in MeOH

### Results:

| First eluting enantiomer P18-A | Second eluting enantiomer P18-B |
|---|---|
| Retention time (min) ~2.74 | Retention time (min) ~3.99 |
| Chemical purity | Chemical purity |
| (area % at 230 nm) >99 | (area % at 230 nm) >99 |
| Enantiomeric excess (%) >99 | Enantiomeric excess (%) >99 |

### Example P19: Preparation of racemic 2-[[6-[5-ethyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile (Compound P19) and its Individual Enantiomers (Compounds P19-A and P19-B)

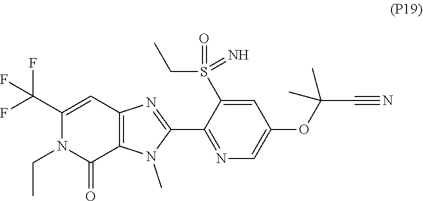 [SMILES: CCn1c(C(F)(F)F)cc2nc(-c3ncc(OC(C)(C)C#N)cc3S(=N)(=O)CC)n(C)c2c1=O]

### Step 1: Preparation of 2-[[6-[5-ethyl-3-methyl-4-oxo-6-(trifluoromethyl)imidazo[4,5-c]pyridin-2-yl]-5-ethylsulfanyl-3-pyridyl]oxy]-2-methyl-propanenitrile

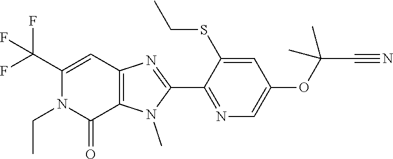 [SMILES: CCSc1cc(OC(C)(C)C#N)cnc1-c1nc2cc(C(F)(F)F)n(CC)c(=O)c2n1C]

This compound was prepared in analogy to methods described in WO2020/084075.

LCMS (method 4): m/z 466 [M+H]⁺; retention time: 1.10 min.

### Step 2: Preparation of racemic 2-[[6-[5-ethyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile (Compound P19)

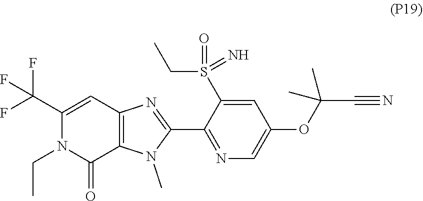 [SMILES: CCn1c(C(F)(F)F)cc2nc(-c3ncc(OC(C)(C)C#N)cc3S(=N)(=O)CC)n(C)c2c1=O]

2-[[6-[5-Ethyl-3-methyl-4-oxo-6-(trifluoromethyl)imidazo[4,5-c]pyridin-2-yl]-5-ethylsulfanyl-3-pyridyl]oxy]-2-methyl-propanenitrile (prepared as described above) was treated under analogous conditions as described in step 2 of Example P12 and in analogy to methods described in WO2020/084075 to afford the desired compound P19. LCMS (method 4): m/z 497 [M+H]⁺; retention time: 0.93 min.

### Step 3: Preparation of the Individual Enantiomer Compounds P19-A and P19-B

The racemic 2-[[6-[5-ethyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile (compound P19) mixture was submitted to chiral resolution by preparative SFC using the conditions outlined hereafter.

### Analytical SFC Method:

SFC: Waters Acquity UPC²/QDa
PDA Detector Waters Acquity UPC²
Column: Daicel SFC CHIRALPAK® IA, 3 m, 0.3 cm×10 cm, 40° C.
Mobile phase: A: CO₂ B: iPrOH isocratic: 20% B in 4.8 min
ABPR: 1800 psi
Flow rate: 2.0 ml/min
Detection: 220 nm
Sample concentration: 1 mg/mL
Injection: 1 μL

### Preparative SFC Method:

Sepiatec Prep SFC 100
Column: Daicel CHIRALPAK® IA, 5 m, 2.0 cm×25 cm
Mobile phase: A: CO₂ B: iPrOH isocratic: 20% B in 14 min
Backpressure: 150 bar
Flow rate: 60 ml/min
GLS pump: 2 ml MeOH
Detection: UV 220 nm
Sample: in MeOH/DCM

### Results:

| First eluting enantiomer P19-A | Second eluting enantiomer P19-B |
|---|---|
| Retention time (min) ~1.18 | Retention time (min) ~2.18 |
| Chemical purity | Chemical purity |
| (area % at 220 nm) 99 | (area % at 220 nm) 99 |
| Enantiomeric excess (%) >99 | Enantiomeric excess (%) >99 |

TABLE P
Examples of racemic mixture of compounds of formula (I)

| | | | LCMS | | |
|---|---|---|---|---|---|
| No. | IUPAC name | Structures | Rₜ (min) | [M + H]⁺ (measured) | Method |
| P1 | 2-[[6-[5-cyclopropyl-3- methyl-4-oxo-6- (trifluoromethyl)imidazo[4,5- c]pyridin-2-yl]-5- (ethylsulfonimidoyl)-3- pyridyl]oxy]-2-methyl- propanenitrile | 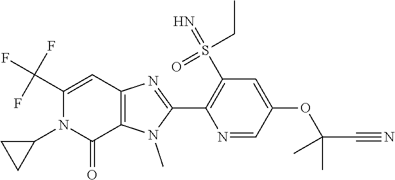 [SMILES: CCS(=N)(=O)c1cc(OC(C)(C)C#N)cnc1-c1nc2cc(C(F)(F)F)n(C3CC3)c(=O)c2n1C] | 0.92 | 509 | 4 |
| P2 | 1-[5-(ethylsulfonimidoyl)-6- [7-methyl-3- (trifluoromethyl)imidazo[4,5- c]pyridazin-6-yl]-3- pyridyl]cyclopropane- carbonitrile | 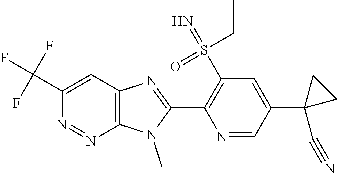 [SMILES: CCS(=N)(=O)c1cc(C2(C#N)CC2)cnc1-c1nc2cc(C(F)(F)F)nnc2n1C] | 0.82 | 436 | 5 |
| P3 | 2-[[5-(ethylsulfonimidoyl)- 6-[7- (trifluoromethyl)imidazo[1,2- a]pyridin-2-yl]-3- pyridyl]oxy]-2-methyl- propanenitrile | 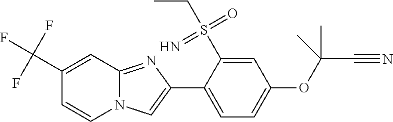 [SMILES: CCS(=N)(=O)c1cc(OC(C)(C)C#N)ccc1-c1cn2ccc(C(F)(F)F)cc2n1] | 0.88 | 438 | 1 |
| P4 | 2-[5-(ethylsulfonimidoyl)-6- [3-methyl-6- (trifluoromethyl)imidazo[4,5- c]pyridin-2-yl]-3-pyridyl]- 2-methyl-propanenitrile | 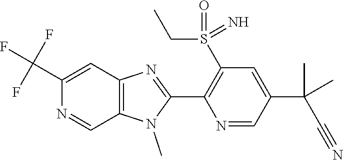 [SMILES: CCS(=N)(=O)c1cc(C(C)(C)C#N)cnc1-c1nc2cc(C(F)(F)F)ncc2n1C] | 0.83 | 437 | 2 |
| P5 | 2-[[5-(ethylsulfonimidoyl)- 2-methyl-6-[3-methyl-6- (trifluoromethyl)imidazo[4,5- b]pyridin-2-yl]-3- pyridyl]oxy]-2-methyl- propanenitrile | 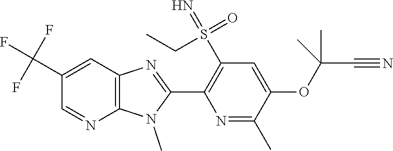 [SMILES: CCS(=N)(=O)c1cc(OC(C)(C)C#N)c(C)nc1-c1nc2cc(C(F)(F)F)cnc2n1C] | 0.97 | 467 | 1 |
| P6 | 1-[3-(ethylsulfonimidoyl)-4- [3-methyl-6- (trifluoromethyl)imidazo[4,5- b]pyridin-2- yl]phenyl]cyclopropane- carbonitrile | 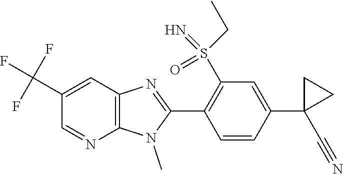 [SMILES: CCS(=N)(=O)c1cc(C2(C#N)CC2)ccc1-c1nc2cc(C(F)(F)F)cnc2n1C] | 0.88 | 434 | 2 |
| P7 | 2-[[5-(ethylsulfonimidoyl)- 6-[7- (trifluoromethylsulfanyl) imidazo[1,2-c]pyrimidin-2-yl]- 3-pyridyl]oxy]-2-methyl- propanenitrile | 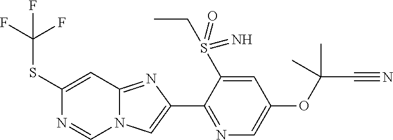 [SMILES: CCS(=N)(=O)c1cc(OC(C)(C)C#N)cnc1-c1cn2cnc(SC(F)(F)F)cc2n1] | 0.89 | 471 | 1 |
| P8 | 1-[5-(ethylsulfonimidoyl)-6- [5-methoxy-3-methyl-4- oxo-6- (trifluoromethyl)imidazo[4,5- c]pyridin-2-yl]-3- pyridyl]cyclopropane- carbonitrile | 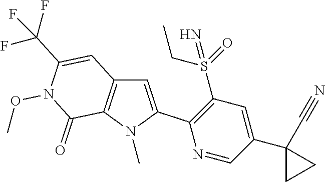 [SMILES: CCS(=N)(=O)c1cc(C2(C#N)CC2)cnc1-c1cc2cc(C(F)(F)F)n(OC)c(=O)c2n1C] | 0.85 | 481 | 5 |
| P9 | 2-[[5-(ethylsulfonimidoyl)- 6-[3-methyl-6- (trifluoromethyl)imidazo[4,5- b]pyridin-2-yl]-3- pyridyl]oxy]-2-methyl- propanenitrile | 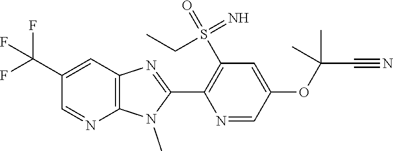 [SMILES: CCS(=N)(=O)c1cc(OC(C)(C)C#N)cnc1-c1nc2cc(C(F)(F)F)cnc2n1C] | 0.93 | 453 | 1 |
| P10 | 1-[5-(ethylsulfonimidoyl)-6- [3-methyl-6- (trifluoromethyl)imidazo[4,5- c]pyridin-2-yl]-3- pyridyl]cyclopropane- carbonitrile | 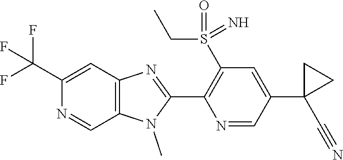 [SMILES: CCS(=N)(=O)c1cc(C2(C#N)CC2)cnc1-c1nc2cc(C(F)(F)F)ncc2n1C] | 0.82 | 435 | 1 |
| P11 | 2-[[5-(ethylsulfonimidoyl)- 6-[7- (trifluoromethyl)imidazo[1,2- c]pyrimidin-2-yl]-3- pyridyl]oxy]-2-methyl- propanenitrile | 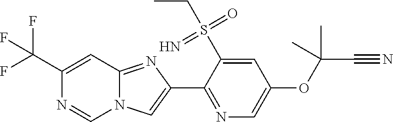 [SMILES: CCS(=N)(=O)c1cc(OC(C)(C)C#N)cnc1-c1cn2cnc(C(F)(F)F)cc2n1] | 0.84 | 439 | 1 |
| P12 | 2-[5-(ethylsulfonimidoyl)-6- [3-methyl-6- (trifluoromethyl)imidazo[4,5- b]pyridin-2-yl]-3-pyridyl]- 2-methyl-propanenitrile | 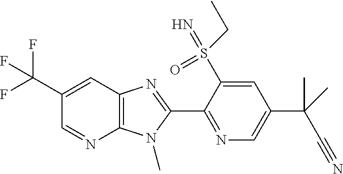 [SMILES: CCS(=N)(=O)c1cc(C(C)(C)C#N)cnc1-c1nc2cc(C(F)(F)F)cnc2n1C] | 0.91 | 437 | 2 |
| P13 | 2-[[5-(ethylsulfonimidoyl)- 6-[3-methyl-6- (trifluoromethyl)imidazo[4,5- c]pyridin-2-yl]-3- pyridyl]oxy]-2-methyl- propanenitrile | 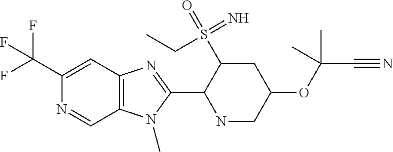 [SMILES: CCS(=N)(=O)C1CC(OC(C)(C)C#N)CNC1c1nc2cc(C(F)(F)F)ncc2n1C] | 0.86 | 453 | 1 |
| P14 | 1-[6-[5-ethyl-3-methyl-4- oxo-6- (trifluoromethyl)imidazo[4,5- c]pyridin-2-yl]-5- (ethylsulfonimidoyl)-3- pyridyl]cyclopropane- carbonitrile | 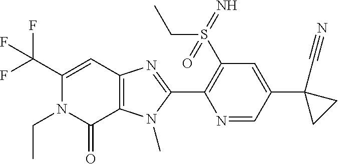 [SMILES: CCn1c(C(F)(F)F)cc2nc(-c3ncc(C4(C#N)CC4)cc3S(=N)(=O)CC)n(C)c2c1=O] | 0.92 | 479 | 5 |
| P15 | 2-[[5-(ethylsulfonimidoyl)- 6-[7-methyl-3- (trifluoromethyl)imidazo[4,5- c]pyridazin-6-yl]-3- pyridyl]oxy]-2-methyl- propanenitrile | 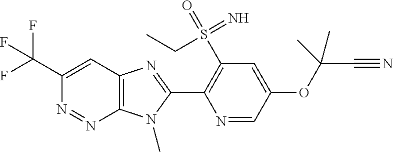 [SMILES: CCS(=N)(=O)c1cc(OC(C)(C)C#N)cnc1-c1nc2cc(C(F)(F)F)nnc2n1C] | 0.89 | 454 | 1 |
| P16 | 1-[5-(ethylsulfonimidoyl)-6- [3-methyl-6- (trifluoromethyl)imidazo[4,5- b]pyridin-2-yl]-3- pyridyl]cyclopropane- carbonitrile | 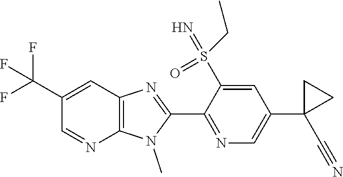 [SMILES: CCS(=N)(=O)c1cc(C2(C#N)CC2)cnc1-c1nc2cc(C(F)(F)F)cnc2n1C] | 0.87 | 435 | 2 |
| P17 | 2-[[5-(ethylsulfonimidoyl)- 2-methyl-6-[3-methyl-6- (trifluoromethyl)imidazo[4,5- c]pyridin-2-yl]-3- pyridyl]oxy]-2-methyl- propanenitrile | 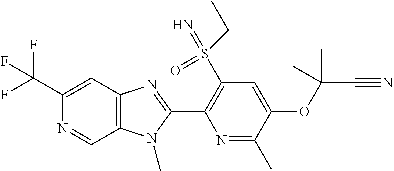 [SMILES: CCS(=N)(=O)c1cc(OC(C)(C)C#N)c(C)nc1-c1nc2cc(C(F)(F)F)ncc2n1C] | 0.91 | 467 | 1 |
| P18 | 1-[3-(ethylsulfonimidoyl)-4- [3-methyl-6- (trifluoromethyl)imidazo[4,5- c]pyridin-2- yl]phenyl]cyclopropane- carbonitrile | 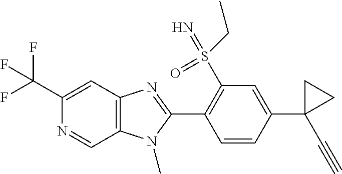 [SMILES: C#CC1(c2ccc(-c3nc4cc(C(F)(F)F)ncc4n3C)c(S(=N)(=O)CC)c2)CC1] | 0.83 | 434 | 1 |
| P19 | 2-[[6-[5-ethyl-3-methyl-4- oxo-6- (trifluoromethyl)imidazo[4,5- c]pyridin-2-yl]-5- (ethylsulfonimidoyl)-3- pyridyl]oxy]-2-methyl- propanenitrile | 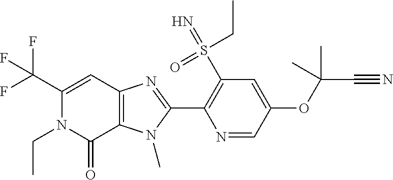 [SMILES: CCn1c(C(F)(F)F)cc2nc(-c3ncc(OC(C)(C)C#N)cc3S(=N)(=O)CC)n(C)c2c1=O] | 0.93 | 497 | 4 |

TABLE P(E)
Examples of compounds of formula (I) as individual enantiomers

| | IUPAC name | | Rₜ | [M + H]⁺ | |
|---|---|---|---|---|---|
| No. | of the racemate | Enantiomer | (min) | (measured) | Method |
| P1-A | 2-[[6-[5-cyclopropyl-3-methyl-4-oxo- | First eluting | 1.81 | 509 | SFC: Waters Acquity UPC²/QDa |
| P1-B | 6-(trifluoromethyl)imidazo[4,5- | Second eluting | 3.75 | 509 | PDA Detector Waters Acquity UPC² |
| | c]pyridin-2-yl]-5-(ethylsulfonimidoyl)- | | | | Column: Daicel SFC CHIRALPAK ® |
| | 3-pyridyl]oxy]-2-methyl- | | | | IA, 3 μm, 0.3 cm × 10 cm, 40° C. |
| | propanenitrile | | | | Mobile phase: A: CO₂ B: iPrOH |
| | | | | | isocratic: 20% B in 4.8 min |
| | | | | | ABPR: 1800 psi |
| | | | | | Flow rate: 2.0 ml/min |
| | | | | | Detection: 220 nm |
| | | | | | Sample concentration: 1 mg/ml |
| | | | | | Injection: 1 μL |
| P2-A | 1-[5-(ethylsulfonimidoyl)-6-[7-methyl- | First eluting | 1.36 | 436 | SFC: Waters Acquity UPC²/QDa |
| P2-B | 3-(trifluoromethyl)imidazo[4,5- | Second eluting | 3.54 | 436 | PDA Detector Waters Acquity UPC² |
| | c]pyridazin-6-yl]-3- | | | | Column: Daicel SFC CHIRALPAK ® |
| | pyridyl]cyclopropanecarbonitrile | | | | IC, 3 μm, 0.3 cm × 10 cm, 40° C. |
| | | | | | Mobile phase: A: CO₂ B: EtOH |
| | | | | | isocratic: 20% B in 4.8 min |
| | | | | | ABPR: 1800 psi |
| | | | | | Flow rate: 2.0 ml/min |
| | | | | | Detection: 265 nm |
| | | | | | Sample concentration: 1 mg/mL |
| | | | | | Injection: 1 mL |
| P3-A | 2-[[5-(ethylsulfonimidoyl)-6-[7- | First eluting | 1.97 | 438 | SFC: Waters Acquity UPC²/QDa |
| P3-B | (trifluoromethyl)imidazo[1,2- | Second eluting | 3.19 | 438 | PDA Detector Waters Acquity UPC² |
| | a]pyridin-2-yl]-3-pyridyl]oxy]-2- | | | | Column: Daicel SFC CHIRALPAK ® |
| | methyl-propanenitrile | | | | IC, 3 μm, 0.3 cm × 10 cm, 40° C. |
| | | | | | Mobile phase: A: CO₂ B: MeOH |
| | | | | | isocratic: 20% B in 4.8 min |
| | | | | | ABPR: 1800 psi |
| | | | | | Flow rate: 2.0 ml/min |
| | | | | | Detection: 250 nm |
| | | | | | Sample concentration: 1 mg/ml |
| | | | | | Injection: 1 μL |
| P4-A | 2-[5-(ethylsulfonimidoyl)-6-[3-methyl- | First eluting | 1.76 | 437 | SFC: Waters Acquity UPC²/QDa |
| P4-B | 6-(trifluoromethyl)imidazo[4,5- | Second eluting | 2.82 | 437 | PDA Detector Waters Acquity UPC² |
| | c]pyridin-2-yl]-3-pyridyl]-2-methyl- | | | | Column: Daicel SFC CHIRALPAK ® |
| | propanenitrile | | | | IG, 3 μm, 0.46 cm × 10 cm, 40° C. |
| | | | | | Mobile phase: A: CO₂ B: MeOH |
| | | | | | isocratic: 30% B in 4.8 min |
| | | | | | ABPR: 1800 psi |
| | | | | | Flow rate: 2.0 ml/min |
| | | | | | Detection: 265 nm |
| | | | | | Sample concentration: 1 mg/ml |
| | | | | | Injection: 1 mL |
| P5-A | 2-[[5-(ethylsulfonimidoyl)-2-methyl- | First eluting | 1.92 | 467 | SFC: Waters Acquity UPC²/QDa |
| P5-B | 6-[3-methyl-6- | Second eluting | 3.12 | 467 | PDA Detector Waters Acquity UPC² |
| | (trifluoromethyl)imidazo[4,5- | | | | Column: Daicel SFC CHIRALPAK ® |
| | b]pyridin-2-yl]-3-pyridyl]oxy]- | | | | IG, 3 μm, 0.3 cm × 10 cm, 40° C. |
| | 2-methyl-propanenitrile | | | | Mobile phase: A: CO₂ B: MeOH |
| | | | | | isocratic: 15% B in 4.8 min |
| | | | | | ABPR: 1800 psi |
| | | | | | Flow rate: 2.0 ml/min |
| | | | | | Detection: 280 nm |
| | | | | | Sample concentration: 1 mg/ml |
| | | | | | Injection: 1 μL |
| P6-A | 1-[3-(ethylsulfonimidoyl)-4-[3-methyl- | First eluting | 2.34 | 434 | SFC: Waters Acquity UPC²/QDa |
| P6-B | 6-(trifluoromethyl)imidazo[4,5- | Second eluting | 3.99 | 434 | PDA Detector Waters Acquity UPC² |
| | b]pyridin-2- | | | | Column: Daicel SFC CHIRALPAK ® |
| | yl]phenyl]cyclopropanecarbonitrile | | | | IG, 3 μm, 0.46 cm × 10 cm, 40° C. |
| | | | | | Mobile phase: A: CO₂ B: iPrOH |
| | | | | | isocratic: 35% B in 4.8 min |
| | | | | | ABPR: 1800 psi |
| | | | | | Flow rate: 2.0 ml/min |
| | | | | | Detection: 290 nm |
| | | | | | Sample concentration: 1 mg/ml |
| | | | | | Injection: 1 mL |
| P7-A | 2-[[5-(ethylsulfonimidoyl)-6-[7- | First eluting | 1.28 | 471 | SFC: Waters Acquity UPC²/QDa |
| P7-B | (trifluoromethylsulfanyl)imidazo[1,2- | Second eluting | 3.63 | 471 | PDA Detector Waters Acquity UPC² |
| | c]pyrimidin-2-yl]-3-pyridyl]oxy]-2- | | | | Column: Daicel SFC CHIRALPAK ® |
| | methyl-propanenitrile | | | | IC, 3 μm, 0.3 cm × 10 cm, 40° C. |
| | | | | | Mobile phase: A: CO₂ B: MeOH |
| | | | | | isocratic: 30% B in 4.8 min |
| | | | | | ABPR: 1800 psi |
| | | | | | Flow rate: 2.0 ml/min |
| | | | | | Detection: 250 nm |
| | | | | | Sample concentration: 1 mg/ml |
| | | | | | Injection: 1 μL |
| P8-A | 1-[5-(ethylsulfonimidoyl)-6- | First eluting | 1.73 | 481 | SFC: Waters Acquity UPC²/QDa |
| P8-B | [5-methoxy-3-methyl-4-oxo-6- | Second eluting | 3.15 | 481 | PDA Detector Waters Acquity UPC² |
| | (trifluoromethyl)imidazo[4,5- | | | | Column: Daicel SFC CHIRALPAK ® |
| | c]pyridin-2-yl]-3- | | | | IA, 3 μm, 0.3 cm × 10 cm, 40° C. |
| | pyridyl]cyclopropanecarbonitrile | | | | Mobile phase: A: CO₂ B: iPrOH |
| | | | | | isocratic: 25% B in 4.8 min |
| | | | | | ABPR: 1800 psi |
| | | | | | Flow rate: 2.0 ml/min |
| | | | | | Detection: 220 nm |
| | | | | | Sample concentration: 1 mg/ml |
| | | | | | Injection: 1 mL |
| P9-A | 2-[[5-(ethylsulfonimidoyl)-6-[3- | First eluting | 1.49 | 453 | SFC: Waters Acquity UPC²/QDa |
| P9-B | methyl-6- | Second eluting | 2.01 | 453 | PDA Detector Waters Acquity UPC² |
| | (trifluoromethyl)imidazo[4,5- | | | | Column: Daicel SFC CHIRALPAK ® |
| | b]pyridin-2-yl]-3-pyridyl]oxy]-2- | | | | IA, 3 μm, 0.3 cm × 10 cm, 40° C. |
| | methyl-propanenitrile | | | | Mobile phase: A: CO₂ B: EtOH |
| | | | | | isocratic: 10% B in 4.8 min |
| | | | | | ABPR: 1800 psi |
| | | | | | Flow rate: 2.0 ml/min |
| | | | | | Detection: 290 nm |
| | | | | | Sample concentration: 1 mg/ml |
| | | | | | Injection: 1 μL |
| P10-A | 1-[5-(ethylsulfonimidoyl)-6-[3-methyl- | First eluting | 1.68 | 435 | SFC: Waters Acquity UPC²/QDa |
| P10-B | 6-(trifluoromethyl)imidazo[4,5- | Second eluting | 2.99 | 435 | PDA Detector Waters Acquity UPC² |
| | c]pyridin-2-yl]-3- | | | | Column: Daicel SFC CHIRALPAK ® |
| | pyridyl]cyclopropanecarbonitrile | | | | IG, 3 μm, 0.46 cm × 10 cm, 40° C. |
| | | | | | Mobile phase: A: CO₂ B: MeOH |
| | | | | | isocratic: 40% B in 4.8 min |
| | | | | | ABPR: 1800 psi |
| | | | | | Flow rate: 2.0 ml/min |
| | | | | | Detection: 250 nm |
| | | | | | Sample concentration: 1 mg/ml |
| | | | | | Injection: 1 mL |
| P11-A | 2-[[5-(ethylsulfonimidoyl)-6-[7- | First eluting | 1.08 | 439 | SFC: Waters Acquity UPC²/QDa |
| P11-B | (trifluoromethyl)imidazo[1,2- | Second eluting | 2.78 | 439 | PDA Detector Waters Acquity UPC² |
| | c]pyrimidin-2-yl]-3-pyridyl]oxy]- | | | | Column: Daicel SFC CHIRALPAK ® |
| | 2-methyl-propanenitrile | | | | IC, 3 μm, 0.3 cm × 10 cm, 40° C. |
| | | | | | Mobile phase: A: CO₂ B: MeOH |
| | | | | | isocratic: 30% B in 4.8 min |
| | | | | | ABPR: 1800 psi |
| | | | | | Flow rate: 2.0 ml/min |
| | | | | | Detection: 240 nm |
| | | | | | Sample concentration: 1 mg/ml |
| | | | | | Injection: 1 μL |
| P12-A | 2-[5-(ethylsulfonimidoyl)-6-[3-methyl- | First eluting | 2.42 | 437 | SFC: Waters Acquity UPC²/QDa |
| P12-B | 6-(trifluoromethyl)imidazo[4,5- | Second eluting | 3.17 | 437 | PDA Detector Waters Acquity UPC² |
| | b]pyridin-2-yl]-3-pyridyl]-2-methyl- | | | | Column: Daicel SFC CHIRALPAK ® |
| | propanenitrile | | | | IG, 3 μm, 0.46 cm × 10 cm, 40° C. |
| | | | | | Mobile phase: A: CO₂ B: EtOH |
| | | | | | isocratic: 20% B in 4.8 min |
| | | | | | ABPR: 1800 psi |
| | | | | | Flow rate: 2.0 ml/min |
| | | | | | Detection: 220 nm |
| | | | | | Sample concentration: 1 mg/ml |
| | | | | | Injection: 1 mL |
| P13-A | 2-[[5-(ethylsulfonimidoyl)-6-[3- | First eluting | 1.70 | 453 | SFC: Waters Acquity UPC²/QDa |
| P13-B | methyl- | Second eluting | 3.89 | 453 | PDA Detector Waters Acquity UPC² |
| | 6-(trifluoromethyl)imidazo[4,5- | | | | Column: Daicel SFC CHIRALPAK ® |
| | c]pyridin-2-yl]-3-pyridyl]oxy]-2- | | | | IG, 3 μm, 0.3 cm × 10 cm, 40° C. |
| | methyl-propanenitrile | | | | Mobile phase: A: CO₂ B: MeOH |
| | | | | | isocratic: 30% B in 4.8 min |
| | | | | | ABPR: 1800 psi |
| | | | | | Flow rate: 2.0 ml/min |
| | | | | | Detection: 270 nm |
| | | | | | Sample concentration: 1 mg/ml |
| | | | | | Injection: 1 μL |
| P14-A | 1-[6-[5-ethyl-3-methyl-4-oxo-6- | First eluting | 2.92 | 479 | SFC: Waters Acquity UPC²/QDa |
| P14-B | (trifluoromethyl)imidazo[4,5- | Second eluting | 4.06 | 479 | PDA Detector Waters Acquity UPC² |
| | c]pyridin-2-yl]-5- | | | | Column: Daicel SFC CHIRALPAK ® |
| | (ethylsulfonimidoyl)-3- | | | | IG, 3 μm, 0.46 cm × 10 cm, 40° C. |
| | pyridyl]cyclopropanecarbonitrile | | | | Mobile phase: A: CO₂ B: iPrOH |
| | | | | | isocratic: 35% B in 4.8 min |
| | | | | | ABPR: 1800 psi |
| | | | | | Flow rate: 2.0 ml/min |
| | | | | | Detection: 220 nm |
| | | | | | Sample concentration: 1 mg/mL |
| | | | | | Injection: 1 mL |
| P15-A | 2-[[5-(ethylsulfonimidoyl)-6-[7- | First eluting | 1.32 | 454 | SFC: Waters Acquity UPC²/QDa |
| P15-B | methyl-3- | Second eluting | 3.86 | 454 | PDA Detector Waters Acquity UPC² |
| | (trifluoromethyl)imidazo[4,5- | | | | Column: Daicel SFC CHIRALPAK ® |
| | c]pyridazin-6-yl]-3-pyridyl]oxy]-2- | | | | IC, 3 μm, 0.3 cm × 10 cm, 40° C. |
| | methyl-propanenitrile | | | | Mobile phase: A: CO₂ B: EtOH |
| | | | | | isocratic: 15% B in 4.8 min |
| | | | | | ABPR: 1800 psi |
| | | | | | Flow rate: 2.0 ml/min |
| | | | | | Detection: 270 nm |
| | | | | | Sample concentration: 1 mg/mL |
| | | | | | Injection: 1 μL |
| P16-A | 1-[5-(ethylsulfonimidoyl)-6-[3-methyl- | First eluting | 2.35 | 435 | SFC: Waters Acquity UPC²/QDa |
| P16-B | 6-(trifluoromethyl)imidazo[4,5- | Second eluting | 3.37 | 435 | PDA Detector Waters Acquity UPC² |
| | b]pyridin-2-yl]-3- | | | | Column: Daicel SFC CHIRALPAK ® |
| | pyridyl]cyclopropanecarbonitrile | | | | IG, 3 μm, 0.46 cm × 10 cm, 40° C. |
| | | | | | Mobile phase: A: CO₂ B: MeOH |
| | | | | | isocratic: 25% B in 4.8 min |
| | | | | | ABPR: 1800 psi |
| | | | | | Flow rate: 2.0 ml/min |
| | | | | | Detection: 290 nm |
| | | | | | Sample concentration: 1 mg/mL |
| | | | | | Injection: 1 mL |
| P17-A | 2-[[5-(ethylsulfonimidoyl)-2- | First eluting | 1.23 | 467 | SFC: Waters Acquity UPC²/QDa |
| P17-B | methyl-6-[3-methyl-6- | Second eluting | 2.36 | 467 | PDA Detector Waters Acquity UPC² |
| | (trifluoromethyl)imidazo[4,5- | | | | Column: Daicel SFC CHIRALPAK ® |
| | c]pyridin-2-yl]-3-pyridyl]oxy]-2- | | | | IG, 3 μm, 0.3 cm × 10 cm, 40° C. |
| | methyl-propanenitrile | | | | Mobile phase: A: CO₂ B: MeOH |
| | | | | | isocratic: 30% B in 4.8 min |
| | | | | | ABPR: 1800 psi |
| | | | | | Flow rate: 2.0 ml/min |
| | | | | | Detection: 270 nm |
| | | | | | Sample concentration: 1 mg/ml |
| | | | | | Injection: 1 μL |
| P18-A | 1-[3-(ethylsulfonimidoyl)-4-[3-methyl- | First eluting | 2.74 | 434 | SFC: Waters Acquity UPC²/QDa |
| P18-B | 6-(trifluoromethyl)imidazo[4,5- | Second eluting | 3.99 | 434 | PDA Detector Waters Acquity UPC² |
| | c]pyridin-2- | | | | Column: Daicel SFC CHIRALPAK ® |
| | yl]phenyl]cyclopropanecarbonitrile | | | | IC, 3 μm, 0.3 cm × 10 cm, 40° C. |
| | | | | | Mobile phase: A: CO₂ B: EtOH |
| | | | | | isocratic: 15% B in 4.8 min |
| | | | | | ABPR: 1800 psi |
| | | | | | Flow rate: 2.0 ml/min |
| | | | | | Detection: 230 nm |
| | | | | | Sample concentration: 1 mg/ml |
| | | | | | Injection: 1 mL |
| P19-A | 2-[[6-[5-ethyl-3-methyl-4-oxo-6- | First eluting | 1.18 | 497 | SFC: Waters Acquity UPC²/QDa |
| P19-B | (trifluoromethyl)imidazo[4,5- | Second eluting | 2.18 | 497 | PDA Detector Waters Acquity UPC² |
| | c]pyridin-2-yl]-5-(ethylsulfonimidoyl)- | | | | Column: Daicel SFC CHIRALPAK ® |
| | 3-pyridyl]oxy]-2-methyl-propanenitrile | | | | IA, 3 μm, 0.3 cm × 10 cm, 40° C. |
| | | | | | Mobile phase: A: CO₂ B: iPrOH |
| | | | | | isocratic: 20% B in 4.8 min |
| | | | | | ABPR: 1800 psi |
| | | | | | Flow rate: 2.0 ml/min |
| | | | | | Detection: 220 nm |
| | | | | | Sample concentration: 1 mg/ml |
| | | | | | Injection: 1 μL |

The characteristic measured molecular ion (M+H)⁺ values in Table P(E) were recorded on a Mass Spectrometer from Waters (QDa) (Polarity: positive and negative ions), Detector Gain 1, Temperature Probe: 500° C., Cone Voltage: 10V, ESI Capillary Positive Voltage 0.8-Negative Voltage 0.8, Sampling Frequency 5 Hz, Mass range: 100 to 850 Da.

### Example E1: Preparation of (R)-2-[[5-(ethylsulfonimidoyl)-6-[3-methyl-6-(trifluoromethyl)imidazo[4,5-b]pyridin-2-yl]-3-pyridyl]oxy]-2-methyl-propanenitrile (Compound (R)-P9)

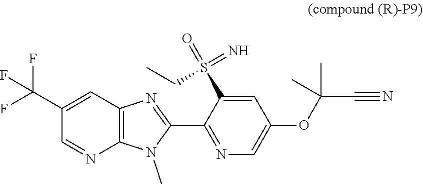 [SMILES: CC[S@@](=N)(=O)c1cc(OC(C)(C)C#N)cnc1-c1nc2cc(C(F)(F)F)cnc2n1C]

Compound (R)-P9 in an enantiomerically enriched form (major (R)-stereoisomer having the absolute configuration depicted in the drawing) was prepared in two steps from 2-[[5-ethylsulfanyl-6-[3-methyl-6-(trifluoromethyl)imidazo[4,5-b]pyridin-2-yl]-3-pyridyl]oxy]-2-methyl-propanenitrile by means of a stereoselective oxidation, followed by a stereospecific imination reaction.

### Step 1: Preparation of enantioenriched 2-[[5-[(R)-ethylsulfinyl]-6-[3-methyl-6-(trifluoromethyl)imidazo[4,5-b]pyridin-2-yl]-3-pyridyl]oxy]-2-methyl-propanenitrile (compound (R)—S09)

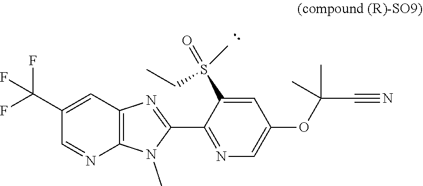

2-[[5-ethylsulfanyl-6-[3-methyl-6-(trifluoromethyl)imidazo[4,5-b]pyridin-2-yl]-3-pyridyl]oxy]-2-methyl-propanenitrile (prepared as described above in Example P9, step 1) (300 mg, 0.680 mmol), iron(III)acetylacetonate (12.2 mg, 0.035 mmol), 2-[(E)-[(1R)-1-(hydroxymethyl)-2,2-dimethyl-propyl]iminomethyl]-4,6-diiodo-phenol (prepared according to Chem Eur J 2005, 11, 1086-1092) (37.1 mg, 0.068 mmol) and 4-methoxybenzoic acid (2.6 mg, 0.017 mmol) were dissolved in toluene (2.7 mL). The solution was cooled to 0° C. and hydrogen peroxide (30% aqueous solution, 0.139 mL, 1.36 mmol) was added. The reaction was stirred at 0° C. for 30 minutes then warmed to 10° C. and stirred overnight, before being warmed to room temperature and stirred for a further 6 hours. The reaction mixture was then poured into a mixture of ethyl acetate and aqueous sodium thiosulfate solution, the layers were separated, and the aqueous phase extracted with ethyl acetate. The combined organic phases were washed with water and a 0.5M aqueous hydrochloric acid solution, dried over sodium sulfate and concentrated in vacuo. The residue was purified by flash column chromatography (silica, ethyl acetate in cyclohexane) to afford the title compound.

LCMS (method 6): m/z 438 [M+H]⁺; retention time: 1.86 min.

Enantiomeric excess was measured according to the following method:

### Analytical SFC Method:

SFC: Waters Acquity UPC²/QDa
PDA Detector Waters Acquity UPC²
Column: Daicel SFC CHIRALPAK® IG, 3□m, 0.46 cm×10 cm, 40° C.
Mobile phase: A: CO₂ B: EtOH isocratic: 20% B in 4.8 min
ABPR: 1800 psi
Flow rate: 2.0 ml/min
Detection: 310 nm
Sample concentration: 1 mg/mL in MeOH/ACN 50/50
Injection: 1 □L

| First eluting enantiomer (R)-SO9 | Second eluting enantiomer (S)-SO9 |
|---|---|
| Retention time (min) ~2.37 | Retention time (min) ~3.54 |
| Chemical purity | Chemical purity |
| (area % at 310 nm) 92.4 | (area % at 310 nm) 7.6 |

Enantiomeric excess (%) 84.8

All chiral sulfinyl compounds (R)—SO1 to (R)—SO19 with (R)-enantiomer configuration at sulfur of Table P(SO), in either enantiomerically enriched or up to pure form, can be prepared in analogy by applying the preparation Example E1, step 1 (or an adaptation thereof known by those skilled in the art) on the respective sulfanyl substrates.

### Step 2: Preparation of Enantioenriched (R)-2-[[5-(ethylsulfonimidoyl)-6-[3-methyl-6-(trifluoromethyl)imidazo[4,5-b]pyridin-2-yl]-3-pyridyl]oxy]-2-methyl-propanenitrile (Compound (R)-P9)

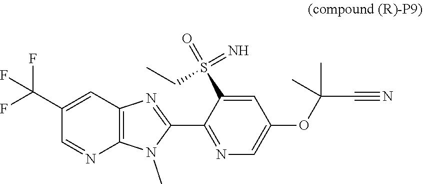 [SMILES: CC[S@@](=N)(=O)c1cc(OC(C)(C)C#N)cnc1-c1nc2cc(C(F)(F)F)cnc2n1C]

Enantioenriched 2-[[5-[(R)-ethylsulfinyl]-6-[3-methyl-6-(trifluoromethyl)imidazo[4,5-b]pyridin-2-yl]-3-pyridyl]oxy]-2-methyl-propanenitrile (compound (R)—S09 prepared as described above) (219 mg, 0.482 mmol) and iron(II)phthalocyanine (6.39 mg, 0.011 mmol) were dissolved in dichloromethane (2 mL). O-(4-nitrobenzoyl)-hydroxylamine triflic acid (prepared according to Chem Eur J 2017, 23, 563-567) (326 mg, 0.981 mmol) was added and the mixture stirred at room temperature for 1 hour. Ethyl acetate and 2M aqueous hydrochloric acid were added and the reaction mixture was filtered through a pad of celite. The filtrate was diluted with water and extracted with ethyl acetate. The organic phase was washed with sodium bicarbonate solution and brine, dried over sodium sulfate and concentrated in vacuo to afford the title compound.

LCMS (method 6) m/z 453 [M+H]⁺; retention time: 1.39 min.

Enantiomeric excess was measured according to the following method:

### Analytical SFC:

SFC: Waters Acquity UPC²/QDa
PDA Detector Waters Acquity UPC²
Column: Daicel SFC CHIRALPAK® IA, 3□m, 0.3 cm×10 cm, 40° C.
Mobile phase: A: CO₂ B: EtOH isocratic: 10% B in 4.8 min
ABPR: 1800 psi
Flow rate: 2.0 ml/min
Detection: 290 nm
Sample concentration: 1 mg/mL in ACN
Injection: 1 □L

### Results:

| First eluting enantiomer (R)-P9 | Second eluting enantiomer (S)-P9 |
|---|---|
| Retention time (min) ~1.47 | Retention time (min) ~1.88 |
| Chemical purity | Chemical purity |
| (area % at 290 nm) 92.4 | (area % at 290 nm) 7.6 |

Enantiomeric excess (%) 84.8

The first eluting major enantiomer (R)-P9 is corresponding to compound P9-A (Table P(E)) obtained via chiral resolution (Example P9 above).

All chiral sulfoximine compounds (R)-P1 to (R)-P19 with (R)-enantiomer configuration at sulfur of Table Y, in either enantiomerically enriched or up to pure form, can be prepared in analogy by applying the preparation Example E1, step 2 (or an adaptation thereof known by those skilled in the art) on the respective sulfinyl substrates (R)—SO1 to (R)—SO19 of Table P(SO).

TABLE P(SO)
^{§} Examples of (S)- or (R)-sulfinyl enantiomers of formula (III)

| | | | Configuration |
|---|---|---|---|
| No. | IUPAC name | Structures | at sulfur |
| (S)-SO1 | 2-[[6-[5-cyclopropyl-3-methyl-4-oxo- 6-(trifluoromethyl)imidazo[4,5- c]pyridin-2-yl]-5-[(S)-ethylsulfinyl]-3- pyridyl]oxy]-2-methyl-propanenitrile | 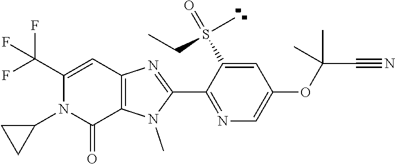 | S |
| (R)-SO1 | 2-[[6-[5-cyclopropyl-3-methyl-4-oxo- 6-(trifluoromethyl)imidazo[4,5- c]pyridin-2-yl]-5-[(R)-ethylsulfinyl]-3- pyridyl]oxy]-2-methyl-propanenitrile | 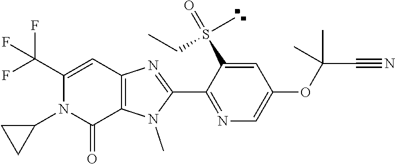 | R |
| (S)-SO2 | 1-[5-[(S)-ethylsulfinyl]-6-[7-methyl-3- (trifluoromethyl)imidazo[4,5- c]pyridazin-6-yl]-3- pyridyl]cyclopropanecarbonitrile | 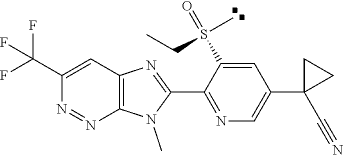 | S |
| (R)-SO2 | 1-[5-[(R)-ethylsulfinyl]-6-[7-methyl-3- (trifluoromethyl)imidazo[4,5- c]pyridazin-6-yl]-3- pyridyl]cyclopropanecarbonitrile | 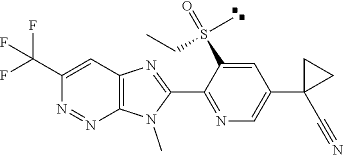 | R |
| (S)-SO3 | 2-[[5-[(S)-ethylsulfinyl]-6-[7- (trifluoromethyl)imidazo[1,2-a]pyridin- 2-yl]-3-pyridyl]oxy]-2-methyl- propanenitrile | 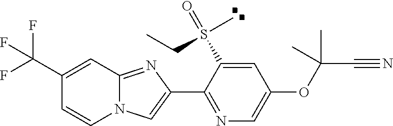 | S |
| (R)-SO3 | 2-[[5-[(R)-ethylsulfinyl]-6-[7- (trifluoromethyl)imidazo[1,2-a]pyridin- 2-yl]-3-pyridyl]oxy]-2-methyl- propanenitrile | 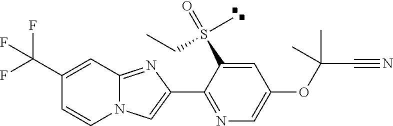 | R |
| (S)-SO4 | 2-[5-[(S)-ethylsulfinyl]-6-[3-methyl-6- (trifluoromethyl)imidazo[4,5-c]pyridin- 2-yl]-3-pyridyl]-2-methyl- propanenitrile | 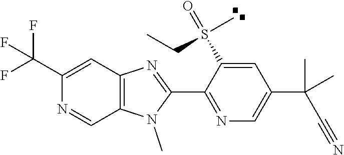 | S |
| (R)-SO4 | 2-[5-[(R)-ethylsulfinyl]-6-[3-methyl-6- (trifluoromethyl)imidazo[4,5-c]pyridin- 2-yl]-3-pyridyl]-2-methyl- propanenitrile | 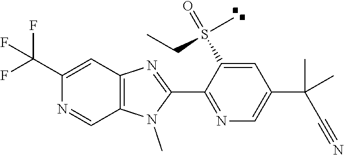 | R |
| (S)-SO5 | 2-[[5-[(S)-ethylsulfinyl]-2-methyl-6-[3- methyl-6-(trifluoromethyl)imidazo[4,5- b]pyridin-2-yl]-3-pyridyl]oxy]-2- methyl-propanenitrile | 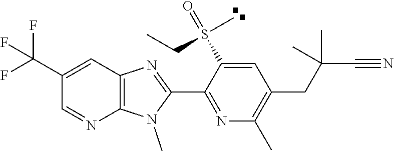 | S |
| (R)-SO5 | 2-[[5-[(R)-ethylsulfinyl]-2-methyl-6-[3- methyl-6-(trifluoromethyl)imidazo[4,5- b]pyridin-2-yl]-3-pyridyl]oxy]-2- methyl-propanenitrile | 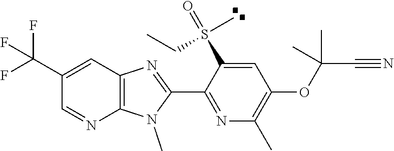 | R |
| (S)-SO6 | 1-[3-[(S)-ethylsulfinyl]-4-[3-methyl-6- (trifluoromethyl)imidazo[4,5-b]pyridin- 2-yl]phenyl]cyclopropanecarbonitrile | 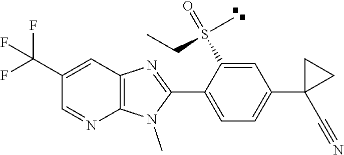 | S |
| (R)-SO6 | 1-[3-[(R)-ethylsulfinyl]-4-[3-methyl-6- (trifluoromethyl)imidazo[4,5-b]pyridin- 2-yl]phenyl]cyclopropanecarbonitrile | 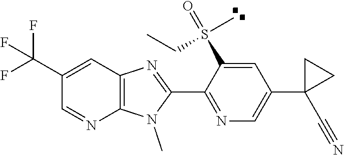 | R |
| (S)-SO7 | 2-[[5-[(S)-ethylsulfinyl]-6-[7- (trifluoromethylsulfanyl)imidazo[1,2- c]pyrimidin-2-yl]-3-pyridyl]oxy]-2- methyl-propanenitrile | 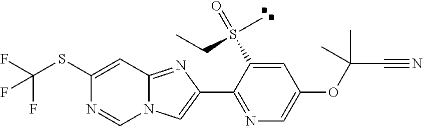 | S |
| (R)-SO7 | 2-[[5-[(R)-ethylsulfinyl]-6-[7- (trifluoromethylsulfanyl)imidazo[1,2- c]pyrimidin-2-yl]-3-pyridyl]oxy]-2- methyl-propanenitrile | 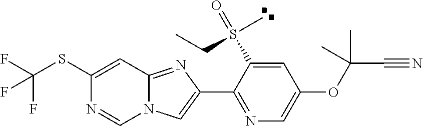 | R |
| (S)-SO8 | 1-[5-[(S)-ethylsulfinyl]-6-[5-methoxy- 3-methyl-4-oxo-6- (trifluoromethyl)imidazo[4,5-c]pyridin- 2-yl]-3- pyridyl]cyclopropanecarbonitrile | 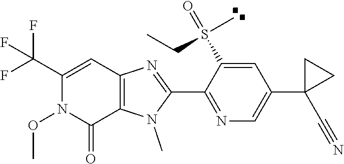 | S |
| (R)-SO8 | 1-[5-[(R)-ethylsulfinyl]-6-[5-methoxy- 3-methyl-4-oxo-6- (trifluoromethyl)imidazo[4,5-c]pyridin- 2-yl]-3- pyridyl]cyclopropanecarbonitrile | 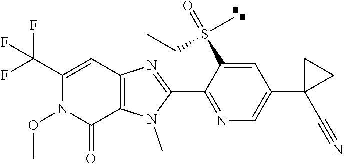 | R |
| (S)-SO9 | 2-[[5-[(S)-ethylsulfinyl]-6-[3-methyl-6- (trifluoromethyl)imidazo[4,5-b]pyridin- 2-yl]-3-pyridyl]oxy]-2-methyl- propanenitrile | 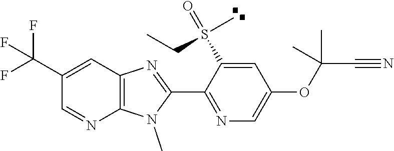 | S |
| (R)-SO9 | 2-[[5-[(R)-ethylsulfinyl]-6-[3-methyl-6- (trifluoromethyl)imidazo[4,5-b]pyridin- 2-yl]-3-pyridyl]oxy]-2-methyl-propanenitrile | 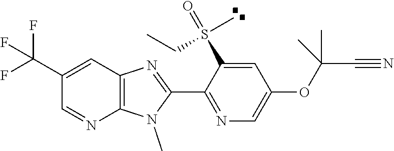 | R |
| (S)-SO10 | 1-[5-[(S)-ethylsulfinyl]-6-[3-methyl-6- (trifluoromethyl)imidazo[4,5-c]pyridin- 2-yl]-3-pyridyl]cyclopropanecarbonitrile | 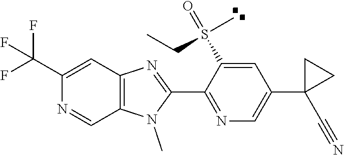 | S |
| (R)-SO10 | 1-[5-[(R)-ethylsulfinyl]-6-[3-methyl-6- (trifluoromethyl)imidazo[4,5-c]pyridin- 2-yl]-3-pyridyl]cyclopropanecarbonitrile | 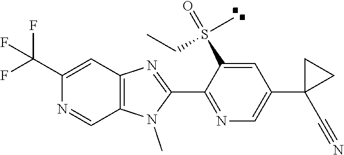 | R |
| (S)-SO11 | 2-[[5-[(S)-ethylsulfinyl]-6-[7- (trifluoromethyl)imidazo[1,2-c]pyrimidin-2- yl]-3-pyridyl]oxy]-2-methyl-propanenitrile | 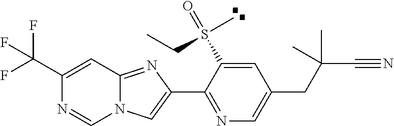 | S |
| (R)-SO11 | 2-[[5-[(R)-ethylsulfinyl]-6-[7- (trifluoromethyl)imidazo[1,2-c]pyrimidin- 2-yl]-3-pyridyl]oxy]-2-methyl-propanenitrile | 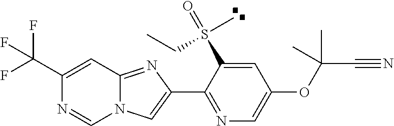 | R |
| (S)-SO12 | 2-[5-[(S)-ethylsulfinyl]-6-[3-methyl-6- (trifluoromethyl)imidazo[4,5-b]pyridin- 2-yl]-3-pyridyl]-2-methyl-propanenitrile | 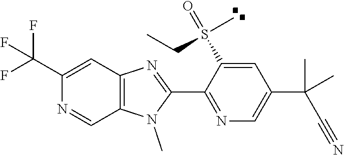 | S |
| (R)-SO12 | 2-[5-[(R)-ethylsulfinyl]-6-[3-methyl-6- (trifluoromethyl)imidazo[4,5-b]pyridin- 2-yl]-3-pyridyl]-2-methyl-propanenitrile | 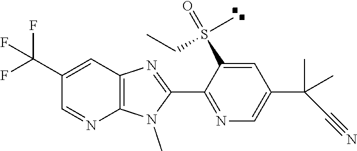 | R |
| (S)-SO13 | 2-[[5-[(S)-ethylsulfinyl]-6-[3-methyl-6- (trifluoromethyl)imidazo[4,5-c]pyridin- 2-yl]-3-pyridyl]oxy]-2-methyl-propanenitrile | 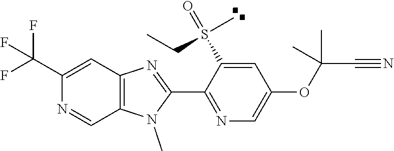 | S |
| (R)-SO13 | 2-[[5-[(R)-ethylsulfinyl]-6-[3-methyl-6- (trifluoromethyl)imidazo[4,5-c]pyridin- 2-yl]-3-pyridyl]oxy]-2-methyl-propanenitrile | 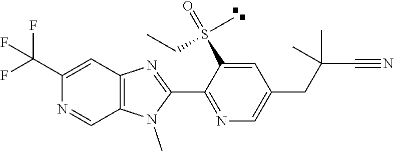 | R |
| (S)-SO14 | 1-[6-[5-ethyl-3-methyl-4-oxo-6- (trifluoromethyl)imidazo[4,5-c]pyridin- 2-yl]-5-[(S)-ethylsulfinyl]-3- pyridyl]cyclopropanecarbonitrile | 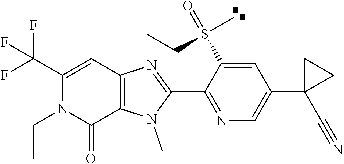 | S |
| (R)-SO14 | 1-[6-[5-ethyl-3-methyl-4-oxo-6- (trifluoromethyl)imidazo[4,5-c]pyridin- 2-yl]-5-[(R)-ethylsulfinyl]-3- pyridyl]cyclopropanecarbonitrile | 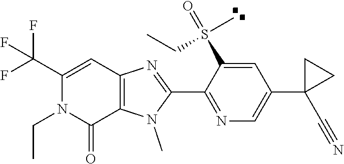 | R |
| (S)-SO15 | 2-[[5-[(S)-ethylsulfinyl]-6-[7-methyl-3- (trifluoromethyl)imidazo[4,5-c]pyridazin- 6-yl]-3-pyridyl]oxy]-2-methyl-propanenitrile | 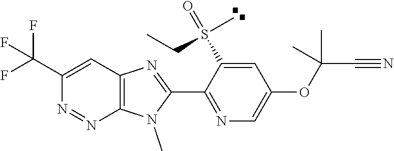 | S |
| (R)-SO15 | 2-[[5-[(R)-ethylsulfinyl]-6-[7-methyl-3- (trifluoromethyl)imidazo[4,5-c]pyridazin- 6-yl]-3-pyridyl]oxy]-2-methyl-propanenitrile | 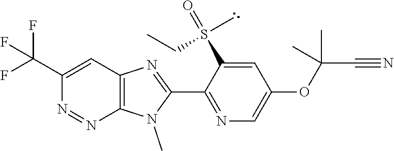 | R |
| (S)-SO16 | 1-[5-[(S)-ethylsulfinyl]-6-[3-methyl-6- (trifluoromethyl)imidazo[4,5-b]pyridin- 2-yl]-3-pyridyl]cyclopropanecarbonitrile | 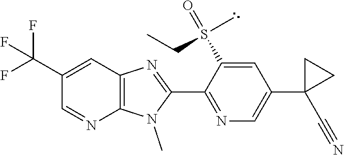 | S |
| (R)-SO16 | 1-[5-[(R)-ethylsulfinyl]-6-[3-methyl-6- (trifluoromethyl)imidazo[4,5-b]pyridin- 2-yl]-3-pyridyl]cyclopropanecarbonitrile | 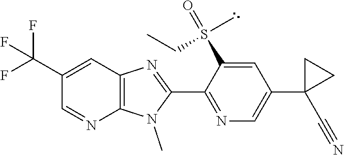 | R |
| (S)-SO17 | 2-[[5-[(S)-ethylsulfinyl]-2-methyl-6-[3- methyl-6-(trifluoromethyl)imidazo[4,5-c] pyridin-2-yl]-3-pyridyl]oxy]-2- methyl-propanenitrile | 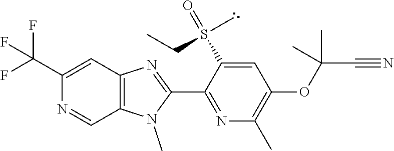 | S |
| (R)-SO17 | 2-[[5-[(R)-ethylsulfinyl]-2-methyl-6-[3- methyl-6-(trifluoromethyl)imidazo[4,5-c] pyridin-2-yl]-3-pyridyl]oxy]-2-methyl- propanenitrile | 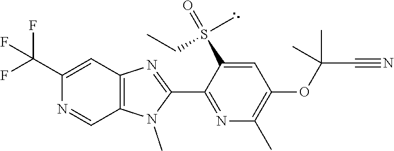 | R |
| (S)-SO18 | 1-[3-[(S)-ethylsulfinyl]-4-[3-methyl-6- (trifluoromethyl)imidazo[4,5-c]pyridin- 2-yl]phenyl]cyclopropanecarbonitrile | 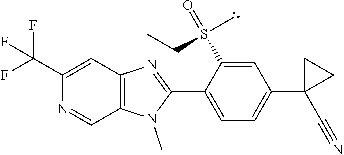 | S |
| (R)-SO18 | 1-[3-[(R)-ethylsulfinyl]-4-[3-methyl-6- (trifluoromethyl)imidazo[4,5-c]pyridin- 2-yl]phenyl]cyclopropanecarbonitrile | 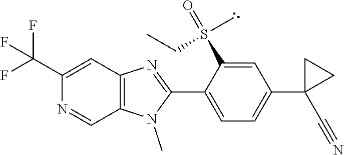 | R |
| (S)-SO19 | 2-[[6-[5-ethyl-3-methyl-4-oxo-6- (trifluoromethyl)imidazo[4,5-c]pyridin-2- yl]-5-[(S)-ethylsulfinyl]-3-pyridyl]oxy]-2- methyl-propanenitrile | 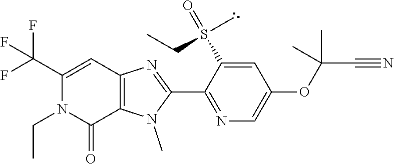 | S |
| (R)-SO19 | 2-[[6-[5-ethyl-3-methyl-4-oxo-6- (trifluoromethyl)imidazo[4,5-c]pyridin-2- yl]-5-[(R)-ethylsulfinyl]-3- pyridyl]oxy]-2-methyl-propanenitrile | 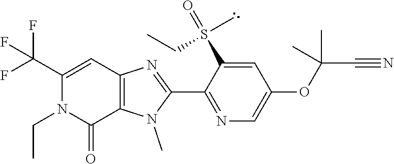 | R |

^{§} In Table P(SO), and in the preparative Examples E1 and E2, the radical “—:” attached to sulfur denotes its lone pair of electrons.

### Example E2: Preparation of (S)-2-[[5-(ethylsulfonimidoyl)-6-[3-methyl-6-(trifluoromethyl)imidazo[4,5-b]pyridin-2-yl]-3-pyridyl]oxy]-2-methyl-propanenitrile (Compound (S)—P9)

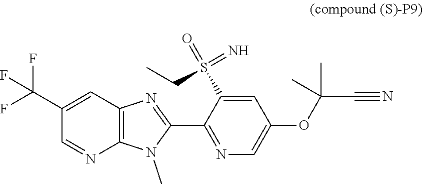 [SMILES: CC[S@](=N)(=O)c1cc(OC(C)(C)C#N)cnc1-c1nc2cc(C(F)(F)F)cnc2n1C]

Compound (S)—P9 in an enantiomerically enriched form (major (S)-stereoisomer having the absolute configuration depicted in the drawing) was prepared by an analogous method using the opposite enantiomer of the chiral ligand in the sulfoxide formation (Example E1, step 1).

### Step 1: Preparation of enantioenriched 2-[[5-[(S)-ethylsulfinyl]-6-[3-methyl-6-(trifluoromethyl)imidazo[4,5-b]pyridin-2-yl]-3-pyridyl]oxy]-2-methyl-propanenitrile (Compound (S)—S09)

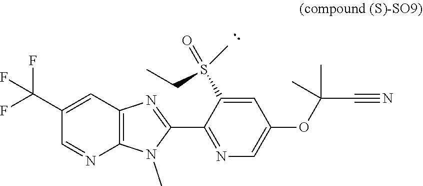

Compound (S)—S09 was prepared from 2-[[5-ethylsulfanyl-6-[3-methyl-6-(trifluoromethyl)imidazo[4,5-b]pyridin-2-yl]-3-pyridyl]oxy]-2-methyl-propanenitrile by an analogous procedure to Example E1, step 1 replacing 2-[(E)-[(1R)-1-(hydroxymethyl)-2,2-dimethylpropyl]iminomethyl]-4,6-diiodo-phenol with 2-[(E)-[(1S)-1-(hydroxymethyl)-2,2-dimethylpropyl]iminomethyl]-4,6-diiodo-phenol.

LCMS (method 6): m/z 438 [M+H]⁺; retention time: 1.87 min.

Enantiomeric excess was measured according to the following method:

### Analytical SFC Method

SFC: Waters Acquity UPC²/QDa
PDA Detector Waters Acquity UPC²
Column: Daicel SFC CHIRALPAK® IG, 3 m, 0.46 cm×10 cm, 40° C.
Mobile phase: A: CO₂ B: EtOH isocratic: 20% B in 4.8 min
ABPR: 1800 psi
Flow rate: 2.0 ml/min
Detection: 310 nm
Sample concentration: 1 mg/mL in MeOH/ACN 50/50
Injection: 1 μL

### Results:

| First eluting enantiomer (R)-SO9 | Second eluting enantiomer (S)-SO9 |
|---|---|
| Retention time (min) ~2.36 | Retention time (min) ~3.53 |
| Chemical purity | Chemical purity |
| (area % at 310 nm) 5.9 | (area % at 310 nm) 94.1 |

Enantiomeric excess (%) 88.2

All chiral sulfinyl compounds (S)—SO1 to (S)—SO19 with (S)-enantiomer configuration at sulfur of Table P(SO), in either enantiomerically enriched or up to pure form, can be prepared in analogy by applying the preparation Example E2, step 1 (or an adaptation thereof known by those skilled in the art) on the respective sulfanyl substrates.

### Step 2: Preparation of enantioenriched (S)-2-[[5-(ethylsulfonimidoyl)-6-[3-methyl-6-(trifluoromethyl)imidazo[4,5-b]pyridin-2-yl]-3-pyridyl]oxy]-2-methyl-propanenitrile (Compound (S)—P9)

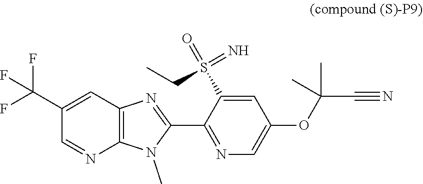 [SMILES: CC[S@](=N)(=O)c1cc(OC(C)(C)C#N)cnc1-c1nc2cc(C(F)(F)F)cnc2n1C]

Compound (S)—P9 was prepared from enantioenriched 2-[[5-[(S)-ethylsulfinyl]-6-[3-methyl-6-(trifluoromethyl)imidazo[4,5-b]pyridin-2-yl]-3-pyridyl]oxy]-2-methyl-propanenitrile (compound (S)—S09 prepared as described above) by an analogous procedure to Example E1, step 2.

LCMS (method 6): m/z 453 [M+H]⁺; retention time: 1.39 min.

Enantiomeric excess was measured according to the following method:

### Analytical SFC

SFC: Waters Acquity UPC²/QDa
PDA Detector Waters Acquity UPC²
Column: Daicel SFC CHIRALPAK® IA, 3 μm, 0.3 cm×10 cm, 40° C.
Mobile phase: A: CO₂ B: EtOH isocratic: 10% B in 4.8 min
ABPR: 1800 psi
Flow rate: 2.0 ml/min
Detection: 290 nm
Sample concentration: 1 mg/mL in ACN
Injection: 1 μL

### Results:

| First eluting enantiomer (R)-P9 | Second eluting enantiomer (S)-P9 |
|---|---|
| Retention time (min) ~1.49 | Retention time (min) ~1.88 |
| Chemical purity | Chemical purity |
| (area % at 290 nm) 5.6 | (area % at 290 nm) 94.4 |

Enantiomeric excess (%) 88.8

The second eluting major enantiomer (S)—P9 is corresponding to compound P9-B (Table P(E)) obtained via chiral resolution (Example P9 above).

All chiral sulfoximine compounds (S)—P1 to (S)—P19 with (S)-enantiomer configuration at sulfur of Table Y, in either enantiomerically enriched or up to pure form, can be prepared in analogy by applying the preparation Example E2, step 2 (or an adaptation thereof known by those skilled in the art) on the respective sulfinyl substrates (S)—SO1 to (S)—SO19 of Table P(SO).

The following mixtures of the compounds of formula I with active ingredients are preferred (the abbreviation “TX” means “one compound selected from the group consisting of the compounds described in Tables A-1 through A-20, Tables B-1 through B-20, Table Y, Table Z and Table P(E) of the present invention”):
an adjuvant selected from the group of substances consisting of petroleum oils (alternative name) (628)+TX;
abamectin+TX, acequinocyl+TX, acetamiprid+TX, acetoprole+TX, acrinathrin+TX, acynonapyr+TX, afidopyropen+TX, afoxolaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, S-bioallethrin+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2095470-94-1+TX, CAS number: 2377084-09-6+TX, CAS number: 1445683-71-5+TX, CAS number: 2408220-94-8+TX, CAS number: 2408220-91-5+TX, CAS number: 1365070-72-9+TX, CAS number: 2171099-09-3+TX, CAS number: 2396747-83-2+TX, CAS number: 2133042-31-4+TX, CAS number: 2133042-44-9+TX, CAS number: 1445684-82-1+TX, CAS number: 1445684-82-1+TX, CAS number: 1922957-45-6+TX, CAS number: 1922957-46-7+TX, CAS number: 1922957-47-8+TX, CAS number: 1922957-48-9+TX, CAS number: 2415706-16-8+TX, CAS number: 1594624-87-9+TX, CAS number: 1594637-65-6+TX, CAS number: 1594626-19-3+TX, CAS number: 1990457-52-7+TX, CAS number: 1990457-55-0+TX, CAS number: 1990457-57-2+TX, CAS number: 1990457-77-6+TX, CAS number: 1990457-66-3+TX, CAS number: 1990457-85-6+TX, CAS number: 2220132-55-6+TX, CAS number: 1255091-74-7+TX, CAS number: RNA (Leptinotarsa decemlineata-specific recombinant double-stranded interfering GS2)+TX, CAS number: 2719848-60-7+TX, CAS number: 1956329-03-5+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cyclobutrifluram+TX, cycloprothrin+TX, cycloxaprid+TX, cyenopyrafen+TX, cyetpyrafen (or etpyrafen)+TX, cyflumetofen+TX, cyfluthrin+TX, cyhalodiamide+TX, cyhalothrin+TX, cypermethrin+TX, cyphenothrin+TX, cyproflanilide+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin (or emamectin benzoate)+TX, empenthrin+TX, epsilon-momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenmezoditiaz+TX, fenitrothion+TX, fenobucarb+TX, fenothiocarb+TX, fenoxycarb+TX, fenpropathrin+TX, fenpyroximate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupentiofenox+TX, flupyradifurone+TX, flupyrimin+TX, fluralaner+TX, fluvalinate+TX, fluxametamide+TX, fosthiazate+TX, gamma-cyhalothrin+TX, guadipyr+TX, halofenozide+TX, halfenprox+TX, heptafluthrin+TX, hexythiazox+TX, hydramethylnon+TX, imicyafos+TX, imidacloprid+TX, imiprothrin+TX, indazapyroxamet+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-Cyhalothrin+TX, lepimectin+TX, lotilaner+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nicofluprole+TX; nitenpyram+TX, nithiazine+TX, omethoate+TX, oxamyl+TX, oxazosulfyl+TX, parathion-ethyl+TX, permethrin+TX, phenothrin+TX, phosphocarb+TX, piperonylbutoxide+TX, pirimicarb+TX, pirimiphos-ethyl+TX, pirimiphos-methyl+TX, Polyhedrosis virus+TX, prallethrin+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyriminostrobin+TX, pyriprole+TX, pyriproxyfen+TX, resmethrin+TX, sarolaner+TX, selamectin+TX, silafluofen+TX, spinetoram+TX, spinosad+TX, spirobudifen+TX; spirodiclofen+TX, spiromesifen+TX, spiropidion+TX, spirotetramat+TX, spidoxamat+TX, sulfoxaflor+TX, tebufenozide+TX, tebufenpyrad+TX, tebupirimiphos+TX, tefluthrin+TX, temephos+TX, tetrachlorantraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tigolaner+TX, tiorantraniliprole+TX; tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, trifluenfuronate+TX, triflumezopyrim+TX, tyclopyrazoflor+TX, zeta-cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B—50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.+TX;
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;
an anthelmintic selected from the group of substances consisting of abamectin (1)+TX, crufomate (1011)+TX, cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;
an avicide selected from the group of substances consisting of chloralose (127)+TX, endrin (1122)+TX, fenthion (346)+TX, pyridin-4-amine (IUPAC name) (23) and strychnine (745)+TX;
a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;
a soil sterilant selected from the group of substances consisting of iodomethane (IUPAC name) (542) and methyl bromide (537)+TX;
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) 40 [CCN]+TX, Gossyplure® (alternative name; 1:1 mixture of the (Z,E) and (Z,Z) isomers of hexadeca-7,11-dien-1-yl-acetate) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure Bi (alternative name) (839)+TX, trimedlure B₂ (alternative name) (839)+TX, trimedlure C (alternative name) (839) and trunc-call (alternative name) [CCN]+TX;
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX;
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;
a nitrification inhibitor selected from the group of substances consisting of potassium ethylxanthate [CCN] and nitrapyrin (580)+TX;
a plant activator selected from the group of substances consisting of acibenzolar (6)+TX, acibenzolar-S-methyl (6)+TX, probenazole (658) and Reynoutria sachalinensis extract (alternative name) (720)+TX;
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX;
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX;
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX;
a virucide selected from the group of substances consisting of imanin (alternative name) [CCN] and ribavirin (alternative name) [CCN]+TX;
a wound protectant selected from the group of substances consisting of mercuric oxide (512)+TX, octhilinone (590) and thiophanate-methyl (802)+TX;
a biologically active substance selected from 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure Bi+TX, trimedlure B₂+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, -sodium fluoroacetate+TX, thallium sulfate+TX, warfarin+TX, -2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole-+TX, epoxiconazole+TX, fenbuconazole+TX, fluquinconazole+TX, flusilazole+TX, flutriafol+TX, furametpyr+TX, hexaconazole+TX, imazalil-+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, -metalaxyl-+TX, Rmetalaxyl+TX, ofurace+TX, oxadixyl+TX, carbendazim+TX, debacarb+TX, fuberidazole-+TX, thiabendazole+TX, chlozolinate+TX, dichlozoline+TX, myclozoline-+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim--methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb-+TX, chloro-tha-lonil+TX, cyflufenamid+TX, cymoxanil+TX, cyclobutrifluram+TX, diclocymet+TX, diclomezine-+TX, dicloran+TX, diethofencarb+TX, dimethomorph-+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, -fenhexamid+TX, fosetyl-aluminium-+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, ethyl 1-[[4-[[2-(trifluoromethyl)-1,3-dioxolan-2-yl]methoxy]phenyl]methyl]pyrazole-3-carboxylate+TX (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate+TX (may be prepared from the methods described in WO 2020/056090), methyl N-[[4-[1-(4-cyclopropyl-2,6-difluoro-phenyl)pyrazol-4-yl]-2-methyl-phenyl]methyl]carbamate+TX (may be prepared from the methods described in WO 2020/097012), methyl N-[[4-[1-(2,6-difluoro-4-isopropyl-phenyl)pyrazol-4-yl]-2-methyl-phenyl]methyl]carbamate+TX (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide+TX (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide+TX (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide+TX (may be prepared from the methods described in WO 2020/109391), N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N—((3R)-1,1,3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N—((3R)-1,1,3-trimethylindan-4-yl) pyridine-3-carboxamide+TX, α-(1,1-dimethylethyl)-α-[4′-(trifluoromethoxy) [1,1′-biphenyl]-4-yl]-5-pyrimidinemethanol+TX, fluoxapiprolin+TX, enoxastrobin+TX, methyl (Z)-3-methoxy-2-[2-methyl-5-[4-(trifluoromethyl)triazol-2-yl]phenoxy]prop-2-enoate+TX, methyl (Z)-3-methoxy-2-[2-methyl-5-(4-propyltriazol-2-yl)phenoxy]prop-2-enoate+TX, methyl (Z)-2-[5-(3-isopropylpyrazol-1-yl)-2-methyl-phenoxy]-3-methoxy-prop-2-enoate+TX, methyl (Z)-3-methoxy-2-[2-methyl-5-(3-propylpyrazol-1-yl)phenoxy]prop-2-enoate+TX, methyl (Z)-3-methoxy-2-[2-methyl-5-[3-(trifluoromethyl)pyrazol-1-yl]phenoxy]prop-2-enoate+TX (these compounds may be prepared from the methods described in WO2020/079111), methyl (Z)-2-(5-cyclohexyl-2-methyl-phenoxy)-3-methoxy-prop-2-enoate+TX, methyl (Z)-2-(5-cyclopentyl-2-methyl-phenoxy)-3-methoxy-prop-2-enoate+TX (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N—[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO 2018/202428);
microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HCO+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus;
Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®);
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate;
Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator;
other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, fatty acids derived from a natural by-product of extra virgin olive oil (FLIPPER®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX;
(1) antibacterial agents selected from the group of:
(1.1) bacteria, examples of which are Bacillus mojavensis strain R₃B (Accession No. NCAIM (P) B001389) (WO 2013/034938) from Certis USA LLC, a subsidiary of Mitsui & Co.+TX; Bacillus pumilus, in particular strain BU F-33, having NRRL Accession No. 50185 (available as part of the CARTISSA® product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661, U.S. Pat. No. 6,060,051)+TX; Bacillus subtilis strain BU1814, (available as VELONDIS® PLUS, VELONDIS® FLEX and VELONDIS® EXTRA from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus subtilis CX-9060 from Certis USA LLC, a subsidiary of Mitsui & Co.+TX; Bacillus sp., in particular strain D747 (available as DOUBLE NICKEL® from Kumiai Chemical Industry Co., Ltd.), having Accession No. FERM BP-8234, U.S. Pat. No. 7,094,592+TX; Paenibacillus sp. strain having Accession No. NRRL B-50972 or Accession No. NRRL B-67129, WO 2016/154297+TX; Paenibacillus polymyxa, in particular strain AC-1 (e.g. TOPSEED® from Green Biotech Company Ltd.)+TX; Pantoea agglomerans, in particular strain E325 (Accession No. NRRL B-21856) (available as BLOOMTIME BIOLOGICAL™ FD BIOPESTICIDE from Northwest Agri Products)+TX; Pseudomonas proradix (e.g. PRORADIX® from Sourcon Padena)+TX; and
(1.2) fungi, examples of which are Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX; Pseudozyma aphidis (as disclosed in WO2011/151819 by Yissum Research Development Company of the Hebrew University of Jerusalem)+TX; Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR;
(2) biological fungicides selected from the group of:
(2.1) bacteria, examples of which are Agrobacterium radiobacter strain K84 (e.g. GALLTROL-A® from AgBioChem, CA)+TX; Agrobacterium radiobacter strain K1026 (e.g. NOGALL™ from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus amyloliquefaciens, in particular strain D747 (available as Double Nickel™ from Kumiai Chemical Industry Co., Ltd., having accession number FERM BP-8234, U.S. Pat. No. 7,094,592)+TX; Bacillus amyloliquefaciens strain F727 (also known as strain MB1110) (NRRL Accession No. B-50768, WO 2014/028521) (STARGUS® from Marrone Bio Innovations)+TX; Bacillus amyloliquefaciens strain FZB42, Accession No. DSM 23117 (available as RHIZOVITAL® from ABiTEP, DE)+TX; Bacillus amyloliquefaciens isolate B246 (e.g. AVOGREEN™ from University of Pretoria)+TX; Bacillus licheniformis, in particular strain SB3086, having Accession No. ATCC 55406, WO 2003/000051 (available as ECOGUARD® Biofungicide and GREEN RELEAF™ from Novozymes)+TX+TX; Bacillus licheniformis FMCH001 and Bacillus subtilis FMCH002 (QUARTZO® (WG) and PRESENCE® (WP) from FMC Corporation)+TX; Bacillus methylotrophicus strain BAC-9912 (from Chinese Academy of Sciences' Institute of Applied Ecology)+TX; Bacillus mojavensis strain R₃B (Accession No. NCAIM (P) B001389) (WO 2013/034938) from Certis USA LLC, a subsidiary of Mitsui & Co.+TX; Bacillus mycoides, isolate, having Accession No. B-30890 (available as BMJ TGAI® or WG and LifeGard™ from Certis USA LLC, a subsidiary of Mitsui & Co.)+TX; Bacillus pumilus, in particular strain QST2808 (available as SONATA® from Bayer CropScience LP, US, having Accession No. NRRL B-30087 and described in U.S. Pat. No. 6,245,551)+TX; Bacillus pumilus, in particular strain GB34 (available as Yield Shield® from Bayer AG, DE)+TX; Bacillus pumilus, in particular strain BU F-33, having NRRL Accession No. 50185 (available as part of the CARTISSA product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051)+TX; Bacillus subtilis Y1336 (available as BIOBAC® WP from Bion-Tech, Taiwan, registered as a biological fungicide in Taiwan under Registration Nos. 4764, 5454, 5096 and 5277)+TX; Bacillus subtilis strain MBI 600 (available as SUBTILEX from BASF SE), having Accession Number NRRL B-50595, U.S. Pat. No. 5,061,495+TX; Bacillus subtilis strain GB03 (available as Kodiak® from Bayer AG, DE)+TX; Bacillus subtilis strain BU1814, (available as VELONDIS® PLUS, VELONDIS® FLEX and VELONDIS® EXTRA from BASF SE)+TX; Bacillus subtilis CX-9060 from Certis USA LLC, a subsidiary of Mitsui & Co.+TX; Bacillus subtilis KTSB strain (FOLIACTIVE® from Donaghys)+TX; Bacillus subtilis IAB/BS03 (AVIV™ from STK Bio-Ag Technologies, PORTENTO® from Idai Nature)+TX; Bacillus subtilis strain Y1336 (available as BIOBAC® WP from Bion-Tech, Taiwan, registered as a biological fungicide in Taiwan under Registration Nos. 4764, 5454, 5096 and 5277)+TX; Paenibacillus epiphyticus (WO 2016/020371) from BASF SE+TX; Paenibacillus polymyxa ssp. plantarum (WO 2016/020371) from BASF SE+TX; Paenibacillus sp. strain having Accession No. NRRL B-50972 or Accession No. NRRL B-67129, WO 2016/154297+TX; Pseudomonas chlororaphis strain AFS009, having Accession No. NRRL B-50897, WO 2017/019448 (e.g., HOWLER™ and ZIO® from AgBiome Innovations, US)+TX; Pseudomonas chlororaphis, in particular strain MA342 (e.g. CEDOMON®, CERALL®, and CEDRESS® by Bioagri and Koppert)+TX; Pseudomonas fluorescens strain A506 (e.g. BLIGHTBAN® A506 by NuFarm)+TX; Pseudomonas proradix (e.g. PRORADIX® from Sourcon Padena)+TX; Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX; Streptomyces lydicus strain WYEC108 (also known as Streptomyces lydicus strain WYCD108US) (ACTINO-IRON® and ACTINOVATE® from Novozymes)+TX; and
(2.2) fungi, examples of which are Ampelomyces quisqualis, in particular strain AQ 10 (e.g. AQ 10® by IntrachemBio Italia)+TX; Ampelomyces quisqualis strain AQ10, having Accession No. CNCM 1-807 (e.g., AQ 10® by IntrachemBio Italia)+TX; Aspergillus flavus strain NRRL 21882 (products known as AFLA-GUARD® from Syngenta/ChemChina)+TX; Aureobasidium pullulans, in particular blastospores of strain DSM14940+TX; Aureobasidium pullulans, in particular blastospores of strain DSM 14941+TX; Aureobasidium pullulans, in particular mixtures of blastospores of strains DSM14940 and DSM 14941 (e.g. Botector® by bio-ferm, CH)+TX; Chaetomium cupreum (Accession No. CABI 353812) (e.g. BIOKUPRUM™ by AgriLife)+TX; Chaetomium globosum (available as RIVADIOM® by Rivale)+TX; Cladosporium cladosporioides, strain H39, having Accession No. CBS122244, US 2010/0291039 (by Stichting Dienst Landbouwkundig Onderzoek)+TX; Coniothyrium minitans, in particular strain CON/M/91-8 (Accession No. DSM9660, e.g. Contans® from Bayer CropScience Biologics GmbH)+TX; Cryptococcus flavescens, strain 3C (NRRL Y-50378), (B2.2.99)+TX; Dactylaria candida+TX; Dilophosphora alopecuri (available as TWIST FUNGUS®)+TX; Fusarium oxysporum, strain Fo47 (available as FUSACLEAN® by Natural Plant Protection)+TX; Gliocladium catenulatum (Synonym: Clonostachys rosea f. catenulate) strain J1446 (e.g. Prestop® by Lallemand)+TX; Gliocladium roseum (also known as Clonostachys rosea f rosea), in particular strain 321 U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), or strain IK726 (Jensen D F, et al. Development of a biocontrol agent for plant disease control with special emphasis on the near commercial fungal antagonist Clonostachys rosea strain ‘IK726’, Australas Plant Pathol. 2007,36:95-101)+TX; Lecanicillium lecanii (formerly known as Verticillium lecanii) conidia of strain KV01 (e.g. Vertalec® by Koppert/Arysta)+TX; Metschnikowia fructicola, in particular strain NRRL Y-30752, (B2.2.3)+TX; Microsphaeropsis ochracea+TX; Muscodor roseus, in particular strain A3-5 (Accession No. NRRL 30548)+TX; Penicillium steckii (DSM 27859, WO 2015/067800) from BASF SE+TX; Penicillium vermiculatum+TX; Phlebiopsis gigantea strain VRA 1992 (ROTSTOP® C from Danstar Ferment)+TX; Pichia anomala, strain WRL-076 (NRRL Y-30842), U.S. Pat. No. 7,579,183+TX; Pseudozyma flocculosa, strain PF-A22 UL (available as SPORODEX® L by Plant Products Co., CA)+TX; Saccharomyces cerevisiae, in particular strain LASO2 (from Agro-Levures et Derives), strain LAS117 cell walls (CEREVISANE® from Lesaffre, ROMEO® from BASF SE), strains CNCM No. 1-3936, CNCM No. 1-3937, CNCM No. 1-3938, CNCM No. 1-3939 (WO 2010/086790) from Lesaffre et Compagnie, FR+TX; Simplicillium lanosoniveum+TX; Talaromyces flavus, strain V117b+TX; Trichoderma asperelloides JM41 R (Accession No. NRRL B-50759) (TRICHO PLUS® from BASF SE)+TX; Trichoderma asperellum, in particular, strain kd (e.g. T-Gro from Andermatt Biocontrol)+TX; Trichoderma asperellum, in particular strain SKT-1, having Accession No. FERM P-16510 (e.g. ECO-HOPE® from Kumiai Chemical Industry), strain T34 (e.g. T34 Biocontrol by Biocontrol Technologies S.L., ES) or strain ICC 012 from Isagro+TX; Trichoderma atroviride, in particular strain SC1 (having Accession No. CBS 122089, WO 2009/116106 and U.S. Pat. No. 8,431,120 (from Bi-PA)), strain 77B (T77 from Andermatt Biocontrol) or strain LU132 (e.g. Sentinel from Agrimm Technologies Limited)+TX; Trichoderma atroviride, strain CNCM 1-1237 (e.g. Esquive® WP from Agrauxine, FR)+TX; Trichoderma atroviride, strain no. V08/002387+TX; Trichoderma atroviride, strain NMI no. V08/002388+TX; Trichoderma atroviride, strain NMI no. V08/002389+TX; Trichoderma atroviride, strain NMI no. V08/002390+TX; Trichoderma atroviride, strain LC52 (e.g. Tenet by Agrimm Technologies Limited)+TX; Trichoderma atroviride, strain ATCC 20476 (IMI 206040)+TX; Trichoderma atroviride, strain T11 (IM1352941/CECT20498)+TX; Trichoderma atroviride, strain SKT-1 (FERM P-16510), JP Patent Publication (Kokai) 11-253151 A+TX; Trichoderma atroviride, strain SKT-2 (FERM P-16511), JP Patent Publication (Kokai) 11-253151 A+TX; Trichoderma atroviride, strain SKT-3 (FERM P-17021), JP Patent Publication (Kokai) 11-253151 A+TX; Trichoderma fertile (e.g. product TrichoPlus from BASF)+TX; Trichoderma gamsii (formerly T. viride), strain ICCO80 (IMI CC 392151 CABI, e.g. BioDerma by AGROBIOSOL DE MEXICO, S.A. DE C.V.)+TX; Trichoderma gamsii (formerly T. viride), strain ICC 080 (IMI CC 392151 CABI) (available as BIODERMA® by AGROBIOSOL DE MEXICO, S.A. DE C.V.)+TX; Trichoderma harmatum+TX; Trichoderma harmatum, having Accession No. ATCC 28012+TX; Trichoderma harzianum strain T-22 (e.g. Trianum-P from Andermatt Biocontrol or Koppert) or strain Cepa SimbT5 (from Simbiose Agro)+TX; Trichoderma harzianum+TX; Trichoderma harzianum rifai T39 (e.g. Trichodex® from Makhteshim, US)+TX; Trichoderma harzianum, strain ITEM 908 (e.g. Trianum-P from Koppert)+TX; Trichoderma harzianum, strain TH35 (e.g. Root-Pro by Mycontrol)+TX; Trichoderma harzianum, strain DB 103 (available as T-GRO®7456 by Dagutat Biolab)+TX; Trichoderma polysporum, strain IMI 206039 (e.g. Binab TF WP by BINAB Bio-Innovation AB, Sweden)+TX; Trichoderma stromaticum, having Accession No. Ts3550 (e.g. Tricovab by CEPLAC, Brazil)+TX; Trichoderma virens (also known as Gliocladium virens), in particular strain GL-21 (e.g. SoilGard by Certis, US)+TX; Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX; Trichoderma viride, strain TV1(e.g. Trianum-P by Koppert)+TX; Trichoderma viride, in particular strain B35 (Pietr et al., 1993, Zesz. Nauk. A R w Szczecinie 161: 125-137)+TX; mixtures of Trichoderma asperellum strain ICC 012 (also known as Trichoderma harzianum I00012), having Accession No. CABI CC IMI 392716 and Trichoderma gamsii (formerly T. viride) strain ICC 080, having Accession No. IMI 392151 (e.g., BIO-TAM™ from Isagro USA, Inc. and BIODERMA® by Agrobiosol de Mexico, S.A. de C.V.)+TX; Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX; Verticillium albo-atrum (formerly V. dahliae), strain WCS850 having Accession No. WCS850, deposited at the Central Bureau for Fungi Cultures (e.g., DUTCH TRIG® by Tree Care Innovations)+TX; Verticillium chlamydosporium+TX;
(3) biological control agents having an effect for improving plant growth and/or plant health selected from the group of:
(3.1) bacteria, examples of which are Azospirillum brasilense (e.g., VIGOR® from KALO, Inc.)+TX; Azospirillum lipoferum (e.g., VERTEX-IF™ from TerraMax, Inc.)+TX; Azorhizobium caulinodans, in particular strain ZB—SK-5+TX; Azotobacter chroococcum, in particular strain H23+TX; Azotobacter vinelandii, in particular strain ATCC 12837+TX; a mixture of Azotobacter vinelandii and Clostridium pasteurianum (available as INVIGORATE® from Agrinos)+TX; Bacillus amyloliquefaciens μm414 (LOLI-PEPTA® from Biofilm Crop Protection)+TX; Bacillus amyloliquefaciens SB3281 (ATCC #PTA-7542, WO 2017/205258)+TX; Bacillus amyloliquefaciens TJ1000 (available as QUIKROOTS® from Novozymes)+TX; Bacillus amyloliquefaciens, in particular strain IN937a+TX; Bacillus amyloliquefaciens, in particular strain FZB42 (e.g. RHIZOVITAL® from ABiTEP, DE)+TX; Bacillus amyloliquefaciens BS27 (Accession No. NRRL B-5015)+TX; Bacillus cereus family member EE128 (NRRL No. B-50917)+TX; Bacillus cereus family member EE349 (NRRL No. B-50928)+TX; Bacillus cereus, in particular strain BPO1 (ATCC 55675, e.g. MEPICHLOR® from Arysta Lifescience, US)+TX; Bacillus firmus, in particular strain CNMC 1-1582 (e.g. VOTIVO® from BASF SE)+TX; Bacillus mycoides BT155 (NRRL No. B-50921)+TX; Bacillus mycoides EE118 (NRRL No. B-50918)+TX; Bacillus mycoides EE141 (NRRL No. B-50916)+TX; Bacillus mycoides BT46-3 (NRRL No. B-50922)+TX; Bacillus pumilus, in particular strain QST2808 (having Accession No. NRRL No. B-30087)+TX; Bacillus pumilus, in particular strain GB34 (e.g. YIELD SHIELD® from Bayer Crop Science, DE)+TX; Bacillus siamensis, in particular strain KCTC 13613T+TX; Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX; Bacillus subtilis, in particular strain AQ30002 (having Accession Nos. NRRL B-50421 and described in U.S. patent application Ser. No. 13/330,576)+TX; Bacillus subtilis, in particular strain AQ30004 (and NRRL B-50455 and described in U.S. patent application Ser. No. 13/330,576)+TX; Bacillus subtilis strain BU1814, (available as TEQUALIS® from BASF SE), Bacillus subtilis rm303 (RHIZOMAX® from Biofilm Crop Protection)+TX; Bacillus thuringiensis BT013A (NRRL No. B-50924) also known as Bacillus thuringiensis 4Q₇+TX; a mixture of Bacillus licheniformis FMCH001 and Bacillus subtilis FMCH002 (available as QUARTZO® (WG), PRESENCE® (WP) from FMC Corporation)+TX; Bacillus subtilis, in particular strain MBI 600 (e.g. SUBTILEX® from BASF SE)+TX; Bacillus tequilensis, in particular strain NII-0943+TX; Bradyrhizobium japonicum (e.g. OPTIMIZE® from Novozymes)+TX; Delftia acidovorans, in particular strain RAY209 (e.g. BIOBOOST® from Brett Young Seeds)+TX; Mesorhizobium cicer (e.g., NODULATOR from BASF SE)+TX; Lactobacillus sp. (e.g. LACTOPLANT® from LactoPAFI)+TX; Rhizobium leguminosarium biovar viciae (e.g., NODULATOR from BASF SE)+TX; Pseudomonas proradix (e.g. PRORADIX® from Sourcon Padena)+TX; Pseudomonas aeruginosa, in particular strain PN1+TX; Rhizobium leguminosarum, in particular bv. viceae strain Z25 (Accession No. CECT 4585)+TX; Paenibacillus polymyxa, in particular strain AC-1 (e.g. TOPSEED® from Green Biotech Company Ltd.)+TX; Serratia marcescens, in particular strain SRM (Accession No. MTCC 8708)+TX; Sinorhizobium meliloti strain NRG-185-1 (NITRAGIN® GOLD from Bayer CropScience)+TX; Thiobacillus sp. (e.g. CROPAID® from Cropaid Ltd UK)+TX; and
(3.2) fungi, examples of which are Purpureocillium lilacinum (previously known as Paecilomyces lilacinus) strain 251 (AGAL 89/030550, e.g. BioAct from Bayer CropScience Biologics GmbH)+TX; Penicillium bilaii, strain ATCC 22348 (e.g. JumpStart® from Acceleron BioAg), Talaromyces flavus, strain V117b+TX; Trichoderma atroviride strain CNCM 1-1237 (e.g. Esquive® WP from Agrauxine, FR), Trichoderma viride, e.g. strain B35 (Pietr et al., 1993, Zesz. Nauk. A R w Szczecinie 161: 125-137)+TX; Trichoderma atroviride strain LC52 (also known as Trichoderma atroviride strain LU132, e.g. Sentinel from Agrimm Technologies Limited)+TX; Trichoderma atroviride strain SC1 described in International Application No. PCT/IT2008/000196)+TX; Trichoderma asperellum strain kd (e.g. T-Gro from Andermatt Biocontrol)+TX; Trichoderma asperellum strain Eco-T (Plant Health Products, ZA), Trichoderma harzianum strain T-22 (e.g. Trianum-P from Andermatt Biocontrol or Koppert)+TX; Myrothecium verrucaria strain AARC-0255 (e.g. DiTera™ from Valent Biosciences)+TX; Penicillium bilaii strain ATCC ATCC20851+TX; Pythium oligandrum strain M1 (ATCC 38472, e.g. Polyversum from Bioprepraty, CZ)+TX; Trichoderma virens strain GL-21 (e.g. SoilGard® from Certis, USA)+TX; Verticillium albo-atrum (formerly V. dahliae) strain WCS850 (CBS 276.92, e.g. Dutch Trig from Tree Care Innovations)+TX; Trichoderma atroviride, in particular strain no. V08/002387, strain no. NMI No. V08/002388, strain no. NMI No. V08/002389, strain no. NMI No. V08/002390+TX; Trichoderma harzianum strain ITEM 908, Trichoderma harzianum, strain TSTh20+TX; Trichoderma harzianum strain 1295-22+TX; Pythium oligandrum strain DV74+TX; Rhizopogon amylopogon (e.g. comprised in Myco-Sol from Helena Chemical Company)+TX; Rhizopogon fulvigleba (e.g. comprised in Myco-Sol from Helena Chemical Company)+TX; Trichoderma virens strain GI-3+TX;
(4) insecticidally active biological control agents selected from
(4.1) bacteria, examples of which are Agrobacterium radiobacter strain K84 (Galltrol from AgBiochem Inc.)+TX; Bacillus amyloliquefaciens, in particular strain PTS-4838 (e.g. AVEO from Valent Biosciences, US)+TX; Bacillus firmus, in particular strain CNMC 1-1582 (e.g. VOTIVO® from BASF SE)+TX; Bacillus mycoides, isolate J. (e.g. BmJ from Certis USA LLC, a subsidiary of Mitsui & Co.)+TX; Bacillus sphaericus, in particular Serotype H5a5b strain 2362 (strain ABTS-1743) (e.g. VECTOLEX® from Valent BioSciences, US)+TX; Bacillus thuringiensis subsp. aizawai, in particular strain ABTS-1857 (SD-1372, e.g. XENTARI® from Valent BioSciences)+TX; Bacillus thuringiensis subsp. aizawai, in particular serotype H-7 (e.g. FLORBAC® WG from Valent BioSciences, US)+TX; Bacillus thuringiensis israelensis strain BMP 144 (e.g. AQUABAC® by Becker Microbial Products IL)+TX; Bacillus thuringiensis subsp. israelensis (serotype H-14) strain AM65-52 (Accession No. ATCC 1276) (e.g. VECTOBAC® by Valent BioSciences, US)+TX; Bacillus thuringiensis subsp. aizawai strain GC-91+TX; Bacillus thuringiensis var. Colmeri (e.g. TIANBAOBTC by Changzhou Jianghai Chemical Factory)+TX; Bacillus thuringiensis var. japonensis strain Buibui+TX; Bacillus thuringiensis subsp. kurstaki strain BMP 123 from Becker Microbial Products, IL+TX; Bacillus thuringiensis subsp. kurstaki strain BMP 123 by Becker Microbial Products, IL, e.g. BARITONE from Bayer CropScience+TX; Bacillus thuringiensis subsp. kurstaki strain HD-1 (e.g. DIPEL® ES from Valent BioSciences, US)+TX; Bacillus thuringiensis var. kurstaki strain EVB-113-19 (e.g., BIOPROTEC® from AEF Global)+TX; Bacillus thuringiensis subsp. kurstaki strain ABTS 351+TX; Bacillus thuringiensis subsp. kurstaki strain PB 54+TX; Bacillus thuringiensis subsp. kurstaki strain SA 11, (JAVELIN from Certis, US)+TX; Bacillus thuringiensis subsp. kurstaki strain SA 12 (THURICIDE from Certis, US)+TX; Bacillus thuringiensis subsp. kurstaki strain EG 2348 (LEPINOX from Certis, US)+TX; Bacillus thuringiensis subsp. kurstaki strain EG 7841 (CRYMAX from Certis, US)+TX; Bacillus thuringiensis subsp. tenebrionis strain NB 176 (SD-5428, e.g. NOVODOR® FC from BioFa DE)+TX; Brevibacillus laterosporus (LATERAL from Ecolibrium Biologicals)+TX; Burkholderia spp., in particular Burkholderia rinojensis strain A396 (also known as Burkholderia rinojensis strain MBI 305) (Accession No. NRRL B-50319+TX; WO 2011/106491 and WO 2013/032693+TX; e.g. MB1206 TGAI and ZELTO® from Marrone Bio Innovations)+TX; Chromobacterium subtsugae, in particular strain PRAA4-1T (MBI-203+TX; e.g. GRANDEVO® from Marrone Bio Innovations)+TX; Lecanicillium muscarium Ve6 (MYCOTAL from Koppert)+TX; Paenibacillus popilliae (formerly Bacillus popilliae+TX; e.g. MILKY SPORE POWDER™ and MILKY SPORE GRANULAR™ from St. Gabriel Laboratories)+TX; Pasteuria nishizawae strain Pn1 (CLARIVA from Syngenta/ChemChina)+TX; Serratia entomophila (e.g. INVADE® by Wrightson Seeds)+TX; Serratia marcescens, in particular strain SRM (Accession No. MTCC 8708)+TX; Trichoderma asperellum (TRICHODERMAX from Novozymes)+TX; Wolbachia pipientis ZAP strain (e.g., ZAP MALES® from MosquitoMate)+TX; and
(4.2) fungi, examples of which are Beauveria bassiana strain ATCC 74040 (e.g. NATURALIS® from Intrachem Bio Italia)+TX; Beauveria bassiana strain GHA (Accession No. ATCC74250, e.g. BOTANIGUARD® ES and MYCONTROL-O® from Laverlam International Corporation)+TX; Beauveria bassiana strain ATPO2 (Accession No. DSM 24665)+TX; Isaria fumosorosea (previously known as Paecilomyces fumosoroseus) strain Apopka 97) PREFERAL from SePRO+TX; Metarhizium anisopliae 3213-1 (deposited under NRRL accession number 67074) (WO 2017/066094+TX; Pioneer Hi-Bred International)+TX; Metarhizium robertsii 15013-1 (deposited under NRRL accession number 67073)+TX; Metarhizium robertsii23013-3 (deposited under NRRL accession number 67075)+TX; Paecilomyces lilacinus strain 251 (MELOCON from Certis, US)+TX; Zoophtora radicans+TX;
(5) Viruses selected from the group consisting of Adoxophyes orana (summer fruit tortrix) granulosis virus (GV)+TX; Cydia pomonella (codling moth) granulosis virus (GV)+TX; Helicoverpa armigera (cotton bollworm) nuclear polyhedrosis virus (NPV)+TX; Spodoptera exigua (beet armyworm) mNPV+TX; Spodoptera frugiperda (fall armyworm) mNPV+TX; Spodoptera littoralis (African cotton leafworm) NPV+TX;
(6) Bacteria and fungi which can be added as ‘inoculant’ to plants or plant parts or plant organs and which, by virtue of their particular properties, promote plant growth and plant health selected from Agrobacterium spp.+TX; Azorhizobium caulinodans+TX; Azospirillum spp.+TX; Azotobacter spp.+TX; Bradyrhizobium spp.+TX; Burkholderia spp., in particular Burkholderia cepacia (formerly known as Pseudomonas cepacia)+TX; Gigaspora spp., or Gigaspora monosporum+TX; Glomus spp.+TX; Laccaria spp.+TX; LactoBacillus buchneri+TX; Paraglomus spp.+TX; Pisolithus tinctorus+TX; Pseudomonas spp.+TX; Rhizobium spp., in particular Rhizobium trifolii+TX; Rhizopogon spp.+TX; Scleroderma spp.+TX; Suillus spp.+TX; Streptomyces spp.+TX;
(7) Plant extracts and products formed by microorganisms including proteins and secondary metabolites which can be used as biological control agents, selected from Allium sativum (NEMGUARD from Eco-Spray+TX; BRALIC from ADAMA)+TX; Armour-Zen+TX; Artemisia absinthium+TX; Azadirachtin (e.g. AZATIN XL from Certis, US)+TX; Biokeeper WP+TX; Brassicaceae extract, in particular oilseed rape powder or mustard powder+TX; Cassia nigricans+TX; Celastrus angulatus+TX; Chenopodium anthelminticum+TX; Chitin+TX; Dryopteris filix-mas+TX; Equisetum arvense+TX; Fortune Aza+TX; Fungastop+TX; Heads Up (Chenopodium quinoa saponin extract)+TX; PROBLAD (naturally occurring Blad polypeptide from Lupin seeds), Certis EU+TX; FRACTURE (naturally occurring Blad polypeptide from Lupin seeds), FMC+TX; Pyrethrum/Pyrethrins+TX; Quassia amara+TX; Quercus+TX; Quillaja extract (QL AGRI 35 from BASF)+TX; Reynoutria sachalinensis extract (REGALLIA/REGALIA MAXX from Marrone Bio)+TX; “Requiem™ Insecticide”+TX; Rotenone+TX; ryania/ryanodine+TX; Symphytum officinale+TX; Tanacetum vulgare+TX; Thymol+TX; Thymol mixed with Geraniol (CEDROZ from Eden Research)+TX; Thymol mixed with Geraniol and Eugenol (MEVALONE from Eden Research)+TX; Triact 70+TX; TriCon+TX; Tropaeulum majus+TX; Melaleuca alternifolia extract (TIMOREX GOLD from STK)+TX; Urtica dioica+TX; Veratrin+TX; and Viscum album+TX; and a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual-A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula I selected from Tables A-1 through A-20, Tables B-1 through B-20, Table Y, Table Z and Table P(E) with active ingredients described above comprises a compound selected from Tables A-1 through A-20, Tables B-1 through B-20, Table Y, Table Z and Table P(E) and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables A-1 through A-20, Tables B-1 through B-20, Table Y, Table Z and Table P(E) and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables A-1 through A-20, Tables B-1 through B-20, Table Y, Table Z and Table P(E) and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula I including those selected from Tables A-1 through A-20, Tables B-1 through B-20, Table Y, Table Z and Table P(E). Further, it is hereby made available, a composition comprising a plant propagation material treated with a compound of formula I including those selected from Tables A-1 through A-20, Tables B-1 through B-20, Table Y, Table Z and Table P(E).

Seed treatment comprises all suitable seed treatment techniques known in the art, such as seed dressing, seed coating, seed dusting, seed soaking and seed pelleting. The seed treatment application of the compound formula I (including those selected from Tables A-1 through A-20, Tables B-1 through B-20, Table Y, Table Z and Table P(E)) can be carried out by any known methods, such as spraying or by dusting the seeds before sowing or during the sowing/planting of the seeds.

### BIOLOGICAL EXAMPLES

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

### Example B1: Activity Against Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: P1, P3, P5, P6, P8, P9, P11, P12, P13, P14, P17, P19, P1-A, P3-A, P5-A, P7-A, P8-A, P9-A, P14-A, P15-A, P19-A, P1-B, P3-B, P5-B, P6-B, P7-B, P9-B, P13-B, P14-B, P16-B, P17-B, P19-B.

### Example B2: Activity Against Plutella xylostella (Diamond Back Moth)

24-well microtiter plates with artificial diet were treated with aqueous test solutions prepared from 10′000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm: P1, P3, P5, P6, P8, P9, P11, P12, P13, P14, P15, P16, P17, P19.

### Example B3: Activity Against Diabrotica Balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: P1, P2, P3, P5, P7, P8, P9, P11, P12, P13, P14, P15, P18, P19, P1-A, P3-A, P5-A, P6-A, P7-A, P8-A, P9-A, P10-A, P11-A, P12-A, P13-A, P14-A, P15-A, P16-A, P17-A, P18-A, P19-A, P1-B, P3-B, P4-B, P5-B, P6-B, P7-B, P8-B, P9-B, P11-B, P12-B, P13-B, P14-B, P15-B, P17-B, P18-B, P19-B.

### Example B4: Activity Against Myzus persicae (Green Peach Aphid) Feeding/Contact Activity

Sunflower leaf discs were placed on agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: P1, P2, P3, P4, P5, P6, P7, P8, P9, P10, P11, P12, P13, P14, P15, P16, P17, P18, P19, P1-A, P2-A, P3-A, P4-A, P5-A, P6-A, P7-A, P8-A, P9-A, P10-A, P11-A, P12-A, P13-A, P14-A, P15-A, P16-A, P17-A, P18-A, P19-A, P1-B, P2-B, P3-B, P4-B, P5-B, P6-B, P7-B, P8-B, P9-B, P10-B, P11-B, P12-B, P13-B, P14-B, P15-B, P16-B, P17-B, P18-B, P19-B.

### Example B5: Activity Against Myzus persicae (Green Peach Aphid) Systemic Activity

Roots of pea seedlings infested with an aphid population of mixed ages were placed directly in the aqueous test solutions prepared from 10′000 DMSO stock solutions. The samples were assessed for mortality 6 days after placing seedlings in test solutions.

The following compounds resulted in at least 80% mortality at a test rate of 24 ppm: P1, P2, P4, P5, P6, P8, P9, P10, P11, P12, P13, P14, P15, P16, P17, P18, P19, P1-A, P2-A, P4-A, P5-A, P6-A, P7-A, P8-A, P9-A, P10-A, P11-A, P12-A, P13-A, P14-A, P15-A, P16-A, P17-A, P18-A, P19-A, P1-B, P2-B, P3-B, P4-B, P5-B, P6-B, P7-B, P8-B, P9-B, P10-B, P11-B, P12-B, P13-B, P14-B, P15-B, P16-B, P17-B, P18-B, P19-B.

### Example B6: Activity Against Bemisia tabaci (Cotton White Fly)

Cotton leaf discs were placed on agar in 24-well microtiter plates and sprayed with aqueous test solutions prepared from 10′000 ppm DMSO stock solutions. After drying the leaf discs were infested with adult white flies. The samples were checked for mortality 6 days after incubation.

The following compounds resulted in at least 80% mortality at an application rate of 200 ppm: P1, P2, P4, P5, P6, P7, P8, P9, P10, P11, P12, P13, P14, P15, P16, P17, P18, P19, P1-A, P2-A, P4-A, P5-A, P6-A, P7-A, P8-A, P9-A, P10-A, P12-A, P13-A, P14-A, P15-A, P16-A, P17-A, P18-A, P19-A, P1-B, P2-B, P4-B, P5-B, P6-B, P7-B, P8-B, P9-B, P10-B, P11-B, P12-B, P13-B, P14-B, P15-B, P16-B, P17-B, P18-B, P19-B.

### Example B7: Activity Against Euschistus heros (Neotropical Brown Stink Bug)

Soybean leaf on agar in 24-well microtiter plates were sprayed with aqueous test solutions prepared from 10′000 ppm DMSO stock solutions. After drying the leaf were infested with N-2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: P1, P2, P5, P6, P7, P8, P9, P13, P14, P15, P18, P19, P1-A, P7-A, P8-A, P14-A, P19-A, P1-B, P5-B, P7-B, P17-B, P18-B, P19-B.

### Example B8: Activity Against Frankliniella occidentalis (Western Flower Thrips)

Sunflower leaf discs were placed on agar in 24-well microtiter plates and sprayed with aqueous test solutions prepared from 10′000 DMSO stock solutions. After drying the leaf discs were infested with a Frankliniella population of mixed ages. The samples were assessed for mortality 7 days after infestation.

The following compounds resulted in at least 80% mortality at an application rate of 200 ppm: P1, P7, P11-A, P1-B, P7-B, P19-B.

### Example B9: Activity Against Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation.

The following compounds gave an effect of at least 80% in at least one of the two categories (mortality or growth inhibition) at an application rate of 200 ppm: P7, P10, P1-A, P3-A, P4-A, P5-A, P6-A, P7-A, P8-A, P9-A, P10-A, P11-A, P12-A, P13-A, P14-A, P15-A, P16-A, P17-A, P18-A, P19-A, P1-B, P3-B, P4-B, P5-B, P6-B, P7-B, P8-B, P9-B, P10-B, P11-B, P12-B, P13-B, P14-B, P15-B, P16-B, P17-B, P19-B.

### Example B10: Activity Against Tetranychus urticae (Two-Spotted Spider Mite)

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: P7, P13, P19-A.

### Example B11: Activity Against Chilo suppressalis (Striped Rice Stemborer)

24-well microtiter plates with artificial diet were treated with aqueous test solutions prepared from 10′000 ppm DMSO stock solutions by pipetting. After drying, the plates were infested with L2 larvae (6-8 per well). The samples were assessed for mortality, anti-feeding effect, and growth inhibition in comparison to untreated samples 6 days after infestation. Control of Chilo suppressalis by a test sample is given when at least one of the categories mortality, anti-feedant effect, and growth inhibition is higher than the untreated sample.

For example, the following compounds resulted in at least 80% control at an application rate of 200 ppm: P10, P2-A, P6-A, P8-A, P12-A, P13-A, P14-A, P16-A, P6-B, P8-B, P10-B, P12-B, P13-B, P14-B, P16-B.

### Example B12: Comparison of the Insecticidal Activity of Single Enantiomer Compounds P18-A/P18-B, P15-A/P15-B, P9-A/P9-B, P5-A/P5-B, P14-A/P14-B, P6-A/P6-B and P13-A/P13-B According to the Invention with the Structurally Comparable Racemic Sulfoximine Compounds from the State of the Art

Activity of compounds P18-A, P18-B, P15-A, P15-B, P9-A, P9-B, P5-A, P5-B, P14-A, P14-B, P6-A, P6-B, P13-A and P13-B according to the preparatory examples and of compound P13 from WO19/234158, compound P2 from WO20/084075, compound P3 from WO20/084075, compound P4 from WO20/084075, compound P14 from WO19/234158, compound P15 from WO19/234158, respectively compound P1 from WO20/084075 against Diabrotica balteata (Example B3) is summarized in Table B12.

TABLE B12

| | Concentration | | Mortality |
|---|---|---|---|
| Compound | (ppm) | Insect | (%) |
| Compound P18-A | 50 | Diabrotica balteata | 100 |
| 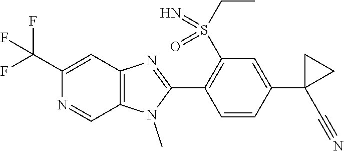 [SMILES: CCS(=N)(=O)c1cc(C2(C#N)CC2)ccc1-c1nc2cc(C(F)(F)F)ncc2n1C] | | | |
| Present invention | | | |
| Compound P18-B | 50 | Diabrotica balteata | 100 |
| 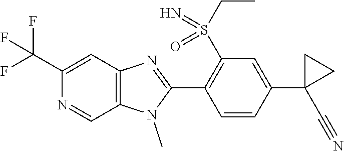 [SMILES: CCS(=N)(=O)c1cc(C2(C#N)CC2)ccc1-c1nc2cc(C(F)(F)F)ncc2n1C] | | | |
| Present invention | | | |
| Described in WO19/234158 as | 50 | Diabrotica balteata | 0 |
| compound P13 | | | |
| 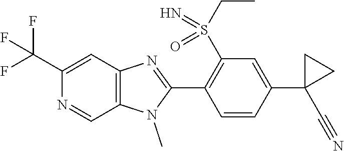 [SMILES: CCS(=N)(=O)c1cc(C2(C#N)CC2)ccc1-c1nc2cc(C(F)(F)F)ncc2n1C] | | | |
| rac | | | |
| State of the art | | | |
| Compound P15-A | 12.5 | Diabrotica balteata | 80 |
| 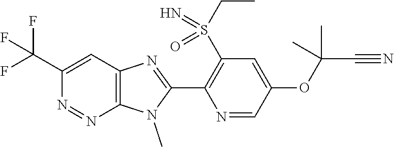 [SMILES: CCS(=N)(=O)c1cc(OC(C)(C)C#N)cnc1-c1nc2cc(C(F)(F)F)nnc2n1C] | | | |
| Present invention | | | |
| Compound P15-B | 12.5 | Diabrotica balteata | 100 |
| 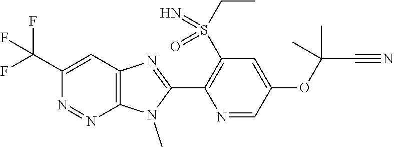 [SMILES: CCS(=N)(=O)c1cc(OC(C)(C)C#N)cnc1-c1nc2cc(C(F)(F)F)nnc2n1C] | | | |
| Present invention | | | |
| Described in WO20/084075 as | 12.5 | Diabrotica balteata | 0 |
| compound P2 | | | |
| 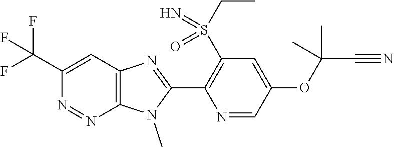 [SMILES: CCS(=N)(=O)c1cc(OC(C)(C)C#N)cnc1-c1nc2cc(C(F)(F)F)nnc2n1C] | | | |
| rac | | | |
| State of the art | | | |
| Compound P9-A | 50 | Diabrotica balteata | 80 |
| 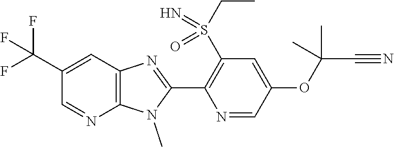 [SMILES: CCS(=N)(=O)c1cc(OC(C)(C)C#N)cnc1-c1nc2cc(C(F)(F)F)cnc2n1C] | | | |
| Present invention | | | |
| Compound P9-B | 50 | Diabrotica balteata | 100 |
| 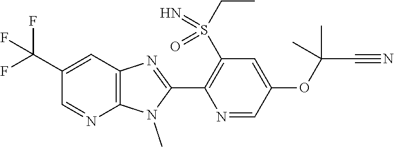 [SMILES: CCS(=N)(=O)c1cc(OC(C)(C)C#N)cnc1-c1nc2cc(C(F)(F)F)cnc2n1C] | | | |
| Present invention | | | |
| Described in WO20/084075 as | 50 | Diabrotica balteata | 80 |
| compound P3 | | | |
| 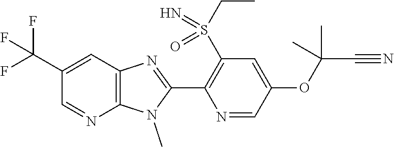 [SMILES: CCS(=N)(=O)c1cc(OC(C)(C)C#N)cnc1-c1nc2cc(C(F)(F)F)cnc2n1C] | | | |
| rac | | | |
| State of the art | | | |
| Compound P5-A | 3.125 | Diabrotica balteata | 80 |
| 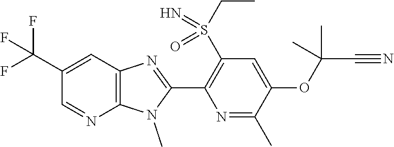 [SMILES: CCS(=N)(=O)c1cc(OC(C)(C)C#N)c(C)nc1-c1nc2cc(C(F)(F)F)cnc2n1C] | | | |
| Present invention | | | |
| Compound P5-B | 3.125 | Diabrotica balteata | 100 |
| 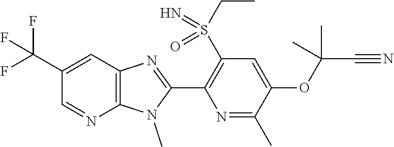 [SMILES: CCS(=N)(=O)c1cc(OC(C)(C)C#N)c(C)nc1-c1nc2cc(C(F)(F)F)cnc2n1C] | | | |
| Present invention | | | |
| Described in WO20/084075 as | 3.125 | Diabrotica balteata | 0 |
| compound P4 | | | |
| 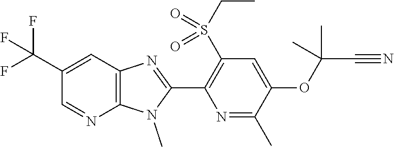 [SMILES: CCS(=O)(=O)c1cc(OC(C)(C)C#N)c(C)nc1-c1nc2cc(C(F)(F)F)cnc2n1C] | | | |
| rac | | | |
| State of the art | | | |
| Compound P14-A | 50 | Diabrotica balteata | 80 |
| 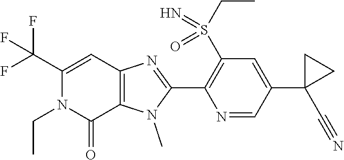 [SMILES: CCn1c(C(F)(F)F)cc2nc(-c3ncc(C4(C#N)CC4)cc3S(=N)(=O)CC)n(C)c2c1=O] | | | |
| Present invention | | | |
| Compound P14-B | 50 | Diabrotica balteata | 50 |
| 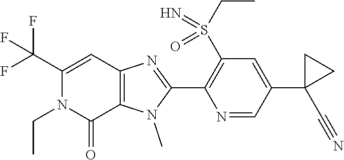 [SMILES: CCn1c(C(F)(F)F)cc2nc(-c3ncc(C4(C#N)CC4)cc3S(=N)(=O)CC)n(C)c2c1=O] | | | |
| Present invention | | | |
| Described in WO19/234158 as | 50 | Diabrotica balteata | 0 |
| compound P14 | | | |
| 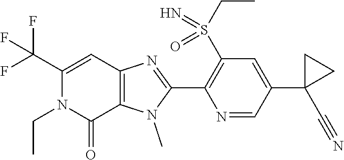 [SMILES: CCn1c(C(F)(F)F)cc2nc(-c3ncc(C4(C#N)CC4)cc3S(=N)(=O)CC)n(C)c2c1=O] | | | |
| rac | | | |
| State of the art | | | |
| Compound P6-A | 50 | Diabrotica balteata | 80 |
| 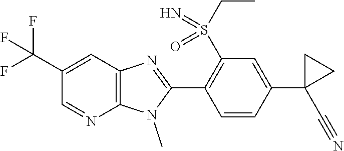 [SMILES: CCS(=N)(=O)c1cc(C2(C#N)CC2)ccc1-c1nc2cc(C(F)(F)F)cnc2n1C] | | | |
| Present invention | | | |
| Compound P6-B | 50 | Diabrotica balteata | 80 |
| 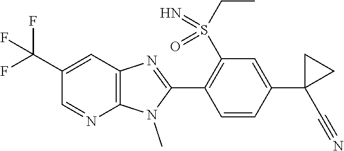 [SMILES: CCS(=N)(=O)c1cc(C2(C#N)CC2)ccc1-c1nc2cc(C(F)(F)F)cnc2n1C] | | | |
| Present invention | | | |
| Described in WO19/234158 as | 50 | Diabrotica balteata | 0 |
| compound P15 | | | |
| 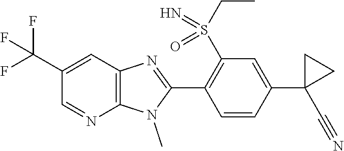 [SMILES: CCS(=N)(=O)c1cc(C2(C#N)CC2)ccc1-c1nc2cc(C(F)(F)F)cnc2n1C] | | | |
| rac | | | |
| State of the art | | | |
| Compound P13-A | 50 | Diabrotica balteata | 80 |
| 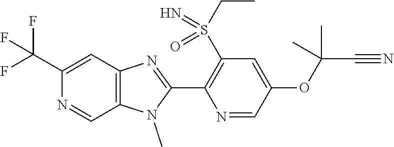 [SMILES: CCS(=N)(=O)c1cc(OC(C)(C)C#N)cnc1-c1nc2cc(C(F)(F)F)ncc2n1C] | | | |
| Present invention | | | |
| Compound P13-B | 50 | Diabrotica balteata | 100 |
| 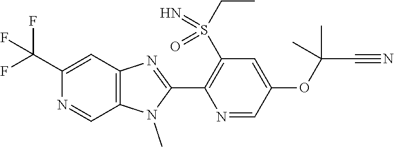 [SMILES: CCS(=N)(=O)c1cc(OC(C)(C)C#N)cnc1-c1nc2cc(C(F)(F)F)ncc2n1C] | | | |
| Present invention | | | |
| Described in WO20/084075 as | 50 | Diabrotica balteata | 0 |
| compound P1 | | | |
| 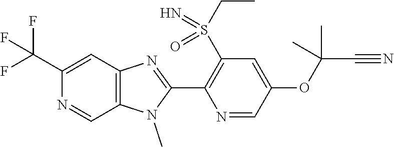 [SMILES: CCS(=N)(=O)c1cc(OC(C)(C)C#N)cnc1-c1nc2cc(C(F)(F)F)ncc2n1C] | | | |
| rac | | | |
| State of the art | | | |

Table B12 shows that single enantiomer compounds P18-A, P18-B, P15-A, P15-B, P9-A, P9-B, P5-A, P5-B, P14-A, P14-B, P6-A, P6-B, P1 3-A and P1 3-B (either first/second eluting following a chiral resolution [Table Z], or enantiopure/enantiomerically enriched following a stereoselective synthesis [Table Y]) according to the invention exert predominantly a substantially better insecticidal action on Diabrotica balteata than the compound from the state of the art.

Example B13: Comparison of the Insecticidal Activity of Single Enantiomer Compounds P18-A/P18-B. P10-A/P10-B. P7-A/P7-B and P16-A/P16-B According to the Invention with the Structurally Comparable Racemic Sulfoximine Compounds from the State of the Art

Activity of compounds P18-A, P18-B, P10-A, P10-B, P7-A, P7-B, P16-A and P16-B according to the preparatory examples and of compound P13 from WO19/234158, compound Y-6.001 from WO19/234158, compound P11 from WO20/084075, respectively compound P6 from WO19/234158 against Bemisia tabaci (Example B6) is summarized in Table B13.

TABLE B13

| | Concentration | | Mortality |
|---|---|---|---|
| Compound | (ppm) | Insect | (%) |
| Compound P18-A | 12.5 | Bemisia tabaci | 100 |
| 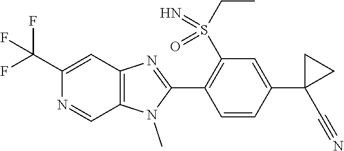 [SMILES: CCS(=N)(=O)c1cc(C2(C#N)CC2)ccc1-c1nc2cc(C(F)(F)F)ncc2n1C] | | | |
| Present invention | | | |
| Compound P18-B | 12.5 | Bemisia tabaci | 80 |
| 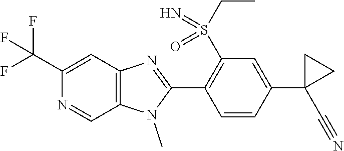 [SMILES: CCS(=N)(=O)c1cc(C2(C#N)CC2)ccc1-c1nc2cc(C(F)(F)F)ncc2n1C] | | | |
| Present invention | | | |
| Described in WO19/234158 as | 12.5 | Bemisia tabaci | 0 |
| compound P13 | | | |
| 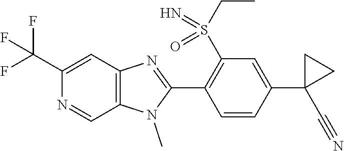 [SMILES: CCS(=N)(=O)c1cc(C2(C#N)CC2)ccc1-c1nc2cc(C(F)(F)F)ncc2n1C] | | | |
| rac | | | |
| State of the art | | | |
| Compound P10-A | 12.5 | Bemisia tabaci | 80 |
| 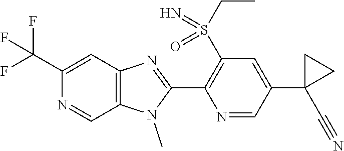 [SMILES: CCS(=N)(=O)c1cc(C2(C#N)CC2)cnc1-c1nc2cc(C(F)(F)F)ncc2n1C] | | | |
| Present invention | | | |
| Compound P10-B | 12.5 | Bemisia tabaci | 100 |
| 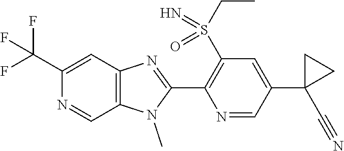 [SMILES: CCS(=N)(=O)c1cc(C2(C#N)CC2)cnc1-c1nc2cc(C(F)(F)F)ncc2n1C] | | | |
| Present invention | | | |
| Described in WO19/234158 as | 12.5 | Bemisia tabaci | 50 |
| compound Y-6.001 | | | |
| 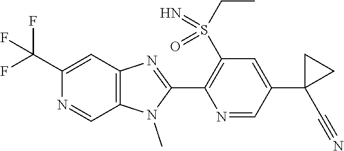 [SMILES: CCS(=N)(=O)c1cc(C2(C#N)CC2)cnc1-c1nc2cc(C(F)(F)F)ncc2n1C] | | | |
| rac | | | |
| State of the art | | | |
| Compound P7-A | 50 | Bemisia tabaci | 80 |
| 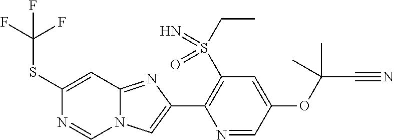 [SMILES: CCS(=N)(=O)c1cc(OC(C)(C)C#N)cnc1-c1cn2cnc(SC(F)(F)F)cc2n1] | | | |
| Present invention | | | |
| Compound P7-B | 50 | Bemisia tabaci | 80 |
| 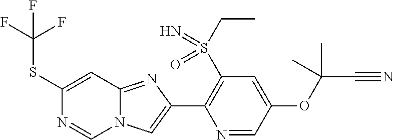 [SMILES: CCS(=N)(=O)c1cc(OC(C)(C)C#N)cnc1-c1cn2cnc(SC(F)(F)F)cc2n1] | | | |
| Present invention | | | |
| Described in WO20/084075 as | 50 | Bemisia tabaci | 50 |
| compound P11 | | | |
| 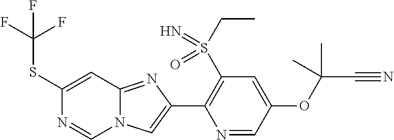 [SMILES: CCS(=N)(=O)c1cc(OC(C)(C)C#N)cnc1-c1cn2cnc(SC(F)(F)F)cc2n1] | | | |
| rac | | | |
| State of the art | | | |
| Compound P16-A | 12.5 | Bemisia tabaci | 100 |
| 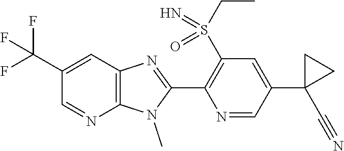 [SMILES: CCS(=N)(=O)c1cc(C2(C#N)CC2)cnc1-c1nc2cc(C(F)(F)F)cnc2n1C] | | | |
| Present invention | | | |
| Compound P16-B | 12.5 | Bemisia tabaci | 80 |
| 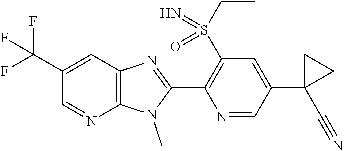 [SMILES: CCS(=N)(=O)c1cc(C2(C#N)CC2)cnc1-c1nc2cc(C(F)(F)F)cnc2n1C] | | | |
| Present invention | | | |
| Described in WO19/234158 as | 12.5 | Bemisia tabaci | 50 |
| compound P6 | | | |
| 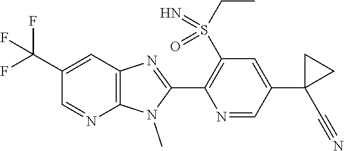 [SMILES: CCS(=N)(=O)c1cc(C2(C#N)CC2)cnc1-c1nc2cc(C(F)(F)F)cnc2n1C] | | | |
| rac | | | |
| State of the art | | | |

Table B13 shows that single enantiomer compounds P18-A, P18-B, P10-A, P10-B, P7-A, P7-B, P1 6-A and P16-B (either first/second eluting following a chiral resolution [Table Z], or enantiopure/enantiomerically enriched following a stereoselective synthesis [Table Y]) according to the invention exert predominantly a substantially better insecticidal action on Bemisia tabaci than the compound from the state of the art.

### Example B14: Comparison of the Insecticidal Activity of Single Enantiomer % Compounds P18-A/P18-B P15-A/P15-B, P6-A/P6-B, P9-A/P9-B, P5-A/P5-B and P11-A/P11-B According to the Invention with the Structurally Comparable Sulfone Compounds from the State of the Art

Activity of compounds P18-A, P18-B, P15-A, P15-B, P6-A, P6-B, P9-A, P9-B, P5-A, P5-B, P11-A and P11-B according to the preparatory examples and of compound P15 from WO16/026848, compound P2 from WO21/219810, compound 6.009 from WO16/096584, compound P3 from WO21/219810, compound P4 from WO21/219810, respectively compound P13 from WO18/206348 against Myzus persicae (systemic, Example BS5 is summarized in Table B14.

TABLE B14

| | Concentration | | Mortality |
|---|---|---|---|
| Compound | (ppm) | Insect | (%) |
| Compound P18-A | 1.5 | Myzus persicae (systemic) | 100 |
| 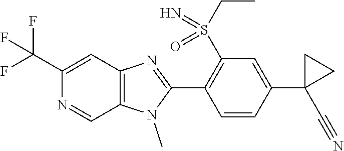 [SMILES: CCS(=N)(=O)c1cc(C2(C#N)CC2)ccc1-c1nc2cc(C(F)(F)F)ncc2n1C] | | | |
| Present invention | | | |
| Compound P18-B | 1.5 | Myzus persicae (systemic) | 80 |
| 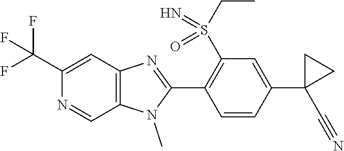 [SMILES: CCS(=N)(=O)c1cc(C2(C#N)CC2)ccc1-c1nc2cc(C(F)(F)F)ncc2n1C] | | | |
| Present invention | | | |
| Described in WO16/026848 as | 1.5 | Myzus persicae (systemic) | 50 |
| compound P15 | | | |
| 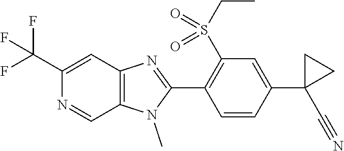 [SMILES: CCS(=O)(=O)c1cc(C2(C#N)CC2)ccc1-c1nc2cc(C(F)(F)F)ncc2n1C] | | | |
| State of the art | | | |
| Compound P15-A | 1.5 | Myzus persicae (systemic) | 80 |
| 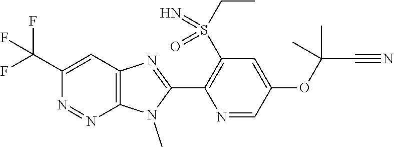 [SMILES: CCS(=N)(=O)c1cc(OC(C)(C)C#N)cnc1-c1nc2cc(C(F)(F)F)nnc2n1C] | | | |
| Present invention | | | |
| Compound P15-B | 1.5 | Myzus persicae (systemic) | 100 |
| 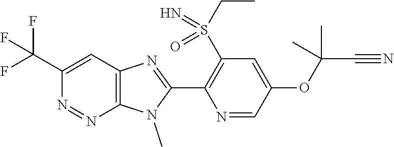 [SMILES: CCS(=N)(=O)c1cc(OC(C)(C)C#N)cnc1-c1nc2cc(C(F)(F)F)nnc2n1C] | | | |
| Present invention | | | |
| Described in WO21/219810 as | 1.5 | Myzus persicae (systemic) | 50 |
| compound P2 | | | |
| 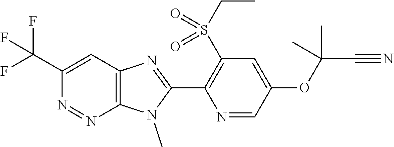 [SMILES: CCS(=O)(=O)c1cc(OC(C)(C)C#N)cnc1-c1nc2cc(C(F)(F)F)nnc2n1C] | | | |
| State of the art | | | |
| Compound P6-A | 6 | Myzus persicae (systemic) | 80 |
| 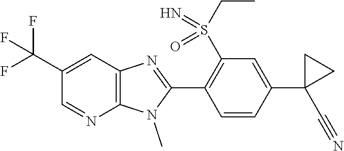 [SMILES: CCS(=N)(=O)c1cc(C2(C#N)CC2)ccc1-c1nc2cc(C(F)(F)F)cnc2n1C] | | | |
| Present invention | | | |
| Compound P6-B | 6 | Myzus persicae (systemic) | 100 |
| 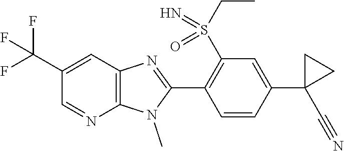 [SMILES: CCS(=N)(=O)c1cc(C2(C#N)CC2)ccc1-c1nc2cc(C(F)(F)F)cnc2n1C] | | | |
| Present invention | | | |
| Described in WO16/096584 as | 6 | Myzus persicae (systemic) | 0 |
| compound 6.009 | | | |
| 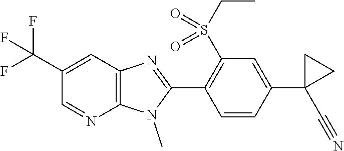 [SMILES: CCS(=O)(=O)c1cc(C2(C#N)CC2)ccc1-c1nc2cc(C(F)(F)F)cnc2n1C] | | | |
| State of the art | | | |
| Compound P9-A | 24 | Myzus persicae (systemic) | 100 |
| 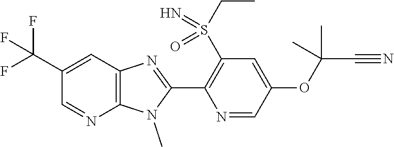 [SMILES: CCS(=N)(=O)c1cc(OC(C)(C)C#N)cnc1-c1nc2cc(C(F)(F)F)cnc2n1C] | | | |
| Present invention | | | |
| Compound P9-B | 24 | Myzus persicae (systemic) | 100 |
| 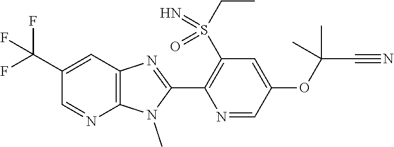 [SMILES: CCS(=N)(=O)c1cc(OC(C)(C)C#N)cnc1-c1nc2cc(C(F)(F)F)cnc2n1C] | | | |
| Present invention | | | |
| Described in WO21/219810 as | 24 | Myzus persicae (systemic) | 50 |
| compound P3 | | | |
| 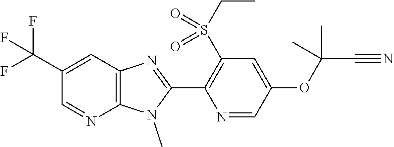 [SMILES: CCS(=O)(=O)c1cc(OC(C)(C)C#N)cnc1-c1nc2cc(C(F)(F)F)cnc2n1C] | | | |
| State of the art | | | |
| Compound P5-A | 24 | Myzus persicae (systemic) | 100 |
| 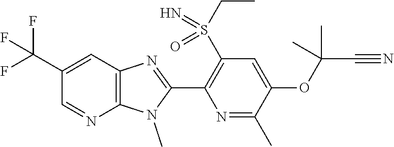 [SMILES: CCS(=N)(=O)c1cc(OC(C)(C)C#N)c(C)nc1-c1nc2cc(C(F)(F)F)cnc2n1C] | | | |
| Present invention | | | |
| Compound P5-B | 24 | Myzus persicae (systemic) | 100 |
| 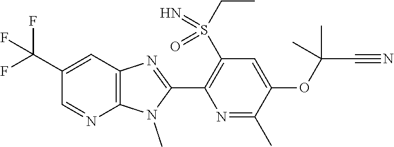 [SMILES: CCS(=N)(=O)c1cc(OC(C)(C)C#N)c(C)nc1-c1nc2cc(C(F)(F)F)cnc2n1C] | | | |
| Present invention | | | |
| Described in WO21/219810 as | 24 | Myzus persicae (systemic) | 0 |
| compound P4 | | | |
| 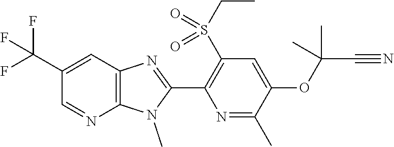 [SMILES: CCS(=O)(=O)c1cc(OC(C)(C)C#N)c(C)nc1-c1nc2cc(C(F)(F)F)cnc2n1C] | | | |
| State of the art | | | |
| Compound P11-A | 24 | Myzus persicae (systemic) | 100 |
| 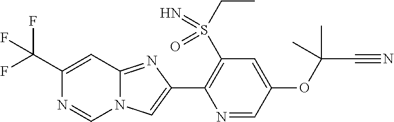 [SMILES: CCS(=N)(=O)c1cc(OC(C)(C)C#N)cnc1-c1cn2cnc(C(F)(F)F)cc2n1] | | | |
| Present invention | | | |
| Compound P11-B | 24 | Myzus persicae (systemic) | 100 |
| 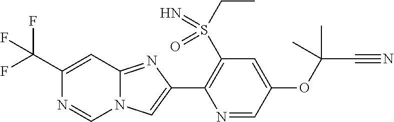 [SMILES: CCS(=N)(=O)c1cc(OC(C)(C)C#N)cnc1-c1cn2cnc(C(F)(F)F)cc2n1] | | | |
| Present invention | | | |
| Described in WO18/206348 as | 24 | Myzus persicae (systemic) | 0 |
| compound P13 | | | |
| 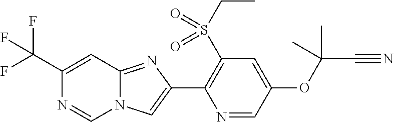 [SMILES: CCS(=O)(=O)c1cc(OC(C)(C)C#N)cnc1-c1cn2cnc(C(F)(F)F)cc2n1] | | | |
| State of the art | | | |

Table B14 shows that single enantiomer compounds P18-A, P18-B, P15-A, P15-B, P6-A, P6-B, P9-A, P9-B, P5-A, P5-B, P11-A and P11-B (either first/second eluting following a chiral resolution [Table Z], or enantiopure/enantiomerically enriched following a stereoselective synthesis [Table Y]) according to the invention exert predominantly a substantially better insecticidal action on Myzus persicae (systemic activity) than the compound from the state of the art.

### Example B15: Comparison of the Insecticidal Activity of Single Enantiomer Compounds P2-A/P2-B, P16-A/P16-B, P13-A/P13-B, P1-A/P1-B and P10-A/P10-B According to the Invention with the Structurally Comparable Sulfone Compounds from the State of the Art

Activity of compound P2-A, P2-B, P16-A, P16-B, P13-A, P13-B, P1-A, P1-B, P1 0-A and P1 0-B according to the preparatory examples and of compound H-1 from WO18/108726, compound P9 from WO16/096584 or compound 11-13 from EP3252046B31, compound P18 from WO18/197315, compound P7 from WO21/21981 0, respectively compound P16 from WO16/026848 or compound 12-13 from EP3252046B31 against Myzus persicae (feeding/contact, Example B34) is summarized in Table B15.

TABLE B15

| | Concentration | | Mortality |
|---|---|---|---|
| Compound | (ppm) | Insect | (%) |
| Compound P2-A | 3.125 | Myzus persicae | 100 |
| | | (feeding/contact) | |
| 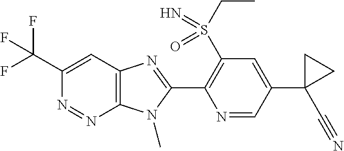 [SMILES: CCS(=N)(=O)c1cc(C2(C#N)CC2)cnc1-c1nc2cc(C(F)(F)F)nnc2n1C] | | | |
| Present invention | | | |
| Compound P2-B | 3.125 | Myzus persicae | 50 |
| | | (feeding/contact) | |
| 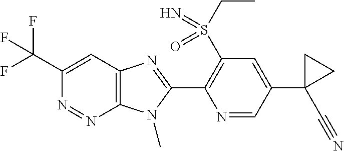 [SMILES: CCS(=N)(=O)c1cc(C2(C#N)CC2)cnc1-c1nc2cc(C(F)(F)F)nnc2n1C] | | | |
| Present invention | | | |
| Described in WO18/108726 as | 3.125 | Myzus persicae | 80 |
| compound H-1 | | (contact) | |
| 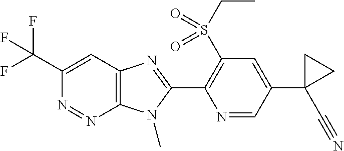 [SMILES: CCS(=O)(=O)c1cc(C2(C#N)CC2)cnc1-c1nc2cc(C(F)(F)F)nnc2n1C] | | | |
| State of the art | | | |
| Compound P16-A | 3.125 | Myzus persicae | 100 |
| | | (feeding/contact) | |
| 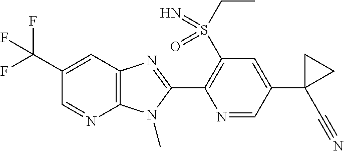 [SMILES: CCS(=N)(=O)c1cc(C2(C#N)CC2)cnc1-c1nc2cc(C(F)(F)F)cnc2n1C] | | | |
| Present invention | | | |
| Compound P16-B | 3.125 | Myzus persicae | 100 |
| | | (feeding/contact) | |
| 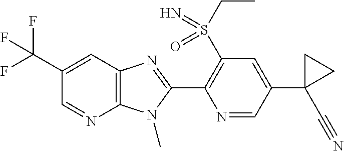 [SMILES: CCS(=N)(=O)c1cc(C2(C#N)CC2)cnc1-c1nc2cc(C(F)(F)F)cnc2n1C] | | | |
| Present invention | | | |
| Described in WO16/096584 as | 3.125 | Myzus persicae | 80 |
| compound P9 or in EP3252046B1 as | | (feeding/contact) | |
| compound 11-13 | | | |
| 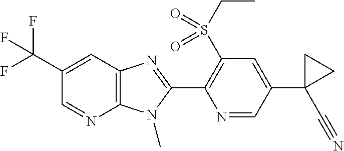 [SMILES: CCS(=O)(=O)c1cc(C2(C#N)CC2)cnc1-c1nc2cc(C(F)(F)F)cnc2n1C] | | | |
| State of the art | | | |
| Compound P13-A | 12.5 | Myzus persicae | 80 |
| | | (feeding/contact) | |
| 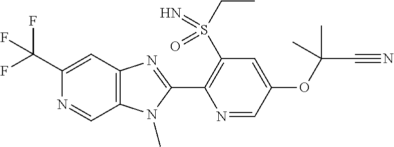 [SMILES: CCS(=N)(=O)c1cc(OC(C)(C)C#N)cnc1-c1nc2cc(C(F)(F)F)ncc2n1C] | | | |
| Present invention | | | |
| Compound P13-B | 12.5 | Myzus persicae | 100 |
| | | (feeding/contact) | |
| 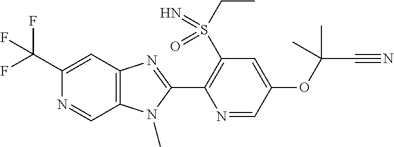 [SMILES: CCS(=N)(=O)c1cc(OC(C)(C)C#N)cnc1-c1nc2cc(C(F)(F)F)ncc2n1C] | | | |
| Present invention | | | |
| Described in WO18/197315 as | 12.5 | Myzus persicae | 65 |
| compound P18 | | (contact) | |
| 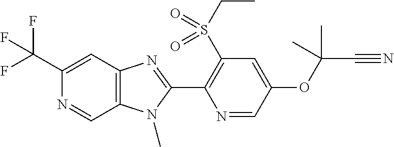 [SMILES: CCS(=O)(=O)c1cc(OC(C)(C)C#N)cnc1-c1nc2cc(C(F)(F)F)ncc2n1C] | | | |
| State of the art | | | |
| Compound P1-A | 50 | Myzus persicae | 100 |
| | | (contact) | |
| 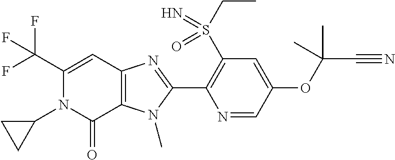 [SMILES: CCS(=N)(=O)c1cc(OC(C)(C)C#N)cnc1-c1nc2cc(C(F)(F)F)n(C3CC3)c(=O)c2n1C] | | | |
| Present invention | | | |
| Compound P1-B | 50 | Myzus persicae | 100 |
| | | (contact) | |
| 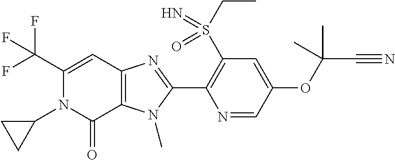 [SMILES: CCS(=N)(=O)c1cc(OC(C)(C)C#N)cnc1-c1nc2cc(C(F)(F)F)n(C3CC3)c(=O)c2n1C] | | | |
| Present invention | | | |
| Described in WO21/219810 as | 50 | Myzus persicae | 0 |
| compound P7 | | (contact) | |
| 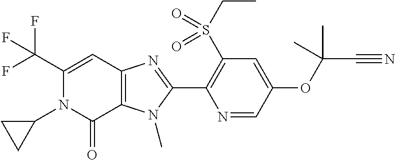 [SMILES: CCS(=O)(=O)c1cc(OC(C)(C)C#N)cnc1-c1nc2cc(C(F)(F)F)n(C3CC3)c(=O)c2n1C] | | | |
| State of the art | | | |
| Compound P10-A | 0.781 | Myzus persicae | 80 |
| | | (feeding/contact) | |
| 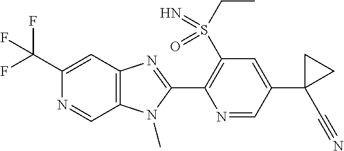 [SMILES: CCS(=N)(=O)c1cc(C2(C#N)CC2)cnc1-c1nc2cc(C(F)(F)F)ncc2n1C] | | | |
| Present invention | | | |
| Compound P10-B | 0.781 | Myzus persicae | 80 |
| | | (feeding/contact) | |
| 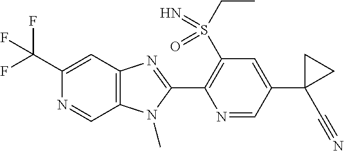 [SMILES: CCS(=N)(=O)c1cc(C2(C#N)CC2)cnc1-c1nc2cc(C(F)(F)F)ncc2n1C] | | | |
| Present invention | | | |
| Described in WO16/026848 as | 0.781 | Myzus persicae | 0 |
| compound P16 or in EP3252046B1 as | | (feeding/contact) | |
| compound 12-13 | | | |
| 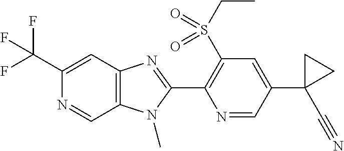 [SMILES: CCS(=O)(=O)c1cc(C2(C#N)CC2)cnc1-c1nc2cc(C(F)(F)F)ncc2n1C] | | | |
| State of the art | | | |

Table B15 shows that single enantiomer compounds P2-A, P2-B, P16-A, P16-B, P13-A, P13-B, P1-A, P1-B, P10-A and P10-B (either first/second eluting following a chiral resolution [Table Z], or enantiopure/enantiomerically enriched following a stereoselective synthesis [Table Y]) according to the invention exert predominantly a substantially better insecticidal action on Myzus persicae (feeding/contact activity) than the compound from the state of the art.

1. A compound of formula (I) 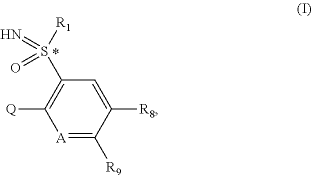
wherein
A is CH or N;
R₁ is C₁-C₄alkyl;
S* is a stereogenic sulfur atom which is in R- or S-configuration;
R₈ is cyanoisopropoxy, cyanoisopropyl or cyanocyclopropyl;
R₉ is hydrogen or C₁-C₄alkyl;
Q is a radical selected from the group consisting of formula Q₁ to Q₅ 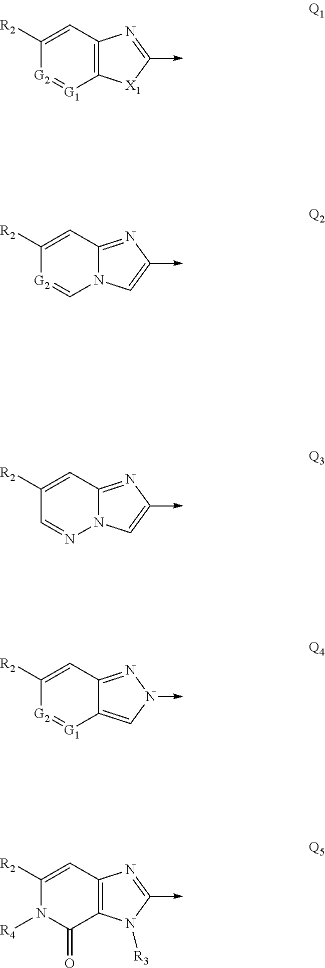
wherein the arrow denotes the point of attachment to the ring incorporating the radical A;
and wherein
X₁ is O, S or NR₃;
R₃ is C₁-C₄alkyl;
R₂ is halogen, C₁-C₆haloalkyl, C₁-C₄haloalkylsulfanyl, C₁-C₄haloalkylsulfinyl, C₁-C₄haloalkylsulfonyl or C₁-C₆haloalkoxy;
G₁ and G₂ are, independently from each other, N or CH;
R₄ is C₁-C₄alkyl, C₁-C₄haloalkyl, C₃-C₆cycloalkyl or C₁-C₄alkoxy; or
an agrochemically acceptable salt, stereoisomer, enantiomer, tautomer or N-oxide of a compound of formula I.

2. A compound of formula I according to claim 1, represented by the compounds of formula I-1 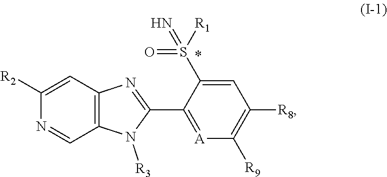
wherein R₁, R₂, R₃, R₈, R₉, S* and A are as defined under formula I in claim 1.

3. A compound of formula I according to claim 1, represented by the compounds of formula I-2 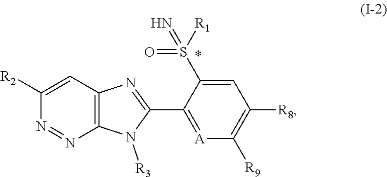
wherein R₁, R₂, R₃, R₈, R₉, S* and A are as defined under formula I in claim 1.

4. A compound of formula I according to claim 1, represented by the compounds of formula I-3 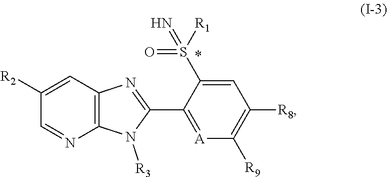
wherein R₁, R₂, R₃, R₈, R₉, S* and A are as defined under formula I in claim 1.

5. A compound of formula I according to claim 1, represented by the compounds of formula I-4 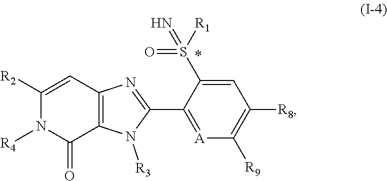
wherein R₁, R₂, R₃, R₄, R₈, R₉, S* and A are as defined under formula I in claim 1.

6. A compound of formula I according to claim 1, represented by the compounds of formula I-5 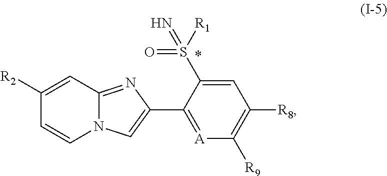
wherein R₁, R₂, R₈, R₉, S* and A are as defined under formula I in claim 1.

7. A compound of formula I according to claim 1, represented by the compounds of formula I-6 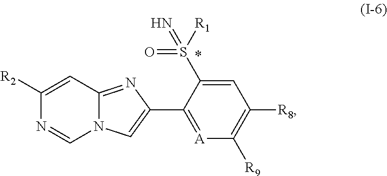
wherein R₁, R₂, R₈, R₉, S* and A are as defined under formula I in claim 1.

8. A compound of formula I according to claim 1, represented by the compounds of formula I-7 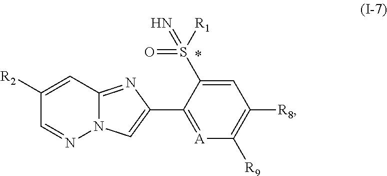
wherein R₁, R₂, R₈, R₉, S* and A are as defined under formula I in claim 1.

9. A compound of formula I according to claim 1, represented by the compounds of formula I-8 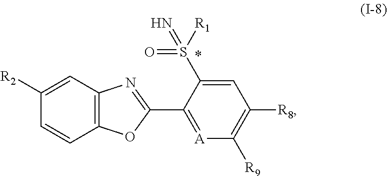
wherein R₁, R₂, R₈, R₉, S* and A are as defined under formula I in claim 1.

10. A compound of formula I according to claim 1, represented by the compounds of formula I-9 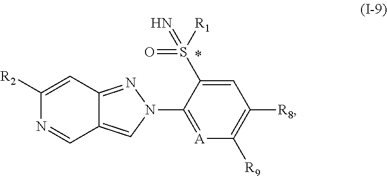
wherein R₁, R₂, R₈, R₉, S* and A are as defined under formula I in claim 1.

11. A compound of formula I according to claim 1, represented by the compounds of formula I-10 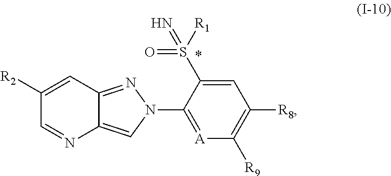
wherein R₁, R₂, R₈, R₉, S* and A are as defined under formula I in claim 1.

12. A compound of formula I according to claim 1, wherein
A is CH or N, preferably A is N;
S* is a stereogenic sulfur atom which is in R- or S-configuration;
R₁ is ethyl, propyl or isopropyl; preferably R₁ is ethyl;
R₂ is trifluoromethyl, pentafluoroethyl or trifluoromethylsulfanyl; preferably R₂ is trifluoromethyl;
R₈ is 1-cyano-1-methyl-ethoxy, 1-cyano-1-methyl-ethyl or 1-cyanocyclopropyl;
R₉ is hydrogen or methyl; preferably R₉ is hydrogen; and
in the case of compounds of formula I wherein Q is Q₁ or Q₄, G₁ is N and G₂ is CH or G₁ is CH and G₂ is N or both G₁ and G₂ are N; and in the case of the compounds wherein Q is Q₂, G₂ is N or CH; and
in the case of the compounds of formula I-1, I-2, I-3, and I-4 R₃ is methyl; and in the case of the compounds of formula I-4 R₄ is ethyl, methoxy or cyclopropyl.

13. A compound of formula I according to claim 1 wherein S* is in the R-configuration in either enantiomerically pure or in enantiomerically enriched form.

14. A compound of formula I according to claim 1 wherein S* is in the S-configuration in either enantiomerically pure or in enantiomerically enriched form.

15. A compound of formula I according to claim 1 selected from the group consisting of:
(S)-2-[[6-[5-cyclopropyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile;
(R)-2-[[6-[5-cyclopropyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile;
(S)-1-[5-(ethylsulfonimidoyl)-6-[7-methyl-3-(trifluoromethyl)imidazo[4,5-c]pyridazin-6-yl]-3-pyridyl]cyclopropanecarbonitrile;
(R)-1-[5-(ethylsulfonimidoyl)-6-[7-methyl-3-(trifluoromethyl)imidazo[4,5-c]pyridazin-6-yl]-3-pyridyl]cyclopropanecarbonitrile;
(S)-2-[[5-(ethylsulfonimidoyl)-6-[7-(trifluoromethyl)imidazo[1,2-a]pyridin-2-yl]-3-pyridyl]oxy]-2-methyl-propanenitrile;
(R)-2-[[5-(ethylsulfonimidoyl)-6-[7-(trifluoromethyl)imidazo[1,2-a]pyridin-2-yl]-3-pyridyl]oxy]-2-methyl-propanenitrile;
(S)-2-[5-(ethylsulfonimidoyl)-6-[3-methyl-6-(trifluoromethyl)imidazo[4,5-c]pyridin-2-yl]-3-pyridyl]-2-methyl-propanenitrile;
(R)-2-[5-(ethylsulfonimidoyl)-6-[3-methyl-6-(trifluoromethyl)imidazo[4,5-c]pyridin-2-yl]-3-pyridyl]-2-methyl-propanenitrile;
(S)-2-[[5-(ethylsulfonimidoyl)-2-methyl-6-[3-methyl-6-(trifluoromethyl)imidazo[4,5-b]pyridin-2-yl]-3-pyridyl]oxy]-2-methyl-propanenitrile;
(R)-2-[[5-(ethylsulfonimidoyl)-2-methyl-6-[3-methyl-6-(trifluoromethyl)imidazo[4,5-b]pyridin-2-yl]-3-pyridyl]oxy]-2-methyl-propanenitrile;
(S)-1-[3-(ethylsulfonimidoyl)-4-[3-methyl-6-(trifluoromethyl)imidazo[4,5-b]pyridin-2-yl]phenyl]cyclopropanecarbonitrile;
(R)-1-[3-(ethylsulfonimidoyl)-4-[3-methyl-6-(trifluoromethyl)imidazo[4,5-b]pyridin-2-yl]phenyl]cyclopropanecarbonitrile;
(S)-2-[[5-(ethylsulfonimidoyl)-6-[7-(trifluoromethylsulfanyl)imidazo[1,2-c]pyrimidin-2-yl]-3-pyridyl]oxy]-2-methyl-propanenitrile;
(R)-2-[[5-(ethylsulfonimidoyl)-6-[7-(trifluoromethylsulfanyl)imidazo[1,2-c]pyrimidin-2-yl]-3-pyridyl]oxy]-2-methyl-propanenitrile;
(S)-1-[5-(ethylsulfonimidoyl)-6-[5-methoxy-3-methyl-4-oxo-6-(trifluoromethyl)imidazo[4,5-c]pyridin-2-yl]-3-pyridyl]cyclopropanecarbonitrile;
(R)-1-[5-(ethylsulfonimidoyl)-6-[5-methoxy-3-methyl-4-oxo-6-(trifluoromethyl)imidazo[4,5-c]pyridin-2-yl]-3-pyridyl]cyclopropanecarbonitrile;
(S)-2-[[5-(ethylsulfonimidoyl)-6-[3-methyl-6-(trifluoromethyl)imidazo[4,5-b]pyridin-2-yl]-3-pyridyl]oxy]-2-methyl-propanenitrile;
(R)-2-[[5-(ethylsulfonimidoyl)-6-[3-methyl-6-(trifluoromethyl)imidazo[4,5-b]pyridin-2-yl]-3-pyridyl]oxy]-2-methyl-propanenitrile;
(S)-1-[5-(ethylsulfonimidoyl)-6-[3-methyl-6-(trifluoromethyl)imidazo[4,5-c]pyridin-2-yl]-3-pyridyl]cyclopropanecarbonitrile;
(R)-1-[5-(ethylsulfonimidoyl)-6-[3-methyl-6-(trifluoromethyl)imidazo[4,5-c]pyridin-2-yl]-3-pyridyl]cyclopropanecarbonitrile;
(S)-2-[[5-(ethylsulfonimidoyl)-6-[7-(trifluoromethyl)imidazo[1,2-c]pyrimidin-2-yl]-3-pyridyl]oxy]-2-methyl-propanenitrile;
(R)-2-[[5-(ethylsulfonimidoyl)-6-[7-(trifluoromethyl)imidazo[1,2-c]pyrimidin-2-yl]-3-pyridyl]oxy]-2-methyl-propanenitrile;
(S)-2-[5-(ethylsulfonimidoyl)-6-[3-methyl-6-(trifluoromethyl)imidazo[4,5-b]pyridin-2-yl]-3-pyridyl]-2-methyl-propanenitrile;
(R)-2-[5-(ethylsulfonimidoyl)-6-[3-methyl-6-(trifluoromethyl)imidazo[4,5-b]pyridin-2-yl]-3-pyridyl]-2-methyl-propanenitrile;
(S)-2-[[5-(ethylsulfonimidoyl)-6-[3-methyl-6-(trifluoromethyl)imidazo[4,5-c]pyridin-2-yl]-3-pyridyl]oxy]-2-methyl-propanenitrile;
(R)-2-[[5-(ethylsulfonimidoyl)-6-[3-methyl-6-(trifluoromethyl)imidazo[4,5-c]pyridin-2-yl]-3-pyridyl]oxy]-2-methyl-propanenitrile;
(S)-1-[6-[5-ethyl-3-methyl-4-oxo-6-(trifluoromethyl)imidazo[4,5-c]pyridin-2-yl]-5-(ethylsulfonimidoyl)-3-pyridyl]cyclopropanecarbonitrile;
(R)-1-[6-[5-ethyl-3-methyl-4-oxo-6-(trifluoromethyl)imidazo[4,5-c]pyridin-2-yl]-5-(ethylsulfonimidoyl)-3-pyridyl]cyclopropanecarbonitrile;
(S)-2-[[5-(ethylsulfonimidoyl)-6-[7-methyl-3-(trifluoromethyl)imidazo[4,5-c]pyridazin-6-yl]-3-pyridyl]oxy]-2-methyl-propanenitrile;
(R)-2-[[5-(ethylsulfonimidoyl)-6-[7-methyl-3-(trifluoromethyl)imidazo[4,5-c]pyridazin-6-yl]-3-pyridyl]oxy]-2-methyl-propanenitrile;
(S)-1-[5-(ethylsulfonimidoyl)-6-[3-methyl-6-(trifluoromethyl)imidazo[4,5-b]pyridin-2-yl]-3-pyridyl]cyclopropanecarbonitrile;
(R)-1-[5-(ethylsulfonimidoyl)-6-[3-methyl-6-(trifluoromethyl)imidazo[4,5-b]pyridin-2-yl]-3-pyridyl]cyclopropanecarbonitrile;
(S)-2-[[5-(ethylsulfonimidoyl)-2-methyl-6-[3-methyl-6-(trifluoromethyl)imidazo[4,5-c]pyridin-2-yl]-3-pyridyl]oxy]-2-methyl-propanenitrile;
(R)-2-[[5-(ethylsulfonimidoyl)-2-methyl-6-[3-methyl-6-(trifluoromethyl)imidazo[4,5-c]pyridin-2-yl]-3-pyridyl]oxy]-2-methyl-propanenitrile;
(S)-1-[3-(ethylsulfonimidoyl)-4-[3-methyl-6-(trifluoromethyl)imidazo[4,5-c]pyridin-2-yl]phenyl]cyclopropanecarbonitrile;
(R)-1-[3-(ethylsulfonimidoyl)-4-[3-methyl-6-(trifluoromethyl)imidazo[4,5-c]pyridin-2-yl]phenyl]cyclopropanecarbonitrile;
(S)-2-[[6-[5-ethyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile; and
(R)-2-[[6-[5-ethyl-3-methyl-4-oxo-6-(trifluoromethyl)imidazo[4,5-c]pyridin-2-yl]-5-(ethylsulfonimidoyl)-3-pyridyl]oxy]-2-methyl-propanenitrile.

16. A pesticidal composition, which comprises at least one compound of formula I as defined in claim 1 or, where appropriate, a tautomer thereof, in each case in free form or in agrochemically utilizable salt form, as active ingredient and at least one auxiliary.

17. A method for controlling pests, which comprises applying to a pest, to a locus of a pest, or to a plant susceptible to attack by a pest, a pesticidally effective amount of a compound of formula I as defined in claim 1.

18. A method for the protection of plant propagation material from the attack by pests, which comprises treating the propagation material or the site, where the propagation material is planted, with a composition according to claim 16.

19. A process for the preparation of compound of formula (I) 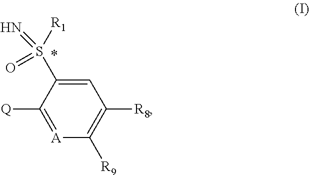
wherein Q, R₁, R₂, G₁, G₂, X₁, R₃, R₄, R₈, R₉ and A are as defined under formula (I) in claim 1, and
wherein S* is a stereogenic sulfur atom in R- or S-configuration, in which said S* center is in either enantiomerically pure or in enantiomerically enriched form;
which process comprises:
(A)stereoselectively oxidizing a sulfanyl compound of formula (II) 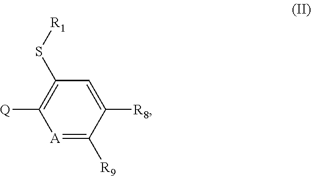
wherein Q, R₁, R₂, G₁, G₂, X₁, R₃, R₄, R₈, R₉ and A are as defined under formula (I), in the presence of an oxidant, in the presence of a metal catalyst, in the presence of a chiral ligand, optionally in the presence of a suitable additive, in an appropriate solvent (or diluent);
to produce a sulfinyl compound of formula (III) 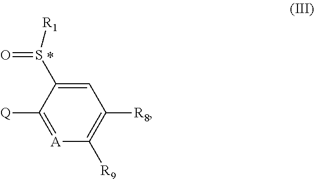
wherein Q, R₁, R₂, G₁, G₂, X₁, R₃, R₄, R₈, R₉ and A are as defined under formula (I), and wherein S* is a stereogenic sulfur atom in R- or S-configuration, in which said S* center is in either enantiomerically pure or in enantiomerically enriched form; and
(B) reacting a sulfinyl compound of formula (III) 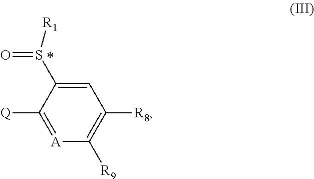
wherein Q, R₁, R₂, G₁, G₂, X₁, R₃, R₄, R₈, R₉ and A are as defined under formula (I), and
wherein S* is a stereogenic sulfur atom in R- or S-configuration, in which said S* center is in either enantiomerically pure or in enantiomerically enriched form;
with an imination reagent, in the presence of a catalyst, optionally in the presence of a suitable additive, in an appropriate solvent (or diluent);
to produce the sulfoximine compound of formula (I) in a stereospecific manner.

20. A compound according to claim 1, whenever prepared or obtainable by a process as claimed in claim 19.
